SEQUENCE LISTING (66 sequences)

SEQ ID NO: 1 - DNA, 25 bp, Artificial Sequence
  Primer
aactggtctcaagtcagtgtacagg

SEQ ID NO: 2 - DNA, 24 bp, Artificial Sequence
  Primer
tcccccaacttgagatgtatgaag

SEQ ID NO: 3 - DNA, 23 bp, Artificial Sequence
  Primer
tttctagagacgcccctaagtca

SEQ ID NO: 4 - DNA, 19 bp, Artificial Sequence
  Primer
ccagctgaggcgctaacag

SEQ ID NO: 5 - DNA, 23 bp, Artificial Sequence
  Primer
cattgatggcaggaactacattg

SEQ ID NO: 6 - DNA, 20 bp, Artificial Sequence
  Primer
ctccagaggctgccacatct

SEQ ID NO: 7 - DNA, 24 bp, Artificial Sequence
  Primer
gccaaattgtgtttgatggattaa

SEQ ID NO: 8 - DNA, 27 bp, Artificial Sequence
  Primer
gacaaaaccgagtcacatcagtaatag

SEQ ID NO: 9 - DNA, 23 bp, Artificial Sequence
  Primer
tggccctgagtactgaactttct

SEQ ID NO: 10 - DNA, 20 bp, Artificial Sequence
  Primer
acccgcaacctgaacatagg

SEQ ID NO: 11 - DNA, 27 bp, Artificial Sequence
  Primer
aaccagggtatttttgttaggttttct

SEQ ID NO: 12 - DNA, 25 bp, Artificial Sequence
  Primer
caaactcatgaaacaacttgaccat

SEQ ID NO: 13 - DNA, 19 bp, Artificial Sequence
  Primer
tccgggcagatttgattga

SEQ ID NO: 14 - DNA, 22 bp, Artificial Sequence
  Primer
cacgttgcgtttcgtagtatcc

SEQ ID NO: 15 - DNA, 18 bp, Artificial Sequence
  Primer
tccggcctctccgacttc

SEQ ID NO: 16 - DNA, 22 bp, Artificial Sequence
  Primer
ctgaggtcacaaggcaggaaag

SEQ ID NO: 17 - DNA, 22 bp, Artificial Sequence
  Primer
gctatagggttggtgggacaga

SEQ ID NO: 18 - DNA, 20 bp, Artificial Sequence
  Primer
actgatacgggaggcagcaa

SEQ ID NO: 19 - DNA, 23 bp, Artificial Sequence
  Primer
gttccgtcagtccaagtcttctc

SEQ ID NO: 20 - DNA, 18 bp, Artificial Sequence
  Primer
tctggccgctcctctgaa

SEQ ID NO: 21 - DNA, 24 bp, Artificial Sequence
  Primer
cagaagatggtgctgggtaataaa

SEQ ID NO: 22 - DNA, 20 bp, Artificial Sequence
  Primer
ttccaacagttggcatttgc

SEQ ID NO: 23 - DNA, 22 bp, Artificial Sequence
  Primer
gcaggctgtcctttataatgca

SEQ ID NO: 24 - DNA, 22 bp, Artificial Sequence
  Primer
tgaaaattcctgttgccattcc

SEQ ID NO: 25 - DNA, 22 bp, Artificial Sequence
  Primer
atgggcttcttgctgtctgtct

SEQ ID NO: 26 - DNA, 24 bp, Artificial Sequence
  Primer
ttgaatgctatctgtgtgggtaca

SEQ ID NO: 27 - DNA, 24 bp, Artificial Sequence
  Primer
aaagttgaggtttccccctaaaat

SEQ ID NO: 28 - DNA, 21 bp, Artificial Sequence
  Primer
tgcaaatgtgtagggctcatg

SEQ ID NO: 29 - DNA, 21 bp, Artificial Sequence
  Primer
atctgcaaacccaccatgtca

SEQ ID NO: 30 - DNA, 20 bp, Artificial Sequence
  Primer
aaagggtgggccatggatag

SEQ ID NO: 31 - DNA, 20 bp, Artificial Sequence
  Primer
ccagaatgcccacaatcaaa

SEQ ID NO: 32 - DNA, 21 bp, Artificial Sequence
  Primer
atctccaggttccatgggaat

SEQ ID NO: 33 - DNA, 24 bp, Artificial Sequence
  Primer
ccattaagtggcagcatcatgtaa

SEQ ID NO: 34 - DNA, 24 bp, Artificial Sequence
  Primer
ccccaattaagtttctgaaaagct

SEQ ID NO: 35 - DNA, 22 bp, Artificial Sequence
  Primer
aagtgtgccagcttcaaaaacc

SEQ ID NO: 36 - DNA, 17 bp, Artificial Sequence
  Primer
cccaggcatcgccctta

SEQ ID NO: 37 - DNA, 27 bp, Artificial Sequence
  Primer
ttcatttaccagctgtattcagaagag

SEQ ID NO: 38 - DNA, 18 bp, Artificial Sequence
  Primer
ccaccctgctgcatgtga

SEQ ID NO: 39 - DNA, 22 bp, Artificial Sequence
  Primer
ggatcacaccaagctcatcttg

SEQ ID NO: 40 - DNA, 18 bp, Artificial Sequence
  Primer
cccgcttctcgtcgatgt

SEQ ID NO: 41 - DNA, 23 bp, Artificial Sequence
  Primer
aaagccagagtgcaagtcatgat

SEQ ID NO: 42 - DNA, 20 bp, Artificial Sequence
  Primer
gaattgtggtgcagccagaa

SEQ ID NO: 43 - DNA, 18 bp, Artificial Sequence
  Primer
ccccctttttggcatcct

SEQ ID NO: 44 - DNA, 20 bp, Artificial Sequence
  Primer
gcaggtgttcccgttgctta

SEQ ID NO: 45 - DNA, 19 bp, Artificial Sequence
  Primer
cggatgcactgctgtgatg

SEQ ID NO: 46 - DNA, 21 bp, Artificial Sequence
  Primer
tgtttccacacggatgattga

SEQ ID NO: 47 - DNA, 22 bp, Artificial Sequence
  Primer
tgagcaagtgcatgactgtgaa

SEQ ID NO: 48 - DNA, 23 bp, Artificial Sequence
  Primer
tcatcatcctgttccaaaaatcc

SEQ ID NO: 49 - DNA, 20 bp, Artificial Sequence
  Primer
cccccgatccaagttgtaga

SEQ ID NO: 50 - DNA, 20 bp, Artificial Sequence
  Primer
gggcttaaggctgccagact

SEQ ID NO: 51 - DNA, 23 bp, Artificial Sequence
  Primer
ccagaaaaccctaaaccacaaga

SEQ ID NO: 52 - DNA, 19 bp, Artificial Sequence
  Primer
gggagcggacgaattctca

SEQ ID NO: 53 - DNA, 19 bp, Artificial Sequence
  Primer
ggagggactcaggcattgg

SEQ ID NO: 54 - DNA, 21 bp, Artificial Sequence
  Primer
agatagcacctcgctgattgc

SEQ ID NO: 55 - DNA, 24 bp, Artificial Sequence
  Primer
aaggatgccaagaagtttaaggaa

SEQ ID NO: 56 - DNA, 22 bp, Artificial Sequence
  Primer
actggcagtttgtccatttgaa

SEQ ID NO: 57 - DNA, 23 bp, Artificial Sequence
  Primer
ttctacccaggtgctaggaacac

SEQ ID NO: 58 - DNA, 22 bp, Artificial Sequence
  Primer
cacagcgtcttgctctctatgg

SEQ ID NO: 59 - DNA, 22 bp, Artificial Sequence
  Primer
catcaacgcactgttgcatatg

SEQ ID NO: 60 - DNA, 20 bp, Artificial Sequence
  Primer
agggccgtccgctaatacac

SEQ ID NO: 61 - DNA, 22 bp, Artificial Sequence
  Primer
gccttcttgctgccaattaaaa

SEQ ID NO: 62 - DNA, 25 bp, Artificial Sequence
  Primer
ctttactggagattccacacacgta

SEQ ID NO: 63 - DNA, 20 bp, Artificial Sequence
  Primer
cgcgctctcgtttcattttc

SEQ ID NO: 64 - DNA, 25 bp, Artificial Sequence
  Primer
tgtccgagtagtagatctgcttgtg

SEQ ID NO: 65 - DNA, 17 bp, Artificial Sequence
  Primer
tcagccccgaggatggt

SEQ ID NO: 66 - DNA, 22 bp, Artificial Sequence
  Primer
tgctgggataggcaaagtcttt

(19) United States
(12) Patent Application Publication
(10) Pub. No.: US 2011/0165566 A1
(43) Pub. Date: Jul. 7, 2011

(54) METHODS OF OPTIMIZING TREATMENT OF BREAST CANCER

(76) Inventors: James L. Wittliff, Louisville, KY (US); Sarah A. Andres, Floyds Knobs, IN (US)

(21) Appl. No.: 12/832,474

(22) Filed: Jul. 8, 2010

Related U.S. Application Data
(60) Provisional application No. 61/224,115, filed on Jul. 9, 2009.

(51) Int. Cl. C12Q 1/68

(52) U.S. Cl. 435/6.11

(57) ABSTRACT

Breast cancer treatment can be optimized by determining the level of expression of genes in a breast sample from a human to identify a human with an increased likelihood of recurrence of the breast cancer that would potentially benefit from a therapy to decrease the likelihood of recurrence of the breast cancer.

### RELATED APPLICATION

This application claims the benefit of U.S. Provisional Application No. 61/224,115, filed on Jul. 9, 2009, the entire teachings of which are incorporated herein by reference.

### BACKGROUND OF THE INVENTION

Breast cancer is a major health concern and one of the most prevalent forms of cancer in woman. Breast cancer has the second highest mortality rate of cancers and about 15% of cancer-related deaths in women are do to breast cancer (SEER Cancer Statistics Review 1975-2005, NCI, Ries, L. A. G., et al., (eds) (2008)). It has been estimated that about 13% of women born in the United States will be diagnosed with breast cancer in their lifetime (SEER Cancer Statistics Review 1975-2005, NCI, Ries, L. A. G., et al., (eds) (2008)). Currently, techniques to diagnosis, in particular, to identify women at an increased likelihood of recurrence of breast cancer, methods of treating breast cancer and methods to monitor progress of treatment regimens for breast cancer include the presence of certain tumor markers in breast tissue biopsies. However, such techniques may be inaccurate in detecting breast cancer and assessing therapy options. Thus, there is a need to develop new, improved and effective methods of identifying a woman having an increased likelihood of recurrence of breast cancer, which may determine a course of therapy selection and prognosis.

### SUMMARY OF THE INVENTION

The present invention related to methods of optimizing treatment of a human having an estrogen-receptor positive breast cancer.

In an embodiment, the invention is a method of optimizing treatment of a human having breast cancer, comprising the step of measuring a level of expression of genes selected from the group consisting of ESR1, GABRP, RABEP1, SLC39A6, TCEAL1, ATAD2, PTP4A2, LRBA and SLC43A3 in a breast cancer tissue sample from the human, wherein underexpression of GABRP, RABEP1, SLC39A6 and PTP4A2 in the sample in combination with overexpression of ESR1, TCEAL1, ATAD2, LRBA and SLC43A3 in the sample identifies a human that has an increased likelihood of recurrence of the breast cancer that would potentially benefit from a therapy to decrease the likelihood of recurrence of the breast cancer.

In another embodiment, the invention is a method of optimizing treatment of a human having breast cancer, comprising the step of measuring a level of expression of genes selected from the group consisting of GABRP, TRIM29, RABEP1, SLC39A6, TCEAL1, PLK1 and CX3CL1 in a breast cancer tissue sample from the human, wherein underexpression of GABRP, TRIM29, RABEP1 and SLC39A6 in the sample in combination with overexpression of TCEAL1, PLK1 and CX3CL1 in the sample identifies a human that has an increased likelihood of recurrence of the breast cancer that would potentially benefit from a therapy to decrease the likelihood of recurrence of the breast cancer.

In a further embodiment, the invention is a method of optimizing treatment of a human having breast cancer, comprising the step of measuring a level of expression of genes selected from the group consisting of TBC109, RABEP1, SLC39A6, FUT8 and PTP4A2 in a breast cancer tissue sample from the human, wherein underexpression of TBC1D9, RABEP1, SLC39A6, FUT8 and PTP4A2 in the sample identifies a human that has an increased likelihood of recurrence of the breast cancer that would potentially benefit from a therapy to decrease the likelihood of recurrence of the breast cancer.

In still another embodiment, the invention is a method of optimizing treatment of a human having breast cancer, comprising the step of measuring a level of expression of genes selected from the group consisting of RABEP1, SLC39A6, FUT8 and PTP4A2 in a breast cancer tissue sample from the human, wherein underexpression of RABEP1, SLC39A6, FUT8 and PTP4A2 in the sample identifies a human that has an increased likelihood of recurrence of the breast cancer that would potentially benefit from a therapy to decrease the likelihood of recurrence of the breast cancer and thereby increase the likelihood of survival of the human.

In an additional embodiment, the invention is a method of optimizing treatment of a human having breast cancer, comprising the step of measuring a level of expression of genes selected from the group consisting of ESR1, GABRP, RABEP1, SLC39A6, TCEAL1, ATAD2, PTP4A2, LRBA and SLC43A3 in a breast cancer tissue sample from the human, wherein overexpression of GABRP, RABEP1, SLC39A6 and PTP4A2 in the sample in combination with underexpression of ESR1, TCEAL1, ATAD2, LRBA and SLC43A3 in the sample identifies a human that has a decreased likelihood of recurrence of the breast cancer that would potentially benefit from a therapy to treat the breast cancer.

In yet another embodiment, the invention is a method of optimizing treatment of a human having breast cancer, comprising the step of measuring a level of expression of genes selected from the group consisting of GABRP, TRIM29, RABEP1, SLC39A6, TCEAL1, PLK1 and CX3CL1 in a breast cancer tissue sample from the human, wherein overexpression of GABRP, TRIM29, RABEP1 and SLC39A6 in the sample in combination with underexpression of TCEAL1, PLK1 and CX3CL1 in the sample identifies a human that has a decreased likelihood of recurrence of the breast cancer that would potentially benefit from a therapy to treat the breast cancer.

An additional embodiment of the invention is a method of optimizing treatment of a human having breast cancer, comprising the step of measuring a level of expression of genes selected from the group consisting of TBC1D9, RABEP1, SLC39A6, FUT8 and PTP4A2 in a breast cancer tissue sample from the human, wherein overexpression of TBC1D9, RABEP1, SLC39A6, FUT8 and PTP4A2 in the sample identifies a human that has a decreased likelihood of recurrence of the breast cancer that would potentially benefit from a therapy to treat the breast cancer.

The methods of the invention can be employed to optimize treatment of breast cancer in a human. Advantages of the claimed invention include, for example, relatively rapid determination of changes in gene expression on small amounts of tissue (e.g., fresh or frozen biopsies) by detecting changes in relatively few genes (e.g., 10, 9, 7, 5 or 4) which can improve the accuracy of identifying humans with an increased risk of recurrence of the breast cancer. The claimed methods can be employed in optimizing treatment of breast cancer, thereby avoiding recurrence of the disease, serious illness consequent the disease and death.

### BRIEF DESCRIPTION OF THE FIGURES

FIG. 1 depicts the protocol for gene expression analyses by qPCR and microarray of frozen tissue sections or of LCM-procured cells.

FIGS. 2A-2C depict representative standard curves of qPCR analyses of ACTB (FIG. 2A), ESR1 (FIG. 2B) and PGR (FIG. 2C) genes measuring relative expression. Dilutions were prepared with cDNA made from Universal Human Reference RNA (Stratagene). Similar amplification efficiencies were illustrated for the three genes: FIG. 2A exhibited a regression line with a slope of −3.48 with an R2=0.99; FIG. 2B shows a regression line with a slope of −3.45 with an R2=0.96; FIG. 2C shows a regression line with a slope of −3.54 with an R2=0.93.

FIGS. 3A-3B depict representative gene expression levels in a single specimen of invasive ductal carcinoma of the breast and demonstrates the reproducibility of results obtained when four serial tissue sections were processed and analyzed concurrently (Mean±SD shown). A comparison of variation between individual tissue sections (FIG. 3A) and results of all qPCR runs for this specimen (FIG. 3B) is depicted.

FIGS. 4A-4B depict representative gene expression levels for a single specimen of invasive ductal carcinoma of the breast and demonstrates the reproducibility of results obtained when three serial tissue sections were processed independently on different days. A comparison of variation between different tissue sections (FIG. 4A) and all qPCR runs for this specimen (FIG. 4B) is depicted.

FIGS. 5A and 5B depict a comparison of gene expression in specific cell types collected by LCM with that of intact tissue sections from a 31-year-old white female with invasive ductal carcinoma (tissue contained 95% carcinoma cells). FIG. 5A shows relative expression of the cancer gene subset in intact tissue compared to that of LCM-procured carcinoma cells. Expression of three of the 14 genes was statistically lower in the intact tissue compared to those in LCM-procured cells. FIG. 5B shows relative expression of the stromal gene subset in intact tissue compared to that of LCM-procured stromal cells. Expression of nine of the 18 genes was statistically higher in the intact tissue compared to those of LCM-procured cells.

FIGS. 6A and 6B depict a comparison of gene expression in specific cell types collected by LCM with that of intact tissue sections from a 44-year-old white female with invasive ductal carcinoma (tissue contained 60% carcinoma cells). FIG. 6A shows relative expression of the cancer gene subset in intact tissue compared to that of LCM-procured carcinoma cells. Expression of four of the 14 genes was statistically different in the intact tissue compared to those in LCM-procured cells. FIG. 6B shows relative expression of the stromal gene subset in intact tissue compared to that of LCM-procured stromal cells. Expression of sixteen of the 18 genes statistically higher in the intact tissue compared to those of LCM-procured cells.

FIGS. 7A and 7B depict a comparison of gene expression in specific cell types collected by LCM with that of intact tissue sections from a 69 year-old white female with invasive ductal carcinoma (tissue contained 30% carcinoma cells). FIG. 7A shows relative expression of the cancer gene subset in intact tissue compared to that of LCM-procured carcinoma cells. Expression of five of the 14 genes was statistically lower in the intact tissue compared to those in LCM-procured cells. FIG. 7B shows relative expression of the stromal gene subset in intact tissue compared to that of LCM-procured stromal cells. Expression of eight of the 18 genes was statistically different in the intact tissue compared to those of LCM-procured cells.

FIGS. 8A-8D depict the influence of the content of a specific cell type in a tissue section on the fold change in gene expression. The distribution of fold changes in expression of representative genes; EVL (FIG. 8A), ST8SIA1 (FIG. 8B), XBP1 (FIG. 8C) and PLK1 (FIG. 8D), in LCM-procured cells (FIGS. 8A and 8B—cancer, FIGS. 8C and 8D—stroma) compared to intact tissue are plotted as a function of cell content. A comparison of EVL expression (FIG. 8A) in tissues containing either 0-60% carcinoma cells (n=17) or greater than 60% carcinoma cells (n=14) revealed no difference. However, the same comparison of ST8SIA1 (FIG. 8B) indicated that expression levels measured by qPCR were related to cancer cell content (P value=0.03). When the same analyses were performed for XBP (FIG. 8C) in tissues containing 0-20% stromal cells (n=14) or greater than 20% stromal cells (n 8), no difference was observed. However, analysis of PLK1 (FIG. 8D) in the two sample types indicated a statistically significant difference related to cell content (P value=0.04).

FIGS. 9A-9B depict a comparison of gene expression in specific cell types collected by LCM and those of intact tissue sections. Representative comparison of relative expression of entire 32 gene set in a 31-year-old patient with invasive ductal carcinoma (same patient as shown in FIG. 5) examining intact tissue, LCM-procured carcinoma cells, and LCM-procured stromal cells. The tissue utilized for this analysis contained 95% carcinoma cells and 5% stromal cells.

FIGS. 10A-10B depict a comparison of gene expression in specific cell types collected by LCM and those of intact tissue sections. Representative comparison of relative expression of entire 32 gene set in a 44-year-old patient with invasive ductal carcinoma (same patient as shown in FIG. 6) examining intact tissue, LCM-procured carcinoma cells, and LCM-procured stromal cells. The tissue utilized for this analysis contained 60% carcinoma cells and 30% stromal cells.

FIGS. 11A-11B depict a comparison of gene expression in specific cell types collected by LCM and those of intact tissue sections. Representative comparison of relative expression of entire 32 gene set in a 69-year-old patient with invasive ductal carcinoma (same patient as shown in FIG. 7) examining intact tissue, LCM-procured carcinoma cells, and LCM-procured stromal cells. The tissue utilized for this analysis contained 30% carcinoma cells and 30% stromal cells.

FIGS. 12A-12F depict a comparison of expression of six of the genes in the 32 gene set using microarray and qPCR. Representative correlations between gene expression analyzed by qPCR of 86 intact tissue sections and microarray results of LCM-procured carcinoma cells are depicted. FIGS. 12A-12F depict the relative expression of six genes with the best correlation between analysis platforms. (FIG. 12A: NAT1, FIG. 12B: SCUBE2, FIG. 12C: ESR1, FIG. 12D: GABRP, FIG. 12E: XBP1, F: EVL)

FIG. 13 is a bar graph depicting the probabilities of survival based on various characteristics of breast cancer patients and their carcinomas. Characteristics analyzed include race, menopausal status, lymph node status, stage, and tumor grade.

FIGS. 14A-F are Kaplan-Meier plots showing disease-free survival (FIGS. 14A, 14C and 14E) and overall survival (FIGS. 14B, 14D and 14F) of patients with known prognostic factors for breast cancer

FIGS. 15A-15D are Kaplan-Meier plots showing disease-free survival (FIGS. 15A and 15C) and overall survival (FIGS. 15B and 15D) of patients as a function of tumor marker levels currently used in breast cancer assessment. Survival plots (FIGS. 15A and 1513) depict the correlation of estrogen receptor protein status and patient survival. FIGS. 15C and 15D depict the correlation of progestin receptor protein status and survival.

FIGS. 16A-16O depict the expression levels and distribution of 15 genes from the 32 gene set analyzed using intact tissue sections of 126 invasive ductal carcinomas. Results show 13 genes with expression levels indicative of non-Gaussian distribution as determined by the D'Agostino-Pearson normality test, which include NAT1 (FIG. 16A), ESR1 (FIG. 16B), GABRP (FIG. 16C), IL6ST (FIG. 16D), CENPA (FIG. 16E), ATAD2 (FIG. 16F), XBP1 (FIG. 16G), MCM6 (FIG. 16H), PTP4A2 (FIG. 16I), LRBA (FIG. 16J), GATA3 (FIG. 16K), GMPS (FIG. 16L) and SLC43A3 (FIG. 16M). Expression of genes (FIG. 16N) and (FIG. 16O) are representative of those exhibiting Gaussian distribution. The horizontal line within distribution pattern indicates median expression level.

FIGS. 17A-17D depict representative correlations of expression of gene pairs determined to be significant from Pearson correlations. Comparisons of ESR1 and NAT1 (FIG. 17A), as well as of SLC39A6 and RABEP1 (FIG. 17B) depict positive correlations of gene expression, while those comparing XBP1 and GABRP (FIG. 17C) and ST8SIA1 and XBP1 (FIG. 17D) depict negative correlations of expression levels.

FIGS. 18A-18D depict the relationship of gene expression and protein expression levels of known breast cancer biomarkers, estrogen receptor (FIGS. 18A and 18C) and progestin receptor (FIGS. 18B and 18D) in 132 patient specimens. Results depicted gave linear regressions with correlation coefficients of 0.70 for ER (FIG. 18A) and 0.38 for PR (FIG. 18B). Since 22 of the specimens shown in FIGS. 18A and 23 of the specimens shown in FIG. 18B had undetectable levels of tumor marker protein in clinical assays, these values were excluded from plots shown in FIG. 18C and FIG. 18D. The relationship between mRNA and protein levels gave higher correlation coefficients of 0.73 for ER (FIG. 18C) and 0.48 for ER (FIG. 18D).

FIGS. 19A-19O depict gene expression differences in tissue biopsies from pre-menopausal and post-menopausal breast cancer patients. Box and whisker plots of expression levels from pre-menopausal (n=30) and post-menopausal (n=51) breast cancer patients are shown. The box represents gene expression levels within the second and third quartiles of values observed. The horizontal line within the box represents the median expression level, while the whiskers extend to the lowest and highest expression level for each gene. Genes shown are those with differences determined significant in t-tests: EVL (FIG. 19A), NAT1 (FIG. 19B), ESR1 (FIG. 19C), GABRP (FIG. 19D), TBC1D9 (FIG. 19E), TRIM29 (FIG. 19F), SCUBE2 (FIG. 19G), RABEP1 (FIG. 19D), SLC39A6 (FIG. 19I), TCEAL1 (FIG. 19J), MELK (FIG. 19K), ATAD2 (FIG. 19L), XBP1 (FIG. 19M), LRBA (FIG. 19N) and GATA3 (FIG. 19O).

FIGS. 20A-20C depict gene expression differences in cancer patients who were tobacco smokers and non-smokers. Box and whisker plots of gene expression levels determined in non-smoking (n=54) and smoking (n=27) breast cancer patients are shown. The box represents gene expression levels within the second and third quartiles of values observed. The horizontal line within the box represents the median expression level, while the whiskers extend to the lowest and highest expression level for each gene. Genes shown are those whose with differences determined significant in t-tests: PFKP (FIG. 20A), YBX1 (FIG. 20B) and SLC43A3 (FIG. 20C).

FIGS. 21A-21O depict gene expression differences in patients with tumors of differing grade. Box and whisker plots of gene expression levels in grade 1 (n=7), grade 2 (n=35), and grades 3 and 4 (n=58) tumors are shown. The box represents gene expression levels within the second and third quartiles of values observed. The horizontal line within the box represents the median expression level, while the whiskers extend to the lowest and highest expression level for each gene. Genes shown are those with differences determined significant in ANOVA: EVL (FIG. 21A), NAT1 (FIG. 21B), ESR1 (FIG. 21C), ST8SIA1 (FIG. 21D), TBC1D9 (FIG. 21E), SCUBE2 (FIG. 21F), RABEP1 (FIG. 21G), SLC39A6 (FIG. 21H), TPBG (FIG. 21I), TCEAL1 (FIG. 21J), CENPA (FIG. 21K), MELK (FIG. 21L), XBP1 (FIG. 21M), BUB1 (FIG. 21N) and GATA3 (FIG. 21O).

FIGS. 22A and 22B depict gene expression differences in cancer patients who were lymph node negative or positive. Box and whisker plots of gene expression levels in node negative (n=62) and node positive (n=57) breast cancer patients are shown. The box represents gene expression levels within the second and third quartiles of values observed. The horizontal line within the box indicates the median expression level, and the whiskers extend to the lowest and highest expression level for each gene. Genes shown are those with differences determined significant in t-tests: GABRP (FIG. 22A) and CENPA (FIG. 22B).

FIGS. 23A-23Y depict gene expression differences in cancer patients whose biopsies were estrogen receptor negative or positive. Box and whisker plots of gene expression levels in ER negative (n=47) and ER positive (n=79) breast cancers are shown. The box represents gene expression levels within the second and third quartiles of values observed. The horizontal line within the box represents the median expression level, and the whiskers extend to the lowest and highest expression level for each gene. Genes shown are those with differences determined significant in t-tests: EVL (FIG. 23A), NAT1 (FIG. 23B), ESR1 (FIG. 23C), GABRP (FIG. 23D), ST8SIA1 (FIG. 23E), TBC1D9 (FIG. 23F), TRIM29 (FIG. 23G), SCUBE2 (FIG. 23H), IL6ST (FIG. 23I), RABEP1 (FIG. 23J), SLC39A6 (FIG. 23K), TPBG (FIG. 23L), TCEAL1 (FIG. 23M), DSC2 (FIG. 23N), FUT8 (FIG. 23O), CENPA (FIG. 23P), MELK (FIG. 23Q), PFKP (FIG. 23R), XBP1 (FIG. 23S), PTP4A2 (FIG. 23T), YBX1 (FIG. 23U), LRBA (FIG. 23V), GATA3 (FIG. 23W), CX3CL1 (FIG. 23X) and SLC43A3 (FIG. 23Y).

FIGS. 24A-24U depict gene expression differences in patients whose breast cancer biopsies were progestin receptor negative or positive. Box and whisker plots of gene expression levels in PR negative (n=43) and PR positive (n=83) breast cancer patients are shown. The box represents gene expression levels within the second and third quartiles of values observed. The horizontal line within the box represents the median expression level, and the whiskers extend to the lowest and highest expression level for each gene. Genes shown are those with differences determined significant in t-tests: EVL (FIG. 24A), NAT1 (FIG. 24B), ESR1 (FIG. 24C), GABRP (FIG. 24D), ST8SIA1 (FIG. 24E), TBC1D9 (FIG. 24F), SCUBE2 (FIG. 24G), IL6ST (FIG. 24H), RABEP1 (FIG. 24I), SLC39A6 (FIG. 24J), TPBG (FIG. 24K), TCEAL1 (FIG. 24L), FUT8 (FIG. 24M), MELK (FIG. 24N), PFKP (FIG. 24O), XBP1 (FIG. 24P), PTP4A2 (FIG. 24Q), LRBA (FIG. 24R), GATA3 (FIG. 24S), CX3CL1 (FIG. 24T) and SLC43A3 (FIG. 24U).

FIGS. 25A-25H are representative Kaplan-Meier plots of patients exhibiting differences in disease-free and overall survival as a function of expression of a single gene in the carcinoma biopsy. Genes shown include GABRP (FIGS. 25A and 25B), SCUBE2 (FIGS. 25C and 25D), SLC39A6 (FIGS. 25E and 25F) and MELK (FIGS. 25G and 25H). Gene expression in the breast tissue biopsy was related to disease-free (FIGS. 25A, 25C, 25E and 25G) and overall survival (FIGS. 25B, 25D, 25F and 25H) of 126 cancer patients with the levels of statistical significance listed in Table 34.

FIGS. 26A-26F are representative Kaplan-Meier plots of disease-free and overall survival of breast cancer patients evaluating GABRP gene expression as a function of lymph node involvement. The relationship of GABRP expression is shown for all patients (FIGS. 26A and 26B), those that are node negative (FIGS. 26C and 26D), and those that are node positive (FIGS. 26E and 26F). Kaplan-Meier plots of the patients' disease-free (FIGS. 26A, 26C and 26E) and overall survival (FIGS. 26B, 26D and 26F) are shown.

FIGS. 27A-27F are representative Kaplan-Meier plots of breast cancer patients evaluating NAT1 gene expression as a function of tumor grade for disease-free and overall survival. The relationship of NAT1 expression is shown for all patients (FIGS. 27A and 27B), patients with grade 1 or 2 tumors (FIGS. 27C and 27D), and patients with grade 3 or 4 tumors (FIGS. 27E and 27F). Kaplan-Meier plots of the patients' disease-free (FIGS. 27A, 27C and 27E) and overall survival (FIGS. 27B, 27D and 27F) are shown.

FIGS. 28A-28F are representative Kaplan-Meier plots of disease-free and overall survival of breast cancer patients evaluating CENPA gene expression as a function of tumor grade. The relationship of CENPA expression is shown for all patients (FIGS. 28A and 28B), patients with grade 1 or 2 tumors (FIGS. 28C and 28D), and patients with grade 3 or 4 tumors (FIGS. 28E and 28F). Kaplan-Meier plots of the patients' disease-free (FIGS. 28A, 28C and 28E) and overall survival (FIGS. 28B, 28D and 28F) are shown.

FIGS. 29A-29F are representative Kaplan-Meier plots of disease-free and overall survival of breast cancer patients evaluating BUB1 gene expression as a function of tumor grade. The relationship of BUB1 expression was shown in all patients (FIGS. 29A and 29B), patients with grade 1 or 2 tumors (FIGS. 29C and 29D), and patients with grade 3 or 4 tumors (FIGS. 29E and 29F). Kaplan-Meier plots of the patients' disease-free (FIGS. 29A, 29C and 29E) and overall survival (FIGS. 29B, 29D and 29F) are shown.

FIGS. 30A-30F are representative Kaplan-Meier plots of breast cancer patients evaluating ESR1 gene expression as a function of estrogen receptor status for disease-free and overall survival. The relationship of ESR1 expression is shown in all patients (FIGS. 30A and 30B), patients with ER-negative tumors (FIGS. 30C and 30D), and patients with ER-positive tumors (FIGS. 30E and 30F). Kaplan-Meier plots of the patients' disease-free (FIGS. 30A, 30C and 30E) and overall survival (FIGS. 30B, 30D and 30F) are shown.

FIGS. 31A-31F are representative Kaplan-Meier plots of breast cancer patients evaluating SCUBE2 gene expression as a function of estrogen receptor status for disease-free and overall survival. The relationship of SCUBE2 expression is shown in all patients (FIGS. 31A and 31B), patients with ER-negative tumors (FIGS. 31C and 31D), and patients with ER-positive tumors (FIGS. 31E and 31F). Kaplan-Meier plots of the patients' disease-free (FIGS. 31A, 31C and 31E) and overall survival (FIGS. 31B, 31D and 31F) are shown.

FIGS. 32A-32F are representative Kaplan-Meier plots of breast cancer patients evaluating RABEP1 gene expression as a function of estrogen receptor status for disease-free and overall survival. The relationship of RABEP1 expression is shown for all patients (FIGS. 32A and 32B), patients with ER-negative tumors (FIGS. 32C and 32D), and patients with ER-positive tumors (FIGS. 32E and F). Kaplan-Meier plots of the patient's disease-free (FIGS. 32A, 32C and 32E) and overall survival (FIGS. 32B, 32D and 32F) are shown.

FIG. 33A-33F are representative Kaplan-Meier plots of breast cancer patients evaluating SLC39A6 gene expression as a function of estrogen receptor status for disease-free and overall survival. The relationship of SLC39A6 expression is shown for all patients (FIGS. 33A and 33B), patients with ER-negative tumors (FIGS. 33C and 33D), and patients with ER-positive tumors (FIGS. 33E and 33F). Kaplan-Meier plots of the patient's disease-free (FIGS. 33A, 33C and 33E) and overall survival (FIGS. 33B, 33D and 33F) are shown.

FIGS. 34A-34F are representative Kaplan-Meier plots of breast cancer patients evaluating TCEAL1 gene expression as a function of estrogen receptor status for disease-free and overall survival. The relationship of TCEAL1 expression is shown for all patients (FIGS. 34A and 34B), patients with ER-negative tumors (FIGS. 34C and 34D), and patients with ER-positive tumors (FIGS. 34E and 34F). Kaplan-Meier plots of the patient's disease-free (FIGS. 34A, 34C and 34E) and overall survival (FIGS. 34B, 34D and 34F) are shown.

FIGS. 35A-35F are representative Kaplan-Meier plots of breast cancer patients evaluating XBP1 gene expression as a function of estrogen receptor status for disease-free and overall survival. The relationship of XBP1 expression is shown for all patients (FIGS. 35A and 35B), patients with ER-negative tumors (FIGS. 35C and 35D), and patients with ER-positive tumors (FIGS. 35E and 35F). Kaplan-Meier plots of the patient's disease-free (FIGS. 35A, 35C and 35E) and overall survival (FIGS. 35B, 35D and 35F) are shown.

FIGS. 36A-36F are representative Kaplan-Meier plots of breast cancer patients evaluating SLC39A6 gene expression as a function of progestin receptor status for disease-free and overall survival. The relationship of SLC39A6 expression is shown for all patients (FIGS. 36A and 36B), patients with PR-negative tumors (FIGS. 36C and 36D), and patients with PR-positive tumors (FIGS. 36E and 36F). Kaplan-Meier plots of the patient's disease-free (FIGS. 36A, 36C and 36E) and overall survival (FIGS. 36B, 36D and 36F) are shown.

FIGS. 37A-37F are representative Kaplan-Meier plots of breast cancer patients evaluating PTP4A2 gene expression as a function of progestin receptor status for disease-free and overall survival. The relationship of PTP4A2 expression is shown for all patients (FIGS. 37A and 37B), patients with PR-negative tumors (FIGS. 37C and 37D), and patients with PR-positive tumors (FIGS. 37E and 37F). Kaplan-Meier plots of the patient's disease-free (FIGS. 37A, 37C and 37E) and overall survival (FIGS. 37B, 37D and 37F) are shown.

FIGS. 38A-38F are Kaplan-Meier plots illustrating the multivariate model of disease recurrence developed from the 80 patient training set population. FIGS. 38A and 38B represent the patients from the training set population, as stratified by relative risk for disease-free (FIG. 38A) and overall (FIG. 38B) survival. FIGS. 38C and 38D represent the patients from the independent 41 patient test set population, as stratified by relative risk for disease-free (FIG. 38C) and overall (FIG. 38D) survival, calculated from the model. Since the survival curves from the low risk and intermediate risk populations appear similar, the two strata were grouped and compared to the high risk population (FIGS. 38E and 38F).

FIGS. 39A-39F are Kaplan-Meier plots illustrating the multivariate model of survival developed from the 83 patient training set population. FIGS. 39A and 3913 represent the analyses of patients from the training set population, as stratified by relative risk for disease-free (FIG. 39A) and overall (FIG. 39B) survival. FIGS. 39C and 39D represent the analyses of patients from the independent 43 patient test set population, as stratified by relative risk for disease-free (FIG. 39C) and overall (FIG. 39D) survival, calculated from the model. Since the survival curves from the low risk and intermediate risk populations appear similar, the two strata were grouped and compared to the high risk population (FIGS. 39E and 39F).

FIGS. 40A-40D are Kaplan-Meier plots illustrating the multivariate model of disease recurrence developed from the entire 121 patient population with the 9 genes from Table 40. FIGS. 40A and 40B represent patients stratified by relative risk for disease-free (FIG. 40A) and overall (FIG. 40B) survival. Since the survival curves from the low risk and intermediate risk populations appear similar, the two strata were grouped and compared to the high risk population (FIGS. 40C and 40D).

FIGS. 41A-41B are ROC curves compiled to depict the sensitivity and specificity of the 9 gene model of breast cancer recurrence developed using the entire patient population (n=121). FIG. 41A represents the comparison of the relative risk as calculated from the model with actual disease recurrence (DFS), and FIG. 41B represents the comparison of the relative risk as calculated from the model with actual patient survival (OS). The diagonal reference line represents the probability that the predictions were made by chance.

FIGS. 42A-42D are Kaplan-Meier plots illustrating the multivariate model of disease recurrence developed from the entire 126 patient population with the 7 genes from Table 41. FIGS. 42A and 42B represent patients stratified by relative risk for disease-free (FIG. 42A) and overall (FIG. 42B) survival. Since the survival curves from the low risk and intermediate risk populations appear similar, the two strata were grouped and compared to the high risk population (FIGS. 42C and 42D).

FIGS. 43A and 43B are ROC curves compiled to depict the sensitivity and specificity of the model of patient survival developed using the entire patient population (n=126). FIG. 43A represents the comparison of the relative risk as calculated from the model with actual disease recurrence, and FIG. 43B represents the comparison of the relative risk as calculated from the model with actual patient survival. The diagonal reference line represents the probability that the predictions were made by chance.

FIGS. 44A-44D are Kaplan-Meier survival curves of two clinically relevant genes measured by qPCR. These plots depict correlations of disease-free and overall survival of breast cancer patients as a function two genes (X=RABEP1; Y=SLC39A6).

FIGS. 45A-45B depict the probability of breast cancer recurrence and survival based on a model developed using gene combinations from the 32 gene set measured by qPCR. The multivariate model for DFS was created using K-Nearest Neighbor classification with a 61 sample training set, and applied to the 41 sample test set as described herein.

FIGS. 46A-46D depict the correlation of expression results from 4 representative genes (EVL, NAT1, ESR1 and GABRP) obtained by qPCR and ZIPLEX® Automated Workstation illustrating similar gene expression results from both analysis platforms.

FIGS. 47A-47D are Kaplan-Meier survival curves of two clinically relevant genes measured by the ZIPLEX® Automated Workstation. These plots illustrate correlations of disease-free and overall survival of breast cancer patients as a function two genes (S=DSC2; F=BUB1).

FIGS. 48A-48B depict the Probability of breast cancer recurrence and survival based on a model developed using gene combinations from the 32 gene set measured by the ZIPLEX® Automated Workstation. The multivariate model for DFS was created using K-Nearest Neighbor classification with a 65 sample training set, and applied to the 44 sample test described herein.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The methods described herein are generally directed to methods of optimizing treatment of a human with breast cancer. Recurrence of breast cancer in a human can lead to prolonged illness, unknown clinical outcome and mortality. The methods described herein can facilitate critical and careful clinical management of optimal treatment of humans with breast cancer, which decreases the likelihood of recurrence of the breast cancer and death consequent to the breast cancer.

In an embodiment, the invention is a method of optimizing treatment of a human having breast cancer, comprising the step of measuring a level of expression of genes selected from the group consisting of ESR1, GABRP, RABEP1, SLC39A6, TCEAL1, ATAD2, PTP4A2, LRBA and SLC43A3 in a breast cancer tissue sample from the human, wherein underexpression of GABRP, RABEP1, SLC39A6 and PTP4A2 in the sample in combination with overexpression of ESR1, TCEAL1, ATAD2, LRBA and SLC43A3 in the sample identifies a human that has an increased likelihood of recurrence of the breast cancer that would potentially benefit from a therapy to decrease the likelihood of recurrence of the breast cancer.

In another embodiment, the invention is a method of optimizing treatment of a human having breast cancer, comprising the step of measuring a level of expression of genes selected from the group consisting of GABRP, TRIM29, RABEP1, SLC39A6, TCEAL1, PLK1 and CX3CL1 in a breast cancer tissue sample from the human, wherein underexpression of GABRP, TRIM29, RABEP1 and SLC39A6 in the sample in combination with overexpression of TCEAL1, PLK1 and CX3CL1 in the sample identifies a human that has an increased likelihood of recurrence of the breast cancer that would potentially benefit from a therapy to decrease the likelihood of recurrence of the breast cancer.

In a further embodiment, the invention is a method of optimizing treatment of a human having breast cancer, comprising the step of measuring a level of expression of genes selected from the group consisting of TBC1D9, RABEP1, SLC39A6, FUT8 and PTP4A2 in a breast cancer tissue sample from the human, wherein underexpression of TBC1D9, RABEP1, SLC39A6, FUT8 and PTP4A2 in the sample identifies a human that has an increased likelihood of recurrence of the breast cancer that would potentially benefit from a therapy to decrease the likelihood of recurrence of the breast cancer.

In still another embodiment, the invention is a method of optimizing treatment of a human having breast cancer, comprising the step of measuring a level of expression of genes selected from the group consisting of RABEP1, SLC39A6, FUT8 and PTP4A2 in a breast cancer tissue sample from the human, wherein underexpression of RABEP1, SLC39A6, FUT8 and PTP4A2 in the sample identifies a human that has an increased likelihood of recurrence of the breast cancer that would potentially benefit from a therapy to decrease the likelihood of recurrence of the breast cancer and thereby increase the likelihood of survival of the human.

In an additional embodiment, the invention is a method of optimizing treatment of a human having breast cancer, comprising the step of measuring a level of expression of genes selected from the group consisting of ESR1, GABRP, RABEP1, SLC39A6, TCEAL1, ATAD2, PTP4A2, LRBA and SLC43A3 in a breast cancer tissue sample from the human, wherein overexpression of GABRP, RABEP1, SLC39A6 and PTP4A2 in the sample in combination with underexpression of ESR1, TCEAL1, ATAD2, LRBA and SLC43A3 in the sample identifies a human that has a decreased likelihood of recurrence of the breast cancer that would potentially benefit from a therapy to treat the breast cancer.

In still another embodiment, the invention is a method of optimizing treatment of a human having breast cancer, comprising the step of measuring a level of expression of genes selected from the group consisting of GABRP, TRIM29, RABEP1, SLC39A6, TCEAL1, PLK1 and CX3CL1 in a breast cancer tissue sample from the human, wherein overexpression of GABRP, TRIM29, RABEP1 and SLC39A6 in the sample in combination with underexpression of TCEAL1, PLK1 and CX3CL1 in the sample identifies a human that has a decreased likelihood of recurrence of the breast cancer that would potentially benefit from a therapy to treat the breast cancer.

An additional embodiment of the invention is a method of optimizing treatment of a human having breast cancer, comprising the step of measuring a level of expression of genes selected from the group consisting of TBC1D9, RABEP1, SLC39A6, FUT8 and PTP4A2 in a breast cancer tissue sample from the human, wherein overexpression of TBC1D9, RABEP1, SLC39A6, FUT8 and PTP4A2 in the sample identifies a human that has a decreased likelihood of recurrence of the breast cancer that would potentially benefit from a therapy to treat the breast cancer.

“Optimizing treatment,” as used herein, means identifying a therapy (e.g., chemotherapy, radiation therapy or any combination of therapies) that has the greatest chance of eliminating the breast cancer or causing remission of the breast cancer as detected by, for example, the presence of breast cancer cells in biopsies, and preventing metastasis of the breast cancer. Malignant breast tumors can form metastases to non-breast tissues and organs by entering the systemic circulatory system (arteries, veins) or lymphatic circulatory system. The methods described herein can be employed to optimize treatment to prevent or minimize metastases of a malignant breast tumor.

“Would potentially benefit,” as used herein, means that the breast cancer may go into remission, is substantially eliminated or palliative remediation of the disease in the human.

“An increased likelihood of recurrence of breast cancer,” as used herein, means that the human had at least one incident of a diagnosis of breast cancer and has an elevated probability of having the breast cancer return. For example, in a meta-analysis (from seven different studies) of more than about 3,500 patients who had received some type of post-surgical adjuvant therapy for breast cancer, risk of cancer recurrence was greatest during the first two years following surgery. After this period, the research showed a steady decrease in the risk of recurrence until year five when the risk of recurrence declined slowly and averaged about 4.3% per year (Saphner T, et al., J Clin Oncol. 14:2738-2746 (1996)). Some proportion of breast cancer recurrences seen in this study occurred more than about five years after surgery, between about six to about 12 years after surgery, even in patients who typically would be considered at low risk for recurrence because their cancer had not spread to the lymph nodes at the time of diagnosis (node-negative). This study shows that through at least about 12 years of follow-up, the risk of breast cancer recurrence remains appreciable and even some patients considered low risk have some risk of the cancer recurring.

“Increased likelihood of survival,” as used herein, means that the human that had at least one incident of a diagnosis of breast cancer has an elevated probability of living.

Expression of the genes in the methods of the invention can be identified by detecting mRNA for the genes or the protein product of the gene (see, for example, U.S. Patent Application Nos. US 2005/0095607, US 2005/0100933 and US 2005/0208500, the teachings of all of which are hereby incorporated by reference in their entirety). In an embodiment, expression of the genes described herein can be assessed by measuring the messenger RNA (mRNA) of the gene in the breast cancer sample. Techniques to identify mRNA are known in the art and include, for example, qPCR, as described infra.

Expression of the genes in the methods described herein can be assessed by Northern Blot analyses. Expression of genes in the methods described here may also be assessed by amplifying a nucleic acid sequence of the gene and detecting the amplified nucleic acid by well-established methods, such as the polymerase chain reaction (PCR), including quantitative PCR (qPCR), reverse transcription PCR (RT-PCR), and real-time PCR (including as a means of measuring the initial amounts of mRNA copies for each sequence in a sample), real-time RT-PCR or real-time Q-PCR. Exemplary techniques to employ such detection methods would include the use of one or two primers that are complementary to portions of a gene of interest, as described herein, where the primers are used to prime nucleic acid synthesis. The newly synthesized nucleic acids are optionally labeled and may be detected directly or by hybridization to a gene or mRNA. The newly synthesized nucleic acids may be contacted with polynucleotides of a breast tissue sample under conditions which allow for their hybridization. Additional methods to detect the expression of genes in the methods described herein include RNAse protection assays, including liquid phase hybridizations and in situ hybridization of cells.

Quantitative polymerase chain reaction (qPCR), also known as real-time PCR, is a modification of the PCR technique that is used to measure the quantity of a specific RNA molecule present in a sample with a high degree of sensitivity (Ding, C., et al. J. Biochem Mol. Biol., 37(1):1-10 (2004)). This is accomplished by first reverse transcribing the RNA to complementary DNA (cDNA), and then amplifying the gene of interest with target specific primers. The amount of DNA is measured after each cycle of PCR by use of fluorescent markers, such as TAQMAN® probes (Applied Biosystems), Sybr green, or molecular beacons. QPCR is one of the most widely used methods of studying specific gene expression in a variety of organisms, tissues, and cells.

Competitive PCR, which utilizes a DNA standard containing a point mutation to differentiate it from the gene of interest, can also be employed to assess expression of genes in the methods of the invention. The point mutation either creates or removes a restriction site, allowing the standard to be distinguished from the target gene. Both the cDNA and DNA standard are co-amplified in the PCR reaction. Resulting products are treated with a restriction enzyme and either subjected to gel electrophoresis, ion pair reversed phase high performance liquid chromatography (IP-RP-HPLC), or matrix assisted laser desorption ionization time of flight mass spectrometry (MALDI-TOF MS) Ding, C., et al., J. Biochem Mol Biol., 37(1):1-10 (2004). Since the amount of DNA standard is known, the concentration of cDNA target can be calculated.

Many genomic questions utilize discovery-based tools to determine global genomic differences between two or more test groups. One of the most widely used methodologies has been microarray gene chips, which span an organism's genome in order to study various aspects, such as gene copy number, single nucleotide polymorphisms (SNPs), comparative genomic hybridization (CGH), and, most commonly, variations in gene expression. Although each type of microarray is designed to study a particular aspect of genomics, they function by similar means. They contain thousands of probes directed at sequences spanning the genome. When a test sample is hybridized with the chip, it can be detected with fluorescence of Cy5/Cy3 or biotin/streptavidin-conjugated to fluorescent compound. With the development of this complicated and powerful technology, there was a great need for bioinformatics tools to analyze the vast amounts of information obtained. Software programs, such as GeneSpring GX (Agilent Technologies), GENECHIP™ (Affymetrix), and Partek GS (Partek Incorporated), have been developed to help decipher the massive data sets obtained from global gene expression studies.

Gene expression for use in the methods described herein can also be assessed by differential display. In this technique mRNA is reverse transcribed using three anchored oligo(dT) primers that differ in the base adjacent to the poly(dT) sequence. The resulting cDNA is then further amplified with short (about 13 bp) random primers. The resulting PCR products are labeled with either radioisotopes or fluorescent dyes and separated by polyacrylamide gel electrophoresis (PAGE). When two cDNA samples are displayed on the gel side-by-side, changes in gene expression can be detected (Ding, C., et al., J Biochem Mol Biol., 37(1):1-10 (2004)). By utilizing laboratory automation technologies, the entire genome can be covered with a few hundred reactions. Another technique is serial analysis of gene expression (SAGE), which utilizes double stranded cDNA sequences made with biotinylated oligo(dT) primers. These are then digested with a restriction enzyme, and the 3′ ends are recovered with streptavidin beads. The cDNA is then ligated to linker sequences containing a specific restriction site which cleaves 14 by downstream of the site. This yields a linker attached to a 10 base gene-specific tag, which is then cloned into a plasmid and sequenced. The frequencies of gene-specific tags are utilized to estimate the gene expression levels.

Increases (up-regulation of expression, also referred to an “overexpression”) and decreases (down-regulation of expression, also referred to a “underexpression”) of genes in the method described herein may be expressed in the form of a ratio between expression in a cancerous breast cell or a Universal Human Reference RNA (Stratagene, La Jolla, Calif.) (also referred to herein as a “control”). For example, a gene can be considered up-regulated if the median expression value relative to a control, such as a Universal Human Reference RNA, is above one (1). Likewise, a gene can be considered down-regulated if the median expression value relative to a control, such as a Universal Human Reference RNA, is less than one (1), as described herein. Expression levels can be readily determined by quantitative methods as described herein, such as nucleic acid amplification assays. The methods described herein can identify over-expression (increases) or under-expression (decreases) of genes compared to a Universal Human reference RNA control. Over-expression or under-expression can be correlated with patient characteristics (e.g., age, menopausal stage, disease-free) and breast cancer characteristics (e.g., grade stage, estrogen receptor status, progesterone receptor status).

Over and under expression of genes described herein can be assessed by determining the Hazard Ratio (HR) by the methods described herein. HR less than one (1) indicates that the gene is overexpression and HR over one (1) indicates that the gene is underexpressed.

Expression of the genes described herein can be assessed as a ratio of the expression of the gene in a breast tissue sample from the mammal and a control tissue sample, such as from another mammal with breast cancer, from a sample of the same mammal from a previous breast cancer incident, or a mammal without breast cancer (also referred to herein as “normal” or “non-cancerous”). For example, an increase in the ratio of expression of the gene in the breast tissue sample from the mammal compared to a non-cancerous sample, may indicate an increased likelihood of recurrence of the breast cancer. The ratios of increased expression can be about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 15, about 20, about 30, about 40, about 50, about 60, about 70, about 80, about 90, about 100, about 150, about 200, about 300, about 400, about 500, about 600, about 700, about 800, about 900 or about 1000. For example, a ratio of 2 is a 100% (or a two-fold) increase in expression. Likewise, a decrease in gene expression can be indicated by ratios of about 0.9, about 0.8, about 0.7, about 0.6, about 0.5, about 0.4, about 0.3, about 0.2, about 0.1, about 0.05, about 0.01, about 0.005, about 0.001, about 0.0005, about 0.0001, about 0.00005, about 0.00001, about 0.000005 or about 0.000001, which may indicate a decreased likelihood of recurrence of breast cancer in the mammal.

Similarly, increases and decreases in expression of the genes described herein can be expressed based upon percent or fold changes over expression in non-cancerous cells or a control, such as a Universal Human Reference RNA. Increases can be, for example, about 10, about 20, about 30, about 40, about 50, about 60, about 70, about 80, about 90, about 100, about 120, about 140, about 160, about 180 or about 200% relative to expression levels in non-cancerous cells or a control. Alternatively, fold increases may be of about 1, about 1.5, about 2, about 2.5, about 3, about 3.5, about 4, about 4.5, about 5, about 5.5, about 6, about 6.5, about 7, about 7.5, about 8, about 8.5, about 9, about 9.5 or about 10 fold over expression levels in non-cancerous cells. Likewise, decreases may be of about 10, about 20, about 30, about 40, about 50, about 55, about 60, about 65, about 70, about 75, about 80, about 85, about 90, about 95, about 98, about 99 or 100% relative to expression levels in non-cancerous cells or a control.

Exemplary methods to assess relative gene expression analyses include employing the ΔΔCt method, in which the threshold cycle number (CT value) is the cycle of amplification at which the OCR instrument system recognizes an increase in the signal (e.g., SYBR® green florescence) associated with the exponential increase of the PCR product during the log-linear phase of nucleic acid amplification. These CT values are compared to those of a housekeeping gene, such as glyceraldehyde phosphate dehydrogenase (GAPDH) or β-actin to obtain the ΔCt value, which is used to normalize for variation in the amount of RNA between different samples. The ΔCt value of each gene is then compared to that present in a calibrator, such as Universal Human Reference RNA (Stratagene, La Jolla, Calif.), in order to obtain a ΔΔCt value. Since each cycle of amplification doubles the amount of PCR product, the expression level of a target gene relative to that of the calibrator is calculated from 2^{-ΔΔCt}, expressed as relative gene expression.

In one embodiment, the breast tissue sample is a laser capture microdissection (LCM) breast tissue sample. LCM is known in the art and is described herein. LCM can result in collections of varying cell types (e.g., epithelial, stromal, smooth muscle) in varying numbers, such as about 100 cells, about 1000 cells, about 2000 cells or about 5000 cells. LCM can be employed to prepare a breast tissue sample that includes relatively pure populations of a single cell type, such as an epithelial cell, a stroma cell or a smooth muscle cell.

Systems include the PIXCELL IIe™ LCM System and Image Archiving Workstation (Arcturus Bioscience, Inc.), which utilizes a thermal-sensitive film that is placed over the cells of interest. When the infra-red laser is fired from above, the film is melted onto the cells of interest and resolidifies encapsulating those cells. Sluka P, et al., Prog Histochem Cytochem; 42(4):173-201 (2008).

The P.A.L.M. (P.A.L.M. Microlaser Technologies, Bernried, Germany) instrument utilizes both laser microdissection and pressure catapulting (Burgemeister, R., J. Histochem. Cytochem 53(3):409-412 (2005)). This is performed by an ultraviolet laser firing from below the tissue to cut through the region containing the cells of interest, with a second firing that catapults the cells up off the slide. The Leica (Wetzlar, Germany) AS laser microdissection (LMD) instrument does not utilize a glass slide, and the dissected cells drop into a collection tube. Molecular Machine & Industries (MMI, Glattbrugg, Switzerland) has developed two instruments, the mmi CELLCUT™ and the mmi SMARTCUT™. These instruments both allow microdissection of single cells or groups of cells collected using an adhesive cap rather than by catapulting. The VERITAS™ is a relatively new instrument from Molecular Devices (Sunnyvale, Calif.), combines the technologies of laser capture and laser cutting and utilizes both an ultraviolet and infrared laser to perform the microdissection [46].

In another embodiment, the breast tissue sample is an intact tissue section breast tissue sample. Intact tissue section can be prepared employing established techniques. For example, an intact tissue section can be prepared by freezing a breast tissue sample obtained from a biopsy in O.C.T. (Optimum Cutting Temperature) and cryo-sectioning the intact breast tissue sample. The frozen intact tissue section is then placed on a glass slide and stained with hematoxylin and eosin to assess structural integrity. Additional frozen intact tissue sections are prepared for total RNA extraction, purification and analyzed by quantitative polymerase chain reaction (qPCR), as described infra.

The breast tissue sample can be a biopsy sample that includes at least one member selected from the group consisting of breast epithelial cells, breast stromal cells, breast smooth muscle cells, which can include breast cancer cells of these tissue types. The breast tissue sample can be a breast biopsy that includes a carcinoma (ductal, lobular, medullary and/or tubular carcinoma). The breast tissue sample can be a breast biopsy that includes stroma. The breast tissue sample can be subjected to laser capture microdissection (LCM) in which relatively pure populations of carcinoma cells (cancerous cells of breast epithelium) and/or relatively pure populations of stromal cells are obtained. “Relatively pure,” as used herein in reference to a carcinoma or stromal breast tissue sample, means that the sample is about 95%, about 98%, about 99% or about 100% one cell type (e.g., carcinoma or stroma).

The breast tissue sample employed in the methods described herein can include homogenates of breast cancer biopsies, which include populations of different cell types (e.g., epithelial, stromal, smooth muscle).

The breast cancer tissue sample can be from a pre-menopausal human or a post-menopausal human.

The breast cancer tissue sample employed in the methods of the invention can be a breast cancer tissue sample, such as a primary breast cancer tissue sample, from a human that is lymph node negative (i.e., the breast cancer has not spread to the lymph node) and the breast cancer is estrogen receptor positive; or can be a breast cancer tissue sample from a human that is lymph node positive breast cancer (i.e., the breast cancer has spread to the lymph node) and the breast cancer is estrogen receptor positive.

The breast cancer tissue sample can be from a human with stage 1 (I), 2 (II), 3 (III) or 4 (IV) estrogen-receptor breast cancer or a human with stage 1, 2, 3 or 4 estrogen-receptor positive and progesterone-receptor positive breast cancer.

The American Joint Committee on Cancer (AJCC) staging of breast cancer is based on a scale of 0-4, with 0 having the best prognosis and 4 having the worst. There are multiple sub-classifications within each Stage classification (Robbins and Cotran, Pathological Basis of Disease, 7th ed., Kumar, V., et al. (eds), Elsevier Saunders (2005)). Patients that present with ductal carcinoma in situ (DCIS) or lobular carcinoma in situ (LCIS) are considered stage 0. An invasive carcinoma of less than about 2 cm in the greatest dimension and no lymph node involvement is considered Stage I. An invasive carcinoma of less than about 5 cm in the greatest dimension and about 1 to about 3 positive lymph nodes is considered Stage II. Stage III refers to an invasive carcinoma of less than about 5 cm in the greatest dimension and four or more axillary lymph nodes involved or to an invasive carcinoma no greater than about 5 cm in the greatest dimension with nodal involvement or to an invasive carcinoma with at least about 10 axillary lymph nodes involved or invasive carcinoma with involvement of ipsilateral internal lymph nodes or invasive carcinoma with skin involvement, chest wall fixation or inflammatory carcinoma. Stage 1V refers to a breast carcinoma with distant metastases (Robbins and Cotran Pathological Basis of Disease, 7th Edition, eds. V. Kumar, et al., A. K. Abbas and N. Fausto, Elsevier Saunders (2005)).

Clinical staging of breast cancer is an estimate of the extent of the cancer based on the results of a physical exam, imaging tests (e.g., x-rays, CT scans) and often biopsies of affected areas. Blood tests can also be used in staging.

Pathological staging can be done on patients who have had surgery to remove or explore the extent of the cancer, which can be combined with clinical staging (e.g., physical exam, imaging tests). In some cases, the pathological stage may be different from the clinical stage. For example, surgery may reveal that the cancer has spread beyond that predicted from a clinical exam.

In an embodiment, the methods of the invention measure expression of genes in breast cancer sample is from a human that has an estrogen-receptor positive breast cancer (referred to herein as “ER⁺”). In a further embodiment, the breast cancer sample is from a human that has a progesterone-receptor positive breast cancer (referred to herein as “PR⁺”). In still another embodiment, the breast cancer sample is from a human that has an estrogen-receptor positive and a progesterone-receptor positive (referred to herein as “ER⁺/PR⁺”) breast cancer. Estrogen Receptor (ER) is also referred to herein as “ESR.” Progesterone Receptor is also referred to herein as “PGR” or “PR.”

The ESR measured can be expression of at least one member selected from the group consisting of ESR1 (also referred to as “estrogen receptor alpha”) gene expression and ESR2 (also referred to as “estrogen receptor beta”) gene expression.

“Estrogen-receptor positive breast cancer,” as used herein, means that the levels of estrogen receptor protein in the breast cancer sample or biopsy are greater than about 10 fmol/mg protein (e.g., about 10 fmol/mg protein by ligand binding assay or about 15 fmol/mg protein by EIA) by established techniques, such as at least one member selected from the group consisting of radioligand binding, Enzyme ImmunoAssay (EIA) and semi-quantitative immunohistochemical assay (see, for example, Wittliff, J. L., et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W. B. Saunders Co. (1998)).

“Progestin-receptor positive breast cancer,” as used herein, means that the levels of progestin receptor protein in the breast cancer sample or biopsy measure greater than about 10 fmol/mg protein (e.g., about 10 fmol/mg protein by ligand binding assay or about 15 fmol/ng by EIA) by established techniques, such as at least one member selected from the group consisting of radioligand binding, EIA and semi-quantitative immunohistochemical assay (see, for example, Wittiff, J. L., et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W. B. Saunders Co. (1998)).

Humans whose treatment is optimized by the methods described herein can have an estrogen-receptor positive breast cancer that is a primary estrogen-receptor positive breast cancer (i.e., cancer arising from breast tissue, such as epithelial tissue) or a secondary estrogen-receptor positive breast cancer (i.e., cancer arising from an organ other than breast tissue that metastases to breast tissue).

The methods described herein can further include the step of treating the human with a therapy that decreases the likelihood of recurrence of the breast cancer. The therapy may increase the likelihood of survival of the human. The selection of therapy will depend on, for example, the stage of the breast cancer, the expression of particular genes, age of the human, overall health status, current treatment, ER status of the breast cancer and PR status of the breast cancer. Therapies can include at least one member selected from the group consisting of surgery radiation therapy, chemotherapy and, for ER⁺, PR⁺ or ER⁺/PR⁺ breast cancers, endocrine therapy. For example, polychemotherapy with at least 4 cycles of one member selected from the group consisting of cyclophosphamide in combination with methotrexate and fluorouracil (CMF); doxorubicin in combination with fluorouracil and cyclophosphamide (FAC); and fluoruracil in combination with epirubicin and cyclophosphamide (see, for example, Early Breast Cancer Trialists' Collaborative Group (EBCTCG), Lancet 365(9472):1687-717 (2005)) may be used as a therapy to optimize treatment of humans with ER⁺ and PR⁺ breast cancers. Chemotherapy may be combined with radiation therapy and/or endocrine therapy. Endocrine therapy, such as treatment with at least one member selected from the group consisting of at least one estrogen receptor antagonist, at least one aromatase inhibitor and at least one selective estrogen receptor modulator (“SERM”), could be employed in humans having ER positive breast cancer. Alternatively, to optimize treatment of the breast cancer, chemoendocrine therapies may be employed in combination with endocrine adjuvant therapies, for example, in humans identified by the methods of the invention that have lymph node negative breast cancers.

“Selective estrogen receptor modulator (SERM),” as used herein, refers to nonsteroidal and steroidal compounds that interact with the estrogen receptor to thereby affect or mediate the action of estrogens, such as 17β-estradiol. The administration of a SERM may provide the benefits of estrogens without the potentially adverse risk of increased cell proliferation in estrogen-responsive tissues, such as breast and uterine epithelium. Selective estrogen receptor modulator, such as a 2-[4-[1,2-di(phenyl)but-1-enyl]phenoxy]-N,N-dimethylethanamine therapy (e.g., TAMOXIFEN™ therapy), can be employed alone or in combination with other treatments (e.g., chemotherapy, radiation therapy) when the methods of the invention identify a human that has an increased likelihood of recurrence and have or had an ER positive breast cancer.

Radiation therapy, has generally be employed as a treatment for relatively large breast cancer tumors and breast cancers from humans with at least four (4) positive lymph nodes. Humans identified by the methods described herein that can potentially benefit from a therapy to decrease the likelihood of recurrence of the breast cancer, in particular cancers that are from lymph node-negative humans (also referred to herein as “patients”) may have optimized therapies that include more aggressive therapy, such as radiation even if the clinical profile, for example, small tumor, low lymph node involvement, would not otherwise lead itself to radiation therapy.

For ER⁺ breast cancers, the methods of the invention can identify humans with increased risks of recurrence of the breast cancer can result in treatments that are customized to the patient and may be more clinically aggressive than patients who do not have an increased likelihood of recurrence of the breast cancer. Thus, treatment of humans having an increased likelihood of recurrence of the breast cancer can be a more aggressive therapy.

The methods described herein can further include the step of administering at least one alternative therapy to the human alone or in combination with the 2-[4-[1,2-di(phenyl)but-1-enyl]phenoxy]-N,N-dimethylethanamine therapy, thereby treating the human for the estrogen-receptor positive breast cancer. An exemplary alternative therapy can include at least one aromatase inhibitor (Mauri, D., et al., J. Natl. Cancer Inst. 98:1285-1291 (2006)) (e.g., Anastrozol, Arimidex™, 2-[3-(1-cyano-1-methyl-ethyl)-5-(1H-1,2,4-triazol-1-ylmethyl) phenyl]-2-methyl-propanenitrile). Selective estrogen receptor modulator, for example, 2-(para-((Z)-4-chloro-1,2-diphenyl-1-butenyl)phenoxy)-N,N-dimethylethylamine, IUPAC designation) (Pagani, O., et al., Ann. Oncol. 15:1749-1759 (2004)) (TOREMIFENE™) and [6-hydroxy-2-(4-hydroxyphenyl)-1-benzothiophen-3-yl]-4-(2-piperidin-1-ium-1-ylethoxy)phenyl]methanone chloride (RALOXIFENE™, EVISTA® IUPAC designation (2-(4-Hydroxyphenyl)-6-hydroxybenzo(b)thien-3-yl)(4-(2-(1-piperidinyl)ethoxy)phenyl)methanone may be considered.

“Alternative therapy,” as used herein, means a treatment other than treatment with 2-[4-[1,2-di(phenyl)but-1-enyl]phenoxy]-N,N-dimethylethanamine therapy (i.e., TAMOXIFEN™ IUPAC designation (Z)-2-(para-(1,2-Dephenyl-1-butenyl)phenoxyl)-N,N-dimethylamine) also referred to as NOLVADEX™. “Alternative therapy,” is also referred to herein a “therapy that is alternative to.” The alternative therapy can be administered alone or in combination (e.g., before, during or after) with chemotherapy, radiation therapy and therapy with estrogen-receptor antagonists, such as 2-[4-[1,2-di(phenyl)but-1-enyl]phenoxy]-N,N-dimethylethanamine.

Optimization of treatment of human by the methods described herein that have ER⁺ and/or PR⁺, lymph node-negative breast cancers may include the use of TAMOXIFEN™ alone as a maintenance therapy after surgical removal of the tumor or a course of adjuvant chemotherapy (e.g., CMF, FAC, FEC).

Employing the methods described herein, a patient can be identified that has a “high risk” of recurrence (i.e., the breast cancer sample has an expression profile of a particular gene subsets as described herein), indicating that the patient should receive more aggressive therapies (terms used by oncologists to describe, for example, dose escalations). Thus, a patient with the lymph node-negative cancer would be a candidate for therapy regimens selected for patients with lymph node-positive cancer, which include multiple courses of polychemotherapy and/or external beam radiation therapy. Various polychemotherapy regimens are used at the discretion of the oncologist depending upon the collective characteristics of the lesion, the patient parameters and health status and other features and would be within the knowledge and medical expertise of one skilled in the art. The regimens could include TAC (docetaxel plus doxorubicin and cyclophosphamide).

Thus, the methods of the invention can be employed to identify patients who are less likely to have a recurrence of a breast cancer.

In addition, humans having lymph node-positive cancers, that can include breast cancers that are ER⁺ and/or PR⁺, and expression profiles of genes employed in the methods described herein may indicate that the human has a “low risk” of recurrence. Thus, even though the patient is lymph node-positive, they may benefit from a less aggressive treatment (e.g., polychemotherapy alone or radiation therapy alone).

Thus, the expression of the genes described herein may predict the survival and prognosis of the human. For example, the methods described herein identify a human who has an increased likelihood of recurrence of breast cancer, which may indicate an increased likelihood of death. Likewise, employing the methods described herein, a human may be identified who has a relatively low likelihood of recurrence of breast cancer, which may indicate increased survival.

The methods of the invention can be employed to predict, for example, local recurrence of primary breast carcinoma and regional or distant metastases from primary breast carcinoma, which may provide prognostic evaluation of overall survival probabilities at time of diagnosis for primary breast carcinoma. The methods of the invention can be employed to optimize therapeutic regiments for treatment of the breast cancer, which would be customized to the patient by one of skill in the art based on factors such as age, health history, other disease and family history. The gene expression profiles described herein may provide biomarkers assessing disease progression and response in human cancers other than breast (e.g., ovarian, uterine, colon).

Several methods to predict the likelihood of recurrence of breast cancer have been described, including ONCOTYPE DX™, MAMMA PRINT®, BREAST BIOCLASSIFIER™. However, such tests are based on samples obtained for analysis from various methods (e.g., cell lines, fixed tissues) and assess relatively large number of genes (e.g., 21 genes, 97 genes) and, thus, are not suitable for routine screening.

The methods described herein provide clinically relevant subset of genes in a tissue biopsy that predicts breast cancer behavior (gene subset of about 10, 9, 7, 5 or 4 genes is commercially feasible for development of a molecular diagnostic acceptable to clinicians, pathologists and laboratory medicine specialists. The methods of the invention may be performed quickly on tissue biopsies, and the entire panel of genomic biomarkers may be measured simultaneously in conventional formats, e.g., qPCR or hybridization arrays.

Few genomic tests are currently available in the clinical laboratory setting, and few technical staff have experience in the isolation, purification and amplification of labile mRNA for technologies such as qPCR and microarray. Use of molecular diagnostic technologies can provide for standardized methods for tissue collection that preserve the integrity of the biological macromolecules (DNA, RNA, protein) with the cells, allowing for more accurate detection.

“Breast cancer behavior,” as used herein, means, for example, whether the breast cancer will result in an increased likelihood of recurrence of the breast cancer, whether the human has increased likelihood of survival or death and a selection of a course of treatment for the breast cancer.

The methods described herein may be used in combination with other methods of diagnosing breast cancer to thereby more accurately identify a mammal at an increased risk for recurrence of breast cancer. For example, the methods described herein may be employed in combination or in tandem with assessments of the presence or absence of Ki-67, an antigen that is present in all stages of the cell cycle except GO and can be employed as a marker for tumor cell proliferation, and prognostic markers (including oncogenes, tumor suppressor genes, and angiogenesis markers) like p53, p27, Cathepsin D, pS2, multi-drug resistance (MDR) gene, and CD31. Alone or in combination with other clinical correlates of breast cancer, the methods described here may increase the accuracy of detection of breast cancer, in particular, in mammals who have had at least one or more incidents of breast cancer, thereby optimizing treatment of the breast cancer to decrease likelihood of recurrence of the breast cancer.

In an additional embodiment, the invention is an immobilized collection (microarray) of the genes, such as a gene chip for ease of processing in the methods described herein. The gene chips that include the genes described herein can permit high throughput screening of numerous breast tissue samples. The genes identified in the methods described herein can be chemically attached to locations on an immobilized collection, such as a coated quartz surface. Nucleic acids from breast tissue samples can be prepared as described herein and hybridized to the genes and expression of the genes identified.

In another embodiment, the invention includes kits to perform the methods described herein.

The teachings of all patents, published applications and references cited herein; and U.S. patent application Ser. No. 12/630,212 (Publication No: 2010/0112592) and Patent Cooperation Treaty Applicant No: PCT/US2009/060506 (WO 2010/045234) are incorporated by reference in their entirety.

### EXEMPLIFICATION

RNA was isolated from tissue sections of 126 de-identified frozen biopsies of invasive ductal carcinoma using the RNeasy® Mini kit (Qiagen) and analyzed for quality and quantity using the BIOANALYZER (Agilent). cDNA for qPCR measurements was prepared in Tris-HCl buffer containing KCl, MgCl₂, DTT (Invitrogen), dNTPs (Invitrogen), RNasin® (Promega) and Superscript® RT III (Invitrogen). qPCR reactions were performed using Power Sybr® Green PCR Master Mix (Applied Biosystems), forward/reverse primers and cDNA obtained from the reverse transcription reaction. Relative gene expression was calculated with the ddCt method, using β-actin as the reference gene and Universal Human Reference RNA (Stratagene) as a calibrator. qPCR reactions were performed in triplicate with duplicate wells in each 384-well plate, to ensure reproducibility.

Gene expression results from qPCR were correlated with disease-free and overall survival outcome data. Expression of TBC1D9, RABEP1, SLC39A6, FUT8, and PTP4A2 correlated with disease-free survival using univariate Cox proportional hazards analyses (P<0.05). Expression of RABEP1, SLC39A6, FUT8, and PTP4A2 appeared to be related to overall survival using univariate analysis (P<0.05). Multivariate analyses were performed with backwards stepwise selection to predict disease-free survival using expression levels of ESR1, GABRP, RABEP1, SLC39A6, TCEAL1, ATAD2, PTP4A2, LRBA, and SLC43A3. ROC curves were composed to illustrate the sensitivity and specificity of the model for disease-free and overall survival with areas under the curves equal to 0.78 and 0.76, respectively. Consideration of additional parameters, e.g., estrogen and progestin receptor status, menopausal status and lymph node involvement, did not improve the model.

A molecular signature was identified consisting of expression profiles of candidate genes, in a multivariate Cox proportional hazards model of breast cancer recurrence. The model also predicted overall survival.

Use of SPSS statistical software enabled the use of multivariate Cox regressions (using forward and backward stepwise selection) to obtain an optimal model for predicting patient survival (i.e., clinical outcome of breast cancer patients).

Survival analyses of individual genes of both carcinoma and stromal subsets revealed over-expression of TBC1D9 and TPBG in the carcinoma cells were associated with decreased disease-free and overall survival.

Individual expression levels of TBC1D9, CENPA, MELK, ATAD2, MCM6, YBX1, GMPS, and CKS2 in the stromal cells were associated with poor prognosis of breast cancer. These results indicate that over-expression of each of these 8 genes in stromal cells is correlated with an increased likelihood of death due to breast cancer.

Over-expression of TBC1D9 in either LCM-procured carcinoma cells or surrounding stromal cells appears to be associated with poor survival.

Each of the 32 candidate genes was evaluated using clinical follow-up and microarray results from LCM-procured carcinoma cell preparations from 247 patient specimens. Examination of the entire 22,000 gene microarray results from carcinoma cells revealed that individual expression levels of twelve genes in the “stromal subset” (e.g., FUT8, MELK, PFKP, PLK1, ATAD2, XBP1, BUB1, GATA3, MAPRE2, GMPS, CKS2, and SLC43A3) were independently associated with disease-free or overall survival.

Examination of these same results from the entire 22,000 gene microarray results from LCM-procured carcinoma cells) revealed that individual expression levels of ten genes in the “cancer subset” (e.g., EVL, NAT1, ESR1, TBC1D9, SCUBE2, IL6ST, RABEP1, TPBG, TCEAL1, and DSC2) were independently associated with disease-free or overall survival of breast cancer patients.

Expression levels of seven genes (NAT1, ESR1, SCUBE2, FUT8, PTP4A2, LRBA, and MAPRE2) appear to be highly correlated with other genes in the 32 gene set. Each of these seven genes exhibited expression levels related to those of another gene when examined as gene pairs (Pearson correlation used as statistic). Each of the seven genes correlated as pairs with more than 20 of the other genes in the 32 gene set. Expression levels of estrogen and progestin receptor mRNA were highly correlated with ER and PR protein levels of these known tumor markers using Pearson correlations and linear regressions.

When genes were individually stratified by median expression level and individually analyzed by Kaplan-Meier survival plots, SCUBE2 exhibited a median expression level that significantly stratified patients into good and poor prognosis groups for disease-free survival, while GABRP, TBC1D9, SLC39A6, MELK, MCM6, and PTP4A2 associate with disease-free and overall survival (P value less than 0.10).

Several genes (GABRP for nodal status; NAT1, CENPA, and BUB1 for tumor grade; ESR1, SCUBE2, RABEP1, SLC39A6, TCEAL1, and XBP1 for ER status; SLC39A6 and PTP4A2 for PR status) appear to distinguish good and poor prognosis groups in specific patient populations better than in the entire population.

Expression of TBC1D9, RABEP1, SLC39A6, FUT8, and PTP4A2 correlated independently with disease-free survival using univariate Cox Regression analyses (P less than 0.05).

Expression of RABEP1, SLC39A6, FUT8, and PTP4A2 appeared related to overall survival using univariate analysis (P less than 0.05).

Multivariate Cox proportional hazards models, performed with backwards stepwise selection in the entire population, predicted disease-free survival using expression levels of nine genes (ESR1, GABRP, RABEP1, SLC39A6, TCEAL1, ATAD2, PTP4A2, LRBA, and SLC43A3). ROC curves indicated the sensitivity and specificity of the model for disease-free and overall survival.

Results described herein identified small, biologically significant and clinically relevant gene sets that form the basis for a commercial test for assessing risk of breast cancer recurrence. The small number of genes in the clinically relevant subsets and the availability of technology for constructing an instrument for measuring gene expression, allows development of a readily available test to predict risk of recurrence of breast cancer at the time of surgical removal of the primary cancer. The ability to determine a gene expression profile in a hospital laboratory setting avoids the necessity for a “send-out test.”

Gene sets, identified in previous studies distinguishing subtypes, are too complex for routine use in breast cancer management. To assess clinical relevance, smaller sets of 32 candidate genes were identified. Procedures, refined for processing human tissue biopsies for microgenomics, revealed gene expression levels measured by qPCR were similar in LCM-procured carcinoma cells compared to those of intact tissue. However, LCM appeared essential when studying gene expression in stromal cells, since greater differences were observed compared to intact tissue. Survival analyses revealed that over-expression of each of eight genes in stromal cells correlated with decreased patient survival.

Examination of microarray results from carcinoma cells indicated that expression of twelve genes in the “stromal subset” were also clinically relevant, suggesting importance of measuring gene expression in both carcinoma and stromal cells. After qPCR validation, distribution and expression levels of each gene were determined by qPCR in 126 breast carcinoma specimens. Although 7 genes exhibited bimodal distribution, it was insignificant in survival analyses. Expression levels of seven genes were correlated with more than 20 other genes suggesting pathway associations. Expression of TBC1D9, RABEP1, SLC39A6, FUT8, and PTP4A2 correlated independently with disease-free survival using univariate Cox Regression analyses, while that of RABEP1, SLC39A6, FUT8, and PTP4A2 appeared related to overall survival. Several genes, individually stratified by median expression level and Kaplan-Meier analysis, distinguished good and poor prognosis groups in specific patient populations better than in the entire population. Multivariate Cox proportional hazards models predicted disease-free survival using expression levels of nine genes (ESR1, GABRP, RABEP1, SLC39A6, TCEAL1, ATAD2, PTP4A2, LRBA, and SLC43A3). ROC curves illustrated sensitivity and specificity of the model for disease-free and overall survival. Small, clinically relevant gene sets are being developed as a commercial test for assessing risk of breast cancer recurrence. Prediction of risk of recurrence at the time of surgical removal of the primary breast cancer will facilitate treatment planning and disease surveillance resulting in improved clinical care.

Breast cancer represents a prevalent disease in which genomic approaches have been employed, with the hope of improving the understanding, treatment and prevention of the disease. This has become a major health concern, because it is the most prevalent form of cancer in women in the United States. The American Cancer Society estimates that about 192,370 new cases of breast cancer will be diagnosed in 2009, and about 15 percent of cancer deaths (estimated at 40,170) in women will be due specifically to breast cancer in 2009, which is the second highest mortality of all cancer types. It is estimated that about 13.4 percent of women born in the United States today will be diagnosed with breast cancer at some point in their lives.

There are many different prognostic and predictive factors utilized when assessing breast cancer patients, since the outcome varies significantly. Generally, the prognosis is based on the pathological attributes of the primary tumor and the axillary lymph nodes. The major prognostic factors include 1) whether the disease is confined to ducts and lobules by the basement membrane (in situ) or invading the surrounding tissues; 2) whether there are distant metastases present in the patient; 3) whether the carcinoma has spread to the lymph nodes; 4) the size of the primary tumor; 5) presence of local advanced disease; and 6) presence of inflammatory carcinoma [8]. These major prognostic factors are the strongest predictors of death from breast cancer and are incorporated into the American Joint Committee on Cancer (AJCC) staging system [9]. The AJCC staging is on a scale of 0-4, with 0 having the best prognosis and 4 having the worst. Patients that present with ductal carcinoma in situ (DCIS) or lobular carcinoma in situ (LCIS) are considered Stage 0. An invasive carcinoma of less than 2 cm in the greatest dimension and no lymph node involvement is considered Stage 1. An invasive carcinoma of less than 5 cm in the greatest dimension and 1-3 positive lymph nodes or greater than 5 cm in the greatest dimension without lymph node involvement is considered Stage 2, Stage 3 refers to an invasive carcinoma of 5 cm or less in the greatest dimension and four or more axillary lymph nodes involved or to an invasive carcinoma greater than 5 cm in the greatest dimension with nodal involvement or to an invasive carcinoma with 10 or more involved axillary lymph nodes or invasive carcinoma with involvement of ipsilateral internal lymph nodes or invasive carcinoma with skin involvement, chest wall fixation or inflammatory carcinoma. Stage 1V refers to any breast carcinoma with distant metastases present (derived from [8; 9]).

There are additional prognostic factors which are used to determine which therapies may best benefit the patient. These include 1) histology of the primary tumor; 2) tumor grade, which assesses the degree of differentiation in the cells within the tumor; 3) presence of estrogen receptors (ER) & progestin receptors (PR) in the tumor, determine whether a patient is a candidate for hormone therapy, such as tamoxifen (NOLVADEX™), anastrozole (ARIMIDEX™), etc.; 4) over-expression of HER-2/neu oncoprotein, determines if a patient is a candidate for antibody therapy, such as Trastuzumab (HERCEPTIN™); 5) lymphovascular invasion; 5) proliferation rate of the cells; and 6) the DNA content in the tumor cells [8; 10-12].

Applying genomic and proteomic approaches to studying human cancer has been complicated by some fundamental problems of tissue collection and handling, as well as reliable methods for extracting, purifying, amplifying and analyzing RNA for gene expression profiling. These problems are also compounded by the cellular heterogeneity of breast tissue biopsies, which are used in the studies, compared to those involving the use of animal models or homogeneous cell lines grown in culture. For example, analysis of the levels or activities of certain tumor markers are currently performed either using biochemical or immunohistochemistry methodologies (e.g., [10; 11]). If the analyte is measured in a biochemical assay, a tissue biopsy consisting of a heterogeneous cell population is homogenized and the final concentration of the analyte from the cancer cells is reduced by the contamination of other proteins released from non-cancerous cells (e.g., surrounding stroma, epithelium and connective tissue cells). Therefore, a bias of the analyte concentration is likely to be observed due to the surrounding cell types, complicating the results obtained. While some tumor markers present in tissue biopsies have been used with ER positive patients with Tamoxifen and the treatment of patients with tumors over-expressing HER-2/neu with HERCEPTIN™, many questions regarding analyte expression in cancer still remain.

Breast carcinoma tissue biopsies are composed of not only of the carcinoma cells themselves, but also of infiltrating endothelial cells, fibroblasts, macrophages and lymphocytes. The stroma surrounding the cancer cells provides the necessary vascular support and extracellular matrix molecules that are required for tumor growth and progression [12]. There has recently been growing evidence in the importance of stromal cell contributions to the developing tumor (e.g., [12-28]). An early investigation of breast tumor stromal and epithelial cell lines derived from human tissues indicated that the enzyme aromatase is present in stroma within breast tumors and suggests estrogen synthesis from within the tumor may modulate growth by a paracrine mechanism [29]. A study investigated differences in gene expression between breast carcinoma cells and the surrounding stromal cells, in which they detected a number of genes which may aid in the understanding of stromal responses to the presence of a nearby tumor [23]. Cancer progression may involve matrix metalloproteinases (MMPs) ability to degrade the basement membrane.

In many solid tumors, MMPs are produced by the surrounding stromal cells, rather than the tumor cells themselves [27]. It has been determined that small differences in either stromal or tumor expression of certain MMPs (MMP-2/TIMP-2 or MMP-14) are associated with cancer progression [30]. Stromal cells have also shown to promote tumor growth and angiogenesis through secreting an elevated amount of SDF-1/CXCL12, which can bind to its cognate receptor CXCR4 expressed on the surface of tumor cells [24].

Experiments were performed to determine optimal yield and analyses of mRNA obtained from small quantities of cancer tissues. This included tissue preparation, techniques in LCM, RNA extraction, purification and amplification, as well as development of quality control analyses at each step in the procedure.

### Example 1

### Preparation of Breast Cancer Samples

### Methods and Materials

### Processing of Human Tissue Specimens

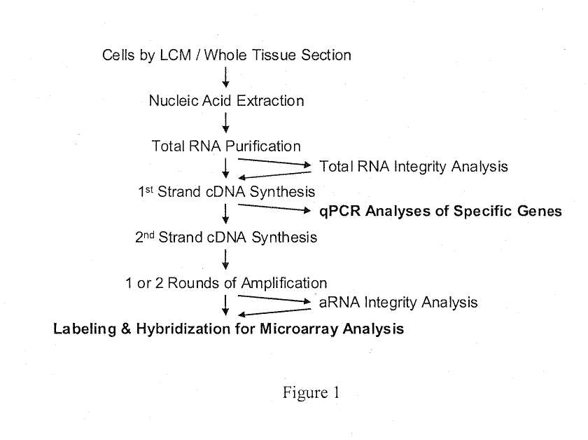

To evaluate differences between cell types, either whole tissue specimens or isolate the cells of interest by LCM was extracted for DNA, RNA or protein analyses [37; 38; 135-137]. FIG. 1 illustrates the protocol used for gene expression analysis of de-identified frozen tissue sections or of LCM-procured cells. The first step in this process was the proper preparation of the tissue, so that optimal results were obtained from downstream applications (i.e., qPCR or microarray).

Before the handling of any patient encoded information or results, Collaborative Institutional Training Initiative (CITI) training and Health Insurance Portability and Accountability Act (HIPAA) certification were obtained. All specimens and follow-up information were de-identified and encoded in the Tumor Marker™ database, and no identifiers were used in any part of this research as indicated in Institutional Review Board (IRB) protocols #334.05 and 583.06. Proper tissue procurement, specimen handling and cryopreservation were essential for the collection of quality information from these analyses (e.g., [11; 135]). As described by Wittliff and Erlander [38], archival biopsy specimens used in this study were expeditiously removed without trauma during the surgical procedure. Specimens were chilled on ice, and then trimmed of obvious necrotic tissue, leaving normal tissue present with the lesion in question. Tissue specimens were either frozen on dry ice in the pathology suite within 20-30 min of collection or rapidly transported chilled in a Petri dish or plastic bag immersed in ice prior to cryopreservation and frozen section preparation in the LCM laboratory, to retain the biological integrity of macromolecules [38]. Procedures avoiding RNase and DNA contamination were employed, i.e., cleaning of bench area and utensils with RNase Away (Molecular BioProducts) or RNase Zap (Ambion). With the sensitive technologies of genomics and proteomics requiring nondestructive isolation of pure cell populations, new surgical pathology approaches and methods have been developed as recommended by Cole et al. [34] and Wittliff et al. [11; 37; 38].

Specimens were processed according to accepted biohazard policies in clean rooms/benches prepared to reduce RNase and DNA contamination and frozen in Optimum Cutting Temperature (O.C.T.). compound (TISSUETEK® OCT medium, VWR Scientific Products Corp.) and stored at −86° C. until sectioning and microdissection. At that time, frozen sections were collected on sterile, uncharged microscope slides that were retained frozen until use.

### Fixation, Staining, and Dehydration

Frozen sections mounted on uncoated glass slides were handled according to established procedures depending upon the type of staining reagent (e.g., [37; 38; 71; 138]). The intercalating dye, ToPro3 (Molecular Probes, Inc., Eugene, Oreg.), which binds to double stranded nuclei acids and exhibits a peak fluorescence at 661 nm, has been used in previous studies to assess the integrity of DNA in vivo in LCM-procured cells [38].

Prior to analyses in an RNase-free setting, the structural status of the tissue was evaluated after sectioning and staining with hematoxylin and eosin (H & E), using a modified staining protocol (Table 1) [38; 138]. This modified protocol was used to shorten the time required, and thus reduce RNA degradation, while adequately staining the sections for visualization of cell types. The slides were prepared for the LCM process by dehydration with absolute ethanol, and coating of the tissue sections with xylenes, which helped prevent re-hydration. In an H & E stained tissue section from a representative breast cancer specimen, where a prevalence of carcinoma cells invaded the adjacent stroma, the structural integrity of the tissue section indicated that the biopsy was acceptable to proceed with LCM and gene expression analyses. Immunohistochemistry (IHC) of protein analytes (e.g., estrogen receptor, progestin receptor, HER-2/neu and epidermal growth factor (EGF) receptor) has been performed in previous studies [38] of invasive ductal carcinoma using mouse monoclonal antibodies TAB250 and AB10 (Clone 111.6) against HER-2/neu protein and EGF Receptor, respectively, to guide selection of cells exhibiting particular protein analytes of clinical interest H &E staining of either analyte occurs primarily at the cell membrane of carcinoma cells. HISTOGENE™ Frozen Tissue Staining Kit (Arcturus Bioscience) and an LCM Staining Kit (Ambion, Austin, Tex.) have been specially developed to aid visualization of cells, while minimizing degradation of RNA for laser capture [139].

TABLE 1
H & E staining protocol utilized in these studies.

| CHEMICAL | INSTRUCTIONS | |
|---|---|---|
| 70% ethanol | Immersed for 60 sec. | |
| RNase-free water | 6 | dips |
| Hematoxylin I from filtered syringe | 5 | sec |
| RNase-free water | 6 | dips |
| 70% ethanol | 6 | dips |
| Eosin Y | 6 | dips |
| 95% ethanol | 6 | dips |
| 100% ethanol | 10 | dips |
| 100% ethanol | 10 | dips |
| 100% ethanol | Immersed for 30 sec | |
| 100% ethanol | Immersed for 1 min | |
| Xylene | 6 dips then immersed for 30 sec | |
| Xylene | Immersed for 1 min | |
| Air dry | 1-2 | min |

### Intact Tissue Section Analyses

Analysis of the intact tissue section is vitally important to ensure extraction of high-quality RNA of sufficient quantity prior to the tedious LCM process. For these quality control studies, tissue was processed in an RNase-free manner and stained by H & E with a protocol identical to that employed for tissue sections used for LCM. This quality control step ensures there is no difference attributable to the staining step in the extent of RNA degradation in each of the sample preparations, i.e., intact tissue section and microdissected cells. However, H & E staining may alter the quantity of RNA extracted relative to that of unstained sections.

Gene expression analyses of intact tissue sections was warranted. Two methods of preparing intact tissue sections from frozen biopsies were refined [38]. The first involved preparation of frozen tissue sections in the cryostat (−20° to −25° C.) without the use of a glass slide. As a tissue section was cut (7-25 μm), it formed a “curl” which was placed directly into an RNase-free microcentrifuge tube for nucleic acid or protein extraction. This simple procedure has the advantage of allowing collection and storage at −80° C. of multiple samples from the same tissue specimen. Additionally, samples from a multitude of specimens may be prepared and stored in order to process them simultaneously for RNA or protein extraction to ensure uniform handling. The other method involved the collection of frozen tissue sections (5-10 μm) on RNase-free, uncharged glass slides in the cryostat (−20° to −25° C.), which were then stored at −80° C. without cover-slips. To ensure there was no contact between frozen tissue sections, slides were stored in 100-count slide boxes.

### RNA Isolation and Characterization

Maintaining the integrity of labile mRNA is paramount to obtaining high-quality results from qPCR and microarray analyses. When using frozen tissue “curls,” 350 μl of extraction buffer (RLT with β-mercaptoethanol) from the QIAGEN (Valencia, Calif.) RNEASY® RNA isolation kit was added to the microcentrifuge tube and incubated on ice for 5 min and mixed briefly using a VORTEX GENIE™, before centrifugation to sediment the cell debris and O.C.T. embedding compound. These and all subsequent RNA isolation and characterization steps were conducted in an RNase-free setting.

As in the procedure for extracting frozen tissue “curls,” it was unnecessary to utilize H & E staining for tissue sections collected on uncharged slides. However, when preparing RNA from tissue sections collected on uncharged slides, the sections were fixed in 70% ethanol for 1 min at 25° C. prior to removing the O.C.T. embedding compound by dipping briefly in RNase-free water. In the absence of H & E staining, the slides were then transferred stepwise into 95% ethanol, then four separate transfers into separate tubes of 100% ethanol before brief exposure to 100% xylene in 2 separate tubes. After drying the slide at room temperature for 2-3 min, the fixed, unstained tissue section was ready for preparation of “scraped” samples.

In contrast to RNA preparation from “curls,” fixed tissue sections from frozen samples collected on slides were “scraped” from the slide surface by placing a small amount (175 μl) of the same extraction buffer onto the tissue section, then scraping the section with an RNase-free pipet tip to loosen it from the slide, while drawing the tissue suspension into the pipet tip. This step was repeated with the same volume of extraction buffer to remove any tissue fragments remaining on the slide.

Using either extraction technique, RNA was extracted using the QIAGEN RNEASY® RNA isolation kit, which included spin columns, a DNase treatment step, a series of washes and an elution to purify the RNA from the samples. Typically, 10-200 ng total RNA were isolated from a single 7 μm gross tissue section (Table 2). If only a small amount of RNA (e.g., less than 1 ng for downstream microarray analyses, or less than 10 ng for downstream qPCR analyses) remained intact in this assessment of sample quality, then subsequent LCM procedures were not warranted.

Quality of RNA was evaluated by a variety of procedures, including with the Agilent RNA 6000 Nano or Pico Kits and the BIOANALYZER™ Instrument (Agilent Technologies). The BIOANALYZER™ can provide a numerical RNA Integrity Number (RIN) of the total RNA after electrophoretic separation, which utilizes 18S and 28S rRNA profiles to provide a quantitative assessment of the quality of RNA in the sample [140]. In general, a RIN value of greater than 7 is correlated with high quality RNA acceptable for genomic analyses.

The NANODROP™ (Nanodrop Technologies, Wilmington, Del.) Instrument determines RNA quantity and purity based on absorbance at 260 nm and 280 nm with the added feature that only 1 ul of sample is required. Analysis of intact RNA can also be performed using reverse transcription and qPCR. Since fragment gene sequences contained in degraded mRNA will not amplify, an estimate of total intact RNA can be determined from a standard curve of Universal Human Reference RNA (Stratagene, La Jolla, Calif.).

TABLE 2
Representative quantities of total RNA extracted from
intact breast carcinoma tissue sections before and after
H & E treatment illustrating the influence of staining.

| | RNA | RECOVERY (%) |
|---|---|---|
| SAMPLE | EXTRACTED | AFTER H & E |
| 1A (unstained) | 18.8 | |
| 1B (H&E | 17.1 | 91.0% |
| 2A (unstained) | 5.5 | |
| 2B (H&E | 4.1 | 74.5% |
| 3A (unstained) | 34.1 | |
| 3B (H&E | 14.6 | 42.8% |
| 4A (unstained) | 435.1 | |
| 4B (H&E | 344.9 | 79.3% |
| 5A (unstained) | 14.1 | |
| 5B (H&E | 2.7 | 19.1% |
| 6A (unstained) | 2.9 | |
| 6B (H&E | 0.8 | 27.6% |

Serial sections of each frozen tissue biopsy were either stained with H & E or left unstained. Total RNA was extracted as described in using each pair of sections and the mRNA quantity for each preparation was determined by qPCR, using β-actin as a reference gene, to evaluate the influence of H & E staining. Results are representative of the range of RNA recoveries in the H & E stained sections compared to those of unstained sections.

### Steps in Laser Capture Microdissection

Cells of interest were microdissected using the PIXCELL IIe™ with CAPSURE™ LCM Caps (Molecular Devices), which permitted collection of intact cells on the surface transfer film of the cap. For documentation purposes, a “Map” image was taken at 10× magnification, while LCM was performed at 20× magnification. The complete removal of carcinoma or stromal cells by LCM, were deposited on the surface of the LCM cap

Carcinoma and stromal cells were removed independently from heterocellular regions and procured cleanly for retention on the LCM caps. If necessary, CAPSURE™ Pads were utilized to remove cellular debris from the CAPSURE™ LCM Caps prior to nucleic acid extraction. CAPSURE™ pads (Arcturus Bioscience) were used to eliminate contaminating cells and debris during LCM. Stromal cells were transferred loosely bound to the LCM cap during collection of carcinoma cells.

The stromal cells adhered to the LCM-procured carcinoma cells bound to the film surface, showing that only carcinoma cells were retained on the cap surface after treatment of the specimen with a CAPSURE™ Pad.

RNA Isolation and Characterization from LCM-Procured Cells

Total RNA from laser captured cells was isolated using the PICOPURE® RNA Isolation kits (Molecular Devices), which were optimized for cells procured by LCM. This procedure utilizes a DNase (Qiagen) digestion step to eliminate DNA contamination. Typically, 1-6 ng of total RNA were extracted from LCM-procured cells using 50 μl XB BUFFER™ (Arcturus), compared to 10-200 ng total RNA from a single 7 μm intact tissue section, in agreement with earlier studies [37; 38]. To demonstrate the yield and integrity of RNA obtained from either tissue sections or LCM, serial sections of a single specimen of representative invasive ductal carcinoma of the breast were prepared and one section was left unstained, while another was stained with H & E (Table 3). The third section was subjected to LCM for procurement of cancer cells only (2221 laser pulses). The representative results shown in Table 3 are typical of the greatest differences observed between total RNA quantities extracted from H & E stained sections compared to unstained sections. As predicted, the quantity of total RNA in the LCM-procured cell preparation varied with the number of cells captured. Other kits designed for isolation of total RNA from small samples (e.g., those obtained by LCM) are also commercially available, including RNAQUEOUS™-MicroKit (Ambion), ARRAYPURE™ (Epicentre, Madison, Wis.), PURELINK™ (Invitrogen, Carlsbad, Calif.) and CELLSDIRECT™ (Invitrogen). Although their use was explored, the PICOPURE® kits provided optimal and reproducible results. After total RNA was isolated from the sample, characterization analyses (e.g., quality and quantity) were performed before proceeding to gene expression analyses, such as qPCR or microarray.

TABLE 3
Representative results showing the quantity of total
RNA extracted under different conditions using tissue
sections from a de-identified breast cancer specimen.

| | EXTRACTED RNA |
|---|---|
| SAMPLE | (ng) |
| Unstained | 19.7 |
| H & E stained | 12.3 |
| Cancer cells on LCM cap | 5.6 |

Serial sections of a single specimen of representative invasive ductal carcinoma of the breast were prepared and one section was left unstained, while another was stained with H & E. The third section was subjected to LCM for procurement of cancer cells only (2221 laser pulses). The representative results are typical of the greatest differences observed between total RNA quantities extracted from H & E stained sections compared to unstained sections. The quantity of total RNA in the LCM-procured cell preparation varied with the number of cells captured.

### First Strand Synthesis

In order to analyze gene expression by qPCR, cDNA must be reverse transcribed from the isolated total RNA. Two types of primers may be utilized for reverse transcription reactions: random hexamers or oligo (dT) primers (e.g., [84]). Random hexamers amplify most RNA species, including mRNA, tRNA and rRNA, while oligo (dT) primers preferentially amplify mRNA due to the presence of poly (A) tails [84]. A study by Hembruff et al. [84] found that oligo (dT) primers were superior to random hexamers after RNA isolation by the RNEASY® method, because of less variability in expression of the S28 reference gene that is independent of the method of qPCR detection (i.e., Sybr green or TAQMAN® probes). Oligo (dT) primers were utilized with LCM procured cells because of the need for linear amplification prior to microarray [37; 38].

Total RNA extracted from either the intact tissue section or LCM-procured cells was reverse transcribed in a solution of 250 mM Tris-HCl buffer, pH 8.3 containing 375 mM KCl, and 15 mM MgCl₂ (Invitrogen), 0.1 M DTT (dithiothreitol, Invitrogen), 10 mM dNTPs (Invitrogen), 20 U/reaction of RNASIN™ ribonuclease inhibitor (Promega, Madison, Wis.) and 200 U/REACTION OF SUPERSCRIPT™ III RT (reverse transcriptase, Invitrogen) with 5 ng T7 primers. The cDNA obtained from this reverse transcription reaction was diluted 10-fold in 2 ng/ul polyinosinic acid and used in qPCR reactions. Other commercial kits for cDNA synthesis: ISCRIPT™ (Biorad), TRANSCRIPTOR™ (Roche Diagnostics, Indianapolis, Ind.) and MONSTERSCRIPT™ (Epicentre) were explored. A methodology designed by Miltenyi Biotech, which utilizes a magnetic bead-based isolation of RNA and reverse transcription reaction (μMACS™), provides cDNA in a simple procedure over a significantly shorter period of time. However, SUPERSCRIPT™ III RT (Invitrogen) provided the greatest latitude in preparation and use of cDNA for a variety of applications.

qPCR Analyses of Gene Expression The qPCR reactions were performed in either a 96-well plate using a total volume of 25 μl/well or in a 384-well plate using a total volume of 10 μl/well. The reactions contained POWER SYBR™ Green PCR Master Mix (Applied Biosystems, Foster City, Calif.), forward primer, reverse primer and diluted cDNA obtained from the reverse transcription reaction. SYBR green is a fluorophore that binds to double-stranded DNA that is produced during each cycle of amplification [84]. Many other SYBR Green master mixes are also commercially available, such as FASTSTART™ (Roche Diagnostics), ISCRIPT™ (BioRad) and TAQURATE™ (Epicentre). Reactions can also be performed utilizing fluorescent probes, such as TAQMAN® (Applied Biosystems), which provide a high degree of sensitivity and specificity. However, studies performed by Hembruff et al. determined that the sensitivity of Sybr green was sufficiently high and was the preferred method of product detection due to its lower cost [84]. Although primers used in these investigations were designed with PRIMER EXPRESS™ (Applied Biosystems), both primers and probes were purchased pre-designed from a commercial source, such as Applied Biosystems. Primers were designed for sequences closer to the 3′ end of the transcript when using a T7 (oligo (dT)) primer in the reverse transcription reaction, due to degradation which may occur near the 5′ terminus.

The threshold cycle number (Ct value) was the cycle of amplification at which the qPCR system recognizes an increase in the signal (i.e., Sybr green) associated with the exponential growth of the PCR product during the log-linear phase. These Ct values were compared to those of a reference gene, such as glyceraldehyde phosphate dehydrogenase (GAPDH) or β-actin (ACTB), to obtain a ΔCt value [141; 142]. Amplification of the reference gene also serves as a positive control for efficiency of the qPCR reaction. Expression of the gene of interest (as a ΔCt value) was then compared to that of the same gene in the calibrator, i.e., Universal Human Reference RNA (Stratagene, La Jolla, Calif.), in order to obtain a ΔΔCt value. This ΔΔCt value is then converted to a relative expression level for the gene of interest (relative gene expression=2^{-ΔΔCt}). This method of analyses is known as the ΔΔCt method of calculating relative gene expression [141].

### Results and Discussion

### Assessment of RNA Yield and Integrity

In preparation for genomics studies utilizing LCM-procured cells, RNA yield and integrity analyses of the cognate intact tissue section must be performed. If a direct comparison is to be made between LCM-procured cells and intact tissue, the specimens should be treated identically, including the thickness of the tissue section and staining protocol. However, if gene expression is to be determined only on intact tissue sections, it is preferable to use “tissue curls” as described maintaining consistent procedures with each tissue biopsy. Although considerable variation was noted in the cellular content and contaminating elements of the various human breast carcinoma biopsies investigated, using the tissue preparation and processing protocols appeared to enhance the reproducibility of the results.

Since there are many techniques for determining quality and quantity of total RNA, experiments were conduced to select the optimal method (Table 4) to obtain the minimal yield of RNA of high quality necessary for downstream application, e.g., qPCR. As shown in Table 4, measurements of quantity and quality of RNA obtained from eleven different representative breast tissue specimens were performed using three independent methods: Agilent BIOANALYZER™, NANODROP™, and qPCR with a known Universal Human Reference RNA (Stratagene). A comparison of these methods gave highly variable results, as expected with these completely different technologies (Table 4). However, there appeared to be greater agreement in the estimates of total RNA using the Agilent BIOANALYZER™ compared to those from the NANODROP™ Instrument. Values obtained from qPCR were much lower, apparently due to the fact that only mRNA that has been reverse transcribed is measured. For the examples shown in Table 4, 8 of the 11 samples evaluated had sufficient intact RNA (about >10 ng/ul estimated by the BIOANALYZER) for either qPCR analysis of specific genes, amplification for microarray hybridization, or proceeding to LCM and RNA extraction.

TABLE 4
Comparison of the quantity and quality of RNA obtained from
eleven different breast tissue specimens using three independent
methods: Agilent BIOANALYZER ™, NANODROP ™, and
qPCR with a known Universal Human Reference RNA (Stratagene).
Briefly, total RNA was extracted and purified from 7 □m tissue
sections as described in Methods and Materials, then evaluated by
each of the three methods. Profile evaluation, while subjective, was
based on the comparison with appearance of 18S and 28S rRNA
in a reference sample.

| | AGILENT | | | |
|---|---|---|---|---|
| | BIOANALYZER ™ | | NANODROP ™ | qPCR |
| SAMPLE | PROFILE | [RNA] ng/ul | [RNA] ng/ul | [RNA] ng/ul |
| 1 | poor | 9.7 | 4.2 | 1.5 |
| 2 | good | 21.4 | 11.7 | 9.1 |
| 3 | good | 16.2 | 10.5 | 13.3 |
| 4 | good | 19.6 | 11.9 | 8.6 |
| 5 | good | 16.5 | 12.8 | 10.4 |
| 6 | good | 10.1 | 6.8 | 3.7 |
| 7 | good | 24.9 | 11.9 | 6.6 |
| 8 | poor | 5.4 | 2.3 | 0.6 |
| 9 | good | 54.4 | 30.6 | 9.2 |
| 10 | poor | 2.2 | 3.3 | 0.1 |
| 11 | good | 22.9 | 10.3 | 5.1 |

Since results from the BIOANALYZER™ provide a reproducible estimate of RNA quality and quantity in a sample, unlike those of the NANODROP™ instrument, and use of the BIOANALYZER™ is considerably less expensive and time consuming compared to qPCR, the Bioanalyzer was employed in the standardized protocol. Representative BIOANALYZER™ profiles from analyses of total RNA extracted from tissue sections of four different human breast carcinoma specimens showed varying yields and quality. For example, one extract produced a low RNA yield (10 ng/ul) of high quality (28S/18S=1.1), a second produced a low RNA yield (12 ng/ul) of poor quality (28S/18S=0.0), a third produced a high RNA yield (195 ng/ul) of the highest quality (28S/18S=1.0) RNA, and a fourth produced a high RNA yield (157 ng/ul) that was degraded (28S/18S=0.3). A similar instrument, EXPERION (BioRad, Hercules, Calif.), also provides a rapid, and reproducible separation and analysis of protein and nucleic acid samples, and provides similar data analyses including a concentration, 28S/18S ratio, and a RQI (RNA quality indicator) value.

If the yield of RNA is low or of marginal quality, additional tissue sections or LCM-procured cells may be processed from serial tissue sections in different regions of the O.C.T. block, and the RNA extracted may be pooled. Using this approach, few human breast carcinoma specimens have been rejected. If necessary, the isolated RNA may be concentrated using a SPEEDVAC™ (Savant), or similar product.

Assessment of Yield and Integrity of RNA from LCM-Procured Cells

The ability to procure homogeneous cell sub-populations of normal stromal and malignant cell types, and to generate genomic and proteomic results from each cell type advances the understanding of the underlying causes of tumor formation. Furthermore, this approach permits the tracking of cell progression into a metastatic phenotype at the molecular level. To examine gene expression in carcinoma and stromal cells from a breast cancer biopsy, frozen tissue blocks were processed as serial 7 μm sections as shown in FIG. 1. At least 1000 breast carcinoma cells and 1000-2000 breast stromal cells were procured from tissue sections for RNA extraction and analyses. Multiple cell captures were performed on many samples, and RNA was pooled to obtain sufficient quantities for qPCR reactions (Table 5). Firstly, it should be noted that a single LCM pulse cannot be equated with the capture of a single cell, since both cell size and the dimension of the LCM laser-induced spot can be adjusted to 7.5, 15 and 30 μm depending on the power and duration of the laser [38]. In the majority of studies described, the 7.5 μm spot was utilized, because it allowed greater definition during cell collection. As shown in Table 5, the yield of RNA per laser pulse was similar for carcinoma and stromal cells, regardless of the number of pulses used in a single tissue section. BIOANALYZER™ analyses confirmed the integrity of the extracted RNA, although the RNA profiles from stromal cell extracts indicated increased amounts of RNA species with molecular weights lower that 18S rRNA. It is unknown if these low molecular weight RNA species are related to the presence of native RNA molecules or simply to RNA degradation during LCM collection. Regardless, RNA of the qualities illustrated provided reproducible results when gene expression was measured by qPCR.

Although the PIXCELL IIe™ LCM System and Image Archiving Workstation (Arcturus Bioscience, Inc.) was employed because it was the only instrument available, other systems have been developed for cell collection from tissue sections. The P.A.L.M. (P.A.L.M. Microlaser Technologies, Bernried, Germany) instrument utilizes both laser microdissection and pressure catapulting. Molecular Machine & Industries (MMI, Glattbrugg, Switzerland) has developed two instruments, the mmi CELLCUT™ and the mmi SMARTCUT™, which procure cells of quality similar to that of the PIXCELL IIe™. A new generation LCM instrument, the VERITAS™, was developed by Arcturus Bioscience to combine the technologies of laser capture and laser cutting, utilizing both an ultraviolet and infrared laser [46]. Each of these instruments allows microdissection of either single cells or groups of cells [45].

TABLE 5
Representative quantities of RNA extracted from LCM-procured cells.
Individual populations of either carcinoma or stromal cells were
obtained by LCM from tissue sections processed as described in
Methods and Materials.

| | | TOTAL No. OF | | |
|---|---|---|---|---|
| | No. LCM | LASER | [RNA] | [RNA]/ |
| SAMPLE | CAPS | PULSES | (ng/ul) | PULSE (×10⁴) |
| 1 - cancer cells | 2 | 7,730 | 3.7 | 4.8 |
| 1 - stromal | 2 | 8,282 | 1.3 | 1.6 |
| cells | | | | |
| 2 - cancer cells | 2 | 12,824 | 7.1 | 5.5 |
| 2 - stromal | 2 | 7,522 | 4.7 | 6.3 |
| cells | | | | |
| 3 - cancer cells | 2 | 4,790 | 4.2 | 8.8 |
| 3 - stromal | 2 | 2,024 | 1.8 | 8.9 |
| cells | | | | |
| 4 - cancer cells | 3 | 9,565 | 7.2 | 7.5 |
| 4 - stromal | 3 | 5,042 | 2.7 | 5.4 |
| cells | | | | |
| 5 - cancer cells | 2 | 7,779 | 10.1 | 13.0 |
| 5 - stromal | 2 | 5,265 | 5.6 | 10.6 |
| cells | | | | |
| 6 - cancer cells | 1 | 8,250 | 3.8 | 4.6 |
| 6 - stromal | 1 | 4,230 | 1.5 | 3.5 |
| cells | | | | |
| 7 - cancer cells | 2 | 8,378 | 13.6 | 16.2 |
| 7 - stromal | 1 | 5,562 | 9.8 | 17.6 |
| cells | | | | |

RNA was extracted with the PicoPure RNA Isolation kit and characterized with the Agilent BIOANALYZER ™.

Gene Expression Analyses by qPCR

The choice of a reference gene is vitally important for normalizing data obtained in qPCR reactions. The reference gene chosen must be evenly expressed across samples and amplify with the same efficiency as the genes of interest, in order to ensure that differences observed in the genes of interest reflect the biological status of the specimen. Although an investigation [142] reported that greater than 90% of published gene expression studies in high impact journals prior to 1999 utilized GAPD, ACTB, 18S and 28S rRNA as single genes for normalization, other investigators question whether any single gene is ideal (e.g., [84; 143]). Their suggestions include the use of total RNA or panels of reference genes. Although most studies focused on identification of genes whose expression levels remained constant in a variety of cell types, use of a single tissue or cell type suggests the reference gene should remain constant in that particular tissue (e.g., [143]). This may be confirmed by analyses of several tissue samples, each with known RNA concentrations, as suggested by Suzuki [142]. In order to assess this quality, the following study was performed. Each of eight RNA samples of a breast tissue panel was diluted to the same concentration and re-quantified by spectroscopy (NANODROP™) to confirm the concentrations. The RNA was reverse transcribed and subjected to qPCR for the reference gene of interest, such as ACTB (Table 6). Results from these eight samples gave an average Ct value of 18.58 with a standard deviation of 0.54, indicating a relatively low amount of variation of ACTB expression among samples. Thus ACTB was employed as the reference gene in the standardized protocol for breast tissue.

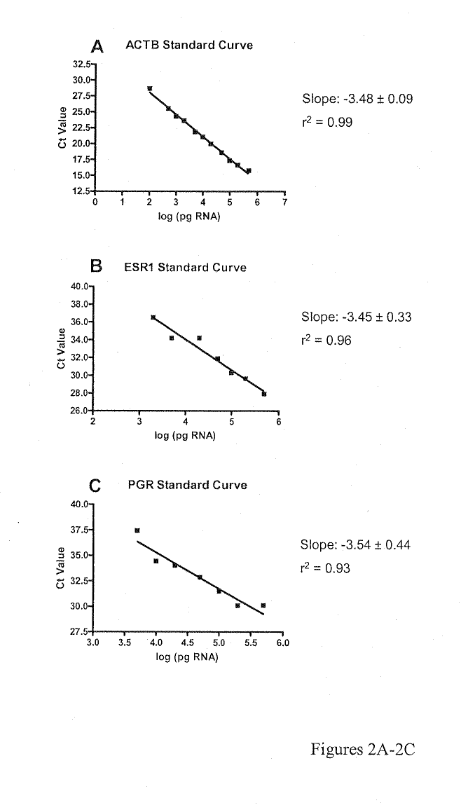

To ensure accuracy of gene expression measurements, genes of interest should have similar amplification efficiencies. Representative standard curves (FIG. 2) of qPCR analyses of ACTB (FIG. 2A), ESR1 (FIG. 2B) and PGR (FIG. 2C) genes measuring relative expression are shown. Dilutions were prepared with cDNA made from Universal Human Reference RNA (Stratagene) resulting in linear relationships (FIG. 2). Similar amplification efficiencies were illustrated for the three genes: FIG. 2A exhibited a regression line with a slope of −3.48 with an r²=0.99; FIG. 213 shows a regression line with a slope of −3.45 with an r²=0.96; Graph C shows a regression line with a slope of −3.55 with an r²=0.93. The similar slopes of these graphs illustrate that the genes examined have similar amplification efficiencies (i.e., slope of ACTB±0.1), which is vital for normalization of gene expression [144]. Efficiency is calculated using the equation: E=10^{(-1/slope)}. Ideally, genes should amplify with a slope of −3.3, which results in efficiency of 2, indicating a perfect doubling of template DNA during the PCR amplification [144].

TABLE 6
Representative results evaluating ACTB as a normalizing gene
for use in gene expression studies of human tissue. Tissue
biopsies from eight de-identified invasive ductal carcinomas were
sectioned and total RNA was extracted as described in Methods
& Materials.

| | AVERAGE |
|---|---|
| SAMPLE | Ct VALUE |
| 1 | 18.90 |
| 2 | 18.05 |
| 3 | 17.99 |
| 4 | 19.01 |
| 5 | 18.73 |
| 6 | 19.39 |
| 7 | 17.92 |
| 8 | 18.65 |

Average Ct value 18.58 ± 0.54 SD
Total RNA from each of these samples was diluted to the same concentration and re-quantified to confirm the concentrations. RNA in each sample was reverse transcribed, then expression of the ACTB gene was determined in duplicate by qPCR and recorded as average Ct

Another validation of qPCR results was performed using a dissociation curve analysis. At the conclusion of PCR amplification of target genes, an additional anneal and melt cycle was performed on the PCR products over an extended period of time with fluorescence measured over the entire cycle. The presence of a single peak in fluorescence indicated a single PCR product), while multiple peaks) indicated formation of products, such as primer dimerization or non-specific products as suggested by Bookout [144].

It is widely accepted that many investigations of genomics and proteomics of human tissues utilized biopsy specimens collected, stored, and processed using a variety of conditions, many of which were unstandardized. The concern is of such a magnitude that the National Cancer Institute has established “Best Practices for Biospecimen Resources” focusing on collection of human tissue specimens and associated data for research purposes. In the current investigation, procedures and conditions were refined [37; 38] for processing de-identified human tissue biopsies in preparation for microgenomic-based investigations in an RNase-free setting. These include the establishment of standardized protocols for RNA purification and amplification using both frozen tissue sections and LCM-procured cells.

It was demonstrated that the total RNA extracted from either thin tissue sections of individual cell populations (e.g., carcinoma or stromal cells) was of high quality providing meaningful results. Furthermore, standardized conditions were developed to improve RNA yields from LCM-procured cells, as well as from thin (7-10 μm) intact tissue sections, such that microgenomic analyses could be performed reproducibly. Results were obtained demonstrating that ACTB was a valid reference gene for normalization of qPCR results, since its expression levels remained constant among a wide variety of human breast carcinomas, and its efficiency of amplification was similar to those of target genes. Nucleic acid dissociation curve analyses confirmed the quality of PCR products formed for analysis of gene expression. Collectively, these results confirm that the procedures for tissue and cell processing for subsequent isolation of intact mRNA were applicable for assessing the expression of candidate genes.

### Development of a Small Gene Set Related to Clinical Significance

Global gene expression using microarrays has been explored as a means to determine molecular profiles reflecting breast cancer behavior (e.g., [41; 47; 48; 50-73]). Expression profiles are proposed to provide a more accurate prediction of the clinical course of breast cancers than indicated by conventional tumor markers. However, there is great variation in methods and platforms utilized to obtain these gene expression profiles of cancer, including the use of breast cancer cell lines (e.g., [55; 134]), whole tissue extraction (e.g., [65; 73]), and LCM-procured cells (e.g., [41; 57; 70; 71]). In an attempt to identify a small, clinically relevant gene set, numerous “molecular signatures” of breast cancer reported to be related to clinical behavior were investigated (e.g., [41; 47; 48; 54; 55; 62-65; 67; 70]).

The eleven gene signatures described supra, without bias of gene selection, were investigated to derive a subset of candidate genes for development of a predictive test of risk of breast cancer recurrence.

### Example 2

### Gene Expression in Breast Tissue Samples

### Methods and Materials

GenBank Accession numbers (NCBI) of genes from studies of interest [47; 48; 54; 55; 62-64; 67; 70; 71; 75] were entered into the UniGene database (NCBI), which separates the GenBank sequences into a non-redundant set of gene-oriented clusters. There are 123,891 sequence entries for Homo sapiens. Each UniGene Cluster contains sequences that represent a unique gene, which has a specific identifier. Once the appropriate UniGene identifier is known, the gene sets can be sorted by the UniGene identifier and analyzed. For example, epidermal growth factor receptor (EGFR) has a GenBank Accession number of NM_{—}201284. Entry of this Accession number into the UniGene database identifies UniGene Cluster Hs.488293 Homo sapiens Epidermal growth factor receptor (erythroblastic leukemia viral (v-erb-b) oncogene homolog, avian) (EGFR). Twenty-six mRNA sequences have been entered including NM_{—}201284. In addition 335 expressed sequence tag (EST) sequences have been entered. Using this approach, one may identify a variety of sequences associated with a single gene (Table 7).

TABLE 7
Representative unigene analyses of three independent gene sets.

| GENBANK | | | | |
|---|---|---|---|---|
| ACCESSION | | | Wittliff, et al. 2003 | |
| NUMBER OF | UNIGENE | GENE | van't Veer, et al. 2002 | |
| GENE ID | ID | NAME | Sorlie, et al. 2003 | |
| AW473119 | NM_000125 | ESR1 | Hs.208125 | Estrogen receptor- |
| AL050116 | | | | alpha (ESR1) |
| U95089 | AK000106 | | Hs.488293 | Epidermal growth |
| AK026818 | | | | factor receptor |
| | | | | (ERBB3) |
| BF108852 | | ERBB2 | Hs.446352 | ERBB2 |
| | | | | (HER-2/neu) |

To illustrate the sequence relationship described in the three independent studies, GenBank Accession Numbers or gene IDs were matched to the cognate gene. Once the UniGene identifiers were compiled into a Microsoft Excel spreadsheet, they were imported into Microsoft Access, where they were analyzed collectively. A Tier 1 level of comparison identified any gene that appeared in at least two molecular signatures, while a Tier 2 comparison identified any gene that appeared in at least three signatures. To identify genes that appear most relevant in breast carcinoma cells compared to those of surrounding stromal cells, the Tier 2 genes were separated into two groups. One group contains genes which appeared in that gene sets described by Wittliff and co-workers [41; 70] using only carcinoma cells procured by LCM, while another group, derived by elimination, was composed of genes that did not appear their “cancer” gene sets. This latter group of genes, which was tentatively assigned to stromal cells, was explored for their contribution to breast cancer behavior.

Comparisons of the 12 molecular signatures [47; 48; 54; 55; 62-65; 67; 70] reporting 2604 total Unigene sequences were analyzed. While 354 genes appeared in at least two of the signatures reported to be clinically relevant, only 32 genes appeared in at least three of these signatures (Table 8). Of the 32 genes present in at least three signatures, only 14 were reported in studies utilizing LCM-procured carcinoma cells (Table 9), while 18 were not (Table 10). This supports the suggestion that cells surrounding a malignant lesion are important in cancer progression (e.g., [12-30; 32]), since the 18 genes were identified as clinically relevant in at least three independent investigations using intact tissue. Some of these genes are reported (e.g., [11; 148-152]) to play a role in tumorigenesis or progression (e.g., ESR1 and NAT1), while others appear to be genes that are not associated with tumorigenesis (Table 11).

TABLE 8
Genes appearing in at least three molecular signatures of the eleven reports
identify 32 genes.

| GENE ID | MOLECULAR SIGNATURES REPORTED | | | |
|---|---|---|---|---|
| ESR1 | Sorlie | Sotiriou | Wittliff-ER | Vant Veer-ER |
| BUB1 | Sotiriou | Ma-high grade | Vant Veer-ER | Vant Veer-Prog. |
| TRIM29 | Sotiriou | Sorlie | Wittliff-ER | Vant Veer-ER |
| SCUBE2 | Wittliff-ER | Vant Veer-ER | Vant Veer-Prog. | Sorlie |
| SLC39A6 | Wittliff-ER | Sotiriou | Sorlie | Vant Veer-ER |
| FUT8 | Vant Veer-ER | Sotiriou | Vant Veer-Prog. | |
| EVL | Vant Veer-ER | Vant Veer-Prog. | Wittliff-ER | |
| NAT | Vant Veer-ER | Sorlie | Wittliff-ER | |
| CENPA | Vant Veer-ER | Vant Veer-Prog. | Ma-high grade | |
| MELK | Sotiriou | Vant Veer-ER | Vant Veer-Prog. | |
| PFKP | Vant Veer-ER | Sotiriou | Vant Veer-Prog. | |
| GABRP | Wittliff-ER | Vant Veer-ER | Sotiriou | |
| PLK1 | Sotiriou | Ma-high grade | Wang | |
| ATAD2 | Vant Veer-Prog. | Ma-high grade | Wang | |
| ST8SIA1 | Sotiriou | Vant Veer-ER | Wittliff-ER | |
| XBP1 | Vant Veer-ER | Sorlie | Sotiriou | |
| MCM6 | Sotiriou | Vant Veer-Prog. | Vant Veer-ER | |
| PTP4A2 | Sorlie | Vant Veer-ER | Sotiriou | |
| YBX1 | Sorlie | Sotiriou | Vant Veer-ER | |
| TBC1D9 | Wittliff-ER | Vant Veer-ER | Vant Veer-Prog. | |
| LRBA | Sotiriou | Sorlie | Vant Veer-ER | |
| GATA3 | Sotiriou | Sorlie | Vant Veer-ER | |
| CX3CL1 | Vant Veer-ER | Sorlie | Sotiriou | |
| IL6ST | Vant Veer-ER | Sotiriou | Wittliff-ER | |
| MAPRE2 | Vant Veer-Prog. | Sotiriou | Vant Veer-ER | |
| GMPS | Sotiriou | Vant Veer-Prog. | Vant Veer-ER | |
| RABEP1 | Wittliff-ER | Jansen | Sotiriou | |
| TPBG | Wittliff-ER | Vant Veer-ER | Wittliff-Recur. | |
| CKS2 | Vant Veer-Prog. | Ma-high grade | Sotiriou | |
| TCEAL1 | Wittliff-ER | Sotiriou | Vant Veer-ER | |
| DSC2 | Wittliff-ER | Sotiriou | Vant Veer-ER | |
| SLC43A3 | Vant Veer-ER | Wittliff-ER | Vant Veer-Prog. | |

References: Jansen, et al. [54] (“Jansen”), Ma, et al. [47] (“Ma”), Sorlie, et al. [63] (“Sorlie”), Sotiriou, et al. [64] (“Sotiriou”), van't Veer, el al. [65] (“van't Veer”), Wang, et al. [67] (“Wang”), Wittliff, et al. [70] (“Wittliff”)

TABLE 9
Gene set proposed for breast carcinoma cells derived by filtering
expression results describing the twelve molecular signatures.

| | UNIGENE ID | GENE NAME |
|---|---|---|
| 1 | Hs.125867 | EVL: Enah/Vasp-like |
| 2 | Hs.591847 | NAT1: N-acetyltransferase 1 (arylamine |
| | | N-acetyltransferase) |
| 3 | Hs.208124 | ESR1: Estrogen receptor 1 |
| 4 | Hs.26225 | GABRP: Gamma-aminobutyric acid (GABA) |
| | | A receptor, pi |
| 5 | Hs.408614 | ST8SIA1: ST8 alpha-N-acetyl-neuraminide |
| | | alpha-2,8-sialyltransferase 1 |
| 6 | Hs.480819 | TBC1D9: TBC1 domain family, member 9 |
| | | (with GRAM domain) |
| 7 | Hs.504115 | TRIM29: Tripartite motif-containing 29 |
| 8 | Hs.523468 | SCUBE2: Signal peptide, CUB domain, EGF-like 2 |
| 9 | Hs.532082 | IL6ST: Interleukin 6 signal transducer |
| | | (gp130, oncostatin M receptor) |
| 10 | Hs.592121 | RABEP1: Rabaptin, RAB GTPase binding |
| | | effector protein 1 |
| 11 | Hs.79136 | SLC39A6: Solute carrier family 39 (zinc |
| | | transporter), member 6 |
| 12 | Hs.82128 | TPBG: Trophoblast glycoprotein |
| 13 | Hs.95243 | TCEAL1: Transcription elongation factor A |
| | | (SII)-like 1 |
| 14 | Hs.95612 | DSC2: Desmocollin 2 |

TABLE 10
Gene set proposed for breast stromal cells derived by filtering
expression results describing the twelve molecular signatures.

| | UNIGENE ID | GENE NAME |
|---|---|---|
| 1 | Hs.1594 | CENPA: Centromere protein A |
| 2 | Hs.184339 | MELK: Maternal embryonic leucine zipper kinase |
| 3 | Hs.26010 | PFKP: Phosphofructokinase, platelet |
| 4 | Hs.592049 | PLK1: Polo-like kinase 1 |
| 5 | Hs.370834 | ATAD2: ATPase family, AAA domain containing 2 |
| 6 | Hs.437638 | XBP1: X-box binding protein 1 |
| 7 | Hs.444118 | MCM6: MCM6 minichromosome maintenance |
| | | deficient 6 |
| 8 | Hs.469649 | BUB1: BUB1 budding uninhibited by |
| | | benzimidazoles 1 homolog |
| 9 | Hs.470477 | PTP4A2: Protein tyrosine phosphatase type |
| | | IVA, member 2 |
| 10 | Hs.473583 | YBX1: Y box binding protein 1 |
| 11 | Hs.480938 | LRBA: LPS-responsive vesicle trafficking, |
| | | beach and anchor containing |
| 12 | Hs.524134 | GATA3: GATA binding protein 3 |
| 13 | Hs.531668 | CX3CL1: Chemokine (C—X3—C motif) ligand 1 |
| 14 | Hs.532824 | MAPRE2: Microtubule-associated protein, |
| | | RP/EB family, member 2 |
| 15 | Hs.591314 | GMPS: Guanine monphosphate synthetase |
| 16 | Hs.118722 | FUT8: Fucosyltransferase 8 (alpha (1,6) |
| | | fucosyltransferase) |
| 17 | Hs.83758 | CKS2: CDC28 protein kinase regulatory subunit 2 |
| 18 | Hs.99962 | SLC43A3: Solute carrier family 43, member 3 |

TABLE 11
Genes.

| | MOLECULAR/CELLULAR | |
|---|---|---|
| GENE ID | FUNCTION | |
| FUT8 | FUT8 transfers a fucose residue to | Glycosylation of cell |
| | N-linked oligosaccharides on | surface proteins is |
| | glycoproteins by an α1,6-linkage | important for biological |
| | [153]. The extracellular domain of | processes involved in |
| | the epidermal growth factor | cancer, such as |
| | receptor (EGFR), which contains | proliferation and |
| | 11 possible N-glycosylation sites, | metastasis [153]. |
| | can be fucosylated, and the | Increased FUT8 |
| | remodeling of N-glycan on EGFR | expression is associated |
| | have been shown to modulate its activity | with the progression of |
| | and function [153; 154]. | papillary carcinoma of the thyroid |
| | | [155]. |
| | | Fucosylation of EGFR |
| | | and sensitivity to gefitinib |
| | | was investigated, and |
| | | determined that cells with |
| | | over-expression of FUT8 |
| | | were more sensitive that |
| | | control cells [153]. |
| EVL | EVL is part of a family of multi- | EVL expression was up- |
| | functional proteins involved in | regulated in human breast |
| | actin-based motility. It has been | cancers compared to |
| | shown to be phosphorylated by | normal breast, and levels |
| | protein kinase D and concentrated | are correlated with clinical |
| | in cellular regions associated with | stages [158]. The |
| | movement and adhesion, included | microRNA hsa-miR-342 |
| | in the leading edge of lamellipodia, | located in an intron of |
| | filopodia, focal adhesions and | EVL is commonly |
| | adheren junctions [156; 157]. | suppressed in colorectal |
| | | cancers, and it was |
| | | suggested that hsa-miR- |
| | | 342 could function as a |
| | | proapoptotic tumor |
| | | suppressor [159]. |
| NAT | NAT1 metabolically activates | The high frequency of |
| | aromatic and heterocyclic amines | NAT1 acetylators |
| | to electrophilic intermediates that | genotypes are important |
| | initiate carcinogenesis [150; 160]. | modulators of cancer |
| | | susceptibility [150]. |
| | | Breast cancer tissues have |
| | | lower promoter |
| | | methylation rates than |
| | | normal breast, and DNA |
| | | hypomethylation of the |
| | | NAT1 gene plays a |
| | | significant role in breast |
| | | carcinogenesis [161]. |
| CENPA | CENPA is a centromere-specific | CENPA was differentially |
| | protein, which resides at the | expressed between ER- |
| | centromere at all stages on the cell | positive and ER-negative |
| | cycle, and it is essential for correct | breast cancer cell lines |
| | kinetochore assembly and function | [163]. It has also been |
| | [162]. | identified as a potential |
| | | biomarker of neoplastic |
| | | germ cells [164]. |
| MELK | MELK is a member of the | MELK was over- |
| | snf1/AMPK family of serine- | expressed in breast cancer |
| | threonine kinases, which are | cells, but not in normal |
| | associated with survival under cell | tissues [151]. Suppression |
| | stress [151; 165]. It has also been | of MELK by siRNA |
| | identified as a cell cycle regulator | significantly inhibited |
| | in cancer cell lines [165]. A pro- | growth of breast cancer |
| | apoptotic member of the Bcl-2 | cells, and it was suggested |
| | family, Bcl-G, was identified as a | that MELK promotes cell |
| | possible substrate for MELK | growth by inhibiting Bcl- |
| | [151]. | G though phosphorylation |
| | | [151]. Levels of MELK |
| | | expression was correlated |
| | | with pathologic grade of |
| | | brain tumors, and may be |
| | | a target for treatment of |
| | | high-grade brain tumors |
| | | [165]. |
| ESR1 | The product of ESR1 (estrogen | The protein product of |
| | receptor-α) binds estrogens, | ESR1 is the most |
| | dimerizes, binds to specific DNA | powerful predictor in |
| | sequences (EREs), and recruits co- | breast cancer for both |
| | activators or co-repressors to the | evaluating prognosis and |
| | transcription complex and either | predicting response to |
| | promote or repress transcription of | hormone therapy [166]. |
| | estrogen target genes [11]. | The estrogen receptor |
| | | pathway is the target of |
| | | the commonly used breast |
| | | cancer drug Tamoxifen. |
| PFKP | PFKP is the platelet-type isoform | It is known that cancer |
| | of 6-phosphofructokinase, which is | cells are highly dependent |
| | found in high levels in normal | on glycolysis. It was |
| | brain and catalyzes the rate- | shown that inhibition of |
| | limiting step of glycolysis [167]. | PFK in breast cancer cells |
| | | decreases viability by |
| | | inducing apoptosis; |
| | | however, the PFKP |
| | | isoform has not been |
| | | investigated [168]. |
| GABRP | GABRP encodes the α-subunit of | GABRP was found to |
| | the g-aminobutyric acid (GABA) | down-regulated in 76% of |
| | receptor, which is a | breast cancers and was |
| | transmembrane protein that is | progressively down- |
| | poorly understood [169; 170]. | regulated with tumor |
| | | progression [170]. |
| PLK1 | PLK1 is a member of a family of | PLK1 was up-regulated in |
| | serine-threonine kinases, which are | many invasive |
| | important regulators of cell cycle | carcinomas, including |
| | events, such as spindle formation, | NSC lung, head and neck, |
| | chromosome segregation, | esophageal, gastric, |
| | centrosome maturation, the | breast, ovarian, |
| | anaphase-promoting complex and | endometrial, colorectal |
| | cytokinesis [171]. | and thyroid cancers |
| | | [171; 172]. High PLK1 |
| | | levels were correlated |
| | | with active proliferation |
| | | and differentiation [172]. |
| | | Studies utilizing siRNA |
| | | against PLK1 in |
| | | combination with the |
| | | breast cancer drugs have |
| | | shown improved |
| | | sensitivity to paclitaxel |
| | | and Herceptin [173]. |
| ATAD2 | ATAD2 is a member of the AAA- | Studies of gene expression |
| | ATPase family, in which many | in osteosarcoma revealed |
| | members catalyze proteolysis, | increased ATAD2 |
| | protein complex disassembly, | expression is correlated |
| | protein unfolding, and cell division | with poor disease-free and |
| | [174]. | overall survival, and was |
| | | determined to be one of |
| | | the most powerful |
| | | predictors of survival in |
| | | these patients [175]. |
| ST8SIA1 | ST8SIA1 encodes GD3 synthase, | ST8SIA1 was shown to |
| | which is a ubiquitously expressed | have higher expression in |
| | type II membrane protein that | ER-negative breast tumors |
| | generates GD3 ganglioside by | [177]. Among ER-positive |
| | catalyzing the addition of a second | tumors, low expression of |
| | sialic acid residue to its immediate | ST8SIA1 is associated |
| | precursor GM3 [176]. | with worse prognosis |
| | | [178]. |
| XBP1 | XBP1 is an alternatively spliced | XBP1 has been shown to |
| | transcription factor that belongs to | be a key factor in anti- |
| | the basic region/leucine zipper | estrogen responsiveness |
| | family and is involved in the | and estrogen dependence |
| | unfolded protein response [179]. | in breast cancer cells, and |
| | | its expression has been |
| | | shown to correlate with |
| | | ESR1 in breast cancer |
| | | [179; 180]. |
| MCM6 | MCM6 is a member of the AAA+ | Both MCM2 and MCM6 |
| | family of proteins, and the MCM2- | are located in |
| | MCM7 complex is involved in | chromosomal regions |
| | initiation and elongation steps of | commonly amplified in |
| | DNA replication, and may be the | tumors, and MCM6 is |
| | replicative helicase [181; 182]. | significantly over- |
| | | expressed in tumors |
| | | compared to normal tissue |
| | | [182]. It has been |
| | | suggested that MCM6 be |
| | | evaluated as a marker and |
| | | predictor of survival in |
| | | lung cancer [182]. |
| BUB1 | BUB1 is a mitotic spindle | Mutations in BUB1 were |
| | assembly checkpoint gene that is | present in colon caner cell |
| | detected at the centromere region | lines, and these mutations |
| | in prophase [183]. It functions to | potentiate growth and |
| | block activity of the anaphase- | transformation [183; 185]. |
| | promoting complex until all | No mutations were found |
| | chromosomes are on the | in a study of breast cancer |
| | metaphase spindle [184]. | cell lines; however, there |
| | | was varying levels of |
| | | BUB1 gene expression |
| | | [185]. |
| PTP4A2 | PTP4A2 (or PRL-2) is a protein | Over-expression of PRL-2 |
| | tyrosine phosphatase that is | transformed mouse |
| | typically associated with the | fibroblasts and pancreatic |
| | plasma membrane and early | epithelial cell and promote |
| | endosome [186]. Its function | tumor growth in nude |
| | remains unclear; however, some | mice [188]. Another |
| | studies have suggested its | member of this family |
| | involvement in cell cycle control | (PRL-3) was significantly |
| | [186; 187] | up-regulated in metastatic |
| | | colorectal cancer and |
| | | neoplastic breast cells; |
| | | however, no difference |
| | | was found for PRL-1 and |
| | | PRL-2 expression levels |
| | | [186; 188]. PRL PTPs |
| | | were able to stimulate Rho |
| | | signaling pathways and |
| | | promote motility and |
| | | invasion [189] |
| YBX1 | YBX1 is a transcription and | In breast cancer models, |
| | translation factor that promotes | inhibition of YBX1 slows |
| | tumor growth and chemotherapy | tumor growth and is |
| | resistance by inducing genes, such | associated with decreased |
| | as HER-2, EGFR, PCNA, MDR-1, | HER-2 and EGFR [190]. |
| | cyclin A and cyclin B [190]. | Nuclear localization of |
| | | YBX1 is associated with |
| | | MDR1 gene expression |
| | | [148]. YBX1 expression |
| | | in mouse mammary |
| | | epithelial cells induces |
| | | proliferation with mitotic |
| | | failure and centrosome |
| | | amplification, and all later |
| | | developed multiple |
| | | mammary tumors |
| | | diagnosed as IDC [148]. |
| | | A study showed that |
| | | expression of YBX1 |
| | | identifies high risk breast |
| | | cancer patients in all |
| | | molecular subtypes [190] |
| TBC1D9 | Although the specific activities of | The role of TBC1D9 is |
| | TBC1D9 are unknown, the TBC1 | unknown in cancer; |
| | domain family of proteins is | however, there is evidence |
| | known to stimulate the GTPase | that alterations in RAB |
| | activity of RAB proteins [191]. | GTPases play a role in |
| | | cancer progression [192]. |
| LRBA | LRBA is a member of the WBW | LRBA was induced by |
| | gene family, and structural features | mitogens in immune cells |
| | suggest that it is part of a signaling | and over-expressed in |
| | pathway that requires interactions | several cancer types |
| | with other proteins, inositol | compared to normal tissue |
| | phospholopids or PKA [193]. It | [193]. |
| | was suggested that it plays a role | |
| | in the EGFR pathway [193]. | |
| TRIM29 | The TRIM family of proteins have | TRIM29 has been shown |
| | been suggested to define a variety | to be under-expressed in |
| | of cellular compartments as a | prostate and breast |
| | consequence of forming large | tumors, but over- |
| | molecular weight structures; | expressed in gastric |
| | however the specific function of | tumors [195]. It has also |
| | TRIM29 remains unknown [194]. | been suggested that |
| | | TRIM29 expression may |
| | | be a marker of lymph |
| | | node metastasis in gastric |
| | | cancer [195]. |
| SCUBE2 | SCUBE2 is a cell-surface protein, | Aberrant activation of the |
| | and although the exact mechanism | Hedgehog signaling |
| | remains unclear, it appears that | pathway was implicated in |
| | Scube2 functions either in the | progression of certain |
| | extracellular transport or | cancers by either ligand- |
| | stabilization of the hedgehog | dependent or ligand- |
| | protein, in the endocytic process, | independent mechanisms, |
| | or by modulating the activity of | and more recent studies |
| | other secreted ligands involved in | have shown increased |
| | the pathway [196-198]. | activity of the Hedgehog |
| | | signaling pathway in |
| | | breast carcinomas |
| | | suggesting that the |
| | | pathway be a new |
| | | therapeutic target |
| | | [199; 200]. |
| GATA3 | The GATA transcription factors | GATA3 has been |
| | are important in gene regulatory | implicated in breast |
| | networks that specify cell fate, and | cancer with up-regulation |
| | GATA3 is the regulator of | being a marker of the |
| | mammary gland formation by | luminal subtypes [201]. |
| | directing differentiation along the | Mutations in GATA3 |
| | luminal cell lineage | have also been identified |
| | [201; 202]. GATA3 also has | in some breast cancers, |
| | essential roles in T-cell | implying a tumor |
| | development, the sympathetic | suppressor role [201]. |
| | nervous system, kidney | Several studies have |
| | development, cochlear function, | shown that GATA3 is |
| | and formation of the root sheath in | highly associated with the |
| | skin [201]. | estrogen receptor pathway |
| | | [180; 203]. |
| CX3CL1 | CX3CL1 is a bifunctional | CX3CL1 secreted by |
| | cytokine. The soluble form acts | tumor cells has been |
| | like a classic chemokine attracting | shown to be capable of |
| | leukocytes through a gradient, | inducing migration [205]. |
| | while the cell surface-bound. | CX3CL1 and its receptor |
| | CX3CL1 promotes strong | CX3CR1 have been |
| | adhesion of leukocytes to the | shown to be expressed in |
| | producing cell without requiring | breast and prostate |
| | additional adhesion molecules | cancers, and may play a |
| | [204]. | role in directing cells to |
| | | specific metastatic sites |
| | | [206]. The expression of |
| | | CX3CL1 in prostate |
| | | cancer has been associated |
| | | with good patient |
| | | prognosis [207]. |
| IL6ST | The IL6ST protein (also known as | Due to the involvement in |
| | gp130) is the common signaling | multiple signaling |
| | subunit of receptors used by IL6 | pathways, both IL6 and |
| | cytokines, and all of the IL6 | gp130 have been |
| | cytokines require gp130 for | implicated in both tumor |
| | functional signaling [208]. After | promoting and |
| | ligand binding, gp130 activates | suppression [209]. It has |
| | receptor-associated tyrosine | been suggested that |
| | kinases, which activate | pharmacological |
| | downstream signaling pathways, | inhibitors of gp130 may |
| | such as MAPKs, PI3Ks, and | be a new approach for |
| | STATs [208; 209]. | breast cancer therapies |
| | | [208]. |
| MAPRE2 | The MAPRE genes encode the | The function of MAPRE2 |
| | EB1 family of proteins, which | in cancer remains |
| | were shown to have roles in | unknown other than it |
| | microtubule dynamics, cytokinesis, | may play a role in cell |
| | positioning of the mitotic spindle, | cyle regulation; however, |
| | and episome segregation [210]. It | alternatively spliced |
| | has been shown that MAPRE2 | transcripts of one EB |
| | protein product (RP1) associates | family member was found |
| | with the anaphase-promoting | in human colon cancer, |
| | complex [210]. | lung cancer, and leukemia |
| | | cell lines [210]. |
| GMPS | GMPS encodes the GMP synthase | In was previously shown |
| | enzyme, which is a G-type | that an imbalance of |
| | amidotransferase that catalyses the | purine metabolism is |
| | amination of XMP to GMP [211]. | correlated with |
| | GMPS plays a key role in de novo | transformation and cancer |
| | synthesis of guanine nucleotides | progression, and GMPS |
| | [212]. | was shown to be increased |
| | | 3.7-fold in chemically- |
| | | induced hepatomas in rats |
| | | [213]. |
| RABEP1 | Although the function of RABEP1 | The role of RABEP1 is |
| | is unknown, Rab GTPases are | unknown in cancer; |
| | known to control many aspects of | however, there is evidence |
| | membrane trafficking by | that alterations in RAB |
| | interacting with various effector | GTPases play a role in |
| | molecules [214]. | cancer progression [192]. |
| SLC39A6 | The product of SLC39A6 (LIV-1) | Increasing evidence that |
| | has been shown to transport zinc | aberrant expression of the |
| | into the cytoplasm from either | SLC39A family of zinc |
| | outside the cell or from stores in | transporters leads to |
| | intracellular compartments | uncontrolled cell growth |
| | [152; 215]. LIV-1 has also been | [152]. Zinc is also known |
| | shown to be regulated by estrogen | to play a role in cellular |
| | [152; 216]. | metabolism, and is |
| | | involved in growth, |
| | | differentiation and gene |
| | | transcription [216]. High |
| | | LIV-1 protein expression |
| | | has been associated with a |
| | | better outcome in breast |
| | | cancer patients [216]. |
| TPBG | The product of TPBG (5T4 | TPBG is considered a |
| | oncotoetal antigen) is a highly | tumor-associated antigen |
| | glycosylated cell surface protein | and is considered a target |
| | found on human placental | for immunotherapy |
| | trophoblast on various types of | [217; 219]. High |
| | cancer cells, but is not significantly | expression of TPBG has |
| | expressed in healthy adult tissues | been associated with poor |
| | [217; 218]. | outcome in gastric and |
| | | colorectal cancer patients |
| | | [218; 220]. |
| CKS2 | CKS2 has been shown to be | Expression of CKS2 is |
| | required for the metaphase to | elevated in a variety of |
| | anaphase transition in meiosis | tumors, and was |
| | [221]. | correlated with poor |
| | | patient survival [221]. It |
| | | has been shown that over- |
| | | expression of CKS2 |
| | | protects from apoptosis, |
| | | and inhibition of CKS2 |
| | | may be a new therapeutic |
| | | strategy for cancer [221]. |
| TCEAL1 | The product of TCEAL1 | Although it has not been |
| | (p21/SIIR) is a Ser/Arg/Pro-rich | well investigated in |
| | nuclear phosphoprotein that is 48% | cancer, one study |
| | similar to transcription elongation | demonstrated that |
| | factor A [222]. P21/SIIR was | differential expression of |
| | shown to repress promoter activity | TCEAL1 occurs in |
| | of Rous sarcoma virus long | esophageal cancers |
| | terminal repeat [222]. | compared to matched |
| | | normal samples [223]. |
| DSC2 | DSC2 is one of three desmocolling | DSC2 was shown to have |
| | cadherins that are membrane- | a wide tissue distribution, |
| | spanning glycoproteins that | while the other |
| | function as Ca2+-dependent cell | desmocollins (DSC1 and |
| | adhesion molecules [224]. | DSC3) are restricted to |
| | | stratified epithelia and |
| | | cardiac muscle; however, |
| | | in several human cancers |
| | | there has been a loss of |
| | | tissue specificity termed |
| | | “desmocollin switching” |
| | | [224; 225]. It was |
| | | suggested that the loss of |
| | | normal cellular adhesions |
| | | may contribute to the |
| | | epithelial-mesenchymal |
| | | transition, which is a |
| | | critical feature of many |
| | | cancers [224]. |
| SLC43A3 | The SLC43 family is an Na⁺- | The product of SLC43A3 |
| | independent, system-L-like amino | is suspected to transport |
| | acid transporter, although very | nutrients in rapidly |
| | little is known of the SLC43A3 | growing or developing |
| | gene itself [226]. It has been | tissues, such as embryonic |
| | shown to be present in a variety of | development and possibly |
| | embryonic epithelial tissues [227]. | cancer [227]. |

To investigate relationships of genes with known biological pathways and functions, the gene lists were imported into INGENUITY® (Ingenuity Systems), which is a software package that builds relevance networks based on published literature. The list of 32 genes was divided into 3 networks of biological interactions. The first network has pathways involved in cancer, respiratory disease and cell death, and includes 13 genes (BUB1, CKS2, EVL, FUT8, GATA3, GMPS, LRBA, PFKP, PTP4A2, RABEP1, SLC43A3, TBC1D9, and TRIM29) out of the 32 gene set. The other genes appearing in this network (CASP3, CLEC4E, CTSC, EGFR, IL6, IL13, JAKMIP2, LPAR3, MIA2, NR3C1, NSMAF, PDGF-CC, RB1, SBNO2, SCGB3A1, SLC16A6, SLC39A14, SLC7A7, TGFB1, TIMD4, TNS4, and TPST2) may be additional candidates for future investigations. Interestingly, IL6 appears in this network, but its receptor IL6ST, which is in the 32 gene set, does not.

The second network involves pathways associated with cellular growth and proliferation, the hematological system, development and function, and hematopoiesis, and includes 12 genes (ATAD2, CENPA, CX3CL1, ESR1, IL6ST, MAPRE2, MCM6, MELK, NAT1, PLK1, ST8SIA1, and XBP1) of the 32 gene set. This network also includes NFkB and the proteasome, which are known to be involved in tumorgenesis [229; 230]. The additional components of this network (5430435G22RIK, APOBEC3G, BCL2L14, CARD10, CDC25B, Cdc25B/C, DOK5, ERK, FSH, HSPA13, IL1F8, IL1F9, MAPK6, MT3, NFkB (complex), PIF, PRKX, Proteasome, RAB33B, SLC12A7, STK10, STK24, and TFF2) may be additional candidates for investigation.

Network 3 includes pathways associated with cancer, cellular compromise, and genetic disorders, and includes 7 genes (DSC2, GABRP, SCUBE2, SLC39A6, TCEAL1, TPBG, and YBX1) of the 32 gene set. The other genes appearing in this network (AATK, ATP6V1F, BAI2, C22ORF28, CD1B, DHRS3, DUSP11, FMR1, GABRE, HECW2, HNF4A, LAD1, MIRN18A, N4BP2L2, OAS3, PEMT, RBM7, RTP3, SCUBE1, SHISA5, TMEM49, TMEM176B, TNF, TP73, TRIM15, ZBTB11, ZNF175, and ZNF318) may be candidates for future investigations.

It was determined that 21 of the 32 genes (ATAD2, BUB1, CENPA, CKS2, CX3CL1, ESR1, GABRP, GATA3, GMPS, IL6ST, MELK, PFKP, PLK1, RABEP1, SCUBE2, SLC39A6, ST8SIA1, TBC1D9, TPBG, XBP1, and YBX1) had known associations with cancer in general, and several were associated with specific cancer types, including six genes (ESR1, GATA3, PLK1, SCUBE2, SLC39A6, and TBC1D9) associated with breast cancer (Table 12). Associations of genes with various cellular functions involved with cancer progression were also determined (Table 13). Six genes

TABLE 12
Ingenuity pathway analysis of the 32 genes derived from the
putative carcinoma and stromal cell subsets reveals
associations with different cancer types.

| CANCER TYPE | GENES |
|---|---|
| Breast cancer | ESR1, GATA3, PLK1, SCUBE2, |
| | SLC39A6, TBC1D9 |
| Prostate cancer | CX3CL1, ESR1, GATA3, PLK1 |
| Lung carcinoma | CKS2, PFKP, PLK1 |
| Adenocarcinoma | CKS2, ESR1, PFKP |
| Endometrial cancer | ESR1, PLK1 |
| Bladder cancer | ESR1, PLK1 |
| Adenoma | ESR1, IL6ST |

TABLE 13
Ingenuity pathway analysis of the 32 genes derived from the
putative carcinoma and stromal cell subsets reveals associations
with different cellular functions involved in tumorigenesis.

| FUNCTION | GENES |
|---|---|
| Growth | ESR1, GABRP, IL6ST, PLK1, ST8SIA1, XBP1 |
| Proliferation | ATAD2, CKS2, ESR1, IL6ST, PLK1, ST8SIA1 |
| Cell cycle progression | CKS2, ESR1, PLK1, XBP1 |
| Apoptosis | ESR1, PLK1, XBP1, YBX1 |
| Differentiation | ESR1, IL6ST, ST8SIA1 |
| Developmental process | ESR1, ST8SIA1 |
| Morphology | IL6ST, ST8SIA1 |

(ESR1, GABRP, IL6ST, PLK1, ST8SIA1, and XBP1) were involved in growth, while six genes (ATAD2, CKS2, ESR1, IL6ST, PLK1, and ST8SIA1) were found to be involved in proliferation pathways. There were four genes (CKS2, ESR1, PLK1, and XBP1) associated with cell cycle progression, two genes (ESR1 and ST8SIA1) associated with development, and two genes (IL6ST and ST8SIA1) involved in cell morphology-related functions. Additionally there were associations with cellular processes that are negative regulators of cancer progression, such as differentiation (ESR1, IL6ST, and ST8SIA1) and apoptosis (ESR1, PLK1, XBP1, and YBX1).

Several reports of the published molecular signatures of breast cancer utilized in development of this 32 gene set also performed pathway analysis of their molecular signatures (e.g., Jansen et al. [54] and Wang et al. [67]) to identify relationships between those gene sets and other published works. Utilization of this pathway analysis software revealed that a number of the genes from the signatures were involved in similar pathways, e.g., cell death, cell cycle, and proliferation, although different genes in the pathways were identified in different molecular signatures. Collectively, this information provides insight into cellular mechanisms by which these genes interact, while providing candidate molecular targets and pathways for devising therapeutic approaches.

Thus the gene signatures described herein were investigated collectively, without bias in gene selection, to derive a subset of candidate genes in order to test their utility as a predictive test of risk of breast cancer recurrence.

In order to evaluate the clinical relevance of gene sets described above, the expression results of those genes were first analyzed for reproducibility to ensure the quality of data used for clinical correlations. Gene expression was measured in intact tissue sections for both levels and distributions, before proceeding to investigate the two gene sets representative of the corresponding cell types procured by LCM [231].

### Methods and Materials

Reproducibility of qPCR Analyses

The technique of real-time quantitative polymerase chain reaction (qPCR) using the ABI Prism 7900HT system (Applied Biosystems) was utilized for quantitative examination of the gene transcripts of interest. Cells from preparations of either intact tissue sections or LCM-procured cells were lysed, and extracts were examined for transcription of candidate genes. RNA from each cell type was extracted and isolated with the Arcturus PICOPURE™ (LCM-procured cells) or QIAGEN RNEASY™ RNA isolation kit (intact tissue section analyses) following procedures described in herein.

After isolation from the LCM-procured cells, the RNA was evaluated with the Agilent RNA 6000 Pico Kit and the BIOANALYZER™ Instrument (Agilent Technologies) for quality and quantity before proceeding to reverse transcription and qPCR. Multiple microdissections (2-3 LCM caps) from a tissue section were pooled to obtain a greater quantity of RNA, so that a linear amplification step was unnecessary prior to qPCR. To accomplish this, the amount of total RNA required from LCM-procured cells for a qPCR reaction was 10 ng from carcinoma cells and 1 ng from stromal cells. Total RNA was then reverse transcribed to cDNA and analyzed by qPCR. The concentration of the calibrator (i.e., cDNA obtained from reverse transcription of Universal Human Reference RNA (Stratagene)), for ΔΔCt calculations was adjusted to be similar to that of the experimental reactions in the qPCR plate.

Extensive quality control experiments were performed to assess reproducibility of the qPCR results. Four serial tissue sections from each of three specimens were prepared and processed concurrently, through scraping, RNA isolation, reverse transcription and qPCR analyses of the genes in the cancer subset. The qPCR reactions were performed in triplicate with duplicate wells in each 384-well plate. A second quality control evaluation involved RNA extraction and qPCR analyses of three tissue sections of each of six different specimens, each section processed and evaluated independently on different days to ascertain inter-assay variation. Furthermore, each specimen was analyzed in triplicate by qPCR with duplicate wells in each 384-well plate.

### Statistical Analyses

T-tests and analysis of variance (ANOVAs) were performed either in MICROSOFT® Excel or GRAPHPAD PRISM® Version 4 (GraphPad Software, La Jolla, Calif.). Univariate cox regressions were performed with SPSS® 17.0 statistical package (SPSS Inc., Chicago, Ill.). This software package is a comprehensive system of advanced statistics and is widely used to extract information from large amounts of population-based data. Survival calculations were performed using log₂ transformations of relative gene expression data.

### Results and Discussion

Intra- and Inter-Assay Reproducibility of qPCR Results

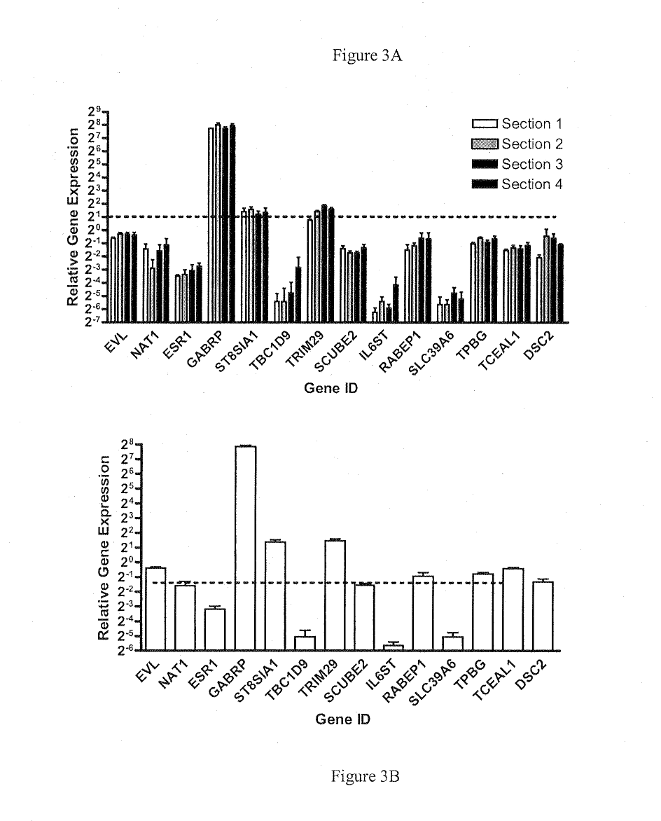

Before undertaking analyses of gene expression in numerous tissue specimens with valuable clinical follow-up, extensive quality control experiments were performed as described herein. The qPCR reactions gave the levels of reproducibility illustrated in FIG. 3A. A one-way ANOVA test (Kruskal-Wallis) was performed on the gene expression results for this representative sample to examine if there was a statistically significant difference among the tissue sections processed [232]. The ANOVA yielded a P value of 0.81, indicating no significant difference was observed (FIG. 3A). These analyses were repeated with two additional breast cancer specimens and gave similar results (data not presented), indicating there was no significant difference in gene expression measurements of multiple tissue sections for each specimen. In FIG. 3B, the collective results from 12 qPCR analyses from the same specimen (analyzed in FIG. 3A) are shown to illustrate the reproducible qPCR determinations using different tissue sections supporting this approach for validation of gene expression.

The coefficient of variation (CV) was calculated for expression of each gene (standard deviation divided by the mean and expressed as a percent) to identify the relative variability (Table 14). The majority of genes analyzed showed less than 50% CV, which illustrates acceptable levels of relative variability for results from this complex platform [233-235]. The results exhibiting greater CV values generally were from genes with low levels of expression, so that any difference measured created a greater CV value. For the representative specimen shown, an average CV of 42% was determined for each of the 14 genes (Table 14). These analyses, which were repeated in two additional breast specimens with similar results exhibiting average CV results of 55% and 33% across the genes examined (data not presented).

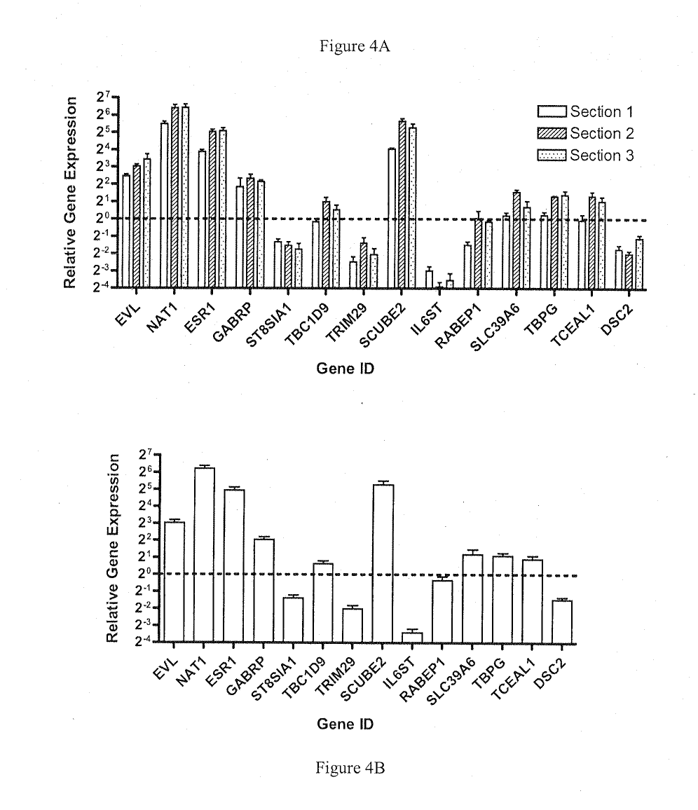

Another level of quality control by undertaken by qPCR analyses of three serial tissue sections of each of six different specimens, each section processed and evaluated independently on different days to ascertain inter-assay variation. RNA from each specimen was analyzed by qPCR as described in Methods and Materials. These data were then evaluated and compared between tissue sections (FIG. 4A) and for all qPCR runs performed with an individual specimen (FIG. 4B). A one-way ANOVA test (Kruskal-Wallis) was performed on the gene expression results for this sample to examine if there was a statistically significant difference among the tissue sections processed [232]. The ANOVA yielded a P value of 0.72, indicating that no significant difference was observed between tissue sections. These gene expression analyses, which were repeated in five additional breast cancer specimens with similar results, indicating no significant difference was observed between the three tissue sections from each specimen (data not presented). The percent CV was calculated for expression of each gene in all qPCR runs to identify the relative variability. The majority of the genes analyzed showed less than 50% CV, which reflected appropriate levels of relative variability [233-235]. For the representative specimen shown in FIG. 4, an average CV of 43% was determined for all genes. These qPCR analyses, repeated in five additional specimens exhibited average CV values of 49%, 51%, 53%, 51% and 68% across the genes examined (data not presented).

TABLE 14
Representative relative variability of multiple qPCR measurements
for a single specimen in which four serial tissue sections were
processed concurrently. qPCR measurements of expression of the
majority of genes analyzed showed a CV of less than 50%, illustrating
the range in variability of the results obtained with this analysis
platform. Note that the genes exhibiting greater CV values generally
had low levels of expression.

| | AVERAGE GENE | STANDARD | |
|---|---|---|---|
| GENE | EXPRESSION VALUE | DEVIATION | CV (%) |
| EVL | 0.76 | 0.13 | 16.9 |
| NAT1 | 0.33 | 0.23 | 69.2 |
| ESR1 | 0.11 | 0.05 | 42.0 |
| GABRP | 231.20 | 39.93 | 17.3 |
| ST8SIA1 | 2.60 | 0.79 | 30.3 |
| TBC1D9 | 0.03 | 0.03 | 105.8 |
| TRIM29 | 2.74 | 0.79 | 28.8 |
| SCUBE2 | 0.34 | 0.09 | 25.8 |
| IL6ST | 0.02 | 0.01 | 50.0 |
| RABEP1 | 0.52 | 0.29 | 54.7 |
| SLC39A6 | 0.03 | 0.02 | 64.9 |
| TBPG | 0.58 | 0.12 | 21.2 |
| TCEAL1 | 0.39 | 0.10 | 24.6 |
| DSC2 | 0.40 | 0.16 | 41.4 |

### Evaluation of Gene Subsets in LCM-Procured Cells

The breast carcinoma specimens selected for this critical study were representative of the biopsies received in a typical hospital pathology laboratory. Specifically, tissues exhibiting a broad range of carcinoma to non-carcinoma elements were examined to insure test development was not biased by cellular composition of the specimen (Table 15).

In order to evaluate expression of the 14 genes in the carcinoma subset and 18 genes in the stromal subsets, tissues containing a variety of cell types were selected for LCM (Table 15). The quantity of each cell type within a tissue section (expressed as a percent) was estimated after H & E staining and light microscopy. The average quantity of carcinoma cells present in the tissues evaluated was 61% of the total cells (range of 10-95% carcinoma cells). The average quantity of stromal cells present in the tissues evaluated was 22% of the total cells (range of 5-50% stroma). Expression levels of the genes in the carcinoma subset are predicted to be similar between intact tissue sections and LCM-procured carcinoma cells if the tissue section contained 95% carcinoma. Similarly, if expression of a gene from the stromal gene subset is indeed principally from the stromal cells, its expression level should be greatly enriched by LCM procurement compared to its levels in the intact tissue section.

Specifically, specimen “u” from Table 15, contained 10% carcinoma cells, 50% stromal cells, and 40% fibrous stroma.; specimen “w,” contained 50% carcinoma cells, 5% inflammatory cells, 40% stromal cells, and 5% fibrous stroma.; specimen “y,” contained 30% carcinoma cells, 15% stromal cells, and 55% fibrous stroma; and specimen “ad,” contained 90% carcinoma cells, 5% inflammatory cells, and 5% stromal cells.

TABLE 15
Analyses of cellular composition in 31 different human breast
carcinoma specimens used for LCM. Cellular composition of a
tissue section was estimated by H & E staining and light microscopy.

| | CANCER | INFLAMMATORY | STROMAL | OTHER |
|---|---|---|---|---|
| SAMPLE ID | CELLS (%) | CELLS (%) | CELLS (%) | (%) |
| a | 95 | 0 | 5 | 0 |
| b | 50 | 10 | 40 | 0 |
| c | 30 | 5 | 30 | 35 |
| d | 15 | 0 | 5 | 80 |
| e | 80 | 10 | 10 | 0 |
| f | 60 | 5 | 25 | 10 |
| g | 80 | 10 | 10 | 0 |
| h | 50 | 10 | 40 | 0 |
| i | 50 | 5 | 30 | 15 |
| j | 75 | 0 | 25 | 0 |
| k | 65 | 10 | 25 | 0 |
| l | 40 | 20 | 40 | 0 |
| m | 75 | 0 | 5 | 20 |
| n | 80 | 5 | 5 | 10 |
| o | 80 | 5 | 10 | 5 |
| p | 60 | 10 | 30 | 0 |
| q | 90 | 5 | 5 | 0 |
| r | 50 | 10 | 20 | 20 |
| s | 75 | 5 | 20 | 0 |
| t | 50 | 0 | 15 | 35 |
| u | 10 | 0 | 50 | 40 |
| v | 70 | 10 | 20 | 0 |
| w | 50 | 5 | 40 | 5 |
| x | 85 | 5 | 10 | 0 |
| y | 30 | 0 | 15 | 55 |
| z | 60 | 20 | 20 | 0 |
| ab | 60 | 0 | 40 | 0 |
| ac | 80 | 0 | 20 | 0 |
| ad | 90 | 5 | 5 | 0 |
| ae | 50 | 10 | 40 | 0 |
| af | 60 | 20 | 20 | 0 |

To investigate these relationships, gene subsets were analyzed using LCM-procured cell populations. Thirty-three samples of LCM-procured carcinoma cells were obtained for OCR analyses of the carcinoma gene subset, and 23 samples of LCM-procured stromal cells were collected for qPCR analyses of the stromal gene subset. Gene expression levels of the two subsets of the intact tissue sections were compared with those of the LCM-procured cell populations (representative specimens shown in FIGS. 5-7) using tissue sections containing a range of carcinoma cell content. Welch t-tests were used to identify any gene in which the expression level was significantly different between the two groups (Tables 16 and 17).

Results from a representative specimen (FIG. 5) illustrate the comparison of gene expression between intact tissue sections and LCM-procured cells from a 31 year old patient with invasive ductal carcinoma, whose tissue specimen contained 95% carcinoma and 5% stromal cells. FIG. 5A shows relative expression of the cancer gene subset from intact tissue compared to that of LCM-procured carcinoma cells. Expression of three of the 14 genes was statistically lower in the intact tissue compared to those of LCM-procured cells. It is expected that few of the genes would be statistically different in the LCM-procured carcinoma cells compared to the intact tissue of this specimen, since the intact tissue contained 95% carcinoma cells. FIG. 5B shows relative expression the stromal gene subset from intact tissue compared to that of LCM-procured stromal cells. Expression of nine of the 18 genes was statistically higher in the intact tissue compared to LCM-procured cells as predicted.

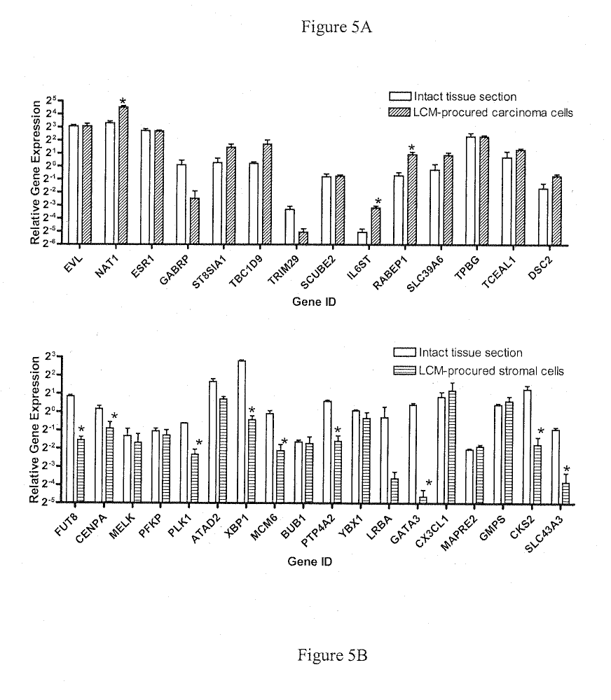

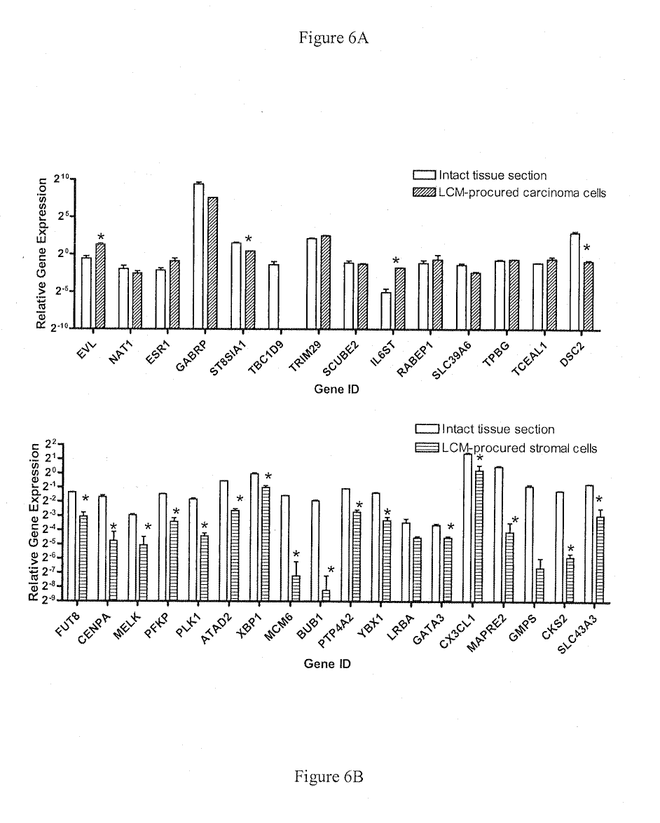

FIG. 6 illustrates the comparison of gene expression levels between intact tissue sections and LCM-procured cells from a 44 year old patient with invasive ductal carcinoma, whose tissue section contained 60% carcinoma, 30% stromal, and 10% inflammatory cells. Expression of four of the 14 genes in carcinoma cells was statistically different in the intact tissue compared to LCM-procured cells (FIG. 6A).

TABLE 16
Results of Welch t-tests illustrating differences in gene expression
levels between intact breast tissue sections and LCM-procured
cancer cells. In order to compare the differences in relative gene
expression of the 14 genes in the cancer subset between intact
tissue and LCM-procured carcinoma cells, t-tests were performed.
The number of specimens analyzed and those that displayed a
significant difference (P < 0.05) in relative gene expression between
the intact breast tissue and the LCM-procured cancer cells are
shown. The average fold change and ranges observed in each of
the 33 samples analyzed are presented.

| | NUMBER OF | | |
|---|---|---|---|
| | SPECIMENS | | |
| | EXHIBITING | AVERAGE | RANGE OF |
| | DIFFERENCES | FOLD | FOLD |
| | IN EXPRESSION | CHANGE | CHANGES |
| GENE | (P < 0.05) | (CA/INTACT) | OBSERVED |
| EVL | 7/33 (21.2%) | 0.74 | −2.56 to 4.17 |
| NAT1 | 4/33 (12.1%) | 0.55 | −9.39 to 43.19 |
| ESR1 | 5/33 (15.2%) | 2.12 | −9.00 to 32.38 |
| GABRP | 4/30 (12.1%) | 1.50 | −51.00 to 116.03 |
| ST8SIA1 | 7/33 (21.2%) | −0.32 | −4.21 to 2.29 |
| TBC1D9 | 10/28 (35.7%) | −1.58 | −16.33 to 23.74 |
| TRIM29 | 7/32 (30.4%) | −0.97 | −4.30 to 10.50 |
| SCUBE2 | 9/33 (27.3%) | −0.85 | −10.83 to 25.05 |
| IL6ST | 12/33 (36.4%) | −0.86 | −23.00 to 14.65 |
| RABEP1 | 8/33 (24.2%) | −0.15 | −7.38 to 4.16 |
| SLC39A6 | 8/33 (24.2%) | −1.31 | −11.07 to 6.52 |
| TPBG | 2/33 (6.1%) | −0.43 | −8.22 to 3.50 |
| TCEAL1 | 3/33 (9.0%) | 0.33 | −3.86 to 4.30 |
| DSC2 | 8/33 (24.2%) | 0.35 | −14.45 to 39.53 |

TABLE 17
Results of Welch t-tests illustrating differences in gene expression
levels between intact breast tissue sections and LCM-procured
stromal cells. In order to compare the differences in relative gene
expression of the 18 genes in the stromal subset between intact
tissue and LCM-procured stromal cells, t-tests were performed.
The number of specimens analyzed and those that displayed a
significant difference (P < 0.05) in relative gene expression between
the intact breast tissue and the LCM-procured stromal cells are
shown. The average fold change and ranges observed in each of the
23 samples analyzed are presented.

| | NUMBER OF | | |
|---|---|---|---|
| | SPECIMENS | | |
| | EXHIBITING | AVERAGE | RANGE OF |
| | DIFFERENCES | FOLD | FOLD |
| | IN EXPRESSION | CHANGE | CHANGES |
| GENE | (P < 0.05) | (ST/INTACT) | OBSERVED |
| FUT8 | 10/23 (43.5%) | −1.48 | −9.31 to 13.00 |
| CENPA | 15/23 (65.2%) | −8.21 | −54.00 to 3.45 |
| MELK | 10/23 (43.5%) | −10.34 | −103.33 to −3.05 |
| PFKP | 14/23 (60.9%) | −10.48 | −102.00 to 2.36 |
| PLK1 | 15/23 (65.2%) | −4.31 | −14.67 to 1.38 |
| ATAD2 | 9/23 (39.1%) | −2.43 | −9.35 to 1.62 |
| XBP1 | 13/23 (56.5%) | −3.53 | −14.16 to 2.50 |
| MCM6 | 8/23 (34.8%) | −6.22 | −48.75 to 3.42 |
| BUB1 | 7/23 (30.4%) | −6.06 | −78.00 to 4.50 |
| PTP4A2 | 11/23 (47.8%) | −3.24 | −37.00 to 3.62 |
| YBX1 | 12/23 (52.2%) | −2.03 | −4.33 to 1.75 |
| LRBA | 8/23 (34.8%) | −2.27 | −42.82 to 21.11 |
| GATA3 | 14/23 (60.9%) | −8.18 | −50.47 to 3.67 |
| CX3CL1 | 13/23 (56.5%) | −4.83 | −35.09 to 2.98 |
| MAPRE2 | 5/23 (21.7%) | −1.42 | −24.29 to 5.20 |
| GMPS | 6/23 (26.1%) | −6.50 | −52.50 to 4.76 |
| CKS2 | 10/23 (43.5%) | −5.52 | −25.40 to 6.00 |
| SLC43A3 | 9/23 (39.1%) | −3.36 | −16.20 to 2.95 |

FIG. 6B shows relative expression of the stromal gene subset in intact tissue compared to that of LCM-procured stromal cells. Expression of sixteen of the 18 genes was statistically higher in the intact tissue compared to LCM-procured cells presumably reflecting the cellular heterogeneity.

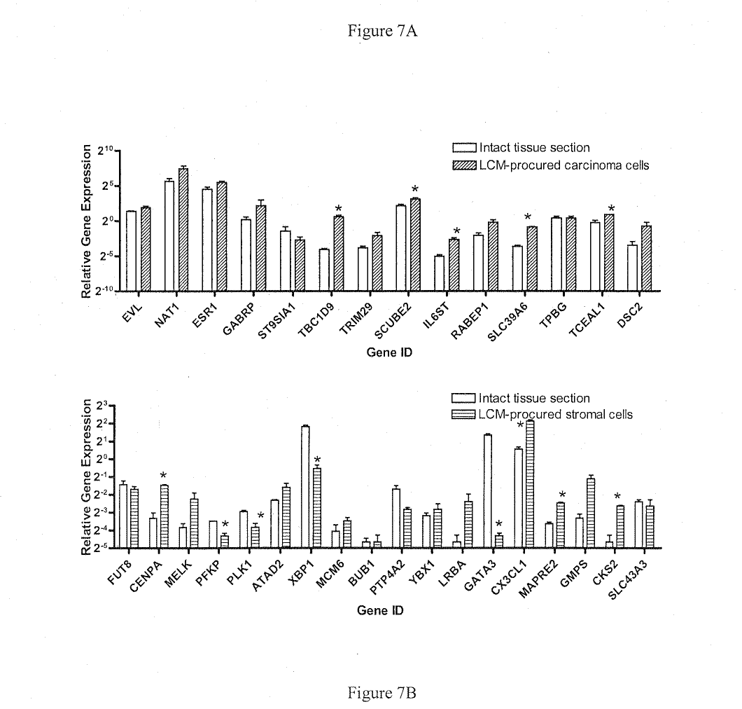

As shown in FIG. 7, a similar comparison of gene expression levels between intact tissue sections and LCM-procured cells was investigated in a tissue specimen containing only 30% carcinoma, 30% stromal, and 5% inflammatory cells (the remaining 35% of the tissue contained fibrous connective tissue). FIG. 7A illustrates the comparison of relative expression the cancer gene subset. Expression of five of the 14 genes was statistically lower in the intact tissue compared to LCM-procured cells. FIG. 7B shows the comparison of relative expression of the stromal gene subset. Eight of the 18 genes exhibited expression levels that were statistically different in the intact tissue compared to LCM-procured cells.

As a result of preliminary observations that gene expression levels of intact tissue compared to that of LCM-procured cells were highly variable among specimens with differing cell contents, the following studies were performed. To evaluate differences in gene expression of intact tissue compared to either LCM-procured carcinoma or stromal cells, a wide variety of breast tissue specimens reflecting the clinical reality were evaluated. Welch t-tests were performed comparing relative expression of the 14 gene subset in intact tissue section with that of the LCM-procured carcinoma cells for 33 specimens in three separate qPCR experiments (Table 16). Welch t-tests were also performed comparing the relative expression of the 18 gene subset in intact tissue section with that of the LCM-procured stromal cells for 23 specimens in three separate qPCR experiments (Table 17). The number of specimens exhibiting a significant difference (P<0.05) in relative gene expression between the intact breast tissue section and that of the LCM-procured cells is shown. Fold change was calculated as the expression of the gene in the LCM-procured cells compared to that of the intact tissue, such that a positive fold change indicates greater expression in the LCM-procured cells. The average and ranges of fold change observed in all samples analyzed are presented in Tables 16 and 17 to illustrate the large range of values observed.

Overall, 21% of the breast biopsies exhibited significant differences in expression of the 14 genes in the carcinoma subset when intact tissue was compared to those of LCM-procured carcinoma cells. In contrast, 46% of the breast tissues exhibited significant differences in expression of the 18 genes in the stromal subset when intact tissue was compared to LCM-procured stromal cells. This implies there is a greater requirement for procuring stromal cells by LCM when comparing their gene expression patterns to those of intact tissue sections than for making the same comparison with carcinoma cells. Noting that tissue specimens utilized in this investigation are representative of those from clinical pathology laboratories, these differences in gene expression may be due to a lower content of stromal cells in biopsies removed to diagnose cancer (Table 15). The complexity of examining gene expression profiles in stromal cells is considerably greater than that of carcinoma cells, apparently due to the differences in ratios of total cell volume (size) to nuclear volume (size). In addition, nuclei from breast carcinoma cells are larger compared to those of stromal cells, usually resulting in a greater quantity of RNA per collection. Of the possible explanations, changes in gene expression relationships observed appear to be directly related to the heterogeneous cell composition of a tissue section.

Another interesting observation from the cancer gene subset is that the average fold change of individual gene expression between LCM-procured carcinoma cells and intact tissue was variable (6 positive and 8 negative relationships). In contrast, this relationship was negative for each of the 18 genes in the stromal subset. These surprising results suggest that either the decreased expression of all 18 genes is simply due to their down-regulation in stromal cells surrounding carcinoma cells, or that the 18 “stromal” genes are highly expressed in the other cell types, particularly in the carcinoma cells of an intact tissue section.

### Influence of Specific Cell Content in a Tissue Section on Gene Expression

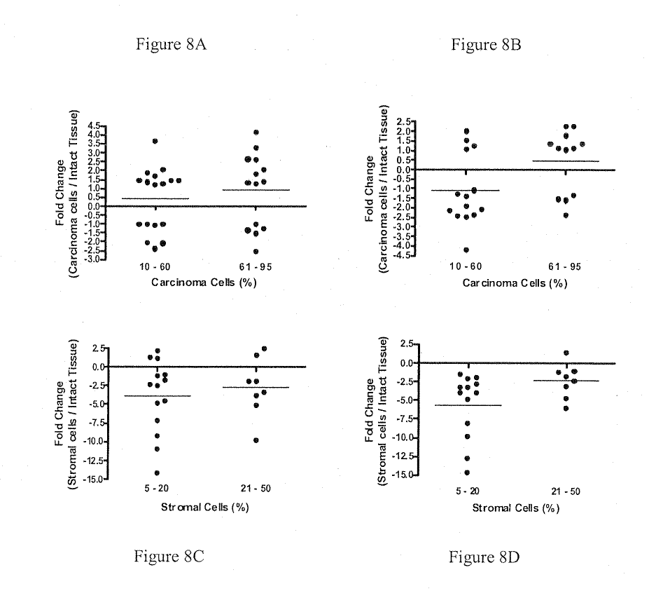

In order to address whether changes in the expression patterns observed in the genes of the carcinoma and stromal subsets are directly related to the cell content of the tissue, distributions of fold change in gene expression between LCM-procured cells and intact tissue were evaluated based on percent cell type present in the tissue specimen (Table 15 and FIG. 8). If the gene expression is specific for a single cell type (cancer or stromal) and the tissue section is composed largely of that cell type, a fold change (relative to the intact tissue section) of 1 is expected. However, if the gene expression is specific for a single cell type (cancer or stromal) and the tissue section contains considerably smaller amounts of that cell type, a fold change (relative to the intact tissue section) difference is expected. Fold change of expression for EVL and ST8SIA1 of the carcinoma gene subset was compared in tissues containing 0-60% carcinoma cells (n=17) and greater than 60% carcinoma cells (n=14) (FIGS. 8A and B). In addition, fold change of expression for XBP1 and PLK1 in the stromal gene subset were compared in tissues containing 0-20% stromal cells (n=14) and greater than 20% stromal cells (n=8) (FIGS. 8C and D). To analyze differences in gene expression, t-tests were performed comparing fold change (LCM-procured cells/intact tissue) according to percent of the specific cell content in the tissue examined (Table 18). FIGS. 8A and 8C illustrate representative genes (EVL and XBP1, respectively) whose fold change was not significantly different among tissues regardless of content of the cell in question (e.g., cancer and stromal). Of the 32 genes examined in the two gene subsets, only ST8SIA1 (P value=0.03) and PLK1 (P value=0.04) exhibited fold changes in expression levels that were significantly different in tissues containing variable quantities of the cell type in question, e.g., cancer and stromal (FIGS. 8B and 8D).

Collectively, these data suggest that expression of ST8SIA1 in carcinoma cells and PLK1 in stromal cells is directly related to the cell type. In addition, expression of TRIM29 (P value=0.07) and IL6ST in carcinoma cells (P value=0.09), as well as PFKP in stromal cells (P value=0.06) approached significance based on t-test analyses (Table 18), suggesting these genes may also be specific to their respective subset. No statistically significant differences were observed in fold changes for a number of genes using this type of analysis (e.g., MELK, MCM6, GATA3 in Table 18), suggesting LCM procurement of specific cell types did not enhance the expression results. However, analyses of other genes in the subsets revealed LCM collection of specific cell types influenced measurements of gene expression, e.g., CENPA, BUB1, YBX1 (Table 18). Gene expression in specific cell types provide a more direct interpretation of their genomic activity in a tissue section, with the exception of tissue sections composed primarily of cells of a single type.

### Assessment of Entire 32 Gene Panel in LGM-Procured Carcinoma and Stromal Cells

A subset of 14 genes was selected as candidates in carcinoma cells, while a subset of 18 genes was predicted to reflect expression in stromal cells. As described in Table 8, the genes evaluated were derived from 12 molecular signatures from 11 studies. The majority of the reports did not indicate if the individual expression level was elevated or diminished. Furthermore, few reports have been published regarding the expression of genes in specific cell types outlined in this Dissertation (e.g., [41; 57; 70]), nor of comparisons of gene expression in specific cell types with intact tissue. In these investigations, expression of each gene in both the putative cancer and stromal subsets was analyzed by qPCR using 12 individual breast tissue specimens to prepare an intact tissue section, LCM-procured carcinoma cells and LCM-procured stromal cells from each. These 12 tissue specimens were representative of the variety of biopsies observed in the clinical setting. Selective results for these analyses are presented using the same three representative breast cancer biopsies described earlier in FIGS. 5-7.

TABLE 18
Summary of results from t-tests comparing fold change (LCM-procured
cells/intact tissue) and cell content in the tissue examined. Fold change
of gene expression was calculated between LCM-procured cells (of the
corresponding gene subset) and intact tissue. Genes in the carcinoma
subset were examined in tissues containing 0-60% carcinoma cells
compared to those containing greater than 60% carcinoma cells. Genes
in the stromal subset were examined in tissues containing 0-20%
stromal cells compared to those containing greater than 20% stromal
cells. P values in bold were statistically significant (less than 0.05).

| | GENE | T-TEST |
|---|---|---|
| GENE | SUBSET | (p value) |
| EVL | carcinoma | 0.47 |
| NAT1 | carcinoma | 0.26 |
| ESR1 | carcinoma | 0.79 |
| GABRP | carcinoma | 0.38 |
| ST8SIA1 | carcinoma | 0.03 |
| TBC1D9 | carcinoma | 0.76 |
| TRIM29 | carcinoma | 0.07 |
| SCUBE2 | carcinoma | 0.24 |
| IL6ST | carcinoma | 0.09 |
| RABEP1 | carcinoma | 0.27 |
| SLC39A6 | carcinoma | 0.70 |
| TPBG | carcinoma | 0.20 |
| TCEAL1 | carcinoma | 0.85 |
| DSC2 | carcinoma | 0.59 |
| FUT8 | stroma | 0.18 |
| CENPA | stroma | 0.11 |
| MELK | stroma | 0.98 |
| PFKP | stroma | 0.06 |
| PLK1 | stroma | 0.04 |
| ATAD2 | stroma | 0.29 |
| XBP1 | stroma | 0.53 |
| MCM6 | stroma | 0.96 |
| BUB1 | stroma | 0.10 |
| PTP4A2 | stroma | 0.85 |
| YBX1 | stroma | 0.12 |
| LRBA | stroma | 0.47 |
| GATA3 | stroma | 0.95 |
| MAPRE2 | stroma | 0.79 |
| GMPS | stroma | 0.68 |
| CKS2 | stroma | 0.79 |
| SLC43A3 | stroma | 0.85 |

### Representative Analyses Using a Tissue Biopsy Containing Primarily Carcinoma Cells

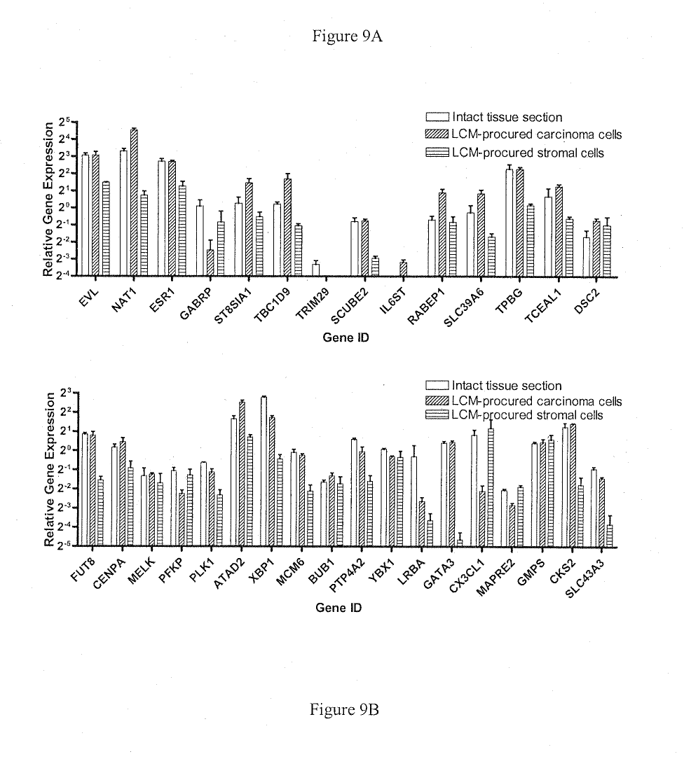

Using the biopsy from a 31 year old patient with invasive ductal carcinoma, tissue sections were prepared which contained 95% carcinoma cells and 5% stromal cells. A comparison of relative expression of each gene in the entire 32 gene set was performed using RNA extracted from intact tissue, LCM-procured carcinoma cells, and LCM-procured stromal cells (FIG. 9 and Table 19). Focusing on the carcinoma gene subset (FIG. 9A), expression levels of only three genes (NAT1, IL6ST, and RABEP1) were statistically different in the LCM-procured carcinoma cell population compared to that of the intact tissue (Table 19). Each of the three genes was over-expressed in the cancer cell population compared to the intact tissue (2.3, 4.3, and 3.1-fold, respectively). Since this specimen was composed of primarily (95%) of carcinoma cells, little difference would be predicted in gene expression levels between the LCM-procured carcinoma cells and the intact tissue.

TABLE 19
Statistical differences in gene expression among intact tissue, LCM-procured
carcinoma cells and LCM-procured stromal cells shown in FIG. 5.

| | DIFFERENCES IN GENE | | DIFFERENCES IN GENE | | DIFFERENCES IN GENE | |
|---|---|---|---|---|---|---|
| | EXPRESSION BETWEEN | | EXPRESSION BETWEEN | | EXPRESSION BETWEEN | |
| | INTACT TISSUE & LCM- | | INTACT TISSUE & LCM- | | LCM-PROCURED CANCER | |
| | PROCURED CANCER CELLS | | PROCURED STROMAL CELLS | | CELLS & STROMAL CELLS | |
| | T-TEST | FOLD CHANGE | T-TEST | FOLD CHANGE | T-TEST | FOLD CHANGE |
| GENE | (P VALUE) | (CA/INTACT) | (P VALUE) | (ST/INTACT) | (P VALUE) | (CA/ST) |
| EVL | 0.991 | 1.0 | 0.014 | −3.0 | 0.047 | 3.0 |
| NAT1 | 0.005 | 2.3 | 0.010 | −5.9 | 0.005 | 13.9 |
| ESR1 | 0.940 | −1.0 | 0.014 | −2.7 | 0.005 | 2.7 |
| GABRP | 0.083 | −5.9 | 0.389 | −1.9 | 0.498 | −3.1 |
| ST8SIA1 | 0.074 | 2.3 | 0.263 | −1.7 | 0.048 | 3.9 |
| TBC1D9 | 0.124 | 2.8 | 0.012 | −2.4 | 0.076 | 6.7 |
| TRIM29 | 0.054 | −3.6 | 0.038 | −7.3 | 0.047 | 2.0 |
| SCUBE2 | 0.830 | 1.0 | 0.038 | −4.3 | 0.006 | 4.4 |
| IL6ST | 0.005 | 4.3 | 0.008 | −8.0 | 0.003 | 34.0 |
| RABEP1 | 0.034 | 3.1 | 0.786 | −1.1 | 0.026 | 3.3 |
| SLC39A6 | 0.071 | 2.2 | 0.203 | −2.6 | 0.031 | 5.7 |
| TPBG | 0.981 | −1.0 | 0.057 | −4.4 | 0.007 | 4.4 |
| TCEAL1 | 0.318 | 1.5 | 0.253 | −2.5 | 0.007 | 3.7 |
| DSC2 | 0.067 | 2.0 | 0.433 | 1.6 | 0.676 | 1.2 |
| FUT8 | 0.784 | −1.1 | 0.001 | −5.2 | 0.027 | 5.0 |
| CENPA | 0.400 | 1.2 | 0.043 | −2.1 | 0.040 | 2.6 |
| MELK | 0.891 | 1.1 | 0.635 | −1.3 | 0.458 | 1.4 |
| PFKP | 0.022 | −2.3 | 0.528 | −1.2 | 0.164 | −1.9 |
| PLK1 | 0.074 | −1.4 | 0.007 | −3.3 | 0.019 | 2.4 |
| ATAD2 | 0.025 | 1.8 | 0.125 | −1.9 | 0.006 | 3.5 |
| XBP1 | 0.001 | −2.1 | 0.000 | −9.3 | 0.002 | 4.5 |
| MCM6 | 0.497 | −1.1 | 0.011 | −4.1 | 0.001 | 3.7 |
| BUB1 | 0.235 | 1.3 | 0.838 | −1.1 | 0.374 | 1.3 |
| PTP4A2 | 0.089 | −1.6 | 0.000 | −4.5 | 0.061 | 2.9 |
| YBX1 | 0.014 | −1.3 | 0.309 | −1.3 | 0.939 | 1.0 |
| LRBA | 0.246 | −4.9 | 0.207 | −10.5 | 0.050 | 2.1 |
| GATA3 | 0.748 | 1.0 | 0.004 | −35.8 | 0.003 | 36.9 |
| CX3CL1 | 0.049 | −7.6 | 0.631 | 1.3 | 0.136 | −9.7 |
| MAPRE2 | 0.001 | −1.7 | 0.177 | 1.2 | 0.008 | −2.0 |
| GMPS | 0.788 | 1.0 | 0.571 | 1.2 | 0.688 | −1.1 |
| CKS2 | 0.553 | 1.1 | 0.024 | −8.1 | 0.000 | 9.0 |
| SLC43A3 | 0.038 | −1.4 | 0.001 | −7.1 | 0.002 | 5.1 |

To determine differences in relative gene expression between intact tissue and LCM-procured cells, t-tests were performed with the results shown. The first 14 genes listed are from the carcinoma subset, while the remaining 18 genes are from the stromal subset. Values shown in bold indicate a P value of less than 0.05, and fold change observed for each gene is also shown.

Interestingly, when expression levels in the 14 cancer gene subset were determined in LCM-procured stromal cells compared to those of intact tissue (FIG. 9A and Table 19), seven genes (EVL, NAT1, ESR1, TBC1D9, TRIM29, SCUBE2, and IL6ST) were under-expressed (P value less than 0.05) relative to the intact tissue (−3.0, −5.9, −2.7, −2.4, −7.3, −4.3, and −1.1-fold, respectively). This result is consistent with the observation that stromal cells composed only 5% of the tissue section.

In the final analyses of the cancer gene subset, expression was compared in the LCM-procured populations of carcinoma and stromal cells. Expression of 11 of the 14 genes (EVL, NAT1, ESR1, ST8SIA1, TRIM29, SCUBE2, IL6ST, RABEP1, SLC39A6, TPBG, and TCEAL1) gave a statistically significant difference (P value less than 0.05, Table 19). Each of these genes was over-expressed in the carcinoma cells compared to the stromal cells (3.0, 13.9, 2.7, 3.9, 2.0, 4.4, 34.0, 3.3, 5.7, 4.4, and 3.7-fold, respectively) as predicted.

For the stromal gene subset, expression levels of seven genes (PFKP, ATAD2, XBP1, YBX1, CX3CL1, MAPRE2, and SLC43A3) was statistically different comparing the LCM-procured carcinoma cell population to the intact tissue (FIG. 9B and Table 19). Six of these genes were under-expressed in the cancer cell population compared to the intact tissue (−2.3, 1.8, −2.1, −1.3, −7.6, −1.7, and −1.4-fold, respectively).

Expression levels of the stromal gene subset was determined in LCM-procured stromal cells compared to those of intact tissue (FIG. 9B). Nine genes (FUT8, CENPA, PLK1, XBP1, MCM6, PTP4A2, GATA3, CKS2, and SLC43A3) were under-expressed (P value less than 0.05) relative to their levels in intact tissue (−5.2, −2.1, −3.3, −9.3, −4.1, −4.5, −35.8, −8.1, and −7.1-fold, respectively, Table 19). This result is consistent with the earlier observation of under-expression of these genes in stromal cells (composing only 5% of the tissue section) apparently due to being masked in the intact tissue analysis.

In the final analysis of this breast tissue specimen, expression of the 18 stromal gene subset was compared in the LCM-procured populations of carcinoma and stromal cells. Expression levels of 10 of the 18 genes (FUT8, CENPA, PLK1, ATAD2, XBP1, MCM6, GATA3, MAPRE2, CKS2, and SLC43A3) were statistically different (P value less than 0.05, Table 19) in the two cell types. Nine of these genes were over-expressed in the carcinoma cells compared to the stromal cells (5.0, 2.6, 2.4, 3.5, 4.5, 3.7, 36.9, −2.0, 9.0, and 5.1-fold, respectively). This observation indicates that the genes of the stromal gene subset are under-expressed in the stromal cells, which may be of clinical relevance.

### Representative Analyses Using a Tissue Biopsy Containing Intermediate Number of Carcinoma Cells

Using a biopsy specimen from a 44 year old patient with invasive ductal carcinoma, serial tissue sections were prepared which contained 60% carcinoma cells and 30% stromal cells. A comparison of relative expression of each gene in the entire 32 gene set was performed with RNA from intact tissue, LCM-procured carcinoma cells, and LCM-procured stromal cells (FIG. 10). Examining the carcinoma gene subset (FIG. 10A), expression levels of four genes (EVL, ST8SIA1, IL6ST, and DSC2) were statistically different comparing the LCM-procured carcinoma cell population to the intact tissue (3.7, −2.1, 8.9, and −14.4-fold, respectively, Table 20).

When expression levels of the 14 cancer gene subset were determined in LCM-procured stromal cells compared to those of intact tissue (FIG. 10A and Table 20), seven genes (GABRP, ST8SIA1, TRIM29, SLC39A6, TPBG, TCEAL1, and DSC2) were under-expressed (−7.1, −2.0, −23.7, −11.3, −2.9, −1.4, and −25.6-fold, respectively with P values less than 0.05. This result is consistent with the observation that stromal cells composed only 30% of each tissue section.

TABLE 20
Statistical differences in gene expression among intact tissue, LCM-procured
carcinoma cells and LCM-procured stromal cells shown in FIG. 6.

| | DIFFERENCES IN GENE | | DIFFERENCES IN GENE | | DIFFERENCES IN GENE | |
|---|---|---|---|---|---|---|
| | EXPRESSION BETWEEN | | EXPRESSION BETWEEN | | EXPRESSION BETWEEN | |
| | INTACT TISSUE & LCM- | | INTACT TISSUE & LCM- | | LCM-PROCURED CANCER | |
| | PROCURED CANCER CELLS | | PROCURED STROMAL CELLS | | CELLS & STROMAL CELLS | |
| | T-TEST | FOLD CHANGE | T-TEST | FOLD CHANGE | T-TEST | FOLD CHANGE |
| GENE | (P VALUE) | (CA/INTACT) | (P VALUE) | (ST/INTACT) | (P VALUE) | (CA/ST) |
| EVL | 0.011 | 3.7 | 0.835 | 1.1 | 0.012 | 3.4 |
| NAT1 | 0.447 | −1.5 | 0.502 | 1.3 | 0.060 | −2.0 |
| ESR1 | 0.144 | 2.3 | 0.297 | −1.5 | 0.098 | 3.6 |
| GABRP | 0.072 | −2.4 | 0.043 | −7.1 | 0.109 | 2.9 |
| ST8SIA1 | 0.037 | −2.1 | 0.022 | −2.0 | 0.807 | −1.1 |
| TBC1D9 | * | * | 0.114 | −19.2 | * | * |
| TRIM29 | 0.071 | 1.3 | 0.005 | −23.7 | 0.034 | 30.3 |
| SCUBE2 | 0.703 | −1.1 | 0.065 | −4.5 | 0.001 | 4.0 |
| IL6ST | 0.000 | 8.9 | 0.211 | −2.0 | 0.000 | 17.7 |
| RABEP1 | 0.678 | 1.4 | 0.231 | −2.0 | 0.363 | 2.8 |
| SLC39A6 | 0.060 | −2.1 | 0.026 | −11.3 | 0.023 | 5.5 |
| TPBG | 0.438 | 1.1 | 0.004 | −2.9 | 0.003 | 3.1 |
| TCEAL1 | 0.274 | 1.5 | 0.015 | −1.4 | 0.140 | 2.0 |
| DSC2 | 0.034 | −14.4 | 0.032 | −25.6 | 0.049 | 1.8 |
| FUT8 | 0.012 | −3.5 | 0.006 | −3.3 | 0.883 | −1.1 |
| CENPA | 0.096 | 2.8 | 0.033 | −8.3 | 0.047 | 23.0 |
| MELK | 0.084 | 3.3 | 0.020 | −4.2 | 0.047 | 14.0 |
| PFKP | 0.172 | −1.5 | 0.003 | −3.8 | 0.141 | 2.5 |
| PLK1 | 0.069 | 2.2 | 0.001 | −6.1 | 0.027 | 13.6 |
| ATAD2 | 0.037 | 3.4 | 0.000 | −4.3 | 0.021 | 14.4 |
| XBP1 | 0.320 | −1.2 | 0.003 | −1.9 | 0.164 | 1.6 |
| MCM6 | 0.094 | 2.1 | 0.000 | −48.8 | 0.029 | 103.5 |
| BUB1 | 0.140 | 2.1 | 0.001 | −78.0 | 0.045 | 161.0 |
| PTP4A2 | 0.235 | −1.5 | 0.003 | −3.1 | 0.247 | 2.0 |
| YBX1 | 0.046 | 1.9 | 0.000 | −3.8 | 0.013 | 7.3 |
| LRBA | 0.727 | 1.1 | 0.250 | −2.1 | 0.060 | 2.3 |
| GATA3 | 0.062 | −1.6 | 0.002 | −1.8 | 0.751 | 1.1 |
| CX3CL1 | 0.471 | −1.2 | 0.028 | −2.3 | 0.087 | 2.0 |
| MAPRE2 | 0.022 | −1.9 | 0.000 | −24.3 | 0.029 | 12.9 |
| GMPS | 0.052 | 2.6 | 0.052 | −52.5 | 0.023 | 135.3 |
| CKS2 | 0.026 | 4.4 | 0.000 | −25.4 | 0.016 | 112.8 |
| SLC43A3 | 0.008 | 1.5 | 0.011 | −4.7 | 0.001 | 7.0 |

In order to determine differences in relative gene expression between intact tissue and LCM-procured cells, t-tests were performed with the results shown. The first 14 genes listed are from the carcinoma subset, while the remaining 18 genes are from the stromal subset. Values shown in bold indicate a P value of less than 0.05, and fold change observed for each gene is also shown. (* indicates expression was undetected)

In the final analyses of the cancer gene subset in this tissue specimen, expression was compared in the LCM-procured populations of carcinoma and stromal cells. Expression levels of 7 of the 14 genes (EVL, TRIM29, SCUBE2, IL6ST, SLC39A6, TPBG, and DSC2) were statistically different (P value less than 0.05, Table 20). Each of these genes was over-expressed in the carcinoma cells compared to the stromal cells (3.4, 30.3, 4.0, 17.7, 5.5, 3.1, and 1.8-fold, respectively) as predicted.

For the 18 stromal gene subset, expression levels of five genes (ATAD2, YBX1, MAPRE2, CKS2, and SLC43A3) was statistically different (3.4, 1.9, −1.9, 4.4, and 1.5-fold, respectively) comparing the LCM-procured carcinoma cell population to the intact tissue (FIG. 10B and Table 20). This result is consistent with the observation that carcinoma cells composed only 60% of each tissue section.

Interestingly, when expression levels in the stromal gene subset were determined in LCM-procured stromal cells compared to those of intact tissue (FIG. 10B and Table 20), 16 genes (FUT8, CENPA, MELK, PFKP, PLK1, ATAD2, XBP1, MCM6, BUB1, PTP4A2, YBX1, GATA3, CX3CL1, MAPRE2, CKS2, and SLC43A3) were under-expressed relative to the intact tissue (−3.3, −8.3, −4.2, −3.8, −6.1, −4.3, −1.9, −48.8, −78.0, −3.1, −3.8, −1.8, −2.3, −24.3, −25.4, and −4.7-fold, respectively). This result is consistent with under-expression of genes in stromal cells of this specimen which contained only 30% of the intact tissue section.

In the final analyses of this tissue specimen, expression of the stomal gene subset was compared in the LCM-procured carcinoma and stromal cell populations. Eleven of the 18 genes (CENPA, MELK, PLK1, ATAD2, MCM6, BUB1, YBX1, MAPRE2, GMPS, CKS2, and SLC43A3) were statistically over-expressed compared to the stromal cells (23.0, 14.0, 13.6, 14.4, 103.5, 161.0, 7.3, 12.9, 135.3, 112.8, and 7.0-fold, respectively, Table 20).

### Representative Analyses Using a Tissue Biopsy Containing an Equal Number of Carcinoma and Stromal Cells

Using a tissue biopsy from a 69 year old patient with invasive ductal carcinoma, tissue sections were prepared which contained 30% carcinoma cells and 30% stromal cells. A comparison of relative expression of entire 32 gene set was performed with RNA from intact tissue, LCM-procured carcinoma cells, and LCM-procured stromal cells (FIG. 11 and Table 21). Examining the carcinoma gene subset (FIG. 11A), expression levels of five genes (TBC1D9, SCUBE2, IL6ST, SLC39A6, and TCEAL1) were statistically different comparing the LCM-procured carcinoma cell population to the intact tissue (Table 21). Each of the five genes was over-expressed in the cancer cell population compared to the intact tissue (23.7, 1.9, 5.2, 6.5, and 2.2-fold, respectively).

When expression levels of the 14 cancer gene subset were determined in LCM-procured stromal cells compared to those of intact tissue (FIG. 11A and Table 21), two genes (EVL and SCUBE2) were under-expressed (−1.8 and −2.4-fold, respectively). This result is consistent with EVL and SCUBE2 expression occurring primarily in the carcinoma cells.

In the final analyses of this 14 gene subset, expression levels were compared in LCM-procured populations of carcinoma and stromal cells. Expression of 5 of the 14 genes (ESR1, TBC1D9, SCUBE2, IL6ST, and TCEAL1) gave a statistically significant difference (Table 21). Each of these genes was over-expressed in the carcinoma cells compared to the stromal cells (2.1, 8.4, 4.7, 3.1, and 2.2-fold, respectively), as predicted.

Focusing on the 18 stromal gene subset (FIG. 11B), expression levels of 6 genes (ATAD2, LRBA, CX3CL1, MAPRE2, CKS2, and SLC43A3) were statistically different comparing the LCM-procured carcinoma cell population to the intact tissue (Table 21). Each of the 6 genes was differentially expressed in the LCM cell population (2.5, 3.1, −3.8, −1.8, 4.4, and −2.1-fold, respectively).

TABLE 21
Statistical differences in gene expression among intact tissue, LCM-procured
carcinoma cells and LCM-procured stromal cells shown in FIG. 7.

| | DIFFERENCES IN GENE | | DIFFERENCES IN GENE | | DIFFERENCES IN GENE | |
|---|---|---|---|---|---|---|
| | EXPRESSION BETWEEN | | EXPRESSION BETWEEN | | EXPRESSION BETWEEN | |
| | INTACT TISSUE & LCM- | | INTACT TISSUE & LCM- | | LCM-PROCURED CANCER | |
| | PROCURED CANCER CELLS | | PROCURED STROMAL CELLS | | CELLS & STROMAL CELLS | |
| | T-TEST | FOLD CHANGE | T-TEST | FOLD CHANGE | T-TEST | FOLD CHANGE |
| GENE | (P VALUE) | (CA/INTACT) | (P VALUE) | (ST/INTACT) | (P VALUE) | (CA/ST) |
| EVL | 0.216 | 1.4 | 0.005 | −1.8 | 0.061 | 2.6 |
| NAT1 | 0.125 | 3.5 | 0.149 | −3.7 | 0.087 | 12.7 |
| ESR1 | 0.053 | 2.0 | 0.862 | −1.1 | 0.036 | 2.1 |
| GABRP | 0.441 | 3.9 | 0.147 | −3.5 | 0.356 | 13.6 |
| ST8SIA1 | 0.413 | −2.4 | 0.769 | 1.2 | 0.256 | −2.9 |
| TBC1D9 | 0.027 | 23.7 | 0.144 | 2.8 | 0.027 | 8.4 |
| TRIM29 | 0.210 | 3.2 | 0.091 | 2.2 | 0.520 | 1.4 |
| SCUBE2 | 0.032 | 1.9 | 0.031 | −2.4 | 0.019 | 4.7 |
| IL6ST | 0.042 | 5.2 | 0.321 | 1.7 | 0.044 | 3.1 |
| RABEP1 | 0.136 | 3.5 | 0.199 | 1.5 | 0.191 | 2.4 |
| SLC39A6 | 0.003 | 6.5 | 0.064 | 4.3 | 0.123 | 1.5 |
| TPBG | 0.958 | 1.0 | 0.150 | −1.8 | 0.115 | 1.9 |
| TCEAL1 | 0.038 | 2.2 | 0.867 | 1.1 | 0.025 | 2.2 |
| DSC2 | 0.177 | 6.6 | 0.068 | 8.9 | 0.561 | −1.3 |
| FUT8 | 0.057 | 1.6 | 0.449 | −1.2 | 0.022 | 1.9 |
| CENPA | 0.359 | 1.3 | 0.002 | 3.5 | 0.000 | −2.7 |
| MELK | 0.422 | 1.2 | 0.129 | 3.1 | 0.156 | −2.5 |
| PFKP | 0.074 | −1.8 | 0.032 | −1.7 | 0.818 | −1.1 |
| PLK1 | 0.842 | −1.0 | 0.021 | −1.8 | 0.046 | 1.8 |
| ATAD2 | 0.042 | 2.5 | 0.319 | 1.6 | 0.184 | 1.5 |
| XBP1 | 0.166 | 1.6 | 0.001 | −5.1 | 0.035 | 8.0 |
| MCM6 | 0.617 | 1.2 | 0.336 | 1.4 | 0.497 | −1.2 |
| BUB1 | 0.256 | 2.4 | 0.930 | −1.1 | 0.247 | 2.5 |
| PTP4A2 | 0.106 | 2.0 | 0.052 | −2.3 | 0.054 | 4.7 |
| YBX1 | 0.173 | −1.3 | 0.677 | 1.2 | 0.475 | −1.6 |
| LRBA | 0.009 | 3.1 | 0.292 | 5.1 | 0.513 | −1.6 |
| GATA3 | 0.080 | 1.5 | 0.004 | −50.5 | 0.011 | 73.4 |
| CX3CL1 | 0.012 | −3.8 | 0.022 | 3.0 | 0.040 | −11.2 |
| MAPRE2 | 0.049 | −1.8 | 0.041 | 2.4 | 0.005 | −4.2 |
| GMPS | 0.254 | 1.4 | 0.142 | 4.8 | 0.157 | −3.5 |
| CKS2 | 0.018 | 4.4 | 0.005 | 4.0 | 0.574 | 1.1 |
| SLC43A3 | 0.022 | −2.1 | 0.638 | −1.2 | 0.433 | −1.7 |

In order to determine differences in relative gene expression between intact tissue and LCM-procured cells, t-tests were performed with the results shown. The first 14 genes listed are from the carcinoma subset, while the remaining 18 genes are from the stromal subset. Values shown in bold indicate a P value of less than 0.05, and fold change observed for each gene is also shown.

When expression levels of the stromal gene subset were determined in LCM-procured stromal cells compared to those of intact tissue (FIG. 11B and Table 21), eight genes (CENPA, PFKP, PLK1, XBP1, GATA3, CX3CL1, MAPRE2, and CKS2) were differentially expressed relative to the intact tissue (3.5, −1.7, −1.8, −5.1, −50.5, 3.0, 2.4, and 4.0, respectively). This result indicates that although the genes are significantly different in the stromal cells, their regulation is to be both over- and under-expressed in the stromal cells, which appears to be inconsistent in each patient specimen analyzed.

In the final analyses for this tissue specimen, expression of the 18 stromal gene subset was compared in the LCM-procured populations of carcinoma and stromal cells. Expression of 7 of the 18 genes (FUT8, CENPA, PLK1, XBP1, GATA3, CX3CL1, and MAPRE2) were statistically different (Table 21). Both over- and under-expression of these genes was observed in the carcinoma cells compared to the stromal cells (1.9, −2.7, 1.8, 8.0, 73.4, −11.2, and −4.2-fold, respectively).

### Summary of Expression Differences in the Carcinoma Gene Subset

In order to evaluate and interpret the vast amount of data collected from these representative specimens and the other tissue sections evaluated, a summary of statistical differences in gene expression among intact tissue, LCM-procured carcinoma cells and stromal cells was composed (Table 22 and 23). Gene expression was compared between the intact tissue section and LCM-procured cell populations corresponding to the cancer and stromal gene subsets, and Welch t-tests were used to identify any gene in which expression was significantly different between the groups. Since genes of the two subsets are expressed differently in each patient specimen, as shown in FIGS. 9-11, specimens that had statistical differences in gene expression between. LCM-procured cells and intact tissue were divided by the total number of specimens evaluated to provide a percentage. The results of fold change observed in all samples analyzed are presented to illustrate the broad range of gene expression levels observed.

TABLE 22
Summary of statistical differences in expression of the carcinoma gene subset among
intact tissue, LCM-procured carcinoma cells and LCM-procured stromal cells.

| | COMPARISON OF INTACT TISSUE & | | | | COMPARISON OF INTACT TISSUE & | | | | COMPARISON OF CANCER CELLS & | | | |
|---|---|---|---|---|---|---|---|---|---|---|---|---|
| | CANCER CELLS | | | | STROMAL CELLS | | | | STROMAL CELLS | | | |
| | PATIENTS | | | | PATIENTS | | | | PATIENTS | | | |
| | WITH DIFFER- | | | | WITH DIFFER- | | | | WITH DIFFER- | | | |
| | ENCES IN | | AVERAGE FOLD | | ENCES IN | | AVERAGE FOLD | | ENCES IN | | AVERAGE FOLD | |
| | EXPRESSION | | CHANGE | | EXPRESSION | | CHANGE | | EXPRESSION | | CHANGE | |
| GENE | (P < 0.05) | | (CA/INTACT) | | (P < 0.05) | | (ST/INTACT) | | (P < 0.05) | | (CA/ST) | |
| EVL | 7/33 | (21.2%) | 0.74 | (−2.6 to 4.2) | 3/14 | (21.4%) | 0.13 | (−3.3 to 8.2) | 6/13 | (46.2%) | 0.99 | (−3.6 to 6.1) |
| NAT1 | 4/33 | (12.1%) | 0.55 | (−9.4 to 43.2) | 4/14 | (28.6%) | −4.16 | (−32.7 to 27.1) | 4/13 | (30.8%) | 5.13 | (−3.9 to 20.6) |
| ESR1 | 5/33 | (15.2%) | 2.12 | (−9.0 to 32.4) | 4/14 | (28.6%) | −4.32 | (−24.1 to 3.4) | 5/13 | (38.5%) | 4.15 | (−8.5 to 16.0) |
| GABRP | 4/30 | (12.1%) | 1.5 | (−51.0 to 116.0) | 4/14 | (28.6%) | −173 | (−1743 to −1.2) | 1/11 | (9.1%) | 48.74 | (−3.1 to 427.5) |
| ST8SIA1 | 7/33 | (21.2%) | −0.32 | (−4.2 to 2.3) | 1/14 | (7.1%) | 0.37 | (−3.8 to 4.2) | 1/13 | (7.7%) | −1.64 | (−8.0 to 3.9) |
| TBC1D9 | 10/28 | (35.7%) | −1.58 | (−16.3 to 23.7) | 5/14 | (35.7%) | −32.7 | (−288.5 to 2.8) | 5/9 | (55.6%) | 4.02 | (−3.4 to 17.7) |
| TRIM29 | 7/32 | (30.4%) | −0.97 | (−4.3 to 10.5) | 5/14 | (35.7%) | −57.13 | (−502 to 2.2) | 6/13 | (46.2%) | 37.80 | (−1.4 to 172.0) |
| SCUBE2 | 9/33 | (27.3%) | −0.85 | (−10.8 to 25.1) | 3/14 | (21.4%) | −0.87 | (−4.5 to 6.9) | 6/13 | (46.2%) | −2.24 | (−20.8 to 9.6) |
| IL6ST | 12/33 | (36.4%) | −0.86 | (−23.0 to 14.7) | 4/14 | (28.6%) | −8.49 | (−70 to 2.27) | 5/13 | (38.5%) | 19.36 | (−5.5 to 142.9) |
| RABEP1 | 8/33 | (24.2%) | −0.15 | (−7.4 to 4.2) | 4/14 | (28.6%) | −1.33 | (−10.8 to 3.1) | 5/13 | (38.5%) | 1.10 | (−5.6 to 6.0) |
| SLC39A6 | 8/33 | (24.2%) | −1.31 | (−11.1 to 6.5) | 3/14 | (21.4%) | −13.46 | (−86.2 to 4.3) | 6/13 | (46.2%) | 3.98 | (−4.0 to 17.0) |
| TPBG | 2/33 | (6.1%) | −0.43 | (−8.2 to 3.5) | 1/14 | (7.1%) | −2.71 | (−10.2 to 1.1) | 5/13 | (38.5%) | 1.67 | (−1.4 to 6.8) |
| TCEAL1 | 3/33 | (9.0%) | 0.33 | (−3.9 to 4.3) | 4/14 | (28.6%) | −1.18 | (−5.3 to 1.9) | 4/13 | (30.8%) | 1.43 | (−3.2 to 5.2) |
| DSC2 | 8/33 | (24.2%) | 0.35 | (−14.5 to 39.5) | 7/14 | (50.0%) | −3.33 | (−26.4 to 8.9) | 4/13 | (30.8%) | 1.10 | (−9.7 to 8.9) |

TABLE 23
Summary of statistical differences in expression of the stromal gene subset among
intact tissue, LCM-procured carcinoma cells and LCM-procured stromal cells.

| | COMPARISON OF INTACT TISSUE & | | | | COMPARISON OF INTACT TISSUE & | | | | COMPARISON OF CANCER CELLS & | | | |
|---|---|---|---|---|---|---|---|---|---|---|---|---|
| | CANCER CELLS | | | | STROMAL CELLS | | | | STROMAL CELLS | | | |
| | PATIENTS | | | | PATIENTS | | | | PATIENTS | | | |
| | WITH DIFFER- | | | | WITH DIFFER- | | | | WITH DIFFER- | | | |
| | ENCES IN | | AVERAGE FOLD | | ENCES IN | | AVERAGE FOLD | | ENCES IN | | AVERAGE FOLD | |
| | EXPRESSION | | CHANGE | | EXPRESSION | | CHANGE | | EXPRESSION | | CHANGE | |
| GENE | (P < 0.05) | | (CA/INTACT) | | (P < 0.05) | | (ST/INTACT) | | (P < 0.05) | | (CA/ST) | |
| FUT8 | 7/13 | (53.8%) | −0.26 | (−4.0 to 10.8) | 10/23 | (43.5%) | −1.48 | (−9.3 to 13.0) | 4/12 | (33.3%) | 1.60 | (−2.2 to 5.5) |
| CENPA | 4/13 | (30.8%) | −1.32 | (−17.3 to 2.8) | 15/23 | (65.2%) | −8.21 | (−54.0 to 3.5) | 6/12 | (50.0%) | 6.62 | (−2.7 to 23.0) |
| MELK | 4/13 | (30.8%) | −0.30 | (−3.4 to 3.3) | 10/23 | (43.5%) | −10.34 | (−103.3 to 3.1) | 3/12 | (25.0%) | 2.73 | (−2.5 to 14.0) |
| PFKP | 6/13 | (46.2%) | −2.94 | (−7.1 to 1.1) | 14/23 | (60.9%) | −10.48 | (−102 to 2.4) | 3/12 | (25.0%) | 3.56 | (−1.9 to 21.0) |
| PLK1 | 3/13 | (23.1%) | −1.04 | (−6.3 to 2.4) | 15/23 | (65.2%) | −4.31 | (−14.7 to 1.4) | 6/12 | (50.0%) | 3.90 | (1.1 to 13.6) |
| ATAD2 | 4/13 | (30.8%) | 1.15 | (−1.3 to 3.9) | 9/23 | (39.1%) | −2.43 | (−9.4 to 1.62) | 6/12 | (50.0%) | 4.36 | (1.2 to 14.4) |
| XBP1 | 5/13 | (38.5%) | −1.28 | (−3.4 to 1.6) | 13/23 | (56.5%) | −3.53 | (−14.2 to 2.5) | 6/12 | (50.0%) | 2.55 | (−3.5 to 11.9) |
| MCM6 | 4/13 | (30.8%) | −1.11 | (−6.0 to 2.1) | 8/23 | (34.8%) | −6.22 | (−48.8 to 3.4) | 4/12 | (33.3%) | 11.36 | (−1.7 to 103.5) |
| BUB1 | 2/13 | (15.4%) | −1.51 | (−14.0 to 2.4) | 7/23 | (30.4%) | −6.06 | (−78.0 to 4.5) | 6/12 | (50.0%) | 15.26 | (−6.0 to 161.0) |
| PTP4A2 | 5/13 | (38.5%) | −1.84 | (−5.9 to 2.0) | 11/23 | (47.8%) | −3.24 | (−37.0 to 3.6) | 4/12 | (33.3%) | 1.76 | (−4.8 to 17.3) |
| YBX1 | 4/13 | (30.8%) | −0.36 | (−2.1 to 1.9) | 12/23 | (52.2%) | −2.03 | (−4.3 to 1.8) | 5/12 | (41.7%) | 2.12 | (−1.6 to 7.3) |
| LRBA | 7/13 | (53.8%) | −5.41 | (−29.2 to 10.4) | 8/23 | (34.8%) | −2.27 | (−42.8 to 21.1) | 5/12 | (41.7%) | −4.88 | (−47.5 to 4.2) |
| GATA3 | 1/13 | (7.7%) | −0.88 | (−5.4 to 2.1) | 14/23 | (60.9%) | −8.18 | (−50.5 to 3.7) | 4/12 | (33.3%) | 11.83 | (−2.2 to 73.4) |
| CX3CL1 | 7/13 | (53.8%) | −2.77 | (−8.3 to 1.8) | 13/23 | (56.5%) | −4.83 | (−35.1 to 3.0) | 4/12 | (33.3%) | −1.06 | (−11.2 to 8.8) |
| MAPRE2 | 5/13 | (38.5%) | −1.19 | (−4.7 to 3.8) | 5/23 | (21.7%) | −1.42 | (−24.3 to 5.2) | 5/12 | (41.7%) | −0.41 | (−4.5 to 12.9) |
| GMPS | 0/13 | (0%) | −0.21 | (−4.0 to 3.9) | 6/23 | (26.1%) | −6.5 | (−52.5 to 4.8) | 5/12 | (41.7%) | 12.98 | (−3.5 to 135.3) |
| CKS2 | 5/13 | (38.5%) | 0.69 | (−8.0 to 4.4) | 10/23 | (43.5%) | −5.52 | (−25.4 to 6.0) | 6/12 | (50.0%) | 17.43 | (−1.9 to 112.8) |
| SLC43A3 | 5/13 | (38.5%) | −1.13 | (−4.2 to 1.8) | 9/23 | (39.1%) | −3.36 | (−16.2 to 3.0) | 2/12 | (16.7%) | 1.61 | (−2.6 to 7.0) |

Genes of the carcinoma subset were expressed at levels that were statistically different between LCM-procured carcinoma cells and intact tissue in 21.4% of the specimens evaluated. Expression of those same 14 genes were also statistically different in the LCM-procured stromal cells compared to intact tissue in 26.5% of the specimens evaluated (Table 22). The average fold change between the two LCM-procured cell populations and the intact tissue section indicated that in general the genes appear to be down-regulated to a greater extent in the stromal cells (average fold change of −21.6 compared to −0.1 in the carcinoma). A few genes of this subset, e.g., TPBG, which was significantly different in only two of the 33 specimens evaluated, and TCEAL1, which was significantly different in only three of the 33 specimens, did not exhibit significant variation comparing carcinoma cells and intact tissue. Expression of ST8SIA1 and TPBG were statistically different in only one of the 14 LCM-procured stromal cell populations compared to the intact tissue.

A similar evaluation was performed directly comparing the expression of genes in each subset in both LCM-procured carcinoma cells and stromal cells (Table 22). Expression of two of 14 genes of the carcinoma subset (GABRP and ST8SIA1) was statistically different in carcinoma cells compared to that of stromal cells, each in only a single tissue specimen. Thus, 12 of the genes in the cancer subset were differentially expressed in the two LCM-procured cell populations of 13 breast carcinoma specimens. The majority of the genes were over-expressed in the carcinoma cells compared to the stromal cells, which would be predicted from the earlier studies from Wittliff and co-workers [41; 57; 70] using LCM-procured carcinoma cells.

### Summary of Expression Differences in the Stromal Gene Subset

The following investigation of LCM-procured stromal cells represents a unique approach that has never been reported. Genes of the stromal subset were statistically different in expression levels observed when comparing LCM-procured carcinoma cells to intact tissue (33.4% of the tissue specimens evaluated). Those 18 genes were also statistically different in the LCM-procured stromal cells and the intact tissue in 45.7% of the specimens (Table 23). The average fold change in gene expression between the two LCM-procured cell populations and intact tissue shows that most of the genes were down-regulated in stromal cells (average fold change of −5.0 compared to −1.2 in the carcinoma). GMPS and GATA3 genes in this stromal subset were expressed similarly in carcinoma cells and intact tissue in 13 specimens. However, many genes of the stromal subset were expressed at levels significantly different in LCM-procured stromal cell populations compared to the intact tissue (Table 23). In order to directly compare expression of the stromal gene subset in the specific cell types, a direct comparison of LCM-procured carcinoma cells and stromal cells was performed (Table 23). Expression of SLC43A3 was statistically different in carcinoma cells compared to stromal cells in only two of 12 patient specimens. However, expression of the other 17 genes was differentially expressed in many tissue specimens. Carcinoma cells appeared to over-express many of the genes identified in the stromal subset.

Clinical Correlations with Gene Expression in Different Cell Types

In general, the genes of both the carcinoma and stromal subsets appear to be over-expressed in the carcinoma cells compared to the stromal cells. However, it should be noted that if under-expression of a gene in either subset is found to be clinically relevant, it is likely that the gene will be under-expressed to a greater extent in the stromal cell population. In order to address the clinical implications of gene expression in the individual cell types, survival analyses (i.e., Cox proportional hazards model) were performed on the expression levels of genes (Tables 24 and 25).

Cox regression survival analyses identified one gene (TBC1D9) whose expression appeared to be related to disease-free survival using univariate analysis (Table 24). In addition, expression levels of TPBG appeared to be related to overall survival. Over-expression of each of these genes was correlated with an increased likelihood of recurrence or death from breast cancer (HR=1.20 and 1.71, respectively. Hazard ratios of greater than 1 indicate an increased likelihood of an event (i.e., breast cancer recurrence or death due to breast cancer). These correlations with survival indicate expression levels of TBC1D9 and TPBG in the carcinoma cells are associated with the clinical outcome of cancer patients.

Investigation of the expression of 32 candidate genes as single variables in LCM-procured stromal cells gave Cox regressions identifying 6 genes (CENPA, MELK, ATAD2, MCM6, YBX1, and GMPS) that appeared to be related to disease-free survival using univariate analysis (Table 25). Over-expression of each of these genes was correlated with an increased likelihood of recurrence (HR=9.47, 16.30, 3.10, 1.92, 4.39, and 2.02, respectively). Expression levels of 5 genes (TBCID9, MCM6, YBX1, GMPS, and CKS2) appeared to be related to overall survival. Over-expression of each of these genes was correlated with an increased likelihood of death due to breast cancer (HR=1.72, 1.77, 3.52, 2.78, and 1.89, respectively). These correlations with overall survival indicate that expression levels of TBC1D9, CENPA, MELK, ATAD2, MCM6, YBX1, GMPS, and CKS2 in the stromal cells were associated with the clinical outcome of cancer patients. Interestingly, over-expression of TBC1D9, a member of a family of proteins known to stimulate the GTPase activity of RAB proteins [191], in either carcinoma cells or surrounding stromal cells appear to be associated with poor survival. Collectively, these results have refined the selection of genes composing molecular signatures for the individual cell types.

TABLE 24
Cox regression survival analyses of the expression of the entire 32 gene
set in LCM-procured carcinoma cells as a function of disease-free and
overall survival. P values represent the level of significance of expression
for each gene, as a continuous variable. Expression of TBC1D9 appears
to be related to disease-free survival using univariate analysis, while
expression of TPBG appears to be related to overall survival. Over-
expression of each of these genes was correlated with an increased
likelihood of recurrence or death from breast cancer (HR = 1.20 and 1.71,
respectively).

| | | Disease-free | | | |
|---|---|---|---|---|---|
| | | Surivival | | Overall Survival | |
| | GENE | | HAZARD | | HAZARD |
| GENE ID | SUBSET | P VALUE | RATIO | P VALUE | RATIO |
| EVL | carcinoma | 0.80 | 0.95 | 0.91 | 0.98 |
| NAT1 | carcinoma | 0.44 | 1.07 | 0.16 | 1.13 |
| ESR1 | carcinoma | 0.82 | 1.02 | 0.47 | 1.05 |
| GABRP | carcinoma | 0.69 | 1.03 | 0.25 | 0.94 |
| ST8SIA1 | carcinoma | 0.11 | 1.37 | 0.40 | 1.16 |
| TBC1D9 | carcinoma | 0.04 | 1.20 | 0.07 | 1.17 |
| TRIM29 | carcinoma | 0.58 | 0.94 | 0.24 | 0.89 |
| SCUBE2 | carcinoma | 0.23 | 1.12 | 0.11 | 1.16 |
| IL6ST | carcinoma | 0.85 | 1.02 | 0.45 | 1.09 |
| RABEP1 | carcinoma | 0.09 | 1.40 | 0.38 | 1.17 |
| SLC39A6 | carcinoma | 0.47 | 1.09 | 0.25 | 1.13 |
| TPBG | carcinoma | 0.44 | 1.21 | 0.03 | 1.71 |
| TCEAL1 | carcinoma | 0.11 | 1.29 | 0.17 | 1.21 |
| DSC2 | carcinoma | 0.20 | 0.73 | 0.72 | 1.06 |
| FUT8 | stromal | 0.21 | 1.68 | 0.11 | 1.73 |
| CENPA | stromal | 0.53 | 1.34 | 0.73 | 0.92 |
| MELK | stromal | 0.69 | 1.23 | 0.77 | 0.90 |
| PFKP | stromal | 0.67 | 1.26 | 0.73 | 0.88 |
| PLK1 | stromal | 0.36 | 1.53 | 0.27 | 1.60 |
| ATAD2 | stromal | 0.21 | 1.71 | 0.12 | 2.01 |
| XBP1 | stromal | 0.62 | 1.16 | 0.35 | 1.26 |
| MCM6 | stromal | 0.39 | 1.38 | 0.31 | 1.44 |
| BUB1 | stromal | 0.32 | 1.57 | 0.85 | 1.06 |
| PTP4A2 | stromal | 0.27 | 1.61 | 0.16 | 1.74 |
| YBX1 | stromal | 0.75 | 1.19 | 0.51 | 1.40 |
| LRBA | stromal | 0.39 | 1.42 | 0.24 | 1.51 |
| GATA3 | stromal | 0.36 | 1.26 | 0.18 | 1.26 |
| CX3CL1 | stromal | 0.39 | 0.74 | 0.30 | 0.73 |
| MAPRE2 | stromal | 0.77 | 1.14 | 0.65 | 1.20 |
| GMPS | stromal | 0.85 | 1.09 | 0.90 | 0.95 |
| CKS2 | stromal | 0.47 | 1.27 | 0.41 | 1.30 |
| SLC43A3 | stromal | 0.96 | 1.02 | 0.81 | 1.08 |

TABLE 25
Cox regression survival analyses of the expression of the entire 32 gene
set in LCM-procured stromal cells as a function of disease-free and
overall survival. P values represent the level of significance of expression
for each gene, as a continuous variable. Expression of CENPA, MELK,
ATAD2, MCM6, YBX1, and GMPS appears to be related to disease-
free survival using univariate analysis, while expression of TBC1D9,
MCM6, YBX1, GMPS, and CKS2 appears to be related to overall
survival. Over-expression of each of these genes was correlated with an
increased likelihood of recurrence or death from breast cancer (HR = 9.47,
16.30, 3.10, 1.92, 4.39, 2.02, 1.72, 1.77, 3.52, 2.78,
and 1.89, respectively).

| | | Disease-free | | | |
|---|---|---|---|---|---|
| | | Surivival | | Overall Survival | |
| | GENE | | HAZARD | | HAZARD |
| GENE ID | SUBSET | P VALUE | RATIO | P VALUE | RATIO |
| EVL | carcinoma | 0.88 | 0.93 | 0.71 | 1.17 |
| NAT1 | carcinoma | 0.65 | 1.10 | 0.38 | 1.16 |
| ESR1 | carcinoma | 0.38 | 1.18 | 0.25 | 0.12 |
| GABRP | carcinoma | 0.61 | 1.07 | 0.93 | 0.99 |
| ST8SIA1 | carcinoma | 0.94 | 0.95 | 0.91 | 1.06 |
| TBC1D9 | carcinoma | 0.08 | 2.05 | 0.05 | 1.72 |
| TRIM29 | carcinoma | 0.43 | 1.23 | 0.92 | 1.02 |
| SCUBE2 | carcinoma | 0.93 | 1.03 | 0.54 | 1.18 |
| IL6ST | carcinoma | 0.71 | 0.88 | 0.35 | 0.78 |
| RABEP1 | carcinoma | 0.89 | 1.11 | 0.51 | 1.44 |
| SLC39A6 | carcinoma | 0.11 | 1.89 | 0.06 | 1.57 |
| TPBG | carcinoma | 0.22 | 2.60 | 0.11 | 2.22 |
| TCEAL1 | carcinoma | 0.38 | 2.83 | 0.17 | 2.20 |
| DSC2 | carcinoma | 0.42 | 1.43 | 0.28 | 1.52 |
| FUT8 | stromal | 0.12 | 3.01 | 0.08 | 2.06 |
| CENPA | stromal | 0.05 | 9.47 | 0.21 | 1.22 |
| MELK | stromal | 0.04 | 16.30 | 0.21 | 1.41 |
| PFKP | stromal | 695.00 | 1.05 | 0.85 | 1.02 |
| PLK1 | stromal | 0.16 | 1.96 | 0.16 | 1.59 |
| ATAD2 | stromal | 0.02 | 3.10 | 0.05 | 1.99 |
| XBP1 | stromal | 0.47 | 0.74 | 0.96 | 0.99 |
| MCM6 | stromal | 0.02 | 1.92 | 0.01 | 1.77 |
| BUB1 | stromal | 0.39 | 1.25 | 0.20 | 1.38 |
| PTP4A2 | stromal | 0.39 | 1.82 | 0.27 | 1.71 |
| YBX1 | stromal | 0.02 | 4.39 | 0.01 | 3.52 |
| LRBA | stromal | 0.71 | 0.88 | 0.88 | 1.04 |
| GATA3 | stromal | 0.98 | 1.01 | 0.26 | 1.19 |
| CX3CL1 | stromal | 0.25 | 1.63 | 0.25 | 1.42 |
| MAPRE2 | stromal | 0.17 | 2.72 | 0.14 | 1.65 |
| GMPS | stromal | 0.04 | 2.02 | 0.01 | 2.78 |
| CKS2 | stromal | 0.23 | 1943.6 | 0.01 | 1.89 |
| SLC43A3 | stromal | 0.63 | 1.26 | 0.55 | 1.26 |

Comparison of Results Obtained by qPCR and Microarray

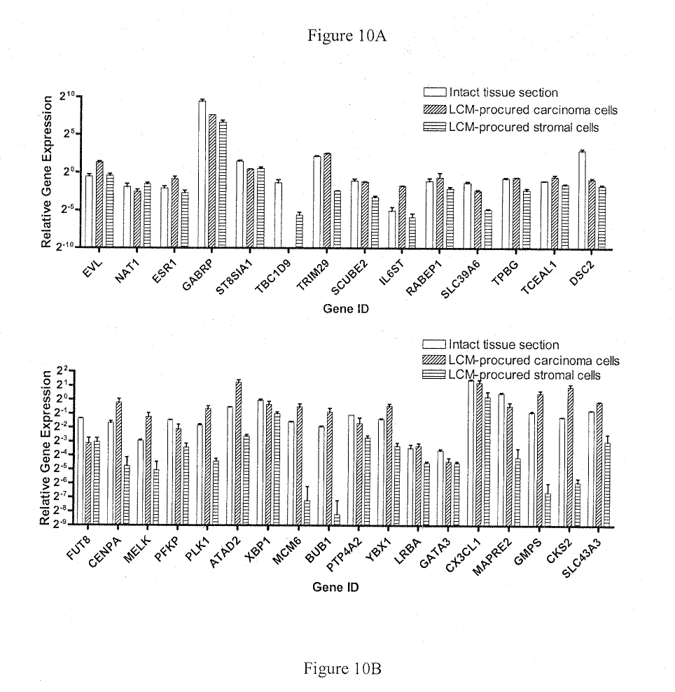

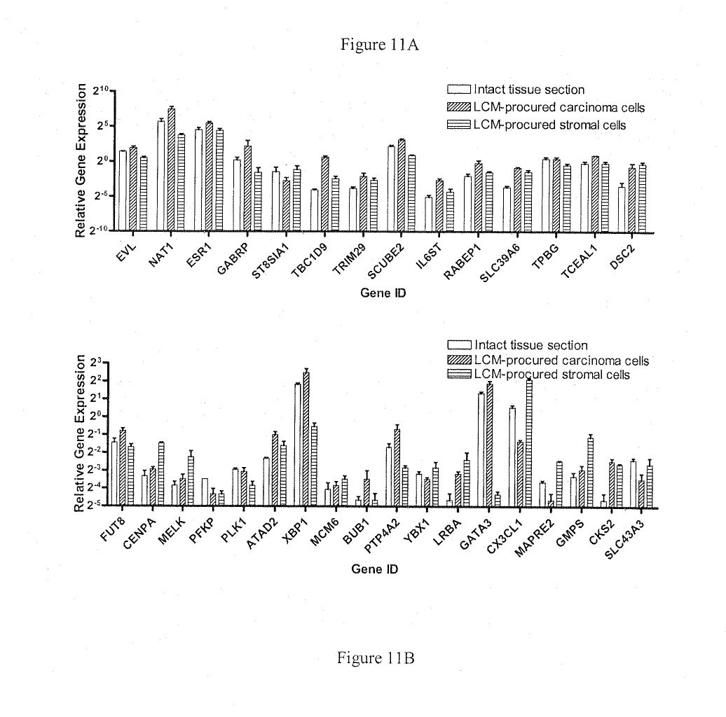

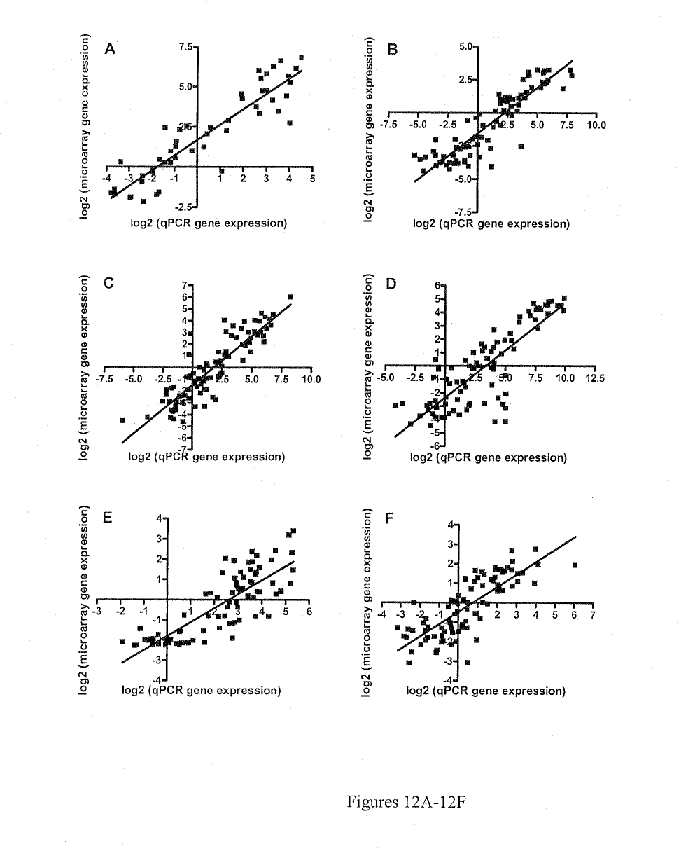

Gene expression in the different cell types was investigated by analyses of both gene subsets using the raw microarray data obtained from the previous LCM studies [41; 57; 70; 71]. While LCM is a technique of considerable use in discovery-based studies (e.g., [37; 40]), the goal of this investigation is to establish a clinically relevant gene subset amenable to development of a commercial laboratory test. An analysis of 86 specimens was performed comparing the gene expression results from qPCR results of intact tissue to those in the microarray data obtained from LCM-procured carcinoma cells (FIG. 12, Table 26). This allows comparisons of gene expression data across platforms, and provides insight as to the requirement for LCM prior to gene expression studies focusing on clinical relevance (i.e., are intact tissue-derived data similar to those obtained from LCM-procured cells?). These analyses are complicated by the variability of gene expression in different cell types present in a tissue biopsy. Therefore, additional data incorporating histology data were also analyzed, i.e., percent carcinoma, stromal and inflammatory cells as described earlier. Note that the microarray analyses used in FIG. 12 from studies reported by Wittliff et al. and Ma et al. [41; 57; 70; 71] were performed with LCM-procured carcinoma cells only. The slope of the linear regressions shown in FIG. 12, indicated consistency of expression measurements using the two platforms of microarray and qPCR. The correlation coefficients (r² values) listed in Table 26 provide evidence of the variability between the two platforms. Expression of several genes evaluated by qPCR of intact tissue sections correlated well with the microarray data from LCM-procured carcinoma cells (FIG. 12, Table 26). For example, NAT1 from the carcinoma gene subset, had a slope of 0.96 with an r² value of 0.83.

The expression results of several genes from the stromal cell subset also correlated reasonably well between qPCR analyses of intact tissue and those by microarray of the LCM-procured carcinoma cells. This implies that several genes within the “stromal cell subset” may, in fact, be expressed in both carcinoma and stromal cell types (e.g., qPCR analyses of XBP1, GATA3, and CENPA correlated with microarray data with an r² value of 0.67, 0.54, and 0.51, respectively). These genes may have been filtered informatically during earlier studies by Wittliff and coworkers [41; 57; 70; 71] resulting in molecular signatures based on the hierarchical clustering and gene filtering algorithms employed.

In general, expression of the genes from the cancer cell subset correlated better with the microarray data than the genes from the stromal cell subset as predicted (Table 26). T-tests of expression levels, performed between correlation coefficients from the genes within the two subsets, provided a P value of 0.001, indicating that there is a significant difference in gene expression between the two groups. T-tests also were performed between slopes of the regression analyses in each gene subset and gave a P value of less than 0.05 suggesting that there is a statistically significant difference between expression of the two gene subsets. The six genes which correlated best with the microarray data are listed in FIG. 12.

TABLE 26
Results from linear regression analyses of comparisons between
gene expression data obtained by qPCR compared to those
from microarray analyses.

| | | SLOPE OF | P-VALUE (SLOPE IS | |
|---|---|---|---|---|
| | GENE | LINEAR | SIGNIFICANTLY | |
| GENE | SUBSET | REGRESSION | NON-ZERO) | R2 |
| NAT1 | cancer | 0.96 | <0.0001 | 0.83 |
| SCUBE2 | cancer | 0.69 | <0.0001 | 0.81 |
| ESR1 | cancer | 0.83 | <0.0001 | 0.78 |
| GABRP | cancer | 0.71 | <0.0001 | 0.69 |
| XBP1 | stroma | 0.69 | <0.0001 | 0.67 |
| EVL | cancer | 0.64 | <0.0001 | 0.63 |
| ST8SIA1 | cancer | 0.70 | <0.0001 | 0.58 |
| TRIM29 | cancer | 0.52 | <0.0001 | 0.58 |
| GATA3 | stroma | 0.49 | <0.0001 | 0.54 |
| TCEAL1 | cancer | 0.59 | <0.0001 | 0.53 |
| CENPA | stroma | 0.72 | <0.0001 | 0.51 |
| TBC1D9 | cancer | 0.49 | <0.0001 | 0.50 |
| PFKP | stroma | 0.67 | <0.0001 | 0.48 |
| SLC39A6 | cancer | 0.29 | <0.0001 | 0.45 |
| RABEP1 | cancer | 0.43 | <0.0001 | 0.44 |
| CX3CL1 | stroma | 0.80 | <0.0001 | 0.42 |
| TPBG | cancer | 0.53 | <0.0001 | 0.41 |
| FUT8 | stroma | 0.47 | <0.0001 | 0.41 |
| SLC43A3 | stroma | 0.41 | <0.0001 | 0.40 |
| MELK | stroma | 0.55 | <0.0001 | 0.36 |
| DSC2 | cancer | 0.45 | <0.0001 | 0.34 |
| YBX1 | stroma | 0.35 | <0.0001 | 0.29 |
| ATAD2 | stroma | 0.57 | <0.0001 | 0.22 |
| BUB1 | stroma | 0.36 | <0.0001 | 0.19 |
| MAPRE2 | stroma | 0.30 | <0.0001 | 0.18 |
| PTP4A2 | stroma | 0.18 | <0.0001 | 0.18 |
| MCM6 | stroma | 0.21 | <0.0001 | 0.17 |
| LRBA | stroma | 0.14 | 0.0002 | 0.15 |
| CKS2 | stroma | 0.21 | 0.001 | 0.13 |
| IL6ST | cancer | 0.12 | 0.005 | 0.09 |
| GMPS | stroma | 0.19 | 0.015 | 0.07 |
| PLK1 | stroma | 0.09 | 0.163 | 0.02 |

### Survival Analyses Using Previously Obtained Microarray Data

Additional analyses were performed using microarray data obtained in a previous study of LCM-procured carcinoma cells for analysis of larger sample size of 247 breast cancer patients [41; 57; 70; 71]. Since a large number of patients were evaluated in that study, there should be greater statistical significance within the larger sample population. Table 27 shows the results of these univariate Cox regressions of patients for analyses of disease-free and overall survival. Expression of fourteen genes (EVL, NAT1, TBC1D9, SCUBE2, TPBG, TCEAL1, DSC2, MELK, PFKP, PLK1, XBP1, GATA3, MAPRE2, and GMPS) were statistically significant (P value less than 0.05) for disease-free survival. Analyses of overall survival determined that expression levels of 21 genes (EVL, NAT1, ESR1, TBC1D9, SCUBE2, IL6ST, RABEP1, TPBG, TCEAL1, DSC2, FUT8, MELK, PLK1, ATAD2, XBP1, BUB1, GATA3, MAPRE2, GMPS, CKS2, and SLC43A3) were statistically significant (Table 27).

Since the gene expression results discussed in Table 27 were obtained in microarray studies using LCM-procured cancer cells, results illustrating the statistical significance of genes from the “stromal subset” lead to a conclusion that several of these genes (e.g., FUT8, MELK, PFKP, PLK1, ATAD2, XBP1, BUB1, GATA3, MAPRE2, GMPS, CKS2, and SLC43A3) are clinically relevant in the carcinoma cells and are not specific to the surrounding stromal cells.

In general, gene expression levels of the candidate genes appeared to be similar in LCM-procured populations of carcinoma cells compared to those of intact tissue. This is likely due to a number of factors, including the observation that most of the carcinoma specimens utilized in these studies were composed of increased numbers of cancer cells compared to other cells types (Table 15). Each of the specimens examined in these investigations was collected as biopsy tissue for assessing the clinical pathology of the specimen to aid in diagnosis and treatment management. In addition, it is accepted (e.g., [8]) that carcinoma cells exhibit increased replication rates leading to an increase in the amount of mRNA present compared to other cell types. Many breast carcinomas are aneuploid or polyploidy and often exhibit larger nuclear to total cell volume ratios than non-cancerous cells. The observation that there are greater gene expression differences in the stromal cells compared to intact tissue implies a requirement for LCM when studying gene expression in stromal cells. However, once a molecular signature is defined from experiments using individual carcinoma cells, use of the intact tissue section is warranted.

Survival analyses of individual genes of both carcinoma and stromal subsets revealed over-expression of TBC1D9 and TPBG in the carcinoma cells were associated with clinical behavior of breast cancer in that disease-free and overall survival were diminished. It was also discovered that individual expression levels of TBC1D9, CENPA, MELK, ATAD2, MCM6, YBX1, GMPS, and CKS2 in the stromal cells were associated with poor prognosis of breast cancer. These results represent a unique finding in that over-expression of each of these 8 genes in stromal cells was correlated with an increased likelihood of death due to breast cancer. Interestingly, over-expression of TBC1D9 in either carcinoma cells or surrounding stromal cells appears to be associated with poor survival. Surprisingly, expression profiles of individual genes had predictive value although the number of samples should be increased to verify the level of confidence necessary for a single gene test.

In order to test the clinical validity of each of the 32 candidate genes validated by qPCR studies of this investigation, two approaches were undertaken. In the first, each of the 32 candidate genes was evaluated using clinical follow-up and microarray results from LCM-procured carcinoma cell preparations from 247 patient specimens [41; 57; 70; 71]. Examination of the entire 22,000 gene microarray results from carcinoma cells revealed expression levels of twelve genes in the “stromal subset” (e.g., FUT8, MELK, PFKP, PLK1, ATAD2, XBP1, BUB1, GATA3, MAPRE2, GMPS, CKS2, and SLC43A3) were clinically relevant. Thus it appears that expression of these genes is not limited to the stromal cells surrounding the carcinoma cells. Gene expression profiles of stromal cells, in addition to those of carcinoma cells, may be assessed. Hence, a molecular signature containing genes from both cell types elevates the power of prediction of clinical behavior of breast carcinoma.

TABLE 27
Relationship of gene expression as a function of survival using
univariate Cox regression of microarray data obtained from
LCM-procured carcinoma cells.

| | | DISEASE-FREE | | OVERALL | |
|---|---|---|---|---|---|
| | | SURVIVAL | | SURVIVAL | |
| | | | HAZARD | | HAZARD |
| GENE ID | SUBSET | P VALUE | RATIO | P VALUE | RATIO |
| EVL | carcinoma | 0.012 | 0.83 | 0.003 | 0.78 |
| NAT1 | carcinoma | 0.003 | 0.89 | 0.002 | 0.87 |
| ESR1 | carcinoma | 0.066 | 0.95 | 0.025 | 0.93 |
| GABRP | carcinoma | 0.755 | 1.01 | 0.281 | 1.05 |
| ST8SIA1 | carcinoma | 0.671 | 1.03 | 0.276 | 1.08 |
| TBC1D9 | carcinoma | 0.005 | 0.87 | 0.002 | 0.85 |
| TRIM29 | carcinoma | 0.269 | 1.06 | 0.296 | 1.07 |
| SCUBE2 | carcinoma | 0.040 | 0.92 | 0.020 | 0.90 |
| IL6ST | carcinoma | 0.166 | 0.86 | 0.019 | 0.74 |
| RABEP1 | carcinoma | 0.089 | 0.85 | 0.018 | 0.78 |
| SLC39A6 | carcinoma | 0.500 | 0.94 | 0.419 | 0.92 |
| TPBG | carcinoma | 0.003 | 0.77 | 0.002 | 0.73 |
| TCEAL1 | carcinoma | 0.040 | 0.86 | 0.008 | 0.80 |
| DSC2 | carcinoma | 0.038 | 1.13 | 0.001 | 1.26 |
| FUT8 | stromal | 0.106 | 0.87 | 0.007 | 0.76 |
| CENPA | stromal | 0.402 | 1.07 | 0.055 | 1.18 |
| MELK | stromal | 0.018 | 1.20 | 0.004 | 1.28 |
| PFKP | stromal | 0.046 | 1.18 | 0.060 | 1.20 |
| PLK1 | stromal | 0.001 | 1.68 | 0.038 | 1.45 |
| ATAD2 | stromal | 0.148 | 1.15 | 0.009 | 1.30 |
| XBP1 | stromal | 0.028 | 0.86 | 0.008 | 0.81 |
| MCM6 | stromal | 0.091 | 1.23 | 0.059 | 1.31 |
| BUB1 | stromal | 0.156 | 1.13 | 0.037 | 1.24 |
| PTP4A2 | stromal | 0.096 | 0.78 | 0.092 | 0.76 |
| YBX1 | stromal | 0.959 | 1.01 | 0.353 | 1.17 |
| LRBA | stromal | 0.462 | 0.91 | 0.352 | 0.88 |
| GATA3 | stromal | 0.007 | 0.86 | 0.002 | 0.83 |
| CX3CL1 | stromal | 0.355 | 1.05 | 0.096 | 1.11 |
| MAPRE2 | stromal | 0.017 | 1.26 | 0.002 | 1.41 |
| GMPS | stromal | 0.015 | 1.34 | 0.001 | 1.57 |
| CKS2 | stromal | 0.094 | 1.17 | 0.020 | 1.27 |
| SLC43A3 | stomal | 0.378 | 1.13 | 0.019 | 1.40 |

P values represent the level of significance of expression for each gene, as a continuous variable. Expression of EVL, NAT1, TBC1D9, SCUBE2, TPBG, TCEAL1, DSC2, MELK, PFKP, PLK1, XBP1, GATA3, MAPRE2, and GMPS appear to be related to disease-free survival using univariate analysis. Expression of EVL, NAT1, ESR1, TBC1D9, SCUBE2, IL6ST, RABEP1, TPBG, TCEAL1, DSC2, FUT8, MELK, PLK1, ATAD2, XBP1, BUB1, GATA3, MAPRE2, GMPS, CKS2, and SLC43A3 appear to be related to overall survival.

### Example 3

### Expression of Genes in Breast Tissue Samples

### Methods and Materials

Using the IRB-approved Biorepository and Database of the Hormone Receptor Laboratory, de-identified specimens of primary invasive ductal carcinoma were examined. Tissue-based properties (e.g., pathology of the cancer, grade, size, and tumor marker expression) and encoded patient-related characteristics (e.g., age, race, smoking status, menopausal status, stage, and nodal status) were utilized to examine the relationships between gene expression results and clinical parameters. One hundred twenty six tissue specimens from biopsies of invasive ductal carcinoma were selected for investigation as described in Table 28. The length of clinical follow-up and use of primary invasive breast carcinoma, as well as a significant division of patients with recurrent disease and disease-free were taken into consideration when selecting tissue specimens for studies predicting risk of recurrence. Tissue sections from breast cancer biopsies utilized for analyses of gene expression contained a median of about 60% carcinoma cells (range of about 10% to about 95%) and about 25% stromal cells (range of about 5% to about 65%).

TABLE 28
Characteristics of the patient population employed in this study.

| Patient Parameters | n |
|---|---|
| Median Age (range) | |
| 56 years (29-89.5) | 126 |
| Median Observation time (range) | |
| 61 months (3-147) | 126 |
| Race | |
| white | 119 |
| black | 7 |
| Histology | |
| Invasive ductal carcinoma | 126 |
| Median Tumor Size (Range) | |
| 30 mm (4-85) | 118 |
| Stage | |
| 1 | 23 |
| 2A | 46 |
| 2B | 35 |
| 3A | 10 |
| 3B | 6 |
| 4 | 6 |
| Grade | |
| 1 | 7 |
| 2 | 35 |
| 3 | 57 |
| 4 | 2 |
| unknown | 25 |
| Estrogen Receptor Status | |
| negative | 47 |
| positive | 79 |
| Lymph Node Status | |
| negative | 63 |
| positive | 52 |
| unknown | 6 |
| Recurrence Status | |
| yes | 46 |
| no | 75 |
| never disease-free | 5 |

### Tumor Marker Detection

Levels of mRNA expression were analyzed, while estrogen and progestin receptor protein levels were determined using either enzyme immunoassay (EIA) or ligand binding assay (LBA) and recorded in the Hormone Receptor Laboratory's Database. Briefly, both methods utilized chilled/frozen specimens that were sliced carefully with a scalpel on a Petri dish chilled on a frozen ice pack to maintain receptor integrity and then homogenized with a mass-to-buffer ratio of 1 g wet weight per 10 ml buffer containing 40 mM Tris-HCl, pH 7.4, containing 1.5 mM EDTA, 10% glycerol, 10 mM sodium molybdate, 10 mM monothioglycerol and 1 mM PMSF [11; 135]. Extracts were prepared by centrifugation at 100,000×g for 30 min. The total protein concentration of the extract is determined with the Bradford method.

A complete ligand binding assay was comprised of duplicates of six increasing concentrations of radiolabeled ligand with and without unlabeled inhibitor [11; 135; 243; 244]. Reactions were incubated overnight (12-18 hours) at 4° C. Unbound ligand was removed by addition of dextran-coated charcoal, incubated for 15 min, and then centrifuged at 3300×g for 15 min at 4° C. Supernatant was removed and radioactivity was detected in a liquid scintillation counter [11; 135; 243; 244]. ER/PR levels, expressed as fmol/mg protein, were recorded using a clinical cutoff value of 10 fmol/mg protein [11; 135; 243; 244].

ER and PR levels were also determined by EIA using a kit formerly distributed by Abbott Laboratories. This protocol utilized beads coated with Anti-ER or Anti-PR monoclonal antibodies, which were incubated with the tissue extracts [11; 135; 245; 246]. Unbound materials were aspirated and washed, before incubation with Anti-receptor antibodies conjugated with horseradish peroxidase. Color was developed and measured with a spectrometer at a wavelength of 492 nm [11; 135; 245; 246]. ER/PR levels, expressed as fmol/mg protein, were recorded using a clinical cutoff value of 15 fmol/mg protein [11; 135; 245; 246].

### Statistical Analyses

Kaplan-Meier analyses calculate the fraction of patients without an event (i.e., disease recurrence or death) from the total number of patients in the study over the range of time points [232; 241]. These calculations result in a plot depicting a decreasing step function, where steps occur when an event is recorded [241]. Comparison of survival curves produced from two strata is most commonly carried out using a log-rank test [232; 238]. This test generates a P value testing the null hypothesis that the survival curves are identical in the population as a whole [232].

A Cox proportional hazards model utilizes continuous variables in either univariate or multivariate models and has the added benefit of creating an equation to fit the survival data of a population (i.e., hi(t)=h0(t) eβxi). An advantage of this form of analysis is that a baseline hazard does not need to be known in order to calculate β, which is the coefficient of the variable being examined [238]. The main application of these survival analyses is to stratify patients by outcome and allow for better patient counseling and treatment decisions [242].

Normality tests, expression distribution plots, and Kaplan-Meier plots were performed in GRAPHPAD PRISM® Version 4 (GraphPad Software, La Jolla, Calif.). Pearson correlations, univariate cox regressions, and multivariate cox regressions were performed with SPSS® 17.0 statistical package (SPSS Inc., Chicago, Ill.). Calculations and model development were performed using log₂ transformations of relative gene expression data. Five patients that were never disease-free (Table 28) were omitted from Cox regressions of gene expression levels with disease-free survival.

### Results and Discussion

### Patient Survival as a Function of Known Prognostic Factors

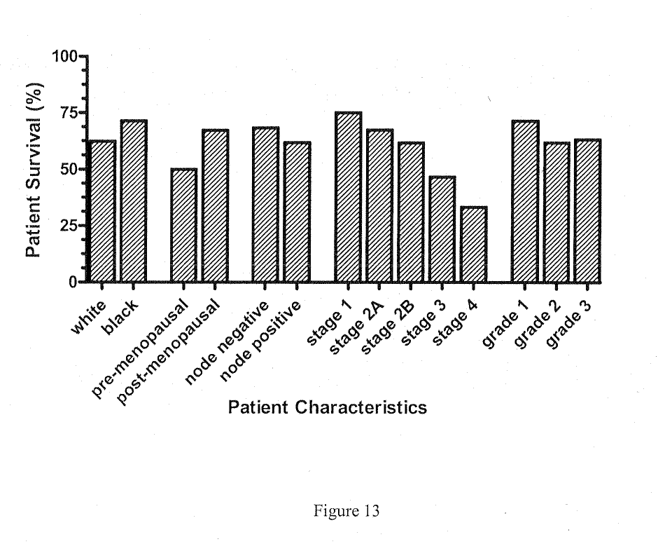

In order to analyze patient survival outcomes with known characteristics of the study population, a percent survival analysis was performed for each category, including race, menopausal status, lymph node involvement, stage of the cancer and tumor grade (FIG. 13). The percent survival for patients with race, menopausal status, clinical stage and grade followed expected outcomes. As previously reported (e.g., [247]), Caucasian patients have better overall survival compared to African-American patients, and post-menopausal patients have better survival compared to pre-menopausal patients. As expected, patients with breast cancer determined to be of a higher stage had significantly worse survival outcomes than patients with lower stage carcinomas, and those survival probabilities progressively declined with increased stage. There was not a large influence of tumor grade on overall patient survival, which was anticipated based on numerous other reports (e.g., [8; 247]). The survival outcome of patients with lymph node involvement was less significant than expected (e.g., [8; 247; 248]). This is due to the selection of patients necessary for completion of the project described in Appendix 1, which included equal numbers of patients with and without disease recurrence in lymph node negative and positive cancers.

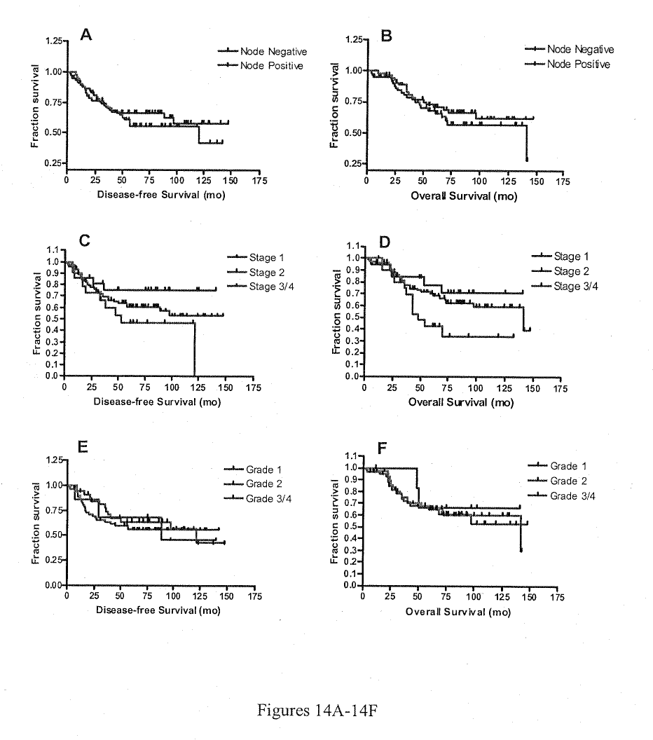

Before gene expression was analyzed for impacting cancer recurrence and survival, known prognostic factors, such as stage, grade and lymph node involvement, were evaluated by Kaplan-Meier survival plots using GRAPHPAD PRISM® software (FIG. 14). These statistical analyses of gene expression and its association with recurrence of the cancer (disease-free survival—DFS) and death of the patient due to breast cancer (overall survival—OS) takes into account “censoring” of patients due to loss of follow-up, as well as the time to event. Lymph node involvement, which is considered one of the most important clinical prognostic factors in breast cancer (e.g., [8; 247; 248]), did not significantly separate patient populations into good prognosis and poor prognosis groups for DFS (P value=0.43) and OS (P value=0.55) (FIGS. 14A and B). These results agree with the survival data shown in FIG. 13. When stage of disease was considered, patient populations were separated into good and poor prognosis groups for DFS (P value=0.19) and OS (P value=0.07). Expected trends were observed for each stage in both DFS (P value=0.07) and OS (P value=0.03) as shown in FIGS. 14C and 14D [20; 23; 169]. Tumor grade appeared to moderately predict DFS (FIGS. 14E and 14F). When analyzing expression of genes related to nodal status, the fact that nodal status did not exhibit expected behavior must be taken into consideration. However, stage, determined by tumor size, lymph node involvement, and presence of metastases, did exhibit the expected outcome, which would indicate no further bias in patient population (e.g., [8; 247; 248]).

Kaplan-Meier analyses were then performed on tumor markers with known importance in breast cancer [20; 22; 24; 94] showing their relationships with disease-free survival (FIGS. 15A and C) and overall survival of the patient (FIGS. 15B and 15D). Survival plots (FIGS. 15A and 15B) illustrate the correlation of estrogen receptor (protein) status and patient survival. Patients with ER-positive tumors had better disease-free and overall survival than patients with ER-negative tumors, although the difference was not statistically significant (DFS P value=0.42; OS P value=0.20) in this small patient population (n=126). Both ER and PR are accepted as biomarkers of breast cancer prognosis and treatment selection [20; 22; 24; 94. Plots labeled FIGS. 15C and 15D illustrate the correlation of progestin receptor (protein) status and survival. Patients with PR-positive tumors had better disease-free and overall survival than patients with PR-negative tumors, with separation approaching significance (DFS P value=0.06; OS P value=0.40). Although these tumor markers are considered useful prognostic factors in breast cancer, they are of greater utility in predicting response to hormonal therapies, such as Tamoxifen (e.g., [20; 22; 24; 94]).

### Gene Expression Levels and Distributions

In order to evaluate the distribution of individual gene expression levels in the biopsies from the patient population, the values were subjected to D'Agostino-Pearson normality tests using GRAPHPAD PRISM® to determine if they were sampled from a Gaussian distribution [232]. Genes with statistically significant P values (less than 0.05) are likely to be expression in a non-Gaussian distribution, while those with larger P values indicate that the gene expression levels were consistent with a Gaussian distribution. Results shown in Table 29 indicate thirteen genes, NAT1, ESR1, GABRP, IL6ST, CENPA, ATAD2, XBP1, MCM6, PTP4A2, LRBA, GATA3, GMPS, and SLC43A3, exhibited distributions consistent with a non-Gaussian population. These genes were then evaluated to determine if their expression exhibited bimodal distributions that identified a clinically relevant cut-off value for survival analyses.

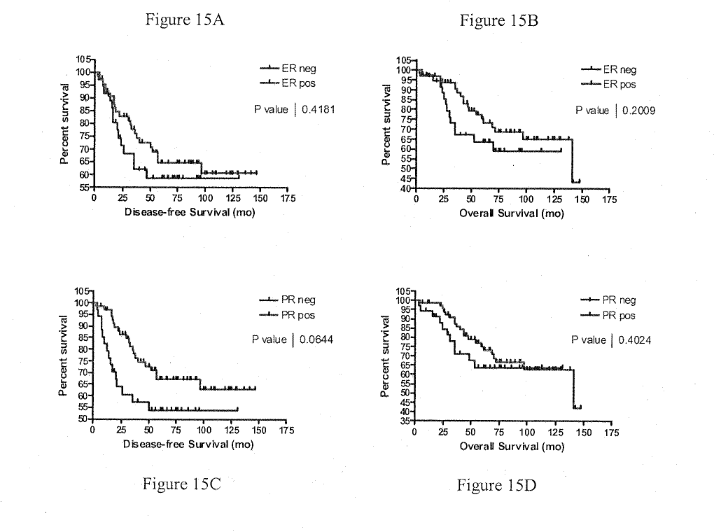

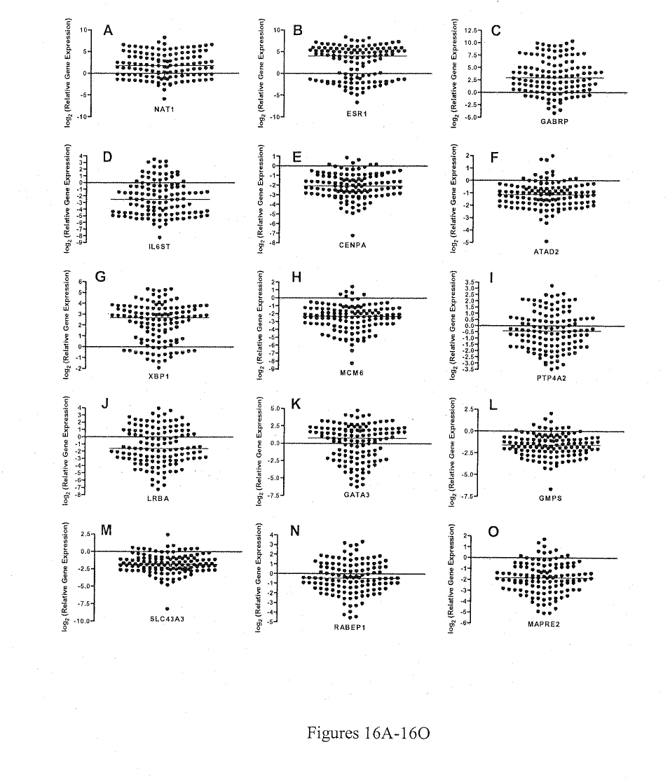

Expression levels and distribution of these thirteen genes from the 32 gene set were analyzed with dot plots [232; 249] using intact tissue sections of 126 invasive ductal carcinomas. FIG. 16 illustrates the results for thirteen genes, NAT1 (A), ESR1 (B), GABRP (C), IL6ST (D), CENPA (E), ATAD2 (F), XBP1 (G), MCM6 (H), PTP4A2 (I), LRBA (3), GATA3 (K), GMPS (L), and SLC43A3 (M), whose expression is consistent with non-Gaussian distribution as determined by the D'Agostino-Pearson normality test. Note that a log₂ relative gene expression value of 0 (shown by the bold horizontal line, FIG. 16) indicates no difference from that of the Universal Human Reference RNA (Stratagene) calibrator. The thin horizontal line on each plot indicates the median expression level. Seven of these genes appeared to exhibit bimodal grouping, and a cut-off value was determined based on separation of bimodal groups. These values were 1.0 for ESR1, 6.0 for GABRP, −4.0 for IL6ST, 1.0 for XBP1, 0.8 for PTP4A2, −1.0 for LRBA, and −0.5 for GATA3. Clinical relevance in survival analyses was evaluated using these cut-off values separating the bimodal grouping in later comparisons.

TABLE 29
Summary of D'Agostino-Pearson normality test results.

| GENE ID | P VALUE | GENE ID | P VALUE |
|---|---|---|---|
| EVL | 0.71 | FUT8 | 0.20 |
| NAT1 | 0.013 | CENPA | 0.021 |
| ESR1 | 0.001 | MELK | 0.76 |
| GABRP | 0.016 | PFKP | 0.21 |
| ST8SIA1 | 0.23 | PLK1 | 0.48 |
| TBC1D9 | 0.16 | ATAD2 | 0.028 |
| TRIM29 | 0.21 | XBP1 | 0.003 |
| SCUBE2 | 0.08 | MCM6 | 0.022 |
| IL6ST | 0.019 | BUB1 | 0.07 |
| RABEP1 | 0.93 | PTP4A2 | 0.047 |
| SLC39A6 | 0.30 | YBX1 | 0.12 |
| TPBG | 0.15 | LRBA | 0.033 |
| TCEAL1 | 0.87 | GATA3 | 0.009 |
| DSC2 | 0.26 | CX3CL1 | 0.12 |
| | | MAPRE2 | 0.94 |
| | | GMPS | 0.037 |
| | | CKS2 | 0.72 |
| | | SLC43A3 | 0.001 |

Gene expression levels were evaluated to determine if they were sampled from a population exhibiting Gaussian distribution using the D'Agostino-Pearson normality test in GRAPHPAD PRISM®. Genes exhibiting statistically significant P values (less than 0.05 and shown in bold) were likely to be from a non-Gaussian distribution, while those with larger P values are consistent with a Gaussian distribution.

### Correlations of Gene Expression Levels Using Various Combinations

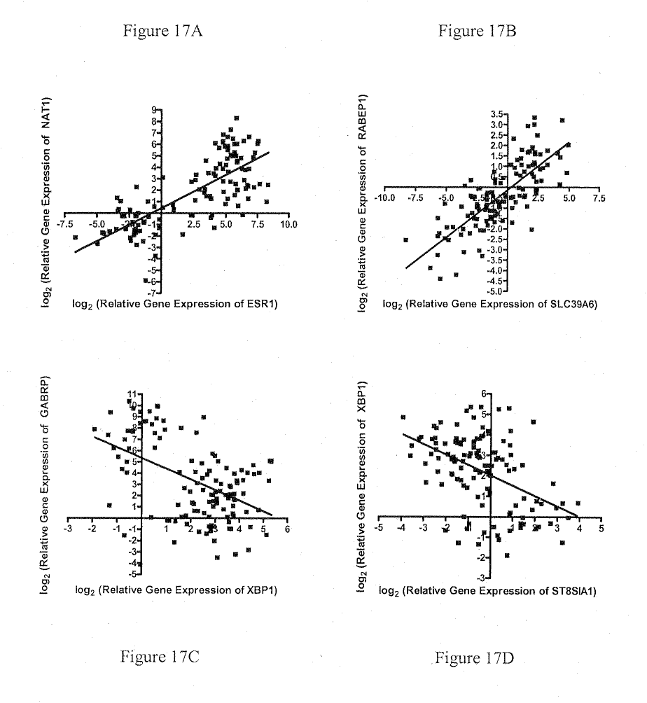

Early indications of shared pathways and potential interaction with multiple pathways influencing cancer growth and behavior led us to investigate correlations of expression levels of combinations of genes in the 32 gene set. Previous studies [180; 203] have shown that genes from subsets identified herein (i.e., GATA3 and XBP1) are co-expressed with ESR1, and play an important roles in development of models predicting clinical outcomes. In order to compare expression patterns among genes in the 32 gene set, Pearson correlations, which indicate relationships between gene pairs, were performed with the results shown in Table 30A-30H. Correlation coefficients above zero indicate a positive relationship between the genes of a pair, and a negative coefficient indicates an inverse relationship between gene expression levels (FIG. 17). The P values shown indicate that the correlations of gene expression did not occur by chance, and values shown in bold indicate a statistically significant correlation of expression levels between gene pairs (P value less than 0.01). As indicated in Table 30A-30H, the majority of genes expressed in the 126 tumors evaluated appear to be related to other genes within the 32 gene set suggesting involvement in similar molecular pathways. Expression of XBP1 was highly correlated ESR1 (Pearson correlation of 0.82, Table 30A-30H) as previously described [180; 203]. Remarkably, several genes, such as NAT1, ESR1, SCUBE2, FUT8, PTP4A2, LRBA, and MAPRE2 had expression levels related to more than 20 of the other genes within the 32 gene set (Table 30A-30H), further supporting the identification of critical molecular pathways dictating breast cancer behavior.

In order to visualize gene associations, expression levels were graphed to visualize the correlations between gene pairs. Representative correlations of gene expression that were significant from Pearson correlations are shown in FIG. 17. Comparisons of ESR1 and NAT1 expression by Pearson correlation gave a coefficient of 0.75 indicating a positive association (Table 30A-30H) with a linear regression of the data that resulted in an r² value of 0.56 (FIG. 17A). Comparisons of SLC39A6 and RABEP1 by Pearson correlation had a coefficient of 0.78 also indicating a positive association between the two genes (Table 30A-30H), and linear regression of the data that resulted in an r² value of 0.61 (FIG. 17B). In FIGS. 17C and D, representative negative correlations of gene expression levels are shown. Comparisons of XBP1 and GABRP by Pearson correlation had a coefficient of −0.49 indicating a moderate negative association (Table 30A-30H). FIG. 17C illustrates the inverse correlation of gene expression between XBP1 and GABRP, and linear regression of the data resulted in an r² value of 0.24. Comparisons of ST8SIA1 and XBP1 by Pearson correlation had a coefficient of −0.46 also indicating a moderate negative association between the two genes (Table 30A-30H), and linear regression of the data resulted in an r² value of 0.21 FIG. 17D.

TABLE 30A
Results from Pearson Correlations indicating relationships of gene expression.

| | EVL | | NAT1 | | ESR1 | | GABRP | |
|---|---|---|---|---|---|---|---|---|
| | Correlation | P value | Correlation | P value | Correlation | P value | Correlation | P value |
| EVL | 1.00 | | 0.62 | 0.000 | 0.72 | 0.000 | −0.36 | 0.000 |
| NAT1 | 0.62 | 0.000 | 1.00 | | 0.75 | 0.000 | −0.44 | 0.000 |
| ESR1 | 0.72 | 0.000 | 0.75 | 0.000 | 1.00 | | −0.40 | 0.000 |
| GABRP | −0.36 | 0.000 | −0.44 | 0.000 | −0.40 | 0.000 | 1.00 | |
| ST8SIA1 | −0.31 | 0.001 | −0.41 | 0.000 | −0.41 | 0.000 | 0.65 | 0.000 |
| TBC1D9 | 0.63 | 0.000 | 0.58 | 0.000 | 0.65 | 0.000 | −0.39 | 0.000 |
| TRIM29 | −0.29 | 0.001 | −0.26 | 0.003 | −0.37 | 0.000 | 0.57 | 0.000 |
| SCUBE2 | 0.63 | 0.000 | 0.75 | 0.000 | 0.80 | 0.000 | −0.37 | 0.000 |
| IL6ST | 0.34 | 0.000 | 0.37 | 0.000 | 0.48 | 0.000 | −0.21 | 0.024 |
| RABEP1 | 0.65 | 0.000 | 0.62 | 0.000 | 0.71 | 0.000 | −0.28 | 0.002 |
| SLC39A6 | 0.55 | 0.000 | 0.61 | 0.000 | 0.70 | 0.000 | −0.27 | 0.003 |
| TPBG | 0.50 | 0.000 | 0.60 | 0.000 | 0.70 | 0.000 | −0.18 | 0.048 |
| TCEAL1 | 0.44 | 0.000 | 0.61 | 0.000 | 0.66 | 0.000 | −0.22 | 0.013 |
| DSC2 | −0.25 | 0.004 | −0.20 | 0.027 | −0.25 | 0.006 | 0.27 | 0.003 |
| FUT8 | 0.57 | 0.000 | 0.53 | 0.000 | 0.61 | 0.000 | −0.33 | 0.000 |
| CENPA | −0.16 | 0.072 | −0.25 | 0.005 | −0.21 | 0.021 | 0.24 | 0.007 |
| MELK | −0.20 | 0.023 | −0.35 | 0.000 | −0.24 | 0.007 | 0.35 | 0.000 |
| PFKP | −0.10 | 0.291 | −0.28 | 0.001 | −0.31 | 0.001 | 0.26 | 0.004 |
| PLK1 | 0.06 | 0.520 | −0.11 | 0.233 | −0.04 | 0.628 | 0.21 | 0.024 |
| ATAD2 | 0.07 | 0.421 | −0.13 | 0.162 | −0.02 | 0.798 | 0.13 | 0.139 |
| XBP1 | 0.63 | 0.000 | 0.66 | 0.000 | 0.82 | 0.000 | −0.49 | 0.000 |
| MCM6 | 0.01 | 0.883 | 0.00 | 0.982 | 0.02 | 0.813 | 0.07 | 0.430 |
| BUB1 | −0.10 | 0.270 | −0.17 | 0.060 | −0.11 | 0.215 | 0.22 | 0.016 |
| PTP4A2 | 0.45 | 0.000 | 0.42 | 0.000 | 0.56 | 0.000 | −0.19 | 0.040 |
| YBX1 | −0.20 | 0.023 | −0.23 | 0.009 | −0.19 | 0.032 | 0.34 | 0.000 |
| LRBA | 0.44 | 0.000 | 0.34 | 0.000 | 0.43 | 0.000 | −0.35 | 0.000 |
| GATA3 | 0.67 | 0.000 | 0.67 | 0.000 | 0.83 | 0.000 | −0.44 | 0.000 |
| CX3CL1 | −0.16 | 0.066 | −0.33 | 0.000 | −0.32 | 0.000 | 0.50 | 0.000 |
| MAPRE2 | 0.06 | 0.491 | 0.11 | 0.206 | 0.13 | 0.160 | 0.15 | 0.097 |
| GMPS | −0.04 | 0.622 | −0.05 | 0.547 | 0.00 | 0.971 | 0.08 | 0.388 |
| CKS2 | −0.16 | 0.073 | −0.09 | 0.303 | −0.02 | 0.825 | 0.20 | 0.030 |
| SLC43A3 | −0.15 | 0.096 | −0.27 | 0.003 | −0.23 | 0.010 | 0.45 | 0.000 |

Pearson correlation coefficients are shown indicating the relationship between gene expression levels of various gene combinations. P values indicate that the correlations of expression by gene pairs did not occur by chance. Values shown in bold indicate a statistically significant correlation of expression levels between gene pairs (P value less than 0.01). Note that Table 30 consists of 8 pages illustrating the various gene combinations for the 32 gene set.

TABLE 30B

| | ST8SIA1 | | TBC1D9 | | TRIM29 | | SCUBE2 | |
|---|---|---|---|---|---|---|---|---|
| | Correlation | P value | Correlation | P value | Correlation | P value | Correlation | P value |
| EVL | −0.31 | 0.001 | 0.63 | 0.000 | −0.29 | 0.001 | 0.63 | 0.000 |
| NAT1 | −0.41 | 0.000 | 0.58 | 0.000 | −0.26 | 0.003 | 0.75 | 0.000 |
| ESR1 | −0.41 | 0.000 | 0.65 | 0.000 | −0.37 | 0.000 | 0.80 | 0.000 |
| GABRP | 0.65 | 0.000 | −0.39 | 0.000 | 0.57 | 0.000 | −0.37 | 0.000 |
| ST8SIA1 | 1.00 | | −0.27 | 0.003 | 0.54 | 0.000 | −0.38 | 0.000 |
| TBC1D9 | −0.27 | 0.003 | 1.00 | | −0.09 | 0.336 | 0.57 | 0.000 |
| TRIM29 | 0.54 | 0.000 | −0.09 | 0.336 | 1.00 | | −0.23 | 0.010 |
| SCUBE2 | −0.38 | 0.000 | 0.57 | 0.000 | −0.23 | 0.010 | 1.00 | |
| IL6ST | −0.12 | 0.204 | 0.80 | 0.000 | 0.13 | 0.153 | 0.43 | 0.000 |
| RABEP1 | −0.20 | 0.028 | 0.78 | 0.000 | 0.00 | 0.981 | 0.63 | 0.000 |
| SLC39A6 | −0.24 | 0.009 | 0.78 | 0.000 | −0.03 | 0.739 | 0.59 | 0.000 |
| TPBG | −0.15 | 0.093 | 0.61 | 0.000 | −0.03 | 0.757 | 0.65 | 0.000 |
| TCEAL1 | −0.22 | 0.017 | 0.59 | 0.000 | −0.14 | 0.137 | 0.61 | 0.000 |
| DSC2 | 0.39 | 0.000 | 0.25 | 0.006 | 0.48 | 0.000 | −0.18 | 0.042 |
| FUT8 | −0.28 | 0.002 | 0.76 | 0.000 | −0.06 | 0.514 | 0.54 | 0.000 |
| CENPA | 0.23 | 0.011 | −0.04 | 0.682 | 0.23 | 0.010 | −0.32 | 0.000 |
| MELK | 0.35 | 0.000 | −0.08 | 0.377 | 0.34 | 0.000 | −0.39 | 0.000 |
| PFKP | 0.31 | 0.001 | −0.06 | 0.517 | 0.50 | 0.000 | −0.24 | 0.008 |
| PLK1 | 0.25 | 0.006 | 0.19 | 0.033 | 0.32 | 0.000 | −0.18 | 0.043 |
| ATAD2 | 0.13 | 0.148 | 0.07 | 0.420 | 0.11 | 0.216 | −0.10 | 0.275 |
| XBP1 | −0.46 | 0.000 | 0.68 | 0.000 | −0.33 | 0.000 | 0.68 | 0.000 |
| MCM6 | 0.16 | 0.079 | 0.40 | 0.000 | 0.38 | 0.000 | −0.10 | 0.288 |
| BUB1 | 0.27 | 0.003 | 0.18 | 0.046 | 0.31 | 0.001 | −0.25 | 0.006 |
| PTP4A2 | −0.15 | 0.094 | 0.75 | 0.000 | 0.02 | 0.866 | 0.43 | 0.000 |
| YBX1 | 0.36 | 0.000 | 0.10 | 0.255 | 0.47 | 0.000 | −0.28 | 0.002 |
| LRBA | −0.14 | 0.120 | 0.81 | 0.000 | 0.04 | 0.664 | 0.37 | 0.000 |
| GATA3 | −0.42 | 0.000 | 0.74 | 0.000 | −0.26 | 0.004 | 0.69 | 0.000 |
| CX3CL1 | 0.56 | 0.000 | −0.12 | 0.197 | 0.62 | 0.000 | −0.29 | 0.001 |
| MAPRE2 | 0.27 | 0.003 | 0.41 | 0.000 | 0.41 | 0.000 | 0.10 | 0.289 |
| GMPS | 0.23 | 0.013 | 0.30 | 0.001 | 0.35 | 0.000 | −0.13 | 0.148 |
| CKS2 | 0.14 | 0.114 | 0.10 | 0.258 | 0.18 | 0.046 | −0.12 | 0.175 |
| SLC43A3 | 0.52 | 0.000 | 0.08 | 0.378 | 0.59 | 0.000 | −0.22 | 0.012 |

TABLE 30C

| | IL6ST | | RABEP1 | | SLC39A6 | | TPBG | |
|---|---|---|---|---|---|---|---|---|
| | Correlation | P value | Correlation | P value | Correlation | P value | Correlation | P value |
| EVL | 0.34 | 0.000 | 0.65 | 0.000 | 0.55 | 0.000 | 0.50 | 0.000 |
| NAT1 | 0.37 | 0.000 | 0.62 | 0.000 | 0.61 | 0.000 | 0.60 | 0.000 |
| ESR1 | 0.48 | 0.000 | 0.71 | 0.000 | 0.70 | 0.000 | 0.70 | 0.000 |
| GABRP | −0.21 | 0.024 | −0.28 | 0.002 | −0.27 | 0.003 | −0.18 | 0.048 |
| ST8SIA1 | −0.12 | 0.204 | −0.20 | 0.028 | −0.24 | 0.009 | −0.15 | 0.093 |
| TBC1D9 | 0.80 | 0.000 | 0.78 | 0.000 | 0.78 | 0.000 | 0.61 | 0.000 |
| TRIM29 | 0.13 | 0.153 | 0.00 | 0.981 | −0.03 | 0.739 | −0.03 | 0.757 |
| SCUBE2 | 0.43 | 0.000 | 0.63 | 0.000 | 0.59 | 0.000 | 0.65 | 0.000 |
| IL6ST | 1.00 | | 0.65 | 0.000 | 0.68 | 0.000 | 0.49 | 0.000 |
| RABEP1 | 0.65 | 0.000 | 1.00 | | 0.78 | 0.000 | 0.71 | 0.000 |
| SLC39A6 | 0.68 | 0.000 | 0.78 | 0.000 | 1.00 | | 0.62 | 0.000 |
| TPBG | 0.49 | 0.000 | 0.71 | 0.000 | 0.62 | 0.000 | 1.00 | |
| TCEAL1 | 0.47 | 0.000 | 0.67 | 0.000 | 0.64 | 0.000 | 0.56 | 0.000 |
| DSC2 | 0.45 | 0.000 | 0.04 | 0.676 | 0.07 | 0.420 | 0.12 | 0.166 |
| FUT8 | 0.67 | 0.000 | 0.72 | 0.000 | 0.69 | 0.000 | 0.52 | 0.000 |
| CENPA | 0.05 | 0.566 | 0.06 | 0.532 | 0.02 | 0.855 | −0.08 | 0.356 |
| MELK | 0.05 | 0.610 | −0.07 | 0.475 | −0.03 | 0.768 | −0.07 | 0.410 |
| PFKP | −0.03 | 0.727 | −0.05 | 0.611 | −0.13 | 0.145 | −0.19 | 0.034 |
| PLK1 | 0.20 | 0.029 | 0.25 | 0.006 | 0.19 | 0.037 | 0.14 | 0.126 |
| ATAD2 | 0.12 | 0.180 | 0.16 | 0.076 | 0.19 | 0.031 | 0.09 | 0.319 |
| XBP1 | 0.50 | 0.000 | 0.67 | 0.000 | 0.67 | 0.000 | 0.57 | 0.000 |
| MCM6 | 0.48 | 0.000 | 0.35 | 0.000 | 0.40 | 0.000 | 0.17 | 0.057 |
| BUB1 | 0.29 | 0.001 | 0.18 | 0.043 | 0.20 | 0.031 | 0.05 | 0.597 |
| PTP4A2 | 0.73 | 0.000 | 0.70 | 0.000 | 0.68 | 0.000 | 0.49 | 0.000 |
| YBX1 | 0.22 | 0.019 | 0.13 | 0.160 | 0.14 | 0.134 | 0.07 | 0.420 |
| LRBA | 0.79 | 0.000 | 0.63 | 0.000 | 0.65 | 0.000 | 0.40 | 0.000 |
| GATA3 | 0.53 | 0.000 | 0.71 | 0.000 | 0.70 | 0.000 | 0.57 | 0.000 |
| CX3CL1 | −0.09 | 0.351 | −0.07 | 0.410 | −0.12 | 0.178 | 0.01 | 0.911 |
| MAPRE2 | 0.45 | 0.000 | 0.41 | 0.000 | 0.36 | 0.000 | 0.40 | 0.000 |
| GMPS | 0.41 | 0.000 | 0.27 | 0.003 | 0.35 | 0.000 | 0.08 | 0.371 |
| CKS2 | 0.20 | 0.028 | 0.13 | 0.139 | 0.15 | 0.090 | 0.07 | 0.417 |
| SLC43A3 | 0.20 | 0.033 | 0.11 | 0.223 | 0.07 | 0.461 | 0.11 | 0.217 |

TABLE 30D

| | TCEAL1 | | DSC2 | | FUT8 | | CENPA | |
|---|---|---|---|---|---|---|---|---|
| | Correlation | P value | Correlation | P value | Correlation | P value | Correlation | P value |
| EVL | 0.44 | 0.000 | −0.25 | 0.004 | 0.57 | 0.000 | −0.16 | 0.072 |
| NAT1 | 0.61 | 0.000 | −0.20 | 0.027 | 0.53 | 0.000 | −0.25 | 0.005 |
| ESR1 | 0.66 | 0.000 | −0.25 | 0.006 | 0.61 | 0.000 | −0.21 | 0.021 |
| GABRP | −0.22 | 0.013 | 0.27 | 0.003 | −0.33 | 0.000 | 0.24 | 0.007 |
| ST8SIA1 | −0.22 | 0.017 | 0.39 | 0.000 | −0.28 | 0.002 | 0.23 | 0.011 |
| TBC1D9 | 0.59 | 0.000 | 0.25 | 0.006 | 0.76 | 0.000 | −0.04 | 0.682 |
| TRIM29 | −0.14 | 0.137 | 0.48 | 0.000 | −0.06 | 0.514 | 0.23 | 0.010 |
| SCUBE2 | 0.61 | 0.000 | −0.18 | 0.042 | 0.54 | 0.000 | −0.32 | 0.000 |
| IL6ST | 0.47 | 0.000 | 0.45 | 0.000 | 0.67 | 0.000 | 0.05 | 0.566 |
| RABEP1 | 0.67 | 0.000 | 0.04 | 0.676 | 0.72 | 0.000 | 0.06 | 0.532 |
| SLC39A6 | 0.64 | 0.000 | 0.07 | 0.420 | 0.69 | 0.000 | 0.02 | 0.855 |
| TPBG | 0.56 | 0.000 | 0.12 | 0.166 | 0.52 | 0.000 | −0.08 | 0.356 |
| TCEAL1 | 1.00 | | 0.04 | 0.669 | 0.58 | 0.000 | −0.01 | 0.915 |
| DSC2 | 0.04 | 0.669 | 1.00 | | 0.10 | 0.281 | 0.25 | 0.005 |
| FUT8 | 0.58 | 0.000 | 0.10 | 0.281 | 1.00 | | 0.06 | 0.506 |
| CENPA | −0.01 | 0.915 | 0.25 | 0.005 | 0.06 | 0.506 | 1.00 | |
| MELK | −0.16 | 0.072 | 0.31 | 0.000 | 0.01 | 0.885 | 0.73 | 0.000 |
| PFKP | −0.17 | 0.065 | 0.18 | 0.042 | −0.05 | 0.585 | 0.31 | 0.000 |
| PLK1 | −0.03 | 0.717 | 0.22 | 0.012 | 0.13 | 0.158 | 0.68 | 0.000 |
| ATAD2 | −0.02 | 0.794 | 0.10 | 0.256 | 0.07 | 0.428 | 0.56 | 0.000 |
| XBP1 | 0.60 | 0.000 | −0.21 | 0.021 | 0.66 | 0.000 | −0.23 | 0.010 |
| MCM6 | 0.16 | 0.070 | 0.47 | 0.000 | 0.54 | 0.000 | 0.58 | 0.000 |
| BUB1 | 0.02 | 0.789 | 0.40 | 0.000 | 0.28 | 0.002 | 0.77 | 0.000 |
| PTP4A2 | 0.49 | 0.000 | 0.23 | 0.010 | 0.76 | 0.000 | 0.18 | 0.049 |
| YBX1 | −0.02 | 0.791 | 0.43 | 0.000 | 0.23 | 0.010 | 0.58 | 0.000 |
| LRBA | 0.33 | 0.000 | 0.33 | 0.000 | 0.65 | 0.000 | 0.12 | 0.200 |
| GATA3 | 0.62 | 0.000 | −0.12 | 0.165 | 0.72 | 0.000 | −0.16 | 0.084 |
| CX3CL1 | −0.25 | 0.005 | 0.27 | 0.002 | −0.10 | 0.252 | 0.12 | 0.174 |
| MAPRE2 | 0.30 | 0.001 | 0.55 | 0.000 | 0.42 | 0.000 | 0.34 | 0.000 |
| GMPS | 0.17 | 0.058 | 0.41 | 0.000 | 0.39 | 0.000 | 0.52 | 0.000 |
| CKS2 | 0.19 | 0.039 | 0.39 | 0.000 | 0.26 | 0.004 | 0.66 | 0.000 |
| SLC43A3 | −0.08 | 0.383 | 0.49 | 0.000 | 0.12 | 0.199 | 0.37 | 0.000 |

TABLE 30E

| | MELK | | PFKP | | PLK1 | | ATAD2 | |
|---|---|---|---|---|---|---|---|---|
| | Correlation | P value | Correlation | P value | Correlation | P value | Correlation | P value |
| EVL | −0.20 | 0.023 | −0.10 | 0.291 | 0.06 | 0.520 | 0.07 | 0.421 |
| NAT1 | −0.35 | 0.000 | −0.28 | 0.001 | −0.11 | 0.233 | −0.13 | 0.162 |
| ESR1 | −0.24 | 0.007 | −0.31 | 0.001 | −0.04 | 0.628 | −0.02 | 0.798 |
| GABRP | 0.35 | 0.000 | 0.26 | 0.004 | 0.21 | 0.024 | 0.13 | 0.139 |
| ST8SIA1 | 0.35 | 0.000 | 0.31 | 0.001 | 0.25 | 0.006 | 0.13 | 0.148 |
| TBC1D9 | −0.08 | 0.377 | −0.06 | 0.517 | 0.19 | 0.033 | 0.07 | 0.420 |
| TRIM29 | 0.34 | 0.000 | 0.50 | 0.000 | 0.32 | 0.000 | 0.11 | 0.216 |
| SCUBE2 | −0.39 | 0.000 | −0.24 | 0.008 | −0.18 | 0.043 | −0.10 | 0.275 |
| IL6ST | 0.05 | 0.610 | −0.03 | 0.727 | 0.20 | 0.029 | 0.12 | 0.180 |
| RABEP1 | −0.07 | 0.475 | −0.05 | 0.611 | 0.25 | 0.006 | 0.16 | 0.076 |
| SLC39A6 | −0.03 | 0.768 | −0.13 | 0.145 | 0.19 | 0.037 | 0.19 | 0.031 |
| TPBG | −0.07 | 0.410 | −0.19 | 0.034 | 0.14 | 0.126 | 0.09 | 0.319 |
| TCEAL1 | −0.16 | 0.072 | −0.17 | 0.065 | −0.03 | 0.717 | −0.02 | 0.794 |
| DSC2 | 0.31 | 0.000 | 0.18 | 0.042 | 0.22 | 0.012 | 0.10 | 0.256 |
| FUT8 | 0.01 | 0.885 | −0.05 | 0.585 | 0.13 | 0.158 | 0.07 | 0.428 |
| CENPA | 0.73 | 0.000 | 0.31 | 0.000 | 0.68 | 0.000 | 0.56 | 0.000 |
| MELK | 1.00 | | 0.49 | 0.000 | 0.70 | 0.000 | 0.54 | 0.000 |
| PFKP | 0.49 | 0.000 | 1.00 | | 0.39 | 0.000 | 0.25 | 0.006 |
| PLK1 | 0.70 | 0.000 | 0.39 | 0.000 | 1.00 | | 0.58 | 0.000 |
| ATAD2 | 0.54 | 0.000 | 0.25 | 0.006 | 0.58 | 0.000 | 1.00 | |
| XBP1 | −0.30 | 0.001 | −0.39 | 0.000 | −0.08 | 0.346 | −0.05 | 0.614 |
| MCM6 | 0.59 | 0.000 | 0.35 | 0.000 | 0.62 | 0.000 | 0.43 | 0.000 |
| BUB1 | 0.73 | 0.000 | 0.37 | 0.000 | 0.75 | 0.000 | 0.55 | 0.000 |
| PTP4A2 | 0.12 | 0.169 | −0.06 | 0.527 | 0.25 | 0.004 | 0.22 | 0.016 |
| YBX1 | 0.57 | 0.000 | 0.36 | 0.000 | 0.60 | 0.000 | 0.49 | 0.000 |
| LRBA | 0.14 | 0.123 | 0.06 | 0.474 | 0.30 | 0.001 | 0.20 | 0.029 |
| GATA3 | −0.18 | 0.040 | −0.21 | 0.017 | 0.04 | 0.670 | 0.00 | 0.974 |
| CX3CL1 | 0.33 | 0.000 | 0.46 | 0.000 | 0.33 | 0.000 | 0.18 | 0.044 |
| MAPRE2 | 0.33 | 0.000 | 0.22 | 0.014 | 0.43 | 0.000 | 0.16 | 0.073 |
| GMPS | 0.59 | 0.000 | 0.42 | 0.000 | 0.55 | 0.000 | 0.44 | 0.000 |
| CKS2 | 0.54 | 0.000 | 0.06 | 0.488 | 0.46 | 0.000 | 0.37 | 0.000 |
| SLC43A3 | 0.51 | 0.000 | 0.49 | 0.000 | 0.54 | 0.000 | 0.29 | 0.001 |

TABLE 30F

| | XBP1 | | MCM6 | | BUB1 | | PTP4A2 | |
|---|---|---|---|---|---|---|---|---|
| | Correlation | P value | Correlation | P value | Correlation | P value | Correlation | P value |
| EVL | 0.63 | 0.000 | 0.01 | 0.883 | −0.10 | 0.270 | 0.45 | 0.000 |
| NAT1 | 0.66 | 0.000 | 0.00 | 0.982 | −0.17 | 0.060 | 0.42 | 0.000 |
| ESR1 | 0.82 | 0.000 | 0.02 | 0.813 | −0.11 | 0.215 | 0.56 | 0.000 |
| GABRP | −0.49 | 0.000 | 0.07 | 0.430 | 0.22 | 0.016 | −0.19 | 0.040 |
| ST8SIA1 | −0.46 | 0.000 | 0.16 | 0.079 | 0.27 | 0.003 | −0.15 | 0.094 |
| TBC1D9 | 0.68 | 0.000 | 0.40 | 0.000 | 0.18 | 0.046 | 0.75 | 0.000 |
| TRIM29 | −0.33 | 0.000 | 0.38 | 0.000 | 0.31 | 0.001 | 0.02 | 0.866 |
| SCUBE2 | 0.68 | 0.000 | −0.10 | 0.288 | −0.25 | 0.006 | 0.43 | 0.000 |
| IL6ST | 0.50 | 0.000 | 0.48 | 0.000 | 0.29 | 0.001 | 0.73 | 0.000 |
| RABEP1 | 0.67 | 0.000 | 0.35 | 0.000 | 0.18 | 0.043 | 0.70 | 0.000 |
| SLC39A6 | 0.67 | 0.000 | 0.40 | 0.000 | 0.20 | 0.031 | 0.68 | 0.000 |
| TPBG | 0.57 | 0.000 | 0.17 | 0.057 | 0.05 | 0.597 | 0.49 | 0.000 |
| TCEAL1 | 0.60 | 0.000 | 0.16 | 0.070 | 0.02 | 0.789 | 0.49 | 0.000 |
| DSC2 | −0.21 | 0.021 | 0.47 | 0.000 | 0.40 | 0.000 | 0.23 | 0.010 |
| FUT8 | 0.66 | 0.000 | 0.54 | 0.000 | 0.28 | 0.002 | 0.76 | 0.000 |
| CENPA | −0.23 | 0.010 | 0.58 | 0.000 | 0.77 | 0.000 | 0.18 | 0.049 |
| MELK | −0.30 | 0.001 | 0.59 | 0.000 | 0.73 | 0.000 | 0.12 | 0.169 |
| PFKP | −0.39 | 0.000 | 0.35 | 0.000 | 0.37 | 0.000 | −0.06 | 0.527 |
| PLK1 | −0.08 | 0.346 | 0.62 | 0.000 | 0.75 | 0.000 | 0.25 | 0.004 |
| ATAD2 | −0.05 | 0.614 | 0.43 | 0.000 | 0.55 | 0.000 | 0.22 | 0.016 |
| XBP1 | 1.00 | | 0.08 | 0.372 | −0.08 | 0.377 | 0.62 | 0.000 |
| MCM6 | 0.08 | 0.372 | 1.00 | | 0.81 | 0.000 | 0.60 | 0.000 |
| BUB1 | −0.08 | 0.377 | 0.81 | 0.000 | 1.00 | | 0.39 | 0.000 |
| PTP4A2 | 0.62 | 0.000 | 0.60 | 0.000 | 0.39 | 0.000 | 1.00 | |
| YBX1 | −0.06 | 0.534 | 0.71 | 0.000 | 0.71 | 0.000 | 0.36 | 0.000 |
| LRBA | 0.48 | 0.000 | 0.53 | 0.000 | 0.31 | 0.000 | 0.72 | 0.000 |
| GATA3 | 0.84 | 0.000 | 0.20 | 0.026 | 0.03 | 0.770 | 0.64 | 0.000 |
| CX3CL1 | −0.26 | 0.003 | 0.22 | 0.013 | 0.18 | 0.043 | −0.13 | 0.149 |
| MAPRE2 | 0.19 | 0.038 | 0.58 | 0.000 | 0.51 | 0.000 | 0.43 | 0.000 |
| GMPS | 0.03 | 0.698 | 0.85 | 0.000 | 0.71 | 0.000 | 0.48 | 0.000 |
| CKS2 | 0.04 | 0.629 | 0.65 | 0.000 | 0.71 | 0.000 | 0.37 | 0.000 |
| SLC43A3 | −0.22 | 0.016 | 0.61 | 0.000 | 0.59 | 0.000 | 0.24 | 0.008 |

TABLE 30G

| | YBX1 | | LRBA | | GATA3 | | CX3CL1 | |
|---|---|---|---|---|---|---|---|---|
| | Correlation | P value | Correlation | P value | Correlation | P value | Correlation | P value |
| EVL | −0.20 | 0.023 | 0.44 | 0.000 | 0.67 | 0.000 | −0.16 | 0.066 |
| NAT1 | −0.23 | 0.009 | 0.34 | 0.000 | 0.67 | 0.000 | −0.33 | 0.000 |
| ESR1 | −0.19 | 0.032 | 0.43 | 0.000 | 0.83 | 0.000 | −0.32 | 0.000 |
| GABRP | 0.34 | 0.000 | −0.35 | 0.000 | −0.44 | 0.000 | 0.50 | 0.000 |
| ST8SIA1 | 0.36 | 0.000 | −0.14 | 0.120 | −0.42 | 0.000 | 0.56 | 0.000 |
| TBC1D9 | 0.10 | 0.255 | 0.81 | 0.000 | 0.74 | 0.000 | −0.12 | 0.197 |
| TRIM29 | 0.47 | 0.000 | 0.04 | 0.664 | −0.26 | 0.004 | 0.62 | 0.000 |
| SCUBE2 | −0.28 | 0.002 | 0.37 | 0.000 | 0.69 | 0.000 | −0.29 | 0.001 |
| IL6ST | 0.22 | 0.019 | 0.79 | 0.000 | 0.53 | 0.000 | −0.09 | 0.351 |
| RABEP1 | 0.13 | 0.160 | 0.63 | 0.000 | 0.71 | 0.000 | −0.07 | 0.410 |
| SLC39A6 | 0.14 | 0.134 | 0.65 | 0.000 | 0.70 | 0.000 | −0.12 | 0.178 |
| TPBG | 0.07 | 0.420 | 0.40 | 0.000 | 0.57 | 0.000 | 0.01 | 0.911 |
| TCEAL1 | −0.02 | 0.791 | 0.33 | 0.000 | 0.62 | 0.000 | −0.25 | 0.005 |
| DSC2 | 0.43 | 0.000 | 0.33 | 0.000 | −0.12 | 0.165 | 0.27 | 0.002 |
| FUT8 | 0.23 | 0.010 | 0.65 | 0.000 | 0.72 | 0.000 | −0.10 | 0.252 |
| CENPA | 0.58 | 0.000 | 0.12 | 0.200 | −0.16 | 0.084 | 0.12 | 0.174 |
| MELK | 0.57 | 0.000 | 0.14 | 0.123 | −0.18 | 0.040 | 0.33 | 0.000 |
| PFKP | 0.36 | 0.000 | 0.06 | 0.474 | −0.21 | 0.017 | 0.46 | 0.000 |
| PLK1 | 0.60 | 0.000 | 0.30 | 0.001 | 0.04 | 0.670 | 0.33 | 0.000 |
| ATAD2 | 0.49 | 0.000 | 0.20 | 0.029 | 0.00 | 0.974 | 0.18 | 0.044 |
| XBP1 | −0.06 | 0.534 | 0.48 | 0.000 | 0.84 | 0.000 | −0.26 | 0.003 |
| MCM6 | 0.71 | 0.000 | 0.53 | 0.000 | 0.20 | 0.026 | 0.22 | 0.013 |
| BUB1 | 0.71 | 0.000 | 0.31 | 0.000 | 0.03 | 0.770 | 0.18 | 0.043 |
| PTP4A2 | 0.36 | 0.000 | 0.72 | 0.000 | 0.64 | 0.000 | −0.13 | 0.149 |
| YBX1 | 1.00 | | 0.23 | 0.011 | −0.02 | 0.822 | 0.47 | 0.000 |
| LRBA | 0.23 | 0.011 | 1.00 | | 0.54 | 0.000 | 0.01 | 0.932 |
| GATA3 | −0.02 | 0.822 | 0.54 | 0.000 | 1.00 | | −0.18 | 0.049 |
| CX3CL1 | 0.47 | 0.000 | 0.01 | 0.932 | −0.18 | 0.049 | 1.00 | |
| MAPRE2 | 0.56 | 0.000 | 0.42 | 0.000 | 0.22 | 0.016 | 0.36 | 0.000 |
| GMPS | 0.69 | 0.000 | 0.44 | 0.000 | 0.16 | 0.070 | 0.20 | 0.026 |
| CKS2 | 0.60 | 0.000 | 0.20 | 0.025 | 0.02 | 0.791 | −0.07 | 0.437 |
| SLC43A3 | 0.70 | 0.000 | 0.17 | 0.050 | −0.10 | 0.249 | 0.58 | 0.000 |

TABLE 30H

| | MAPRE2 | | GMPS | | CKS2 | | SLC43A3 | |
|---|---|---|---|---|---|---|---|---|
| | Correlation | P value | Correlation | P value | Correlation | P value | Correlation | P value |
| EVL | 0.06 | 0.491 | −0.04 | 0.622 | −0.16 | 0.073 | −0.15 | 0.096 |
| NAT1 | 0.11 | 0.206 | −0.05 | 0.547 | −0.09 | 0.303 | −0.27 | 0.003 |
| ESR1 | 0.13 | 0.160 | 0.00 | 0.971 | −0.02 | 0.825 | −0.23 | 0.010 |
| GABRP | 0.15 | 0.097 | 0.08 | 0.388 | 0.20 | 0.030 | 0.45 | 0.000 |
| ST8SIA1 | 0.27 | 0.003 | 0.23 | 0.013 | 0.14 | 0.114 | 0.52 | 0.000 |
| TBC1D9 | 0.41 | 0.000 | 0.30 | 0.001 | 0.10 | 0.258 | 0.08 | 0.378 |
| TRIM29 | 0.41 | 0.000 | 0.35 | 0.000 | 0.18 | 0.046 | 0.59 | 0.000 |
| SCUBE2 | 0.10 | 0.289 | −0.13 | 0.148 | −0.12 | 0.175 | −0.22 | 0.012 |
| IL6ST | 0.45 | 0.000 | 0.41 | 0.000 | 0.20 | 0.028 | 0.20 | 0.033 |
| RABEP1 | 0.41 | 0.000 | 0.27 | 0.003 | 0.13 | 0.139 | 0.11 | 0.223 |
| SLC39A6 | 0.36 | 0.000 | 0.35 | 0.000 | 0.15 | 0.090 | 0.07 | 0.461 |
| TPBG | 0.40 | 0.000 | 0.08 | 0.371 | 0.07 | 0.417 | 0.11 | 0.217 |
| TCEAL1 | 0.30 | 0.001 | 0.17 | 0.058 | 0.19 | 0.039 | −0.08 | 0.383 |
| DSC2 | 0.55 | 0.000 | 0.41 | 0.000 | 0.39 | 0.000 | 0.49 | 0.000 |
| FUT8 | 0.42 | 0.000 | 0.39 | 0.000 | 0.26 | 0.004 | 0.12 | 0.199 |
| CENPA | 0.34 | 0.000 | 0.52 | 0.000 | 0.66 | 0.000 | 0.37 | 0.000 |
| MELK | 0.33 | 0.000 | 0.59 | 0.000 | 0.54 | 0.000 | 0.51 | 0.000 |
| PFKP | 0.22 | 0.014 | 0.42 | 0.000 | 0.06 | 0.488 | 0.49 | 0.000 |
| PLK1 | 0.43 | 0.000 | 0.55 | 0.000 | 0.46 | 0.000 | 0.54 | 0.000 |
| ATAD2 | 0.16 | 0.073 | 0.44 | 0.000 | 0.37 | 0.000 | 0.29 | 0.001 |
| XBP1 | 0.19 | 0.038 | 0.03 | 0.698 | 0.04 | 0.629 | −0.22 | 0.016 |
| MCM6 | 0.58 | 0.000 | 0.85 | 0.000 | 0.65 | 0.000 | 0.61 | 0.000 |
| BUB1 | 0.51 | 0.000 | 0.71 | 0.000 | 0.71 | 0.000 | 0.59 | 0.000 |
| PTP4A2 | 0.43 | 0.000 | 0.48 | 0.000 | 0.37 | 0.000 | 0.24 | 0.008 |
| YBX1 | 0.56 | 0.000 | 0.69 | 0.000 | 0.60 | 0.000 | 0.70 | 0.000 |
| LRBA | 0.42 | 0.000 | 0.44 | 0.000 | 0.20 | 0.025 | 0.17 | 0.050 |
| GATA3 | 0.22 | 0.016 | 0.16 | 0.070 | 0.02 | 0.791 | −0.10 | 0.249 |
| CX3CL1 | 0.36 | 0.000 | 0.20 | 0.026 | −0.07 | 0.437 | 0.58 | 0.000 |
| MAPRE2 | 1.00 | | 0.49 | 0.000 | 0.43 | 0.000 | 0.55 | 0.000 |
| GMPS | 0.49 | 0.000 | 1.00 | | 0.56 | 0.000 | 0.60 | 0.000 |
| CKS2 | 0.43 | 0.000 | 0.56 | 0.000 | 1.00 | | 0.34 | 0.000 |
| SLC43A3 | 0.55 | 0.000 | 0.60 | 0.000 | 0.34 | 0.000 | 1.00 | |

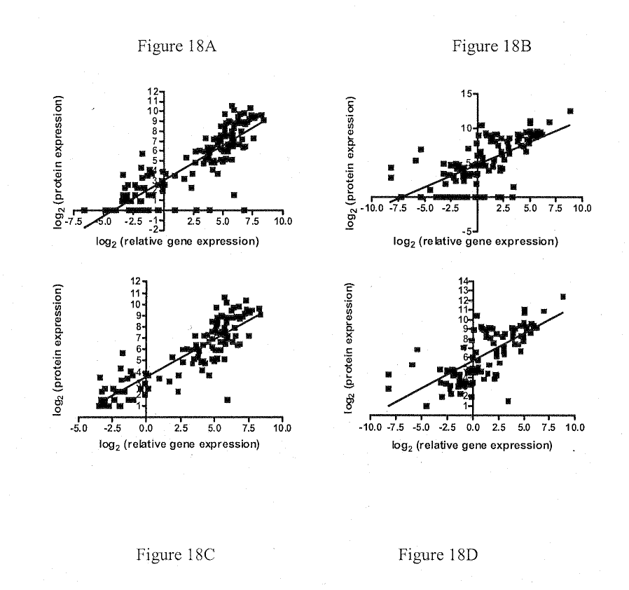

Current clinical tests for ER and PR are based upon measurements of the protein in a tissue biopsy (e.g., [10; 11; 91; 135]). To assess the utility of mRNA measurements and their relationship to ER (FIGS. 18A and 18C) and PR protein levels (FIGS. 18B and 18D), ER and PR gene expression levels from intact tissue sections and protein expression from tissue extracts were evaluated in 132 breast cancer biopsies. Pearson correlations gave positive correlations of 0.84 for ER and 0.61 for PR (P values less than 0.001). The correlation between ER gene expression (measured by qPCR) and protein expression (measured by LBA) was much higher than previously reported by Kim et al. [250]. These investigators reported a correlation of 0.4 for the same comparisons of ER as measured by LBA (as performed in the NSABP clinical trial) and qPCR (as performed in the Oncotype DX assay). Since the same method for measuring ER protein was used in Kim et al., this difference in correlation between gene and protein levels suggests variation in qPCR measurements (e.g., primer selection).

The log₂ expression levels for ER and PR were then plotted for linear regression analyses (FIG. 18), as suggested by Jeong et al. [251]. The relationship between ER mRNA and protein product levels gave a correlation with r²=0.70 (FIG. 18A), while the correlation between PR mRNA and protein product yielded an r²=0.38 (FIG. 18B). Since 22 of the specimens shown in A and 23 of the specimens shown in B did not express the tumor marker protein (thus appear to be outliers in these analyses), values from these specimens were excluded (FIGS. 18C and D). Linear regressions of these plots had higher correlation coefficients of 0.73 for ER (FIG. 18C) and 0.48 for PR (FIG. 18D), indicating that samples with undetectable receptor protein negatively affected the correlations. Although linear regression results appear to correspond to those from Pearson correlation analyses, it is not surprising that levels of gene and protein expression are not correlated perfectly. For example, some mRNA molecules may not be translated into functional protein product, or that the ER and PR protein expressed may be degraded, hence, immeasurable in the LBA.

Relationships of Gene Expression Levels with Clinical Characteristics

The expression of each candidate gene was analyzed for associations with the characteristics of each of 126 patients, such as race, menopausal status, family history of breast cancer, stage of disease, tumor grade, nodal involvement, ER status, and PR status with the use of SPSS software (Table 31). Analysis of race, menopausal status, family history, nodal status, ER status and PR status were performed using a two-tailed t-test (equal variances not assumed), while stage and grade were analyzed by ANOVA. Expression of genes outlined in Table 31 exhibited P values less than 0.05 when correlated with the characteristic indicated. Since t-tests do not provide information as to the levels of expression for each gene analyzed in the different groups, log₂ (relative gene expression) was graphed as box and whisker plots in GRAPHPAD PRISM®.

TABLE 31
Association of the expression of individual genes in the carcinoma and
stromal gene subsets with various patient characteristics.

| CHARACTERISTIC | GENES ASSOCIATED |
|---|---|
| Race | no associations |
| Menopausal Status | EVL, NAT1, ESR1, GABRP, TBC1D9, TRIM29, SCUBE2, RABEP1, |
| | SLC39A6, TCEAL1, MELK, ATAD2, XBP1, LRBA, GATA3 |
| Family History | no associations |
| Smoking Status | PFKP, YBX1, SLC43A3 |
| Stage | no associations |
| Grade | EVL, NAT1, ESR1, ST8SIA1, TBC1D9, SCUBE2, RABEP1, SLC39A6, |
| | TPBG, TCEAL1, CENPA, MELK, XBP1, BUB1, GATA3 |
| Nodal Status | GABRP, CENPA |
| ER Status | EVL, NAT1, ESR1, GABRP, ST8SIA1, TBC1D9, TRIM29, SCUBE2, |
| | IL6ST, RABEP1, SLC39A6, TPBG, TCEAL1, DSC2, FUT8, CENPA, |
| | MELK, PFKP, XBP1, PTP4A2, YBX1, LRBA, GATA3, CX3CL1, SLC43A3 |
| PR Status | EVL, NAT1, ESR1, GABRP, ST8SIA1, TBC1D9, SCUBE2, IL6ST, |
| | RABEP1, SLC39A6, TPBG, TCEAL1, FUT8, MELK, PFKP, XBP1, |
| | PTP4A2, LRBA, GATA3, CX3CL1, SLC43A3 |

Analyses of race, menopausal status, family history, nodal status, ER status and PR status were performed using a two-tailed t-test, while stage and grade were analyzed by ANOVA. The expression levels of genes listed exhibited P values less than 0.05.

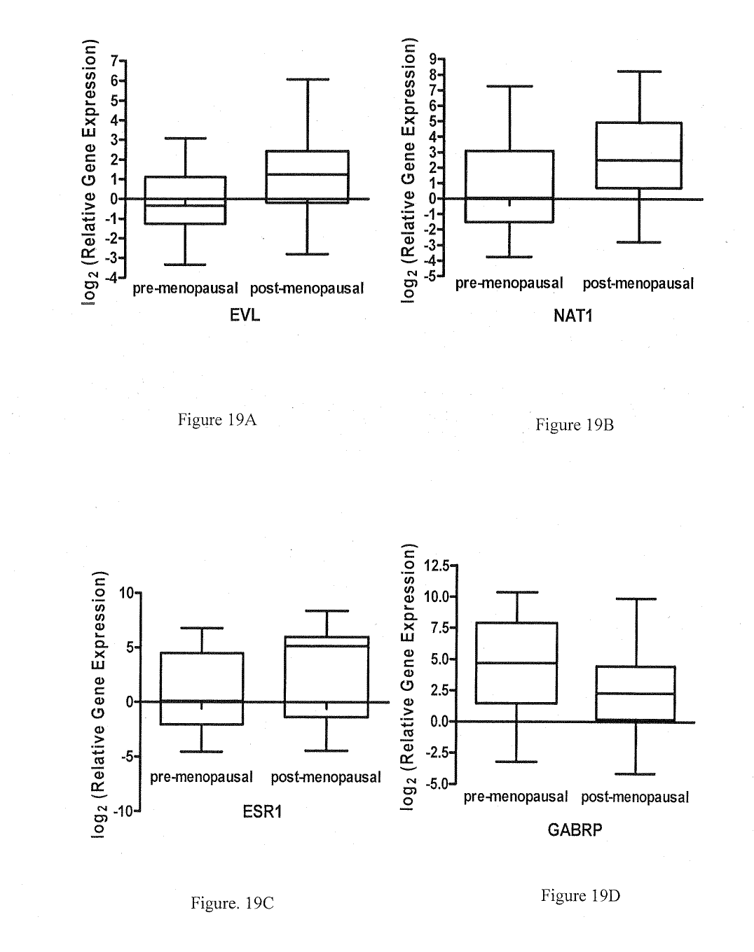

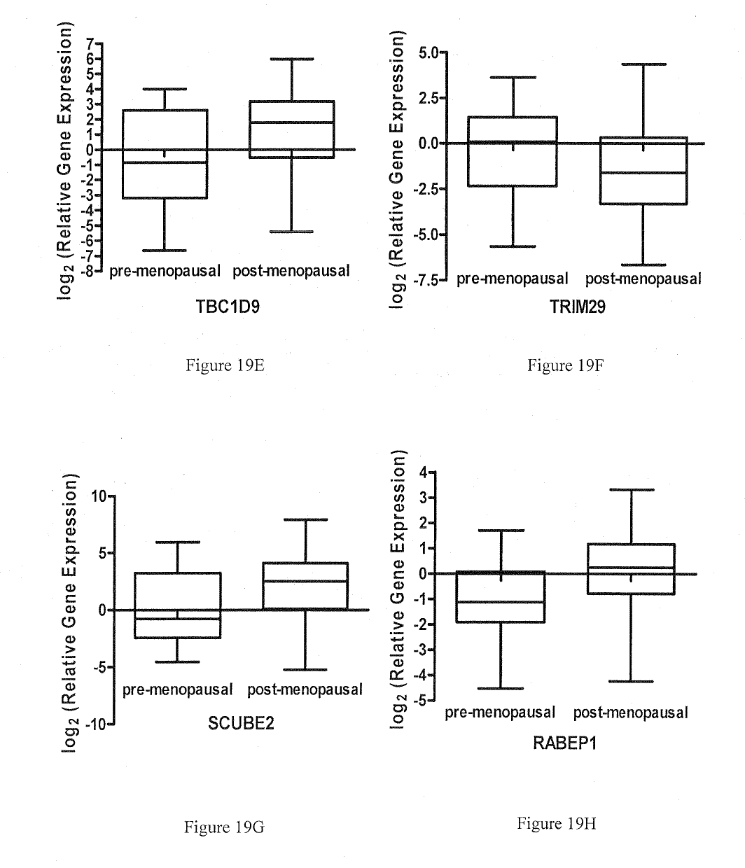

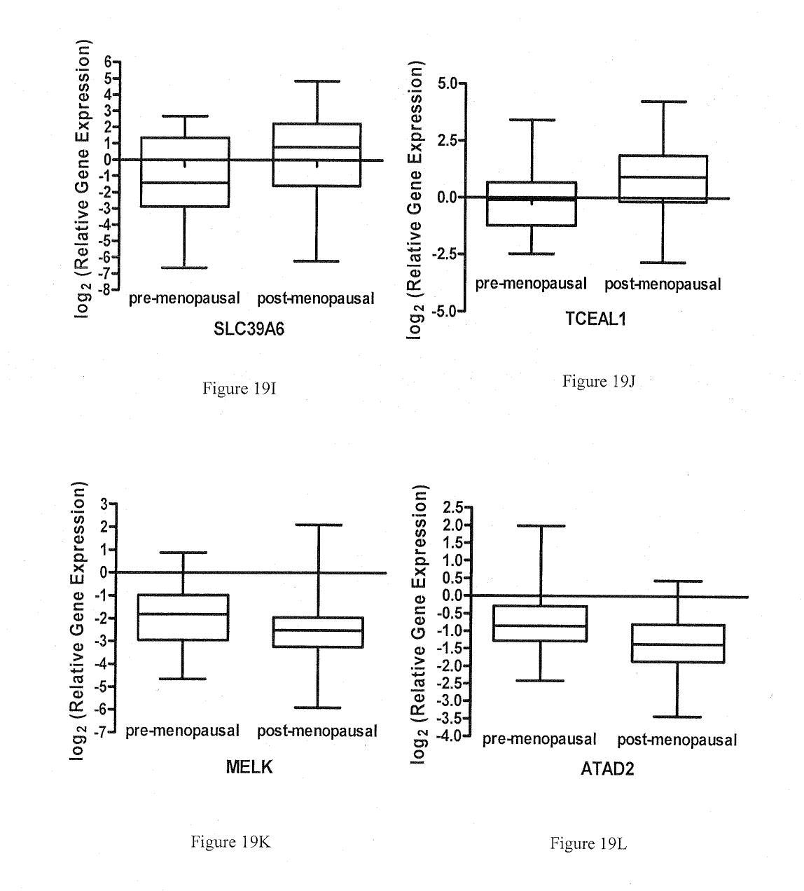

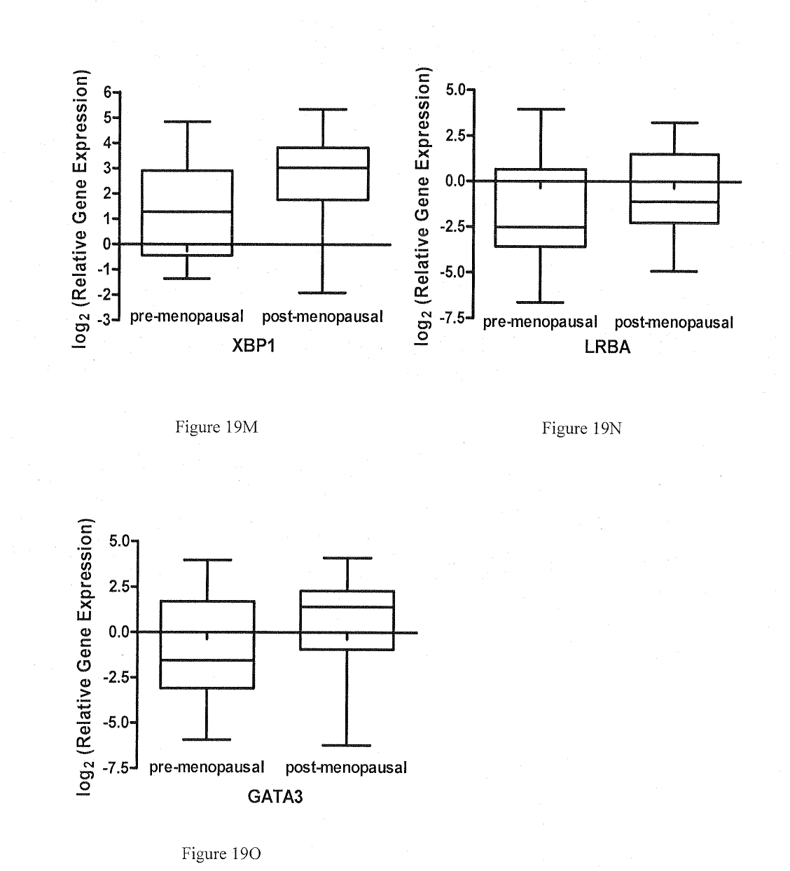

Gene expression differences in pre-menopausal (n=30) and post-menopausal (n=51) breast cancer patients are shown in FIG. 19. The box contains gene expression levels within the second and third quartiles of gene expression values. The horizontal line within the box indicates the median expression level, and the whiskers extend to the lowest and highest expression level for each gene. Genes shown are those with differences determined significant in t-tests, i.e., EVL, NAT1, ESR1, GABRP, TBC1D9, TRIM29, SCUBE2, RABEP1, SLC39A6, TCEAL1, MELK, ATAD2, XBP1, LRBA, and GATA3. Several genes were expressed to a greater extent in the post-menopausal patients (EVL, NAT1, ESR1, TBC1D9, SCUBE2, RABEP1, SLC39A6, TCEAL1, XBP1, LRBA, and GATA3), while others exhibited lower expression compared to that of pre-menopausal patients. When these gene results are considered with those relating association of gene expression with other patient characteristics, particularly ER and PR status, development of breast cancer in an estrogen rich environment, i.e., pre-menopausal years, may influence these gene expression profiles.

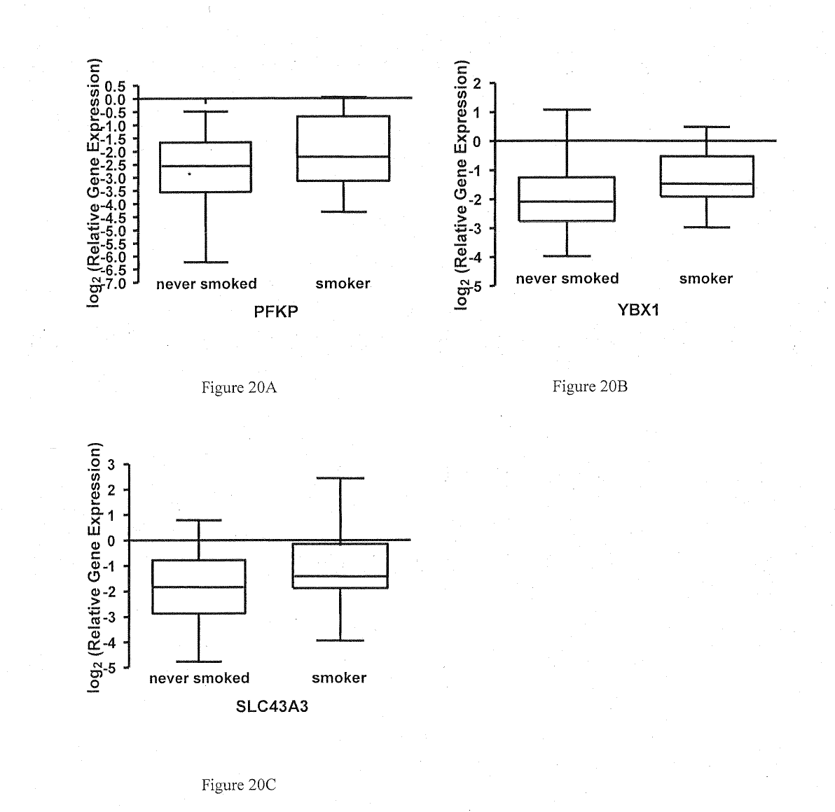

Differences in gene expression levels for cancer patients who were tobacco smokers (n=27) and whose who were non-smokers (n=54) are shown in FIG. 20. Expression levels of each gene shown are those with differences determined significant in t-tests, including PFKP, YBX1, and SLC43A3. Expression of each was higher in the smoking patient cohort compared to the non-smokers.

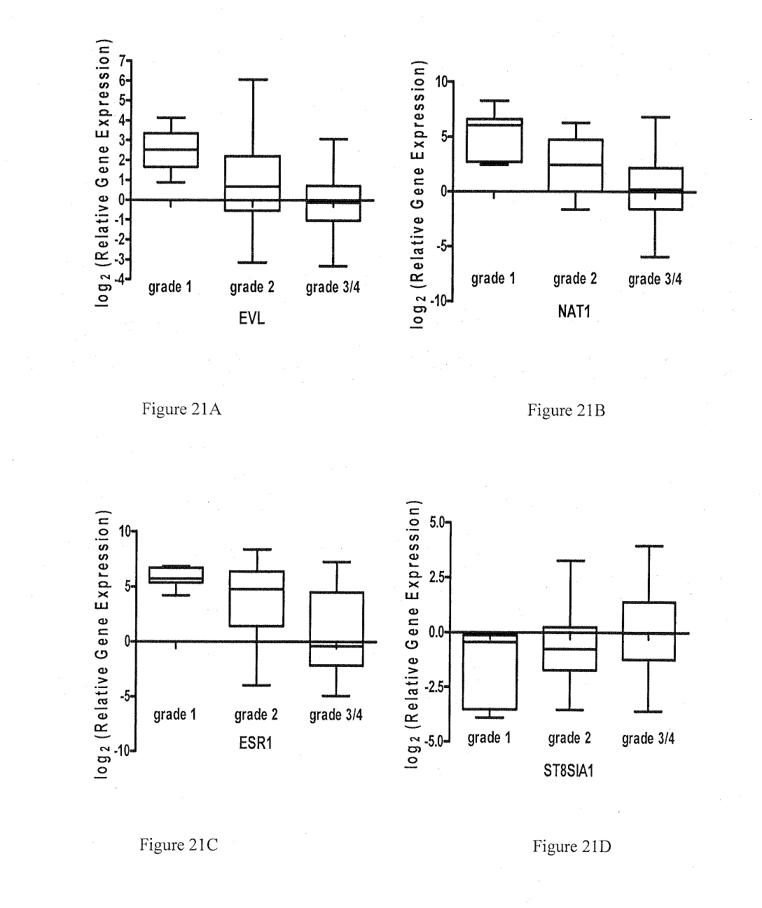

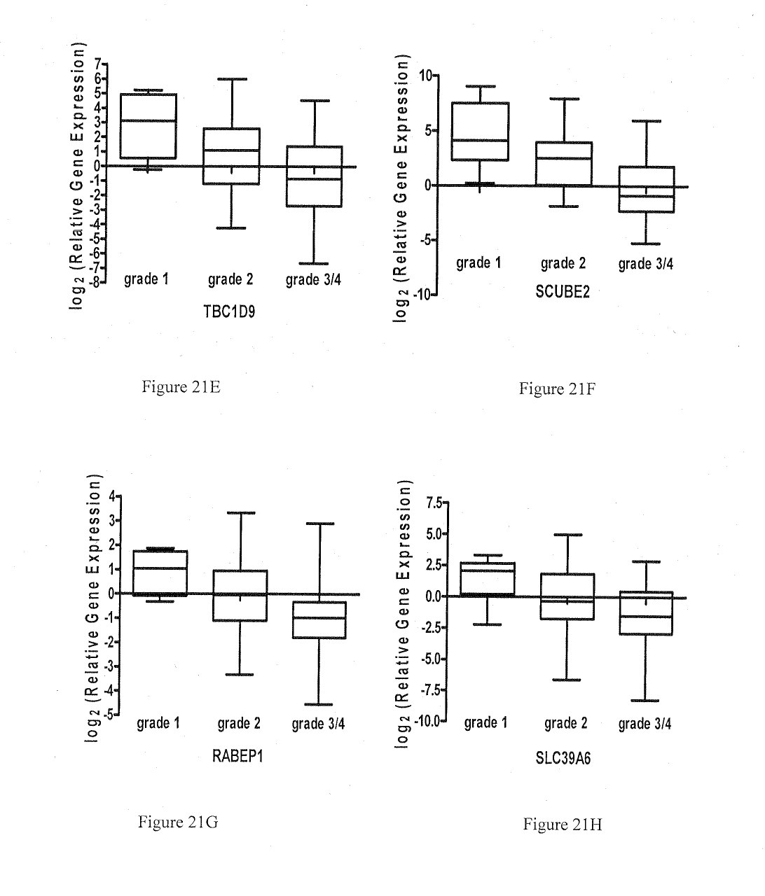

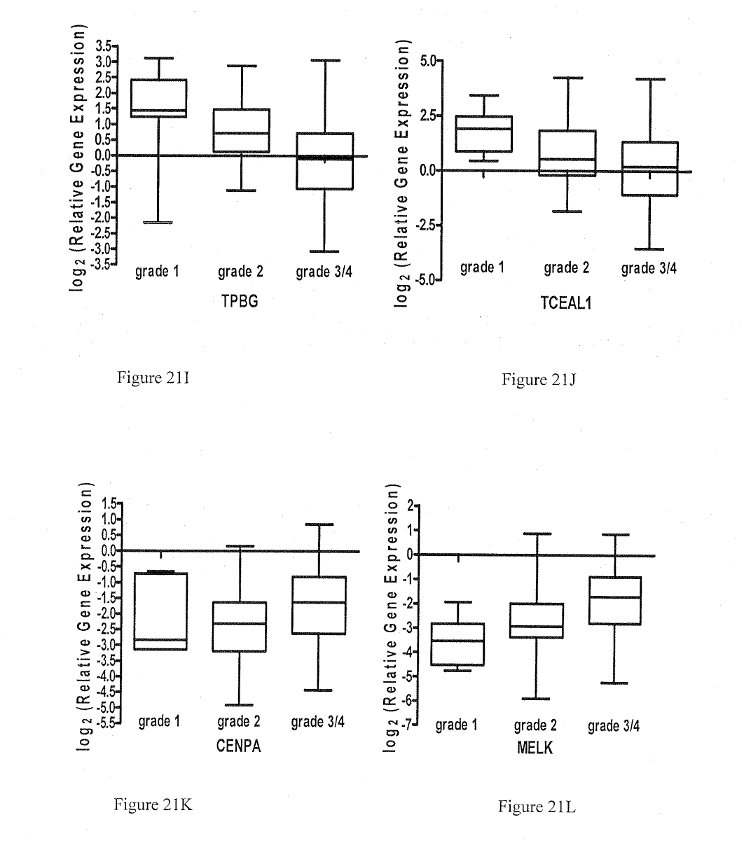

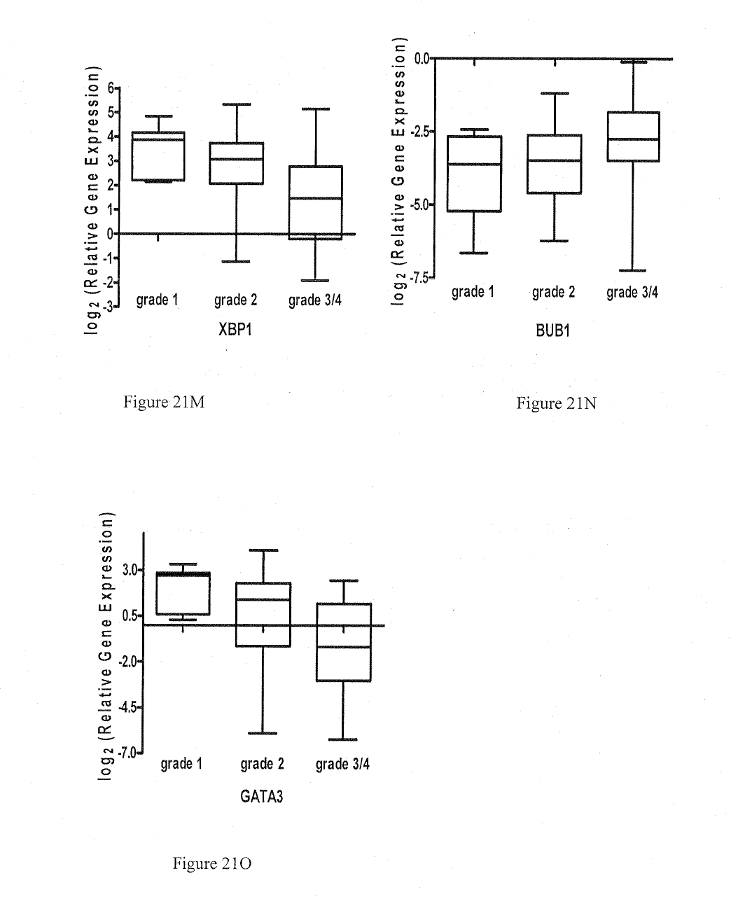

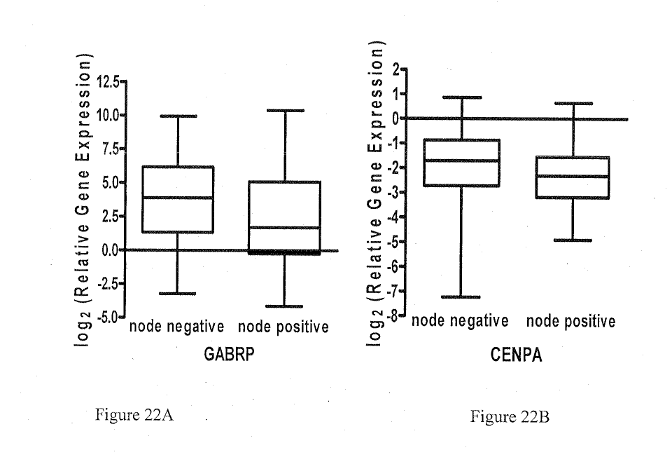

Gene expression as a function of different tumor grades are shown in FIG. 21 (grade 1 (n=7), grade 2 (n=35), and grades 3 and 4 (n=58)). Genes graphed are those with differences determined significant in ANOVA (EVL, NAT1, ESR1, ST8SIA1, TBC1D9, SCUBE2, RABEP1, SLC39A6, TPBG, TCEAL1, CENPA, MELK, XBP1, BUB1, and GATA3). As expected, the grade 2 tumors had relative gene expression levels between those of the grade 1 and grades 3 and 4 tumors. Several of the genes exhibited increased expression levels in carcinomas with increased nuclear grade, e.g., ST8SIA1, CENPA, MELK, and BUB1. Expression of the other genes decreased as a function of increased tumor grade. Since tumor grade is related to the degree of cellular differentiation, these genes may reflect molecular alterations characteristics of the malignant process.

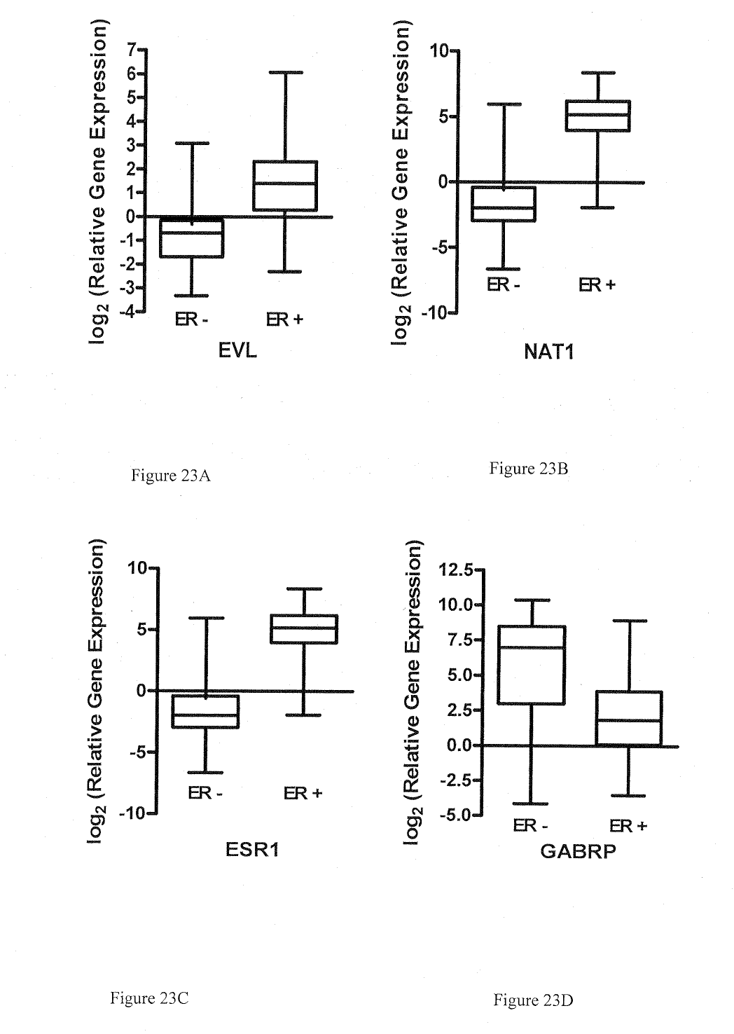

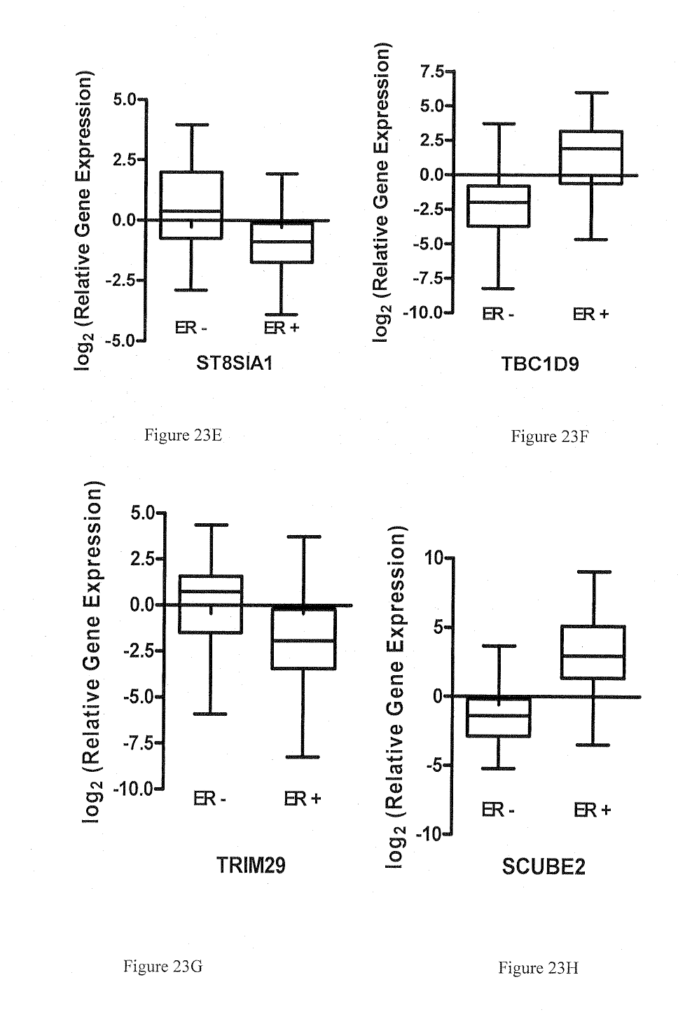

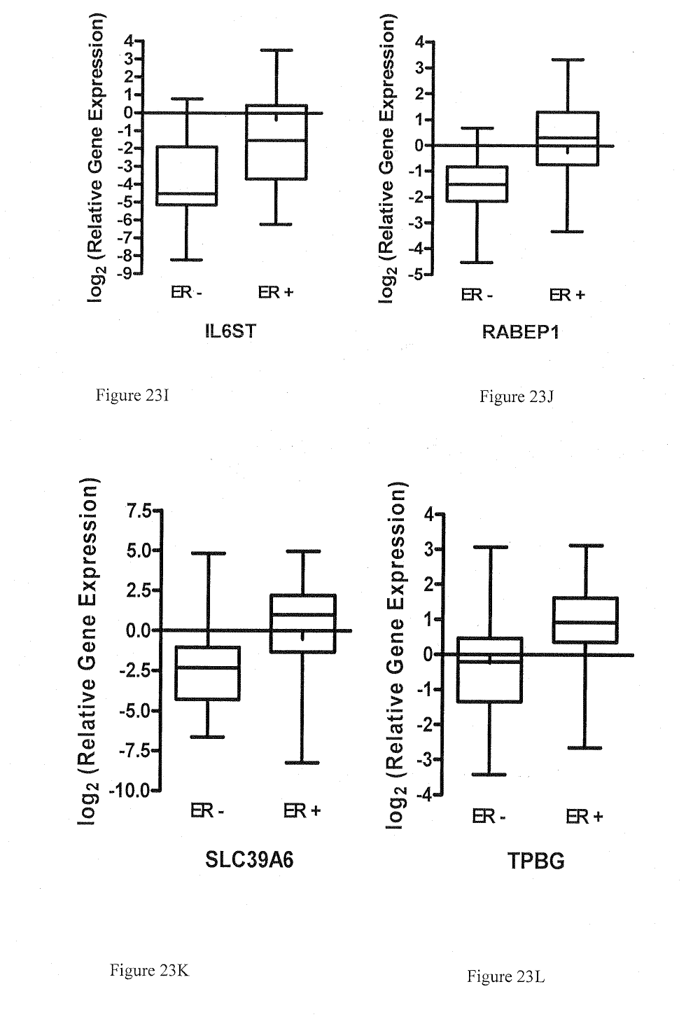

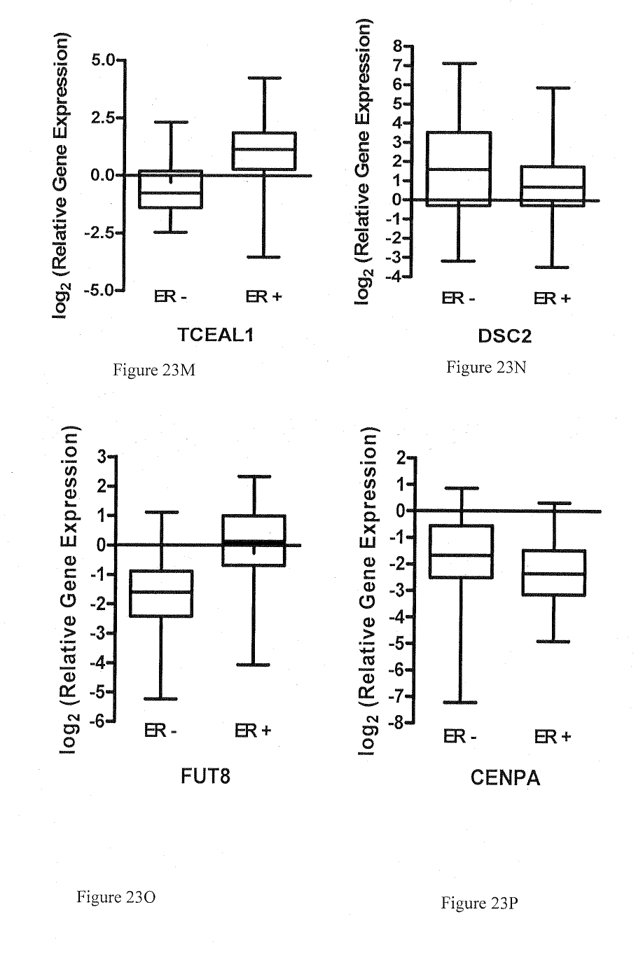

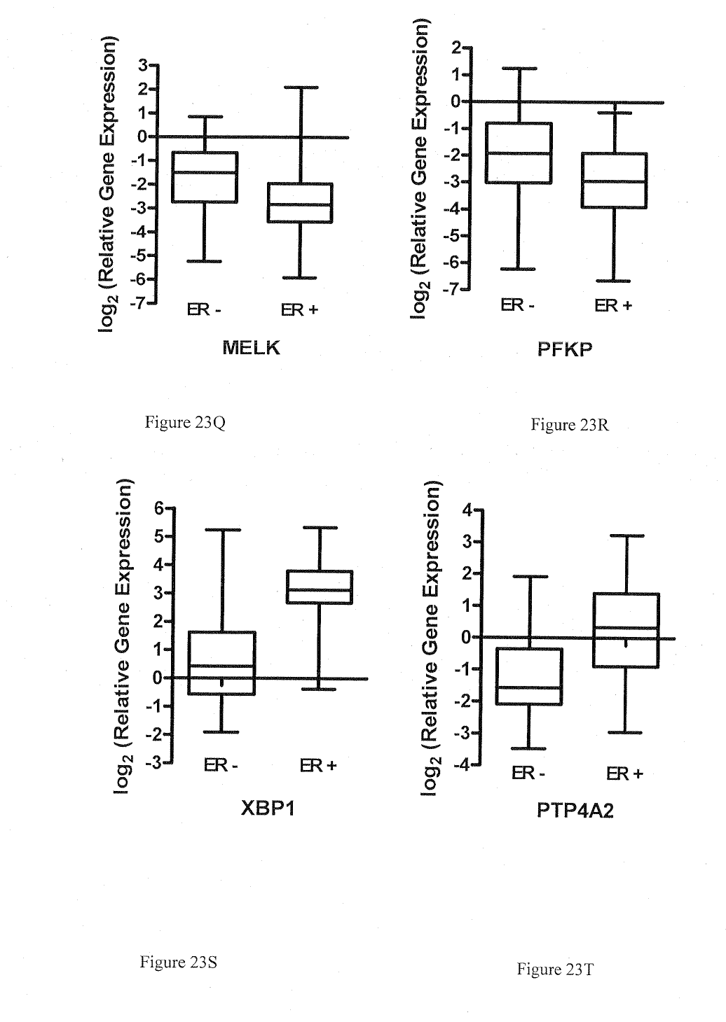

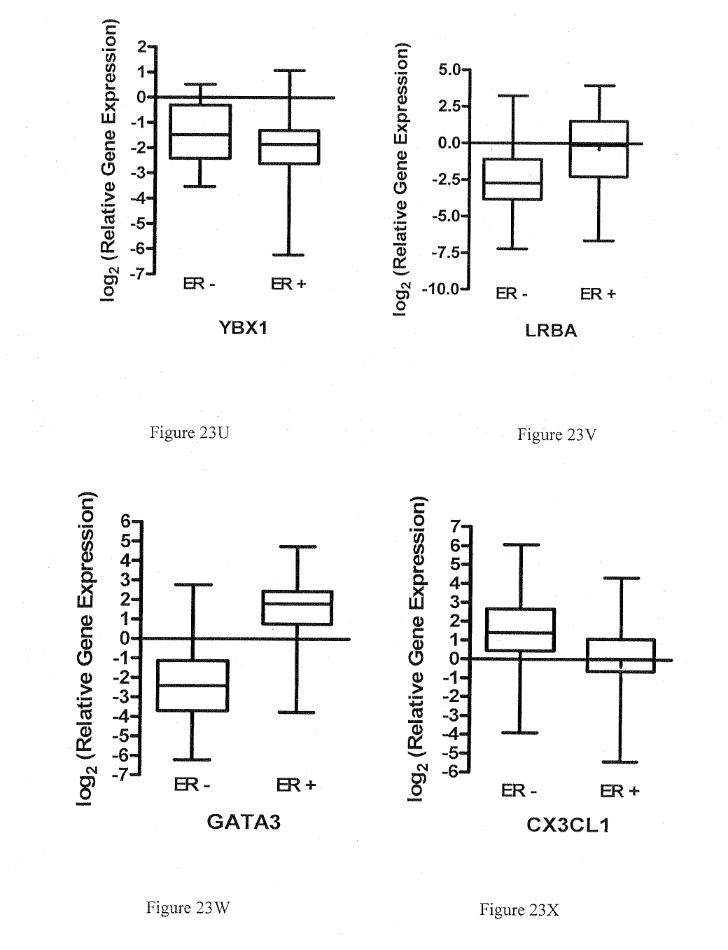

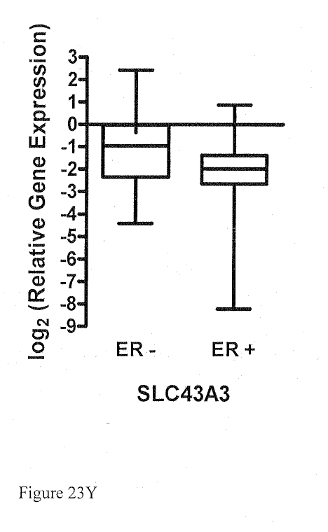

Differences in gene expression levels are shown in FIG. 22 for cancer patients who were lymph node negative (n=62) and positive (n=57). Only GABRP and CENPA exhibited expression differences that were significant in t-tests. Both of these genes had decreased expression levels in patients with node positive cancers compared to patients without lymph node involvement. Results of GABRP and CENPA are interesting because GABRP was reported to be down-regulated in 76% of breast cancers and was progressively down-regulated with tumor progression [170]. Furthermore, CENPA, which is a centromere-specific protein, is essential for correct kinetochore assembly and function [162], also implying its role in cancer progression. There were many differences in gene expression between ER negative (n=47) and ER positive (n=79) breast cancer patients (FIG. 23), which is predicted based on the differences in clinical outcome of these patients. Genes shown are those with differences determined significant in t-tests (i.e., EVL, NAT1, ESR1, GABRP, ST8SIA1, TBC1D9, TRIM29, SCUBE2, IL6ST, RABEP1, SLC39A6, TPBG, TCEAL1, DSC2, FUT8, CENPA, MELK, PFKP, XBP1, PTP4A2, YBX1, LRBA, GATA3, CX3CL1, and SLC43A3). Several genes were over-expressed in ER positive tumors compared to that of ER negative cancers, i.e., EVL, NAT1, ESR1 (as expected), TBC1D9, SCUBE2, IL6ST, RABEP1, SLC39A6, TPBG, TCEAL1, FUT8, XBP1, PTP4A2, LRBA, GATA3. Each of these genes was expected to have a positive correlation with the ER status of the breast cancer, because of their positive Pearson correlations with ESR1 gene expression shown in Table 30A-30H. Several other studies [180; 203] have also shown that GATA3 is highly associated with the estrogen receptor pathway. The genes shown to be under-expressed in ER positive compared to ER negative tumors were GABRP, ST8SIA1, TRIM29, DSC2, CENPA, MELK, PFKP, YBX1, CX3CL1, and SLC43A3. Each of these genes was expected to have an inverse correlation with ER status of the lesion, because of their negative Pearson correlations with ESR1 gene expression (Table 30A-30H). However, expression of CENPA and YBX1 was statistically significant in Pearson correlations at P<0.05. As indicated earlier, CENPA was differentially expressed between ER-positive and ER-negative breast cancer cell lines [163]. ST8SIA1 was previously shown to have higher expression in ER-negative breast tumors [177].

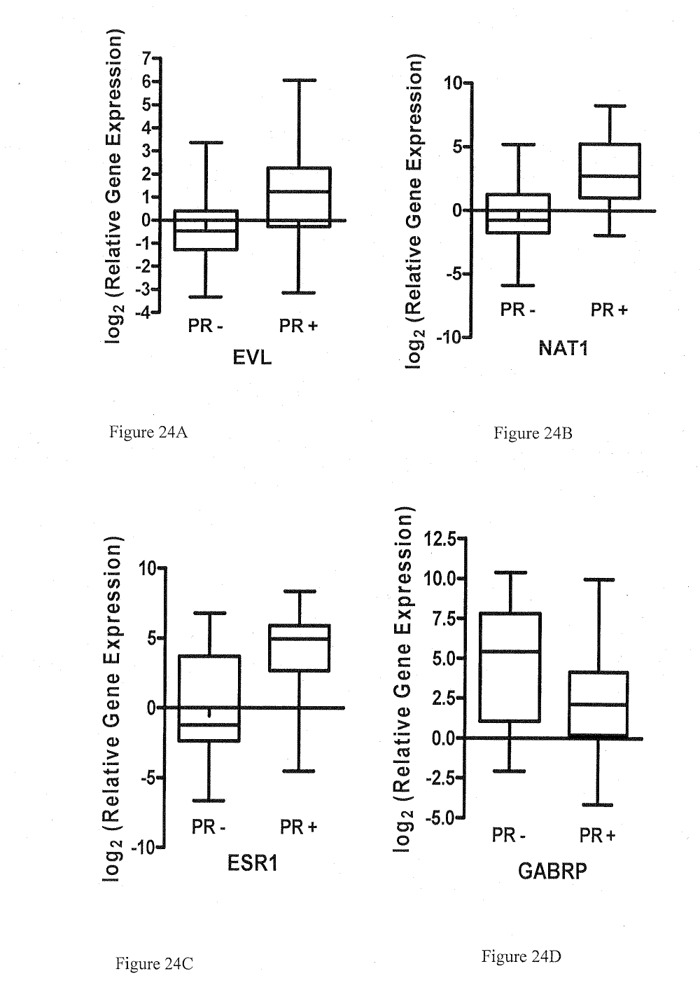

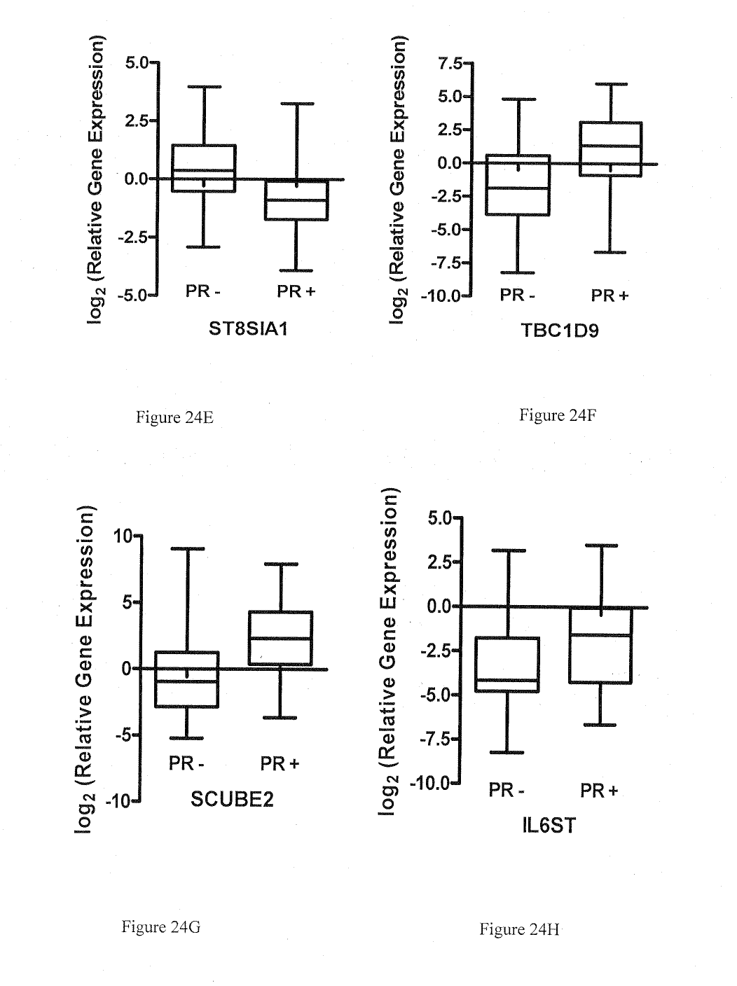

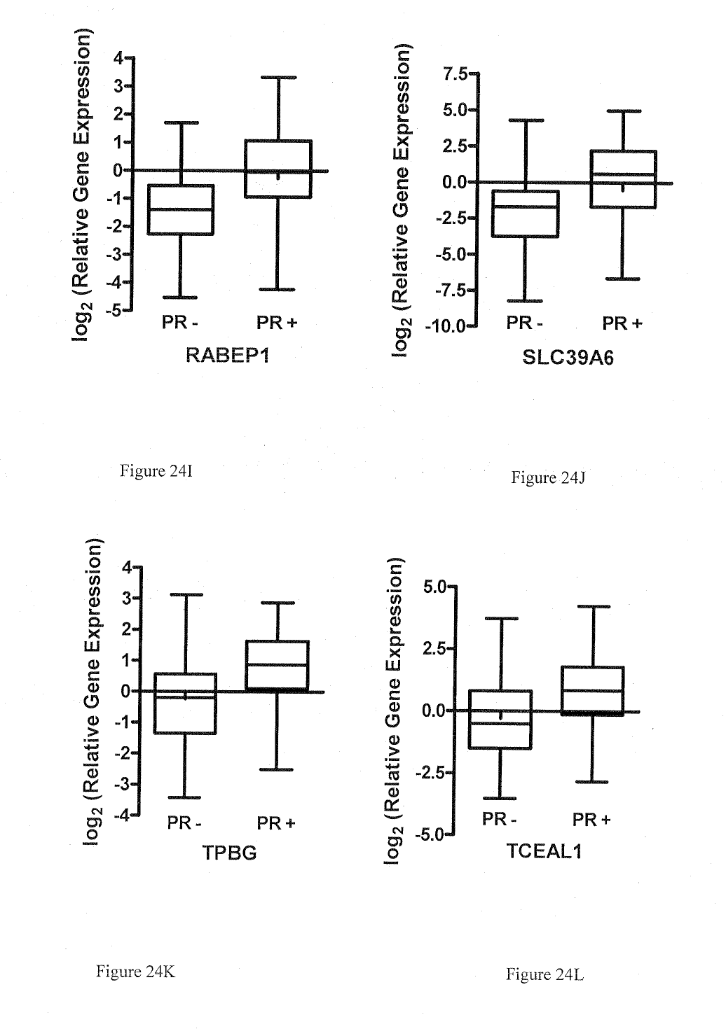

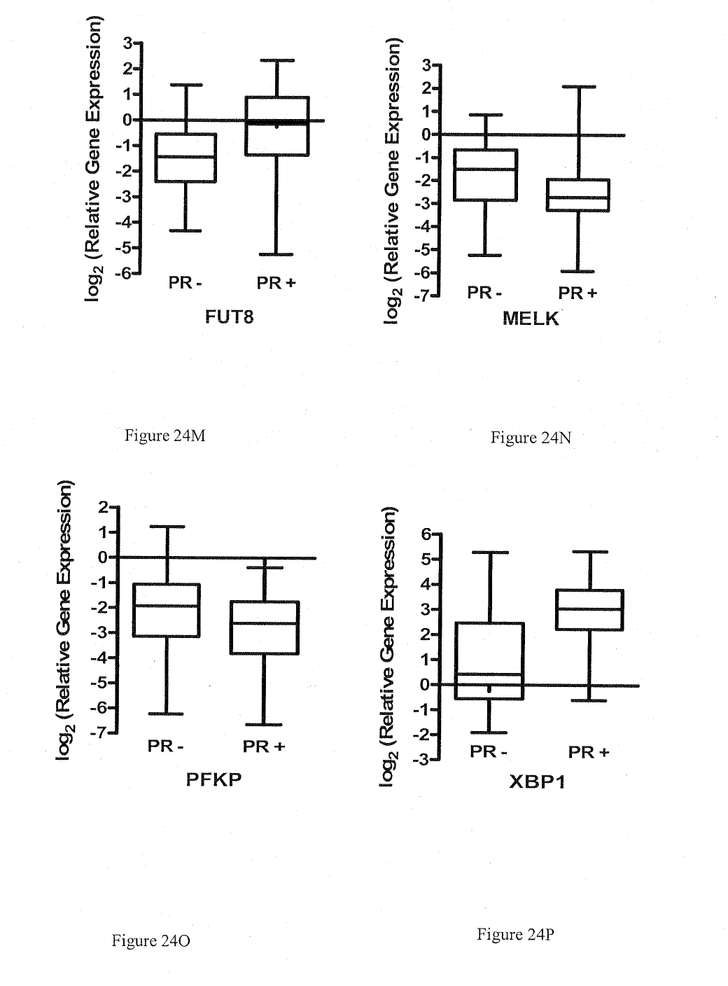

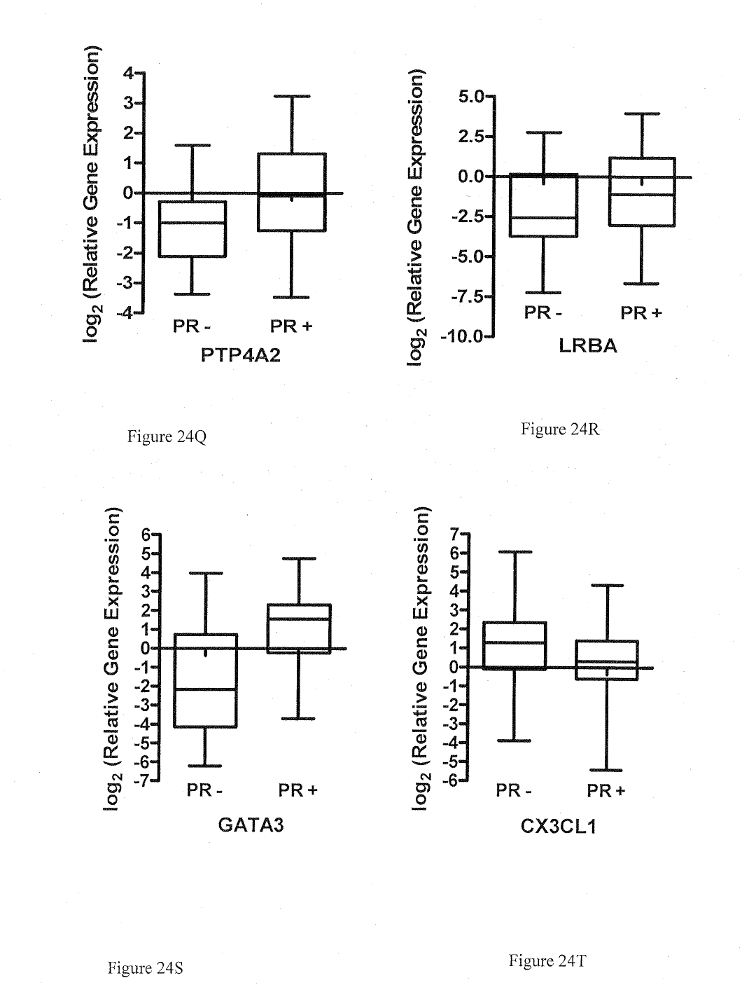

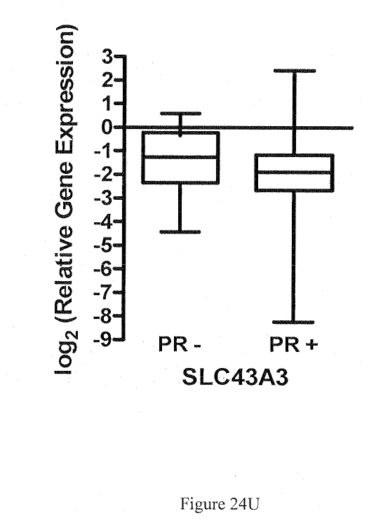

Similar analyses were performed comparing gene expression levels in PR negative (n=43) and PR positive (n=83) patients (FIG. 24). Genes shown are those with differences determined significant in t-tests (EVL, NAT1, ESR1, GABRP, ST8SIA1, TBC1D9, SCUBE2, IL6ST, RABEP1, SLC39A6, TPBG, TCEAL1, FUT8, MELK, PFKP, XBP1, PTP4A2, LRBA, GATA3, CX3CL1, and SLC43A3). Several of these genes were over-expressed in PR positive tumors compared to PR negative tumors, e.g., EVL, NAT1, ESR1, TBC1D9, SCUBE2, IL6ST, RABEP1, SLC39A6, TPBG, TCEAL1, FUT8, XBP1, PTP4A2, LRBA, and GATA3. These genes are similar to those that were over-expressed in ER positive tumors, and this is most likely due to the large degree of influence and cross-talk between the ER and PR pathways, e.g., PR is a target gene in the ER signal transduction pathway [252; 253].

Correlation of Expression Levels of Individual Genes with Clinical Outcome

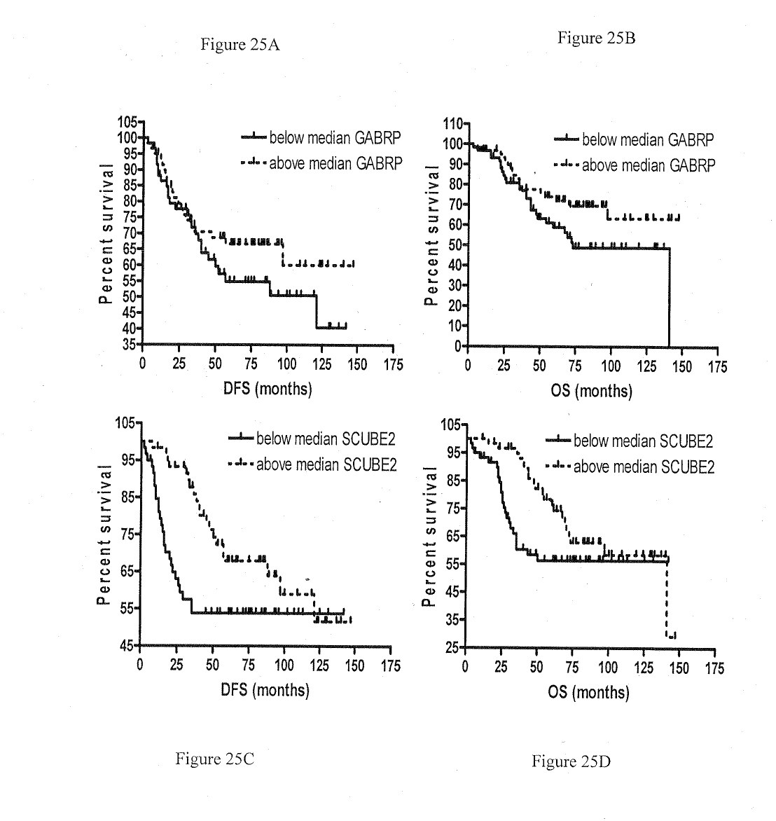

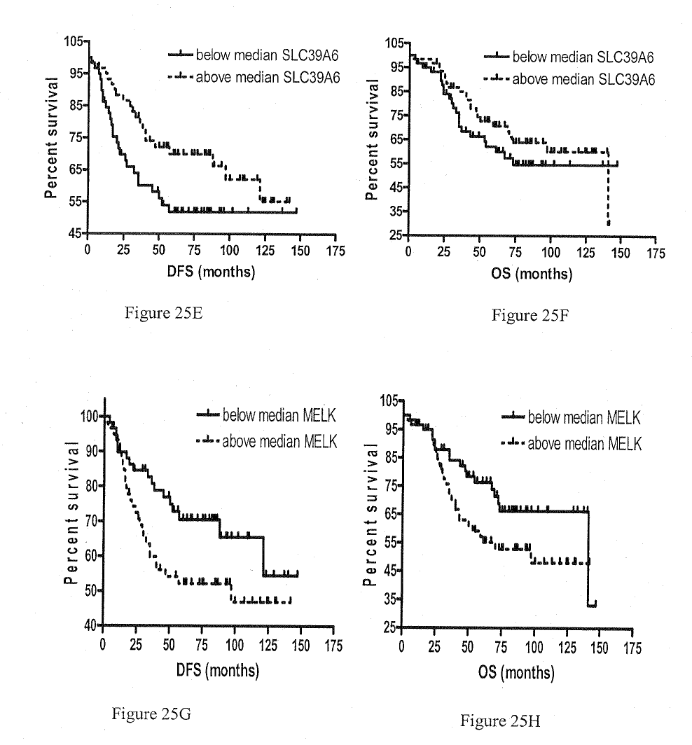

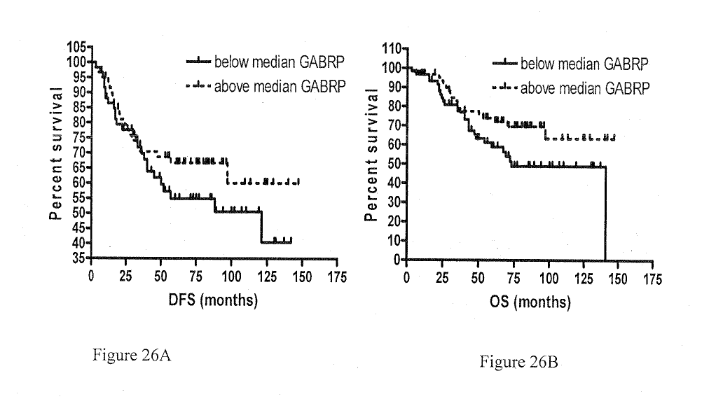

In a preliminary correlation of gene expression with patient outcome, t-tests were preformed comparing expression levels in patients exhibiting breast cancer recurrence with patients that remained disease-free (Table 32). In addition correlations of gene expression were made with patients that did not die from their breast cancer with those that died of breast cancer (Table 33). Analyses of gene expression levels with patient recurrence identified two genes (ATAD2 and CX3CL1) with P values less than 0.1. Both ATAD2 and CX3CL1 exhibited a lower level of expression in patients, who remained disease-free compared to those that had recurrences (Table 32). Similar analyses of gene expression levels with patient survival also identified two genes (PLK1 and CX3CL1) with P values less than 0.1. Both PLK1 and CX3CL1 exhibited a lower level of expression in patients who did not die of breast cancer compared to those that died of their cancer (Table 33). This observation is contradictory to another study [207] in prostate cancer, which expression of CX3CL1 was associated with good patient prognosis. While the P values in these evaluations are not statistically significant in this most basic form of survival analyses, it greatly suggests that these genes may prove useful for predicting disease recurrence and survival using more sophisticated methods. Expression of each gene was evaluated by Kaplan-Meier survival analyses using expression above and below median relative expression values to stratify patients (FIG. 25 and Table 34). This method of analysis incorporates time to event data, and allows use of all available patient survival data by the technique of censoring. Censoring data simply means that after a certain period of time, the patient data are no longer used in the analysis. This may be due to the patient either remaining alive at the end of the follow-up period, died for reasons unrelated to this cancer, or there was a lack of follow-up within the study period [249].

Of the 32 genes evaluated individually in the gene subsets, only SCUBE2 exhibited a median expression level that significantly stratified 126 patients into good and poor prognosis groups for disease recurrence (P value of less than 0.05, Table 34). A hazard ratio of 1.8 was calculated for SCUBE2 expression between the prognosis groups, indicating that the poor prognosis group had a 1.8-fold greater chance of having a recurrence of their breast cancer compared to the good prognosis group. Although most of the individual genes tested did not show statistically significant correlations with recurrence and survival, many appear to indicate trends which separated patients into prognostic groups. Expression of six additional genes (GABRP, TBC1D9, SLC39A6, MELK, MCM6, and PTP4A2) appears to be associated with either disease-free or overall survival (P value less than 0.10). The hazard ratios for each gene are shown (Table 34). It should be noted that these hazard ratios are representative of the patient population only after the gene is determined to be statistically significant. Expression of several of these genes approached significance (GABRP, TBC1D9, SLC39A6, MCM6, and PTP4A2) with hazard ratios above 1, indicating that elevated expression of the gene is related to decreased patient survival. However, elevated expression of MELK was correlated with increased disease-free and overall survival. Representative Kaplan-Meier plots of patients with disease-free and overall survival as a function of expression of single genes (GABRP, SCUBE2, SLC39A6, and MELK) are shown in FIG. 25. As illustrated, there is significant separation of survival curves when the patients are stratified by above or below median expression levels for the particular gene.

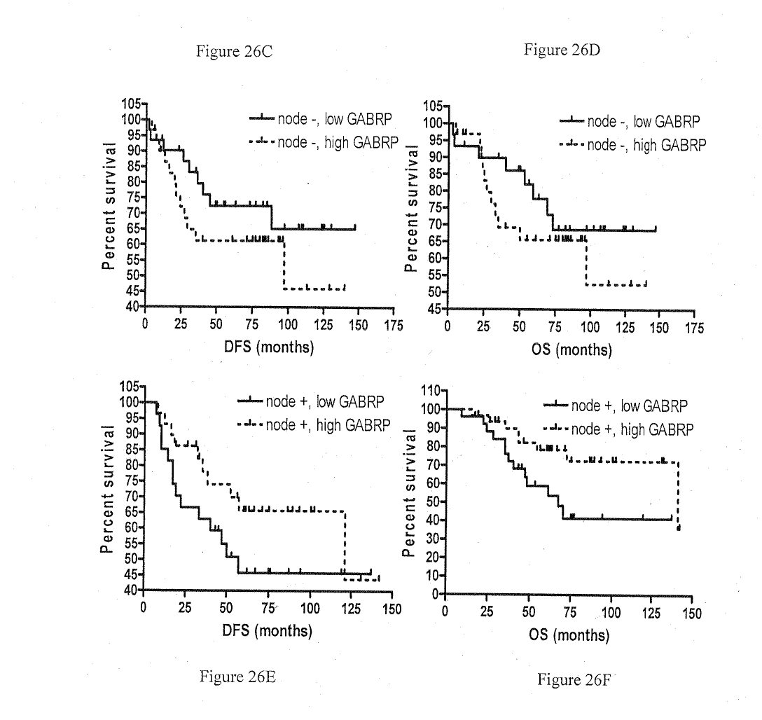

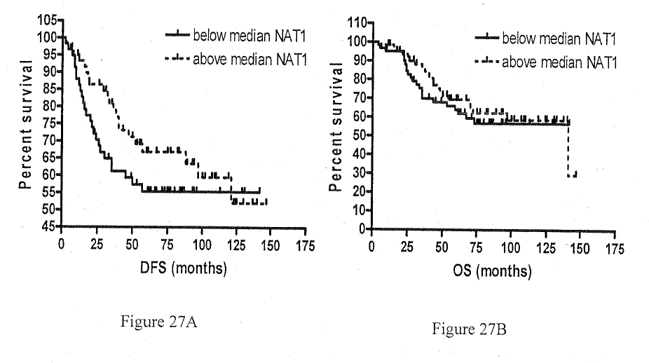

From evaluations of various patient and cancer features (Table 31), genes that were differentially expressed related to a particular characteristic were evaluated in the two populations. Two genes (GABRP and CENPA) had differential expression when comparing patients with lymph node positive or negative cancers were analyzed for patient survival. The relationship of GABRP expression with patient disease-free and overall survival is shown for all patients (FIGS. 26A and B), those that are node negative (FIGS. 26C and D), and those that are node positive (FIGS. 26E and F). GABRP gene expression (above and below median values) was able to better stratify patients that were lymph node positive compared to lymph node negative patients of the entire population. Thus, knowledge of lymph node status is useful when analyzed GABRP expression for predicting clinical outcome. Survival analysis of CENPA expression was not altered in patients separated by nodal status (data not shown).

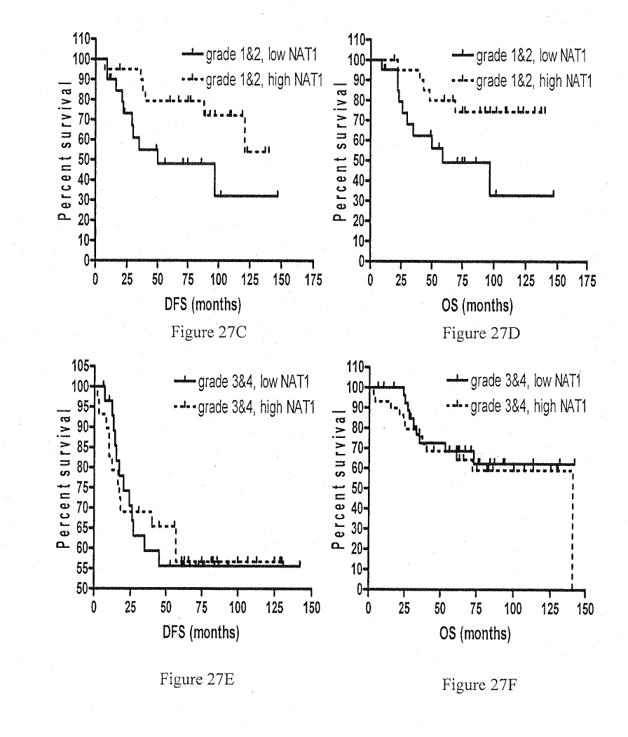

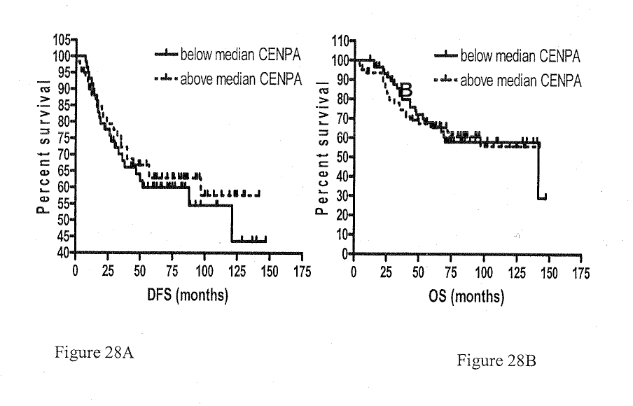

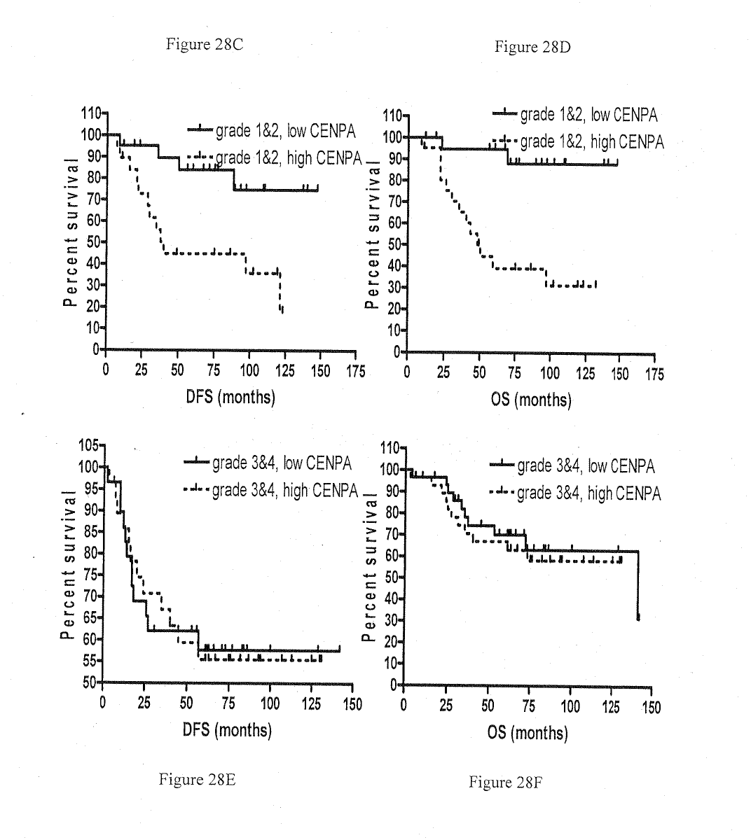

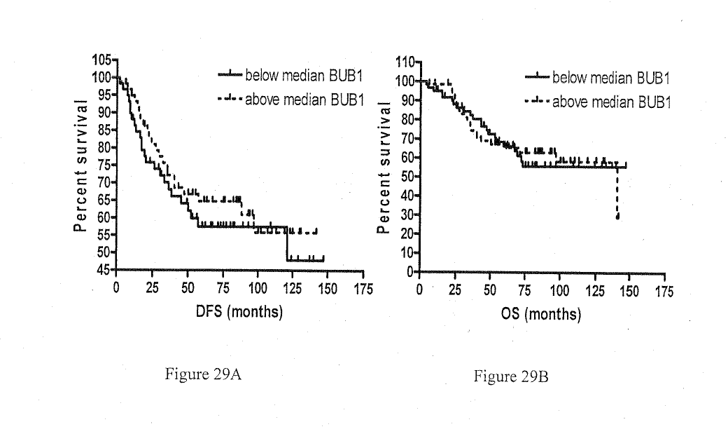

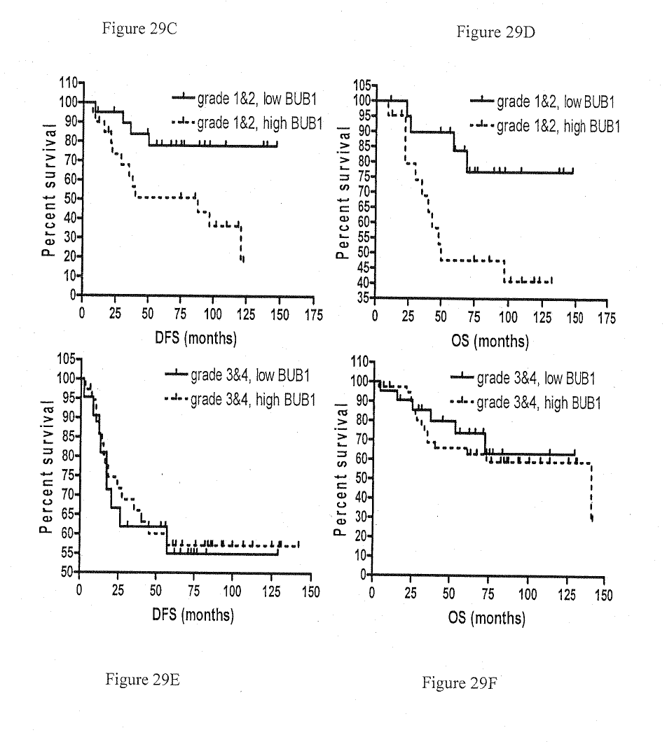

Similar analyses were performed for the genes altered in patients with different tumor grades (FIGS. 27-29-44). FIG. 27 shows survival analyses when NAT1 is evaluated in low grade tumors (grades 1 and 2) verses high grade tumors (grades 3 and 4). There is significantly greater separation between the two survival curves in low grade tumors, compared to that of high grade tumors or of the entire population. CENPA is analyzed in FIG. 28, and BUB1 is analyzed in FIG. 29 showing similar results. Expression of these genes in breast cancer significantly separates the two survival curves of patients with low grade tumors, compared to those with high grade tumors or those of the entire population. This suggests the clinical utility of analyzing expression of these three genes in low grade tumors, although they may be of limited value for predicting clinical outcome in patients with high grade tumors.

TABLE 32
Results of t-test analyses of gene expression levels comparing
patients exhibiting breast cancer disease recurrences with patients
that remained disease-free.

| | | MEAN | |
|---|---|---|---|
| | MEAN (LOG₂ GENE | (LOG₂ GENE | |
| | EXPRESSION) OF | EXPRESSION) OF | |
| | PATIENTS WITHOUT | PATIENTS WITH | T-TEST |
| GENE ID | RECURRENCE | RECURRENCE | (P VALUE) |
| EVL | 0.56 | 0.49 | 0.85 |
| NAT1 | 2.11 | 1.54 | 0.28 |
| ESR1 | 2.60 | 2.40 | 0.77 |
| GABRP | 3.36 | 3.09 | 0.67 |
| ST8SIA1 | −0.48 | −0.26 | 0.51 |
| TBC1D9 | 0.27 | −0.06 | 0.56 |
| TRIM29 | −0.87 | −1.47 | 0.20 |
| SCUBE2 | 1.46 | 1.41 | 0.93 |
| IL6ST | −2.22 | −2.48 | 0.60 |
| RABEP1 | −0.30 | −0.59 | 0.31 |
| SLC39A6 | −0.26 | −1.09 | 0.11 |
| TPBG | 0.33 | 0.47 | 0.59 |
| TCEAL1 | 0.48 | 0.42 | 0.85 |
| DSC2 | 0.87 | 1.22 | 0.39 |
| FUT8 | −0.58 | −0.71 | 0.68 |
| CENPA | −2.09 | −2.03 | 0.82 |
| MELK | −2.39 | −2.21 | 0.47 |
| PFKP | −2.64 | −2.57 | 0.79 |
| PLK1 | −2.67 | −2.44 | 0.29 |
| ATAD2 | −1.30 | −0.96 | 0.07 |
| XBP1 | 2.31 | 2.06 | 0.44 |
| MCM6 | −2.49 | −2.46 | 0.90 |
| BUB1 | −3.20 | −3.15 | 0.83 |
| PTP4A2 | −0.24 | −0.56 | 0.25 |
| YBX1 | −1.81 | −1.65 | 0.46 |
| LRBA | −1.37 | −1.19 | 0.69 |
| GATA3 | 0.24 | −0.04 | 0.56 |
| CX3CL1 | 0.47 | 1.04 | 0.08 |
| MAPRE2 | −1.86 | −1.83 | 0.93 |
| GMPS | −1.62 | −1.58 | 0.89 |
| CKS2 | −2.08 | −1.97 | 0.74 |
| SLC43A3 | −1.83 | −1.62 | 0.40 |

TABLE 33
Results of t-test analyses of gene expression levels in breast
carcinoma comparing patients that died of disease with those that
did not die of breast cancer.

| | MEAN (LOG₂ GENE | MEAN (LOG₂ GENE | |
|---|---|---|---|
| | EXPRESSION) OF | EXPRESSION) OF | |
| | PATIENTS WHO | PATIENTS WHO | |
| | DID NOT DIE OF | DIED OF | T-TEST |
| GENE ID | BREAST CANCER | BREAST CANCER | (P VALUE) |
| EVL | 0.62 | 0.38 | 0.48 |
| NAT1 | 2.00 | 1.65 | 0.52 |
| ESR1 | 2.51 | 2.54 | 0.97 |
| GABRP | 3.28 | 3.20 | 0.91 |
| ST8SIA1 | −0.48 | −0.24 | 0.49 |
| TBC1D9 | 0.19 | 0.03 | 0.77 |
| TRIM29 | −1.04 | −1.25 | 0.65 |
| SCUBE2 | 1.41 | 1.50 | 0.87 |
| IL6ST | −2.32 | −2.32 | 0.99 |
| RABEP1 | −0.34 | −0.55 | 0.47 |
| SLC39A6 | −0.46 | −0.84 | 0.46 |
| TPBG | 0.29 | 0.56 | 0.29 |
| TCEAL1 | 0.43 | 0.50 | 0.81 |
| DSC2 | 0.81 | 1.38 | 0.18 |
| FUT8 | −0.64 | −0.63 | 0.98 |
| CENPA | −2.15 | −1.92 | 0.37 |
| MELK | −2.45 | −2.09 | 0.16 |
| PFKP | −2.70 | −2.46 | 0.37 |
| PLK1 | −2.72 | −2.34 | 0.09 |
| ATAD2 | −1.24 | −1.02 | 0.25 |
| XBP1 | 2.27 | 2.10 | 0.59 |
| MCM6 | −2.57 | −2.32 | 0.39 |
| BUB1 | −3.25 | −3.05 | 0.43 |
| PTP4A2 | −0.26 | −0.55 | 0.31 |
| YBX1 | −1.85 | −1.57 | 0.20 |
| LRBA | −1.40 | −1.11 | 0.54 |
| GATA3 | 0.16 | 0.05 | 0.81 |
| CX3CL1 | 0.47 | 1.10 | 0.06 |
| MAPRE2 | −1.98 | −1.60 | 0.13 |
| GMPS | −1.70 | −1.42 | 0.23 |
| CKS2 | −2.13 | −1.86 | 0.38 |
| SLC43A3 | −1.86 | −1.53 | 0.19 |

TABLE 34
Summary of results from Kaplan-Meier analyses relating individual gene
expression to disease-free and overall survival.

| | DISEASE-FREE | | | |
|---|---|---|---|---|
| | SURVIVAL | | OVERALL SURVIVAL | |
| | P | | P | |
| GENE ID | VALUE | HAZARD RATIO | VALUE | HAZARD RATIO |
| EVL | 0.474 | 1.23 | 0.260 | 1.39 |
| NAT1 | 0.202 | 1.45 | 0.526 | 1.21 |
| ESR1 | 0.112 | 1.59 | 0.357 | 1.31 |
| GABRP | 0.229 | 1.43 | 0.055 | 1.77 |
| ST8SIA1 | 0.916 | 1.03 | 0.407 | 1.28 |
| TBC1D9 | 0.097 | 1.62 | 0.089 | 1.65 |
| TRIM29 | 0.333 | 1.33 | 0.118 | 1.59 |
| SCUBE2 | 0.043 | 1.80 | 0.118 | 1.59 |
| IL6ST | 0.474 | 1.23 | 0.572 | 1.18 |
| RABEP1 | 0.128 | 1.56 | 0.143 | 1.54 |
| SLC39A6 | 0.058 | 1.74 | 0.325 | 1.34 |
| TPBG | 0.193 | 1.47 | 0.198 | 1.46 |
| TCEAL1 | 0.496 | 1.22 | 0.626 | 1.16 |
| DSC2 | 0.106 | 0.62 | 0.126 | 0.64 |
| FUT8 | 0.288 | 1.36 | 0.864 | 1.05 |
| CENPA | 0.588 | 1.17 | 0.896 | 0.96 |
| MELK | 0.060 | 0.57 | 0.088 | 0.60 |
| PFKP | 0.566 | 1.18 | 0.677 | 1.13 |
| PLK1 | 0.326 | 0.75 | 0.284 | 0.73 |
| ATAD2 | 0.158 | 0.66 | 0.318 | 0.74 |
| XBP1 | 0.165 | 1.50 | 0.278 | 1.37 |
| MCM6 | 0.097 | 1.63 | 0.576 | 1.18 |
| BUB1 | 0.475 | 1.23 | 0.893 | 1.04 |
| PTP4A2 | 0.055 | 1.75 | 0.115 | 1.59 |
| YBX1 | 0.860 | 0.95 | 0.572 | 0.85 |
| LRBA | 0.283 | 0.73 | 0.420 | 0.79 |
| GATA3 | 0.150 | 1.52 | 0.233 | 1.42 |
| CX3CL1 | 0.173 | 0.67 | 0.352 | 0.76 |
| MAPRE2 | 0.953 | 1.02 | 0.965 | 0.99 |
| GMPS | 0.861 | 0.95 | 0.619 | 0.86 |
| CKS2 | 0.851 | 1.06 | 0.809 | 0.93 |
| SLC43A3 | 0.880 | 0.96 | 0.628 | 0.87 |

Patients were stratified by median gene expression values, and Kaplan-Meier analyses were performed. P values indicating that either high or low expression of an individual gene was related to survival outcomes of breast cancer patients are shown with the hazard ratios. Values shown in bold indicate a statistically significant difference (P value less than 0.05) was observed in patient survival between the strata.

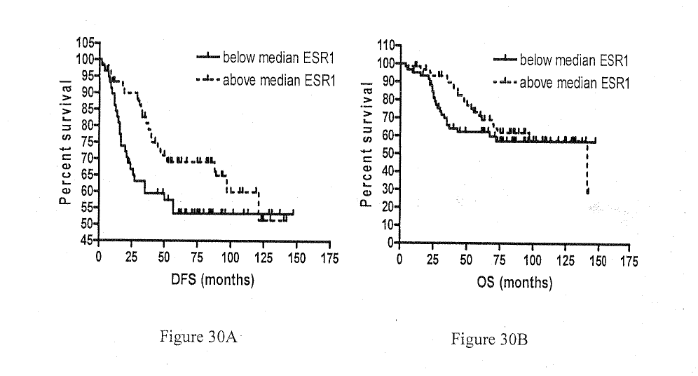

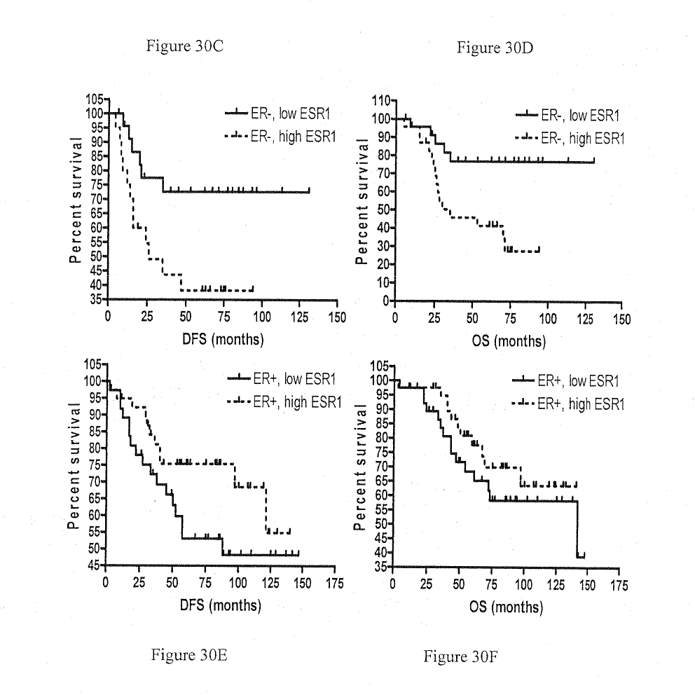

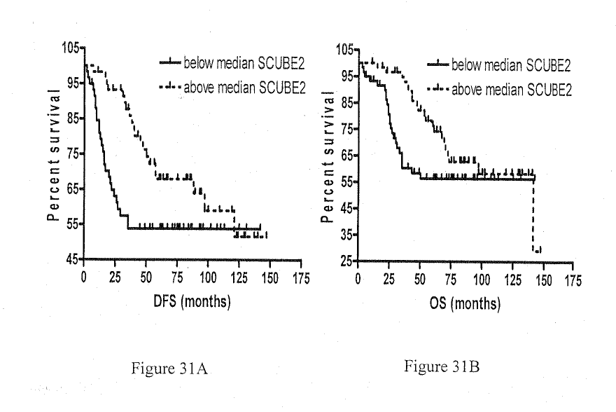

Although 25 of the 32 gene set were associated with ER expression (Table 31), Kaplan-Meier analyses are shown for ESR1, SCUBE2, RABEP1, SLC39A6, TCEAL1, and XBP1 in ER positive or ER negative patients (FIGS. 30-35). As shown in FIGS. 30A and 30B, when only ESR1 gene expression (stratified by median expression level) was evaluated on the entire patient population without consideration of ER status, two groups of patients were identified with different disease-free and overall survival probabilities. Similar results were observed when Kaplan-Meier analyses were performed in the ER (protein) positive cohort as a function of ESR1 gene expression (FIGS. 30E and 30F). These data based on ESR1 mRNA levels are in agreement with those generally reported for ER protein levels [10; 11; 91; 135]. Decreased expression of ST8SIA1 was not found to be associated with worse prognosis in this population of ER-positive tumors, as previously described by Ruckhaberle et al. [178].

However, a surprising result was observed when only the ER (protein) negative population of patients was analyzed by Kaplan-Meier plots as a function of ESR1 gene expression (FIGS. 30C and 30D). Two patient groups with distinct differences in survival probabilities were identified (DFS, P value=0.03; OS, P value=0.01) even though their breast carcinomas were ER negative. Patients with low levels of ESR1 expression had unexpectedly better survival (DFS and OS) outcomes than those with elevated ESR1 expression. This is the considerably different from the predicted result, in which the survival curves would be reversed. An explanation for this phenomena may be a splice variant of the ER protein [254], which could result in an inactive protein (unable to bind estrogen) that would not be measured in the clinical LBA. This observation was analyzed with the microarray data previously obtained from LCM-procured carcinoma cells from 247 patient specimens [41; 57; 70; 71]. Although a similar trend was observed in this larger patient population, the difference in survival curves was not statistically significant (data not shown). Wittliff and co-workers [70; 71] did distinguish two groups of ER negative breast cancer patients with different survival probabilities. This suggests that there are gene networks in certain ER negative breast cancers that result in diminished tumor progression similar to an ER positive lesion. The ability to distinguish prognosis of ER negative breast cancer, which is not only Tamoxifen insensitive [8; 11], but also is a difficult disease to treat, would greatly improve clinical management, since patients with good prognosis would be identified.

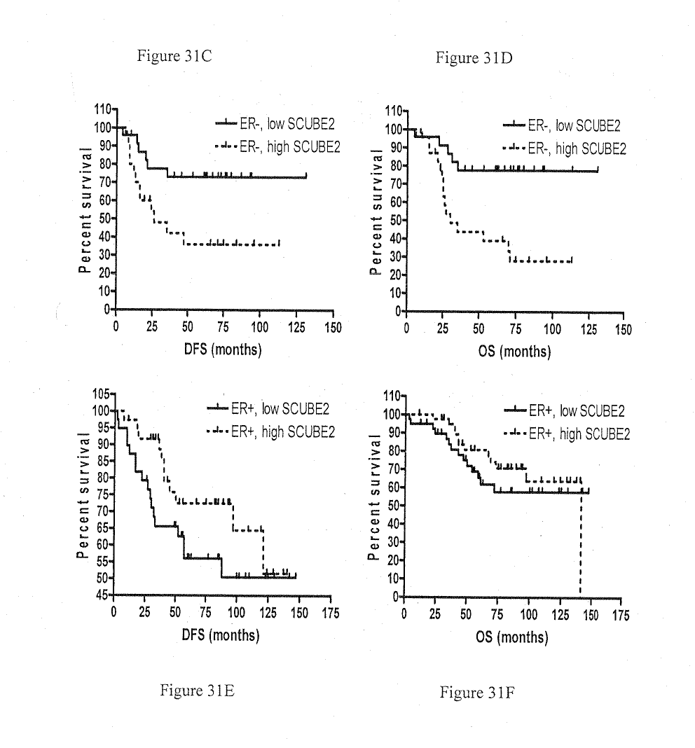

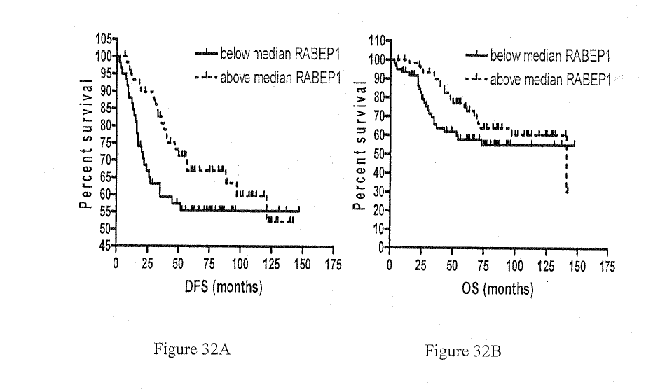

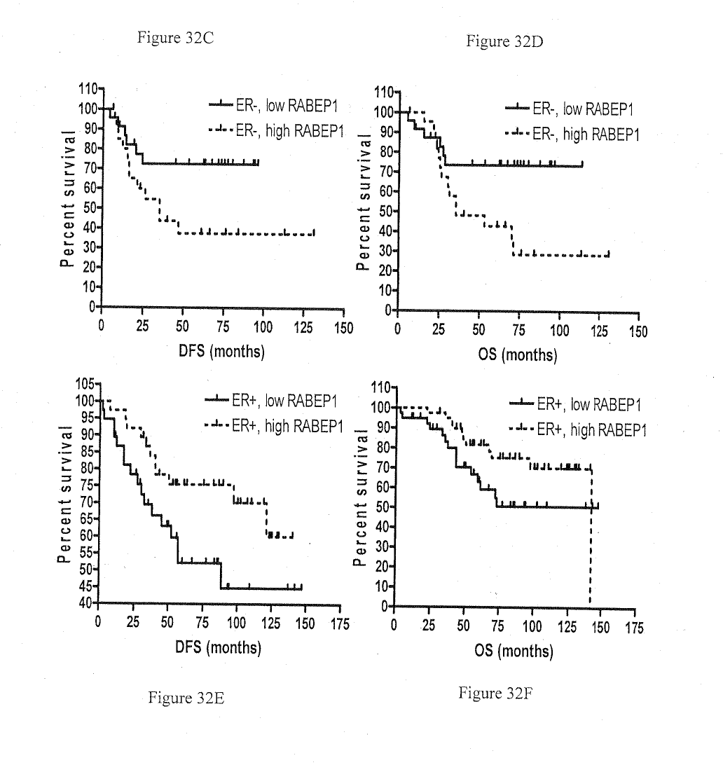

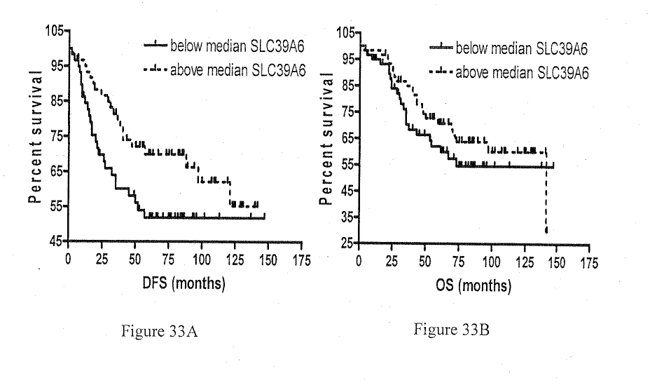

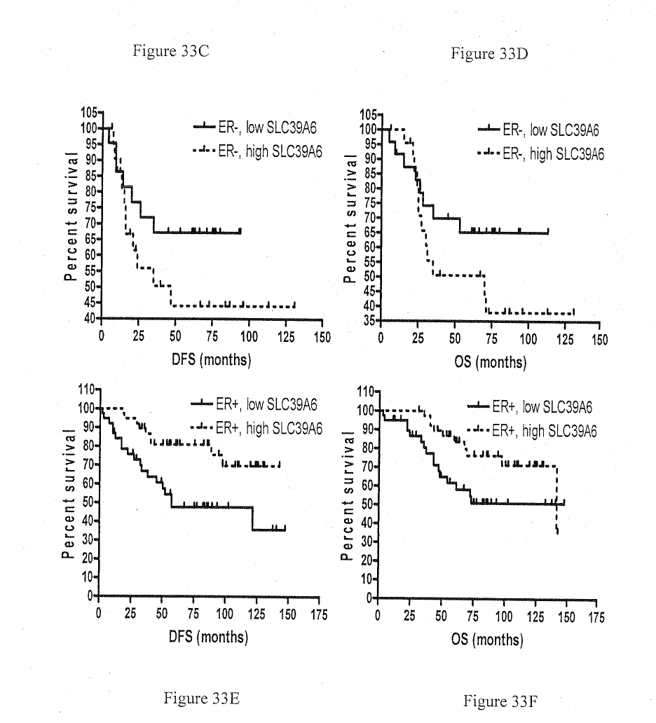

Analyses of SCUBE2 gene expression in ER negative or ER positive patients were performed (FIG. 31). Expression of this gene also separated survival in the ER negative cohort (DFS, P value=0.02; OS, P value <0.01) is a statistically significant manner, although it was not statistically significant in the ER positive cohort. SCUBE2 is one of the 21 genes in the Oncotype DX breast cancer test. Similar analyses were performed in RABEP1, SLC39A6, TCEAL1, and XBP1 with varying results. RABEP1 displayed statistically significant differences in survival in ER negative patients (DFS, P value=0.05; OS, P value=0.03). While its expression was significant for separating disease-free survival in ER positive patients (P value=0.05), it was not significant for overall survival (FIG. 32). SLC39A6 expression distinguished survival outcomes in ER positive patients (DFS, P value=0.01; OS, P value=0.04). Although the survival curves in ER negative patients indicated separation, they were not statistically significant (FIG. 33). The protein product of SLC39A6 (LIV-1) was reported to be regulated by estrogen in other studies [147; 148].

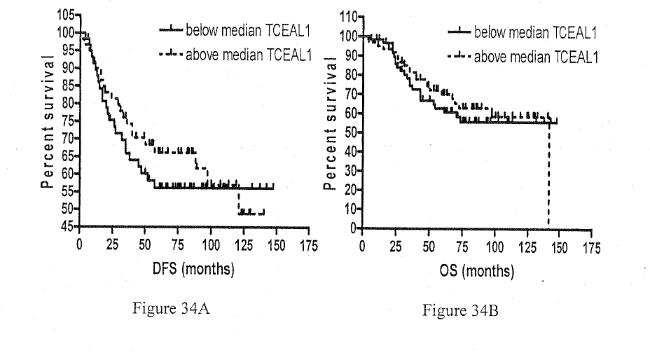

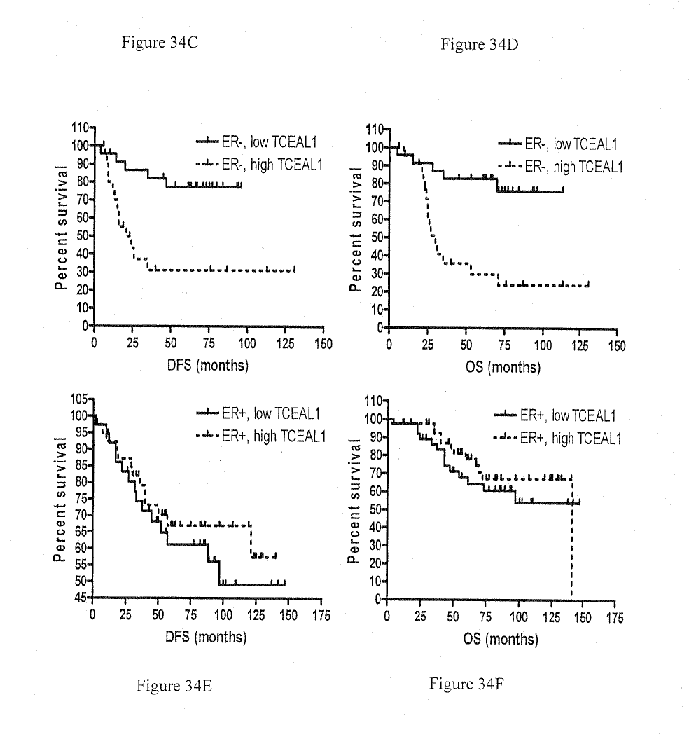

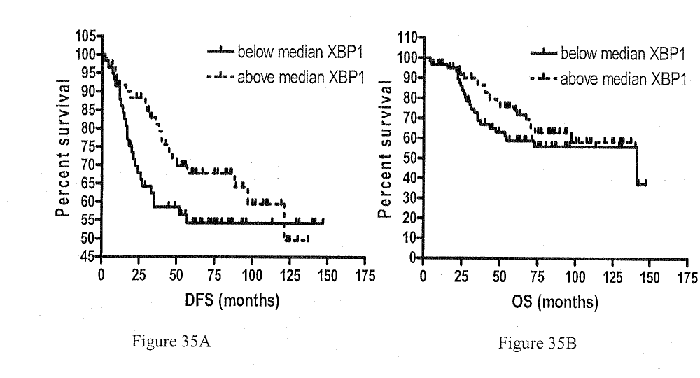

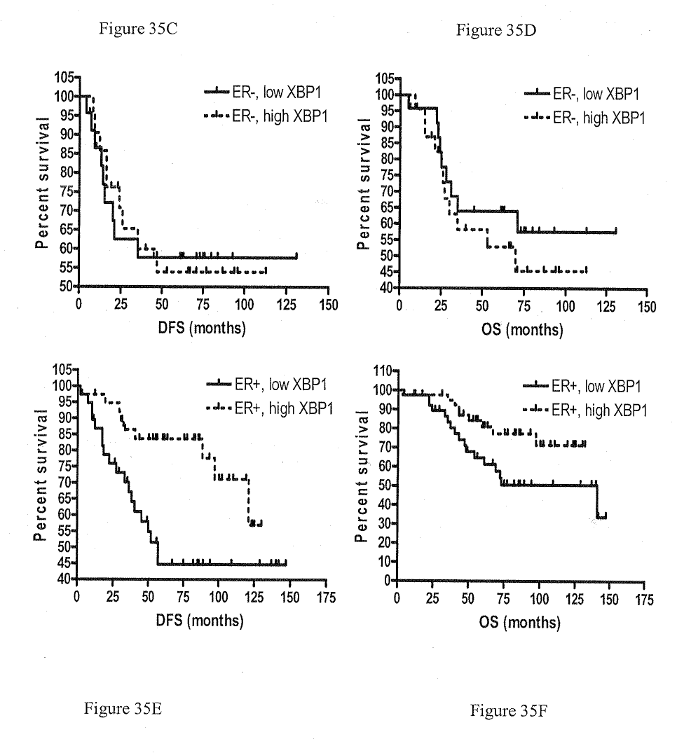

Analyses of TCEAL1 was interesting, because as indicated in the entire population (FIGS. 34A and 34B), it did not distinguish patients with differing outcomes when evaluated for above and below median expression levels. However, there was a highly significant difference in patient survival in the ER negative population (DFS, P value <0.01; OS, P value <0.01). Expression levels of XBP1 predicted outcome in ER positive patients (DFS, P value=0.01; OS, P value=0.04), but not for ER negative breast cancer (FIG. 35).

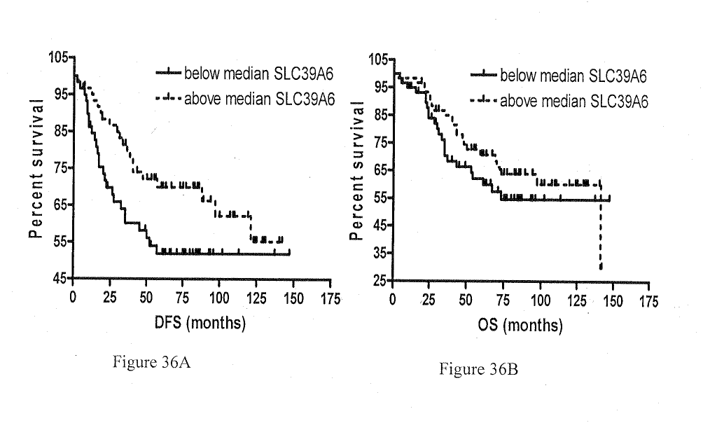

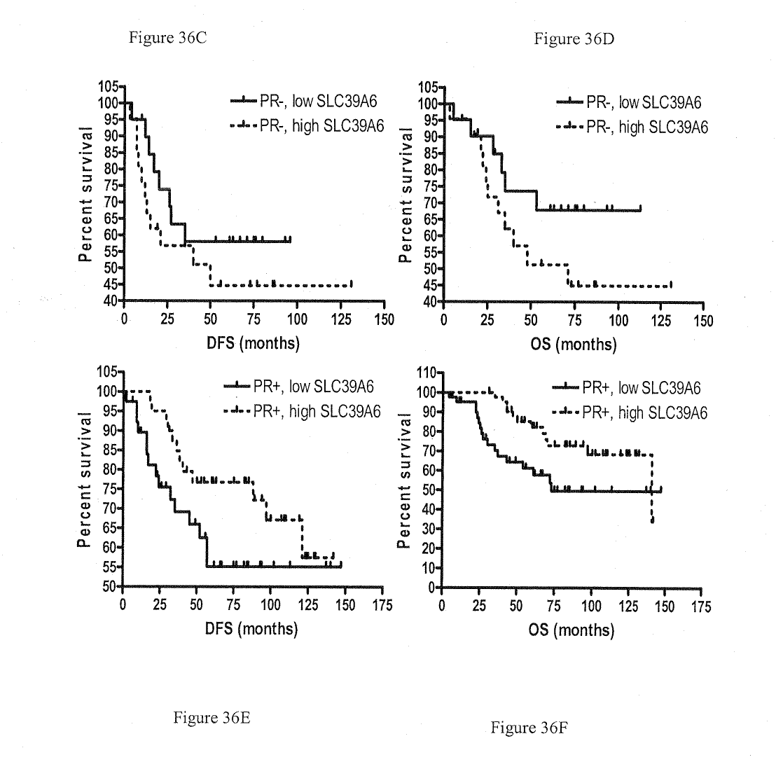

Similar analyses of gene expression in PR negative or PR positive patients were performed for 21 genes differentially expressed between those patient cohorts (Table 31). FIG. 36 illustrates the expression of SLC39A6 (above and below the median level), which was able to better stratify PR positive patients (FIG. 36E) than PR negative patients (FIG. 36C) for disease-free survival. Although the overall survival curves for the entire patient population (FIG. 36B) did not significantly stratify the patients, dividing patients based on PR status prior to Kaplan-Meier analyses yielded significant separation of patients survival curves (FIGS. 36D and F). The protein product of SLC39A6 (LIV-1) was reported to be regulated by estrogen [147; 148], although its control by progestin is only implied.

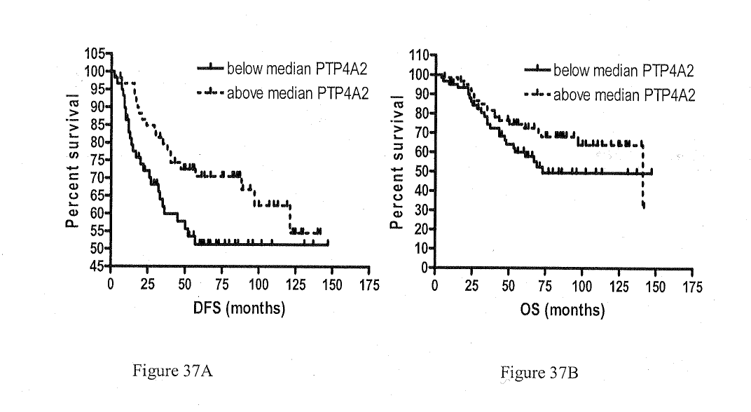

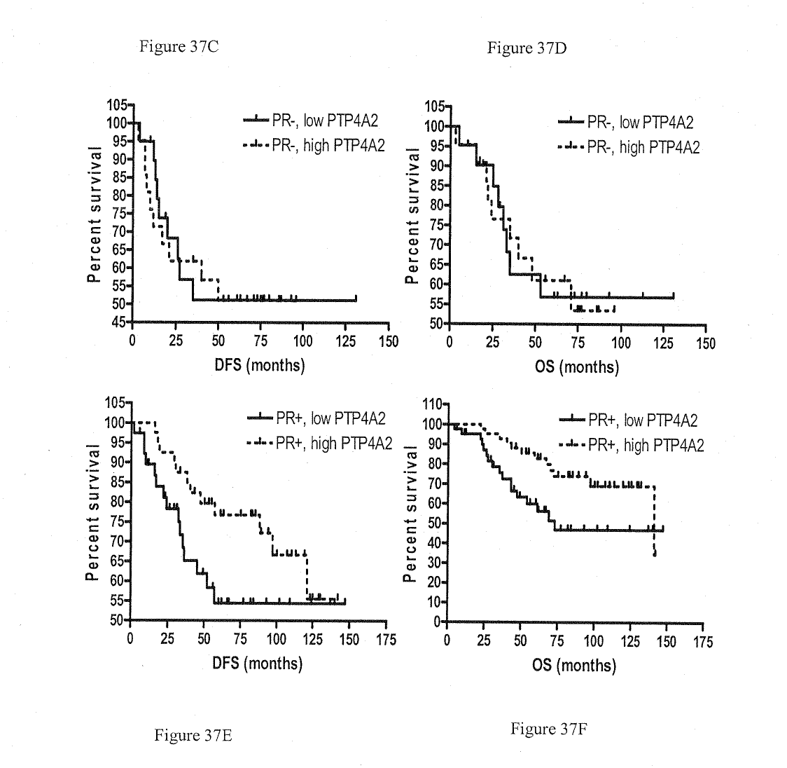

Expression of PTP4A2 was also analyzed based on a patient's PR status (FIG. 37). Expression levels of PTP4A2 did not distinguish between good and poor prognosis groups of patients with PR negative cancers (FIGS. 37C and D). However, significant separation of survival curves was observed in the PR positive patients, suggesting that the product of this gene and gene networks regulated by PR are related to tumor progression. PTP4A2 (or PRL-2) is a protein tyrosine phosphatase that is typically associated with the plasma membrane and the early endosome [186]. Over-expression of PTP4A2 has been found to transform mouse fibroblasts and pancreatic epithelial cell and promote tumor growth in nude mice [188].

Survival Analyses of Genes Determined to have Bimodal Distributions

Since results presented in FIG. 16 indicated bimodal distribution in the expression levels of ESR1, GABRP, IL6ST, XBP1, PTP4A2, LRBA, and GATA3, these seven genes were investigated by Kaplan-Meier analyses. Patients were stratified based on cut-off values of gene expression in the cancer that separated the bimodal groups of each gene, e.g., 1.0 for ESR1, 6.0 for GABRP, −4.0 for IL6ST, 1.0 for XBP1, 0.8 for PTP4A2, −1.0 for LRBA, and −0.5 for GATA3 (Table 35). Grouping the data according to bimodal distribution did not improve the Kaplan-Meier analyses of disease-free survival for these genes, and, in fact, the curve separation for PTP4A2 was less statistically significant than using the median expression value (DFS: 0.19 compared to 0.06). Although stratification of patients in the bimodal groups only moderately improved the separation of overall survival curves (OS: P value of 0.10 compared to 0.23), the survival curves for patients using expression of the other six genes were less significant than when median expression values were used for stratification. Thus the observation of bimodal expression of these genes, did not appear to have clinical relevance.

Analyses of Continuous Survival Data with Univariate Cox Proportional Hazards Model

Cox proportional hazards models using SPSS® software were performed because this modeling approach allows use of continuous gene expression variables, without the requirement of group separation (e.g., above median, below median) for analysis [236-239; 249]. A simple proportional hazards model utilizes the following equation:

h[t(x)]=hₒ(t)exp [βx]

in which “h[t(x)]” is the hazard rate for an individual with co-variate (i.e., gene expression level) “x,” “hₒ(t)” is the baseline hazard rate, and “exp(β)” is the hazard ratio [249]. P values are then calculated to determine if the observed hazard ratio is not due to chance.

TABLE 35
Summary of Kaplan-Meier results of disease-free and overall survival of
breast cancer patients according to gene expression levels exhibiting
bimodal distributions (FIG. 16).

| | STRATIFIED BY | | | |
|---|---|---|---|---|
| | MEDIAN | | STRATIFIED BY BIMODAL | |
| | EXPRESSION LEVEL | | DISTRIBUTION | |
| GENE ID | DFS (P value) | OS (P value) | DFS (P value) | OS (P value) |
| ESR1 | 0.11 | 0.36 | 0.27 | 0.28 |
| GABRP | 0.23 | 0.06 | 0.45 | 0.77 |
| IL6ST | 0.47 | 0.57 | 0.82 | 0.96 |
| XBP1 | 0.17 | 0.28 | 0.20 | 0.55 |
| PTP4A2 | 0.06 | 0.12 | 0.19 | 0.14 |
| LRBA | 0.28 | 0.42 | 0.61 | 0.90 |
| GATA3 | 0.15 | 0.23 | 0.16 | 0.10 |

Patients were stratified based on cut-off values separating the bimodal groups of each gene expressed in the tissue biopsies.
Cut-off values used for these analyses were: 1.0 for ESR1, 6.0 for GABRP, −4.0 for IL6ST, 1.0 for XBP1, 0.8 for PTP4A2, −1.0 for LRBA, and −0.5 for GATA3.

When investigating the 32 genes as single variables, this method yielded 5 genes (TBC1D9, RABEP1, SLC39A6, FUT8, and PTP4A2) with P values less than 0.05 when analyzed for disease-free survival (Table 36). Over-expression of each of these genes was correlated with a decreased likelihood of breast cancer recurrence (HR=0.90, 0.80, 0.85, 0.78, and 0.81, respectively). Expression of RABEP1, SLC39A6, FUT8, and PTP4A2 appears to be related to overall survival using this univariate analysis (P value less than 0.05, Table 37). Over-expression of RABEP1, SLC39A6, FUT8, and PTP4A2 were correlated with a decreased likelihood of death from breast cancer (HR=0.81, 0.87, 0.82, and 0.81, respectively). Thus over-expression of these genes individually forms the basis of a molecular signature predicting decreased risk of recurrence and death due to breast cancer. The ultimate goal of these collective studies is to develop clinically relevant, commercially available tests that may be used in hospital laboratories to aid in breast cancer management.

Analyses of Survival Data with Multivariate Cox Proportional Hazards Model

In order to elucidate a clinically relevant multi-gene signature from the gene expression data obtained, SPSS® 17.0 software was utilized. By importing relative gene expression data, the software performs a multivariate Cox proportional hazards model for particular time to event variable (i.e., time until breast cancer recurrence or time until death due to breast cancer). The proportional hazards model utilizes the following equation:

h[t(x)]=hₒ(t)exp [β₁x₁+β₂x₂+ . . . +βₙxₙ]

in which “h[t(x)]” is the hazard rate for an individual with co-variates (in this case, gene expression level) “x,” “hₒ(t)” is the baseline hazard rate, and “exp(β)” is the hazard ratio [249]. P values are then calculated to determine if the observed hazard ratio is not due to chance. This algorithm can then be used to predict that particular characteristic in additional samples based on their relative gene expression data.

TABLE 36
Relationship of gene expression as a function of disease-free
survival using univariate Cox regression.

| GENE ID | P VALUE | HAZARD RATIO |
|---|---|---|
| EVL | 0.383 | 0.93 |
| NAT1 | 0.063 | 0.91 |
| ESR1 | 0.068 | 0.94 |
| GABRP | 0.206 | 0.95 |
| ST8SIA1 | 0.921 | 0.99 |
| TBC1D9 | 0.015 | 0.90 |
| TRIM29 | 0.316 | 0.95 |
| SCUBE2 | 0.302 | 0.95 |
| IL6ST | 0.094 | 0.92 |
| RABEP1 | 0.009 | 0.80 |
| SLC39A6 | 0.002 | 0.85 |
| TPBG | 0.985 | 1.00 |
| TCEAL1 | 0.285 | 0.91 |
| DSC2 | 0.121 | 1.12 |
| FUT8 | 0.003 | 0.78 |
| CENPA | 0.425 | 0.91 |
| MELK | 0.325 | 1.11 |
| PFKP | 0.751 | 1.03 |
| PLK1 | 0.332 | 1.13 |
| ATAD2 | 0.120 | 1.27 |
| XBP1 | 0.124 | 0.89 |
| MCM6 | 0.789 | 0.98 |
| BUB1 | 0.423 | 0.92 |
| PTP4A2 | 0.039 | 0.81 |
| YBX1 | 0.504 | 1.09 |
| LRBA | 0.950 | 1.00 |
| GATA3 | 0.118 | 0.92 |
| CX3CL1 | 0.145 | 1.13 |
| MAPRE2 | 0.711 | 0.96 |
| GMPS | 0.429 | 0.91 |
| CKS2 | 0.890 | 1.01 |
| SLC43A3 | 0.409 | 1.10 |

P values represent the level of significance of expression for each gene, as a continuous variable.
Expression of TBC1D9, RABEP1, SLC39A6, FUT8, and PTP4A2 appear to be related to disease-free survival using univariate analysis.
Over-expression of each of these genes was correlated with a decreased likelihood of recurrence (HR = 0.90, 0.80, 0.85, 0.78, and 0.81, respectively).

TABLE 37
Relationship of gene expression as a function of overall survival
using univariate Cox regression.

| GENE ID | P VALUE | HAZARD RATIO |
|---|---|---|
| EVL | 0.208 | 0.90 |
| NAT1 | 0.152 | 0.93 |
| ESR1 | 0.090 | 0.94 |
| GABRP | 0.378 | 0.96 |
| ST8SIA1 | 0.844 | 1.02 |
| TBC1D9 | 0.050 | 0.92 |
| TRIM29 | 0.388 | 0.95 |
| SCUBE2 | 0.384 | 0.96 |
| IL6ST | 0.124 | 0.93 |
| RABEP1 | 0.012 | 0.81 |
| SLC39A6 | 0.011 | 0.87 |
| TPBG | 0.719 | 1.04 |
| TCEAL1 | 0.336 | 0.91 |
| DSC2 | 0.131 | 1.12 |
| FUT8 | 0.020 | 0.82 |
| CENPA | 0.590 | 0.94 |
| MELK | 0.235 | 1.13 |
| PFKP | 0.296 | 1.11 |
| PLK1 | 0.170 | 1.19 |
| ATAD2 | 0.665 | 1.07 |
| XBP1 | 0.223 | 0.91 |
| MCM6 | 0.945 | 0.99 |
| BUB1 | 0.561 | 0.94 |
| PTP4A2 | 0.029 | 0.81 |
| YBX1 | 0.380 | 1.12 |
| LRBA | 0.954 | 1.00 |
| GATA3 | 0.233 | 0.94 |
| CX3CL1 | 0.064 | 1.16 |
| MAPRE2 | 0.906 | 1.01 |
| GMPS | 0.823 | 0.97 |
| CKS2 | 0.880 | 1.01 |
| SLC43A3 | 0.226 | 1.15 |

P values represent the level of significance of expression for each gene, as a continuous variable.
Expression of RABEP1, SLC39A6, FUT8, and PTP4A2 appear to be related to overall survival using univariate analysis.
Over-expression of RABEP1, SLC39A6, FUT8, and PTP4A2 was correlated with a decreased likelihood of death from breast cancer (HR = 0.81, 0.87, 0.82, and 0.81, respectively).

SSPS® uses two basic modes of model selection for proportional hazards: forward stepwise selection and backwards stepwise selection. The purpose for both methods of model selection is similar, in that unimportant covariates (i.e., genes) are discarded and ones with a meaningful effect remain in the equation. The forward selection algorithm initially fits all possible linear models of the response with each individual covariate [249]. It selects the covariate with the lowest P value and includes it in the subsequent steps. In the second step it fits all possible models with the covariate from the first step plus one of each of the remaining covariates. It selects the new covariate that has the lowest P value and includes it in the subsequent steps. This is repeated until none of the remaining covariates has a P values less than 0.05. The backwards stepwise selection algorithm begins with all the variables and eliminates the covariate with the least significance in each step [249]. The data are then refitted with the remaining variables, and the process is repeated until all remaining covariates in the 1.0 equation have a P value below 0.1.

In order for unbiased internal validation of models, a Training Set population was used for model development, and a separate Test Set (patients not used for model development) was utilized for validation [242]. Using the log₂ expression data from each of the 32 genes analyzed in intact tissue sections, the patient specimens were randomly placed into Training and Test Sets at a ratio of approximately 67% (80 patients) to 33% (41 patients), respectively. Using the Training Set data to predict disease recurrence, both forward stepwise selection (data not shown) and backwards stepwise selection (Table 38) were performed. Values of β represent the log relative risk, and P values represent the level of significance of expression for each gene, as a continuous variable. Expression levels of ESR1, GABRP, ST8SIA1, TBC1D9, SCUBE2, RABEP1, SLC39A6, TPBG, TCEAL1, BUB1, PTP4A2, LRBA, CX3CL1, MAPRE2, GMPS, CKS2, and SLC43A3 were utilized in this model of disease-free survival. Using the proportional hazards model, the following equation was developed for disease-free survival:

h[t(x)]=hₒ(t)EXP((0.255*x_{ESR1})+(−0.483*x_{GABRP})+(0.792*x_{ST8SIA1})+(−0.34*x_{TBC1D9})+(0.494*x_{SCUBE2})+(−0.745*x_{RABEP1})+(−0.376*x_{SLC39A6})+(−0.476*x_{TPBG})+(0.378*x_{TCEAL1})+(0.528*x_{BUB1})+(−0.716*x_{PTP4A2})+(0.587*x_{LRBA})+(0.387*x_{CX3CL1})+(−0.365*x_{MAPRE2})+(−0.598*x_{GMPS})+(0.823*x_{CKS2})+(0.487*x_{SLC43A3})).

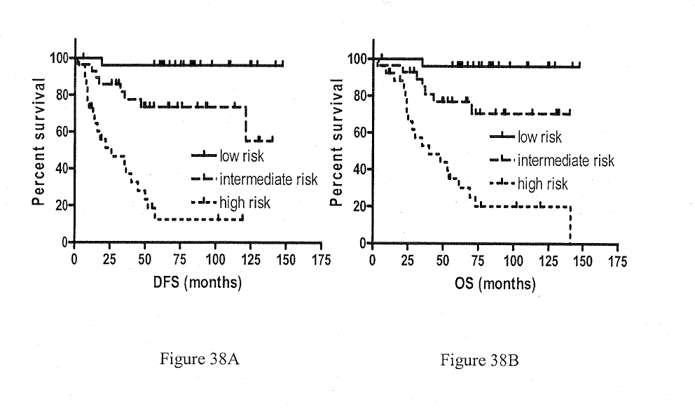

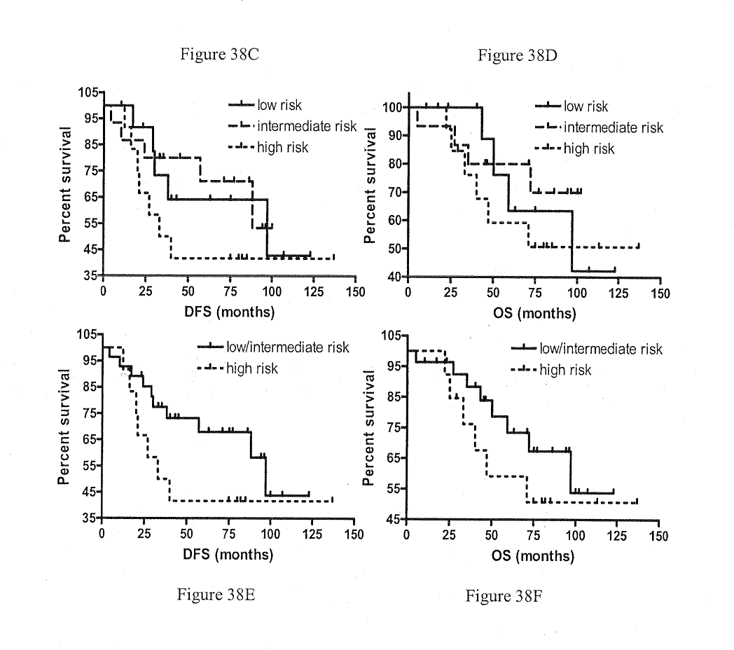

Hazard rates were calculated for each patient specimen in the Training Set, and patients were stratified by thirds into low, intermediate, and high risk populations (as suggested by Paik et al. [76] and Sparano and Paik [93]) and analyzed by Kaplan-Meier plots for disease-free and overall survival which gave P values less than 0.001 for each relationship (FIGS. 38A and B).

Hazard rates were calculated for each patient specimen in the Test Set, and patients were stratified by thirds into low, intermediate, and high risk populations and analyzed by Kaplan-Meier plots for disease-free and overall survival that gave P values of 0.369 and 0.617, respectively (FIGS. 38C and D). Since the low risk and intermediate risk groups in this population appear similar on these plots, they were grouped and re-evaluated (FIGS. 38E and F). Although the low/intermediate risk and the high risk groups separated on the Kaplan-Meier plots, they did not reach statistical significance (P values=0.16 and 0.36, respectively), which was most likely due to the small size in the Test Set population.

TABLE 38
Results from the multivariate Cox regression using backwards stepwise
selection to predict disease-free survival for the training set population.

| GENE ID | β | P VALUE | HAZARD RATIO |
|---|---|---|---|
| ESR1 | 0.255 | 0.06 | 1.29 |
| GABRP | −0.483 | 0.00 | 0.62 |
| ST8SIA1 | 0.792 | 0.00 | 2.21 |
| TBC1D9 | −0.34 | 0.01 | 0.71 |
| SCUBE2 | 0.494 | 0.00 | 1.64 |
| RABEP1 | −0.745 | 0.02 | 0.48 |
| SLC39A6 | −0.376 | 0.02 | 0.69 |
| TPBG | −0.476 | 0.07 | 0.62 |
| TCEAL1 | 0.378 | 0.10 | 1.46 |
| BUB1 | 0.582 | 0.04 | 1.79 |
| PTP4A2 | −0.716 | 0.02 | 0.49 |
| LRBA | 0.587 | 0.00 | 1.80 |
| CX3CL1 | 0.387 | 0.04 | 1.47 |
| MAPRE2 | −0.365 | 0.11 | 0.69 |
| GMPS | −0.598 | 0.04 | 0.55 |
| CKS2 | 0.823 | 0.00 | 2.28 |
| SLC43A3 | 0.487 | 0.06 | 1.63 |

Multivariate Cox models were designed to predict disease-free survival in an 80 patient training set population using backwards stepwise selection. Values of β represent the log relative risk, and P values represent the level of significance of expression for each gene, as a continuous variable. Expression levels of ESR1, GABRP, ST8SIA1, TBC1D9, SCUBE2, RABEP1, SLC39A6, TPBG, TCEAL1, BUB1, PTP4A2, LRBA, CX3CL1, MAPRE2, GMPS, CKS2, and SLC43A3 were utilized in this model of disease-free survival.

Using the Training Set (83 patients) data to predict overall survival, both forward stepwise selection (data not shown) and backwards stepwise selection (Table 39) were performed. Values of β represent the log relative risk, and P values represent the level of significance of expression for each gene, as a continuous variable. Expression levels of TRIM29, SCUBE2, SLC39A6, PTP4A2, LRBA, CX3CL1, and CKS2 were utilized in this model of overall survival. Using the proportional hazards model, the following equation was developed for disease-free survival:

h[t(x)]=hₒ(t)EXP((−0.224*x_{TRIM29})+(0.205*x_{SCUBE2})+(−0.353*x_{SLC39A6})+(−0.557*x_{PTP4A2})+(0.312*x_{LRBA})+(0.378*x_{CX3CL1})+(0.437*x_{CKS2})).

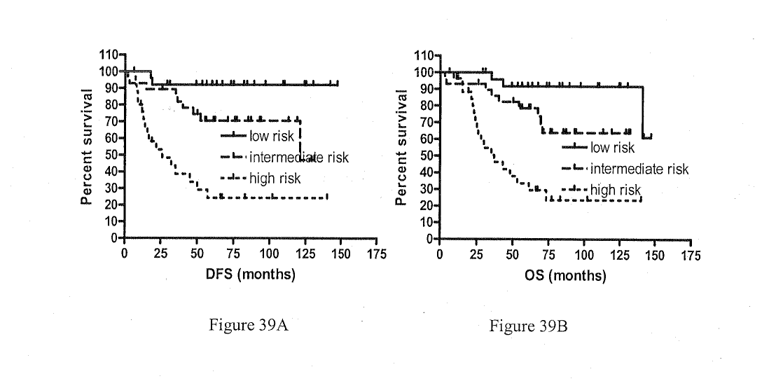

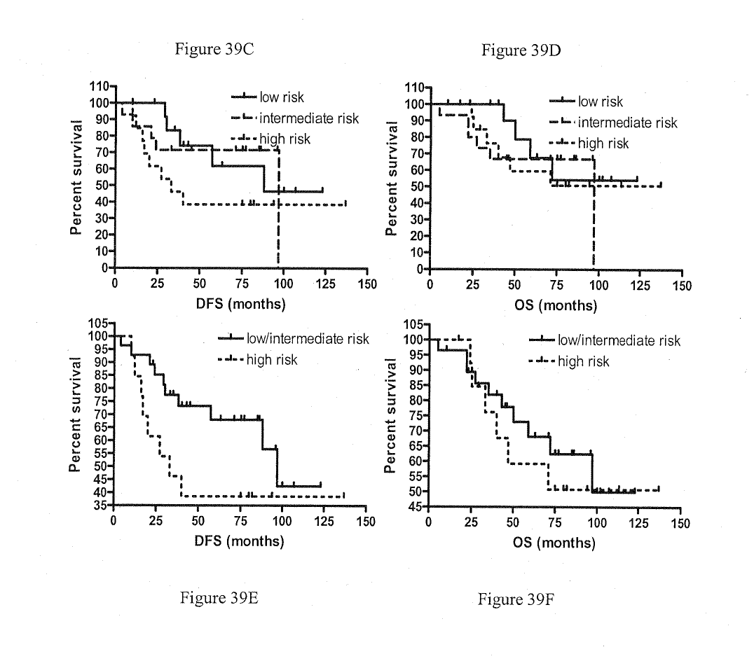

Hazard rates were calculated for each patient specimen in the Training Set, and patients were stratified by thirds into low, intermediate, and high risk populations and analyzed by Kaplan-Meier plots for disease-free and overall survival which gave P values less than 0.001 (FIGS. 39A and 39B).

Hazard rates were calculated for each patient specimen in the Test Set, and patients were stratified by thirds into low, intermediate, and high risk populations and analyzed by Kaplan-Meier plots for disease-free and overall survival giving P values of 0.252 and 0.717, respectively (FIGS. 39C and 39D). Since the low risk and intermediate risk groups in this population appear similar on these plots, they were grouped and re-evaluated (FIGS. 39E and 39F). Although the low/intermediate risk and the high risk groups separated on the Kaplan-Meier plots (DES P value=0.10, OS P value=0.62), they did not reach statistical significance, which was likely due to the small size in the Test Set population. Although internal validation using Training and Test Sets is essential for model development, it is not a replacement for actual external validation [242].

TABLE 39
Results from the multivariate Cox regression as a function of overall
survival for the training set population.

| GENE ID | β | P VALUE | HAZARD RATIO |
|---|---|---|---|
| TRIM29 | −0.224 | 0.01 | 0.80 |
| SCUBE2 | 0.205 | 0.01 | 1.23 |
| SLC39A6 | −0.353 | 0.01 | 0.70 |
| PTP4A2 | −0.557 | 0.01 | 0.57 |
| LRBA | 0.312 | 0.00 | 1.37 |
| CX3CL1 | 0.378 | 0.01 | 1.46 |
| CKS2 | 0.437 | 0.01 | 1.55 |

Multivariate Cox models were designed to predict overall survival in an 83 patient training set population using backwards stepwise selection. Values of β represent the log relative risk, and P values represent the level of significance of expression for each gene, as a continuous variable. Expression levels of TRIM29, SCUBE2, SLC39A6, PTP4A2, LRBA, CX3CL1, and CKS2 were utilized in this model of overall survival.

Multivariate Models Developed from the Entire Population

In order to improve accuracy of the multivariate models predicting recurrence and survival, expression levels from the entire population (121 patients) were used (Table 40). Of the 32 genes, expression levels of ESR1, GABRP, RABEP1, SLC39A6, TCEAL1, ATAD2, PTP4A2, LRBA, and SLC43A3 were utilized in this model of disease-free survival using backwards stepwise selection. Interestingly, these genes, with the exception of ATAD2, were also in the model developed from the Training Set population.

The following equation was developed for disease-free survival of the entire patient population:

h[t(x)]=hₒ(t)EXP((0.147*x_{ESR1})+(−0.119*x_{GABRP})+(−0.537*x_{RABEP1})+(−0.373*x_{SLC49A6})+(0.462*x_{TCEAL1})+(0.445*x_{ATAD2})+(−0.437*x_{PTP4A2})+(0.296*x_{LRBA})+(0.429*x_{SLC43A3})).

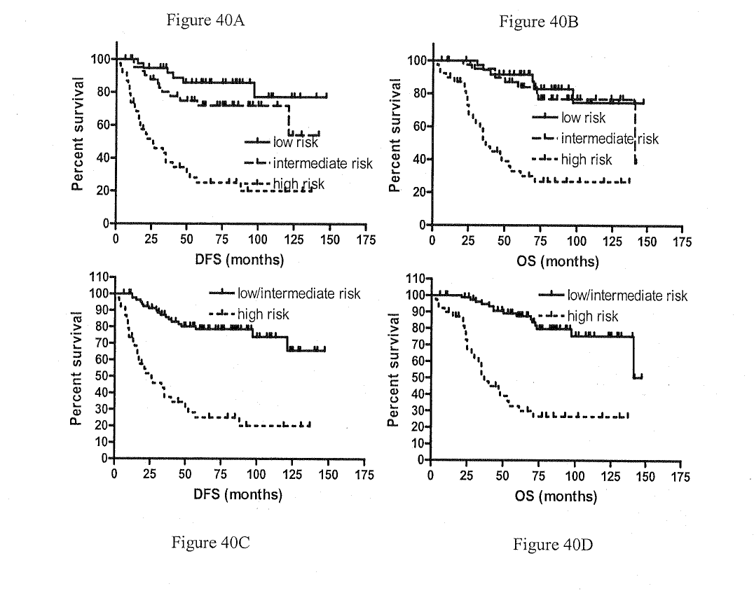

Hazard rates were calculated for each specimen, and patients were stratified by thirds into low, intermediate, and high risk populations and analyzed by Kaplan-Meier plots for disease-free and overall survival which gave P values less than 0.001 (FIGS. 40A and 40B). Since the previous analyses (FIGS. 38 and 39) indicated similar survival curves for low and intermediate risk, these groups were combined and additional Kaplan-Meier plots were performed (FIGS. 40C and D). The difference between the low/intermediate risk group and the high risk group was highly significant (P value less than 0.001), and there was a 5.5-fold greater probability of disease recurrence in the high risk group compared to the low/intermediate risk group. When analyzing this model for overall survival, the two groups were separated (P value less that 0.001), and there was a 6.1-fold greater probability of death from breast cancer in the high risk group compared to the low/intermediate risk group.

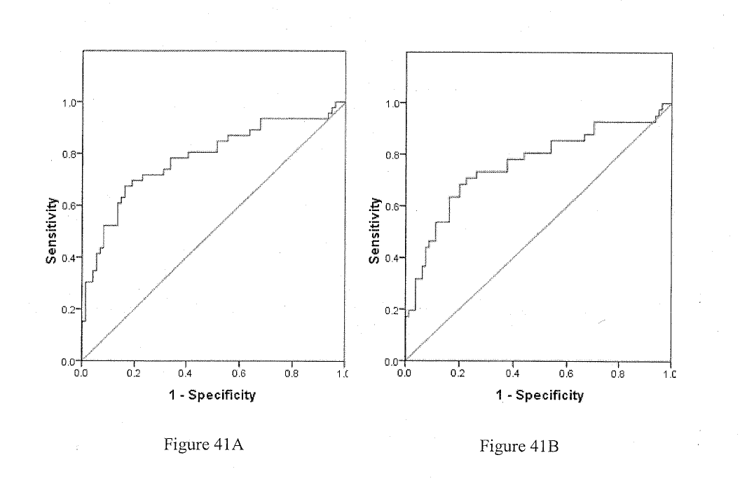

Receiver operating characteristic (ROC) curves (FIG. 41) were composed to illustrate the sensitivity (defined as [number of true-positive test results]/[number of true-positive results+number of false-negative results]) and specificity (1−specificity is defined as [number of false-positive test results]/[number of true-negative results+number of false-positive results]) of the model of disease recurrence developed using the entire patient population [255]. FIG. 41A represents the relative risk as calculated from the model compared to actual disease recurrence (DFS). In an effort to quantify the data shown in the ROC curve [242; 255; 256], the area under the curve (AUC) was determined to be 0.78. FIG. 41B represents the relative risk as calculated from the model compared to actual patient survival (OS), with the AUC determined to be 0.76. The AUC determined from ROC curves may be utilized to compare performance of different predictor models [242; 255; 256].

TABLE 40
Results from the multivariate Cox regression as a function of disease-
free survival for the entire population.

| GENE ID | β | P VALUE | HAZARD RATIO |
|---|---|---|---|
| ESR1 | 0.147 | 0.03 | 1.16 |
| GABRP | −0.119 | 0.02 | 0.89 |
| RABEP1 | −0.537 | 0.00 | 0.58 |
| SLC39A6 | −0.373 | 0.00 | 0.69 |
| TCEAL1 | 0.462 | 0.00 | 1.59 |
| ATAD2 | 0.445 | 0.01 | 1.56 |
| PTP4A2 | −0.437 | 0.01 | 0.65 |
| LRBA | 0.296 | 0.00 | 1.35 |
| SLC43A3 | 0.429 | 0.00 | 1.54 |

Multivariate Cox models were designed to predict disease-free survival in the entire 121 patient cohort using backwards stepwise selection. Values of f3 represent the log relative risk, and P values represent the level of significance of expression for each gene, as a continuous variable. Expression levels of ESR1, GABRP, RABEP1, SLC39A6, TCEAL1, ATAD2, PTP4A2, LRBA, and SLC43A3 were utilized in this model of disease-free survival.

A multivariate Cox model was designed to predict overall survival in the entire 126 patient cohort using backwards stepwise selection (Table 41). Of the 32 genes, expression levels of GABRP, TRIM29, RABEP1, SLC39A6, TCEAL1, PLK1, and CX3CL1 were utilized in this model of overall survival. The following equation was developed for overall survival of the entire patient population:

h[t(x)]=hₒ(t)EXP((−0.121*x_{GABR}1))+(−0.112*x_{TRIM29})+(−0.445*x_{RABEP1})+(−0.173*x_{SLC39A6})+(0.436*x_{TCEAL1})+(0.501*x_{PLK1})+(0.26*x_{CX3CL1})).

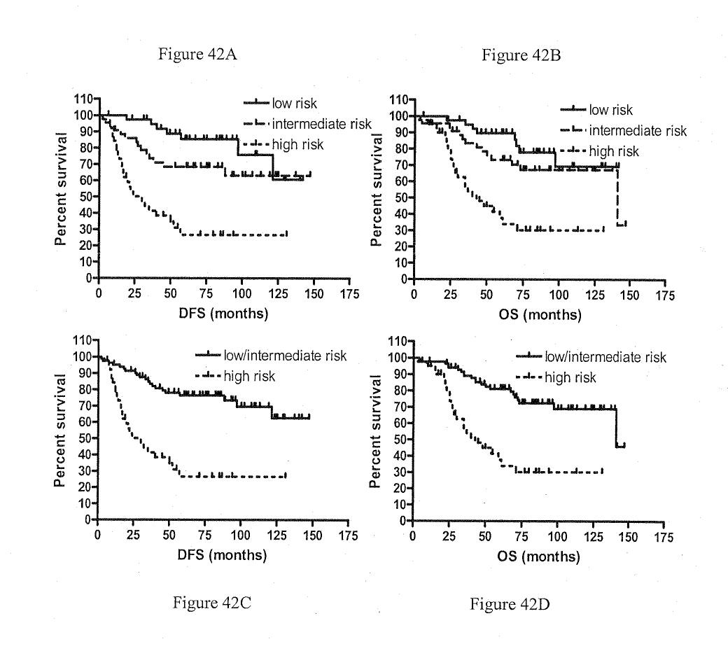

Hazard rates were calculated for each specimen, and patients were stratified by thirds into low, intermediate, and high risk populations and analyzed by Kaplan-Meier plots for disease-free and overall survival giving P values less than 0.001 (FIGS. 42A and B). Since the previous analyses (FIGS. 38 and 39) indicated similar survival curves for low and intermediate risk, these groups were combined and additional Kaplan-Meier plots were performed (FIGS. 42C and D). The difference between the low/intermediate risk group and the high risk group was highly significant (P value less than 0.001). There was a 4.2-fold greater probability of disease recurrence in the high risk group compared to the low/intermediate risk group, and there was a 3.8-fold greater probability of death due to breast cancer in the high risk group compared to the low/intermediate risk group.

TABLE 41
Results from the multivariate Cox regression as a function of overall
survival for the entire study population.

| GENE ID | β | P VALUE | HAZARD RATIO |
|---|---|---|---|
| GABRP | −0.121 | 0.01 | 0.89 |
| TRIM29 | −0.112 | 0.08 | 0.89 |
| RABEP1 | −0.445 | 0.01 | 0.64 |
| SLC39A6 | −0.173 | 0.06 | 0.84 |
| TCEAL1 | 0.436 | 0.00 | 1.55 |
| PLK1 | 0.501 | 0.00 | 1.65 |
| CX3CL1 | 0.260 | 0.01 | 1.30 |

Multivariate Cox models were designed to predict overall survival in the entire 126 patient cohort using backwards stepwise selection.
Values of β represent the log relative risk, and P values represent the level of significance of expression for each gene, as a continuous variable.
Expression levels of GABRP, TRIM29, RABEP1, SLC39A6, TCEAL1, PLK1, and CX3CL1 were utilized in this model of overall survival.

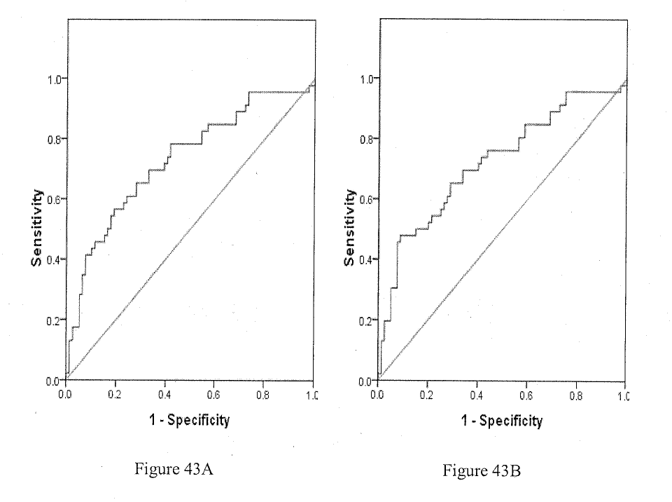

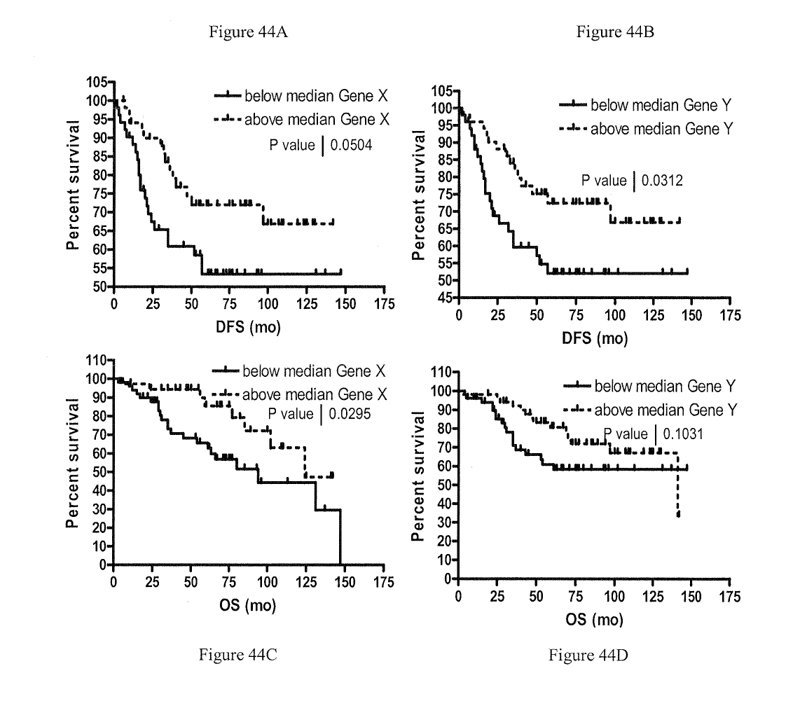

ROC curves (FIG. 43) were composed to illustrate the sensitivity and specificity of the model of overall survival developed using the entire patient population. FIG. 43A represents the relative risk as calculated from the model compared to actual disease recurrence (DFS). In an effort to quantify the data shown in the ROC curve, the AUC was determined to be 0.73. FIG. 43B represents the relative risk as calculated from the model compared to actual patient survival (OS), with the AUC determined to be 0.72. Since the area under the ROC curves (FIG. 41 and FIG. 43) is greater for the model designed to predict DFS, this would indicate that the 9 gene breast cancer recurrence model more accurately predicts both DFS and OS better than the 7 gene model designed to predict overall survival.

Additional patient characteristics (e.g., menopausal status, race, family history, tumor grade, stage of disease, lymph node status, ER/PR status) were converted to numerical values and utilized in multivariate Cox proportional hazards model [237]. This manipulation allowed the Cox proportional hazards model to incorporate all available information, both standard prognostic factors and gene expression combined, to most accurately predict a patient's clinical outcome. However, the backwards stepwise selection eliminated the requirement for including any of the above mentioned characteristics prior to the final model, indicating that these features of the patient and their breast cancer were unnecessary for predicting recurrence and survival when the 9 gene signature was employed. Thus, the 9 gene signature, derived from a broad spectrum of invasive ductal carcinomas, predicted risk of recurrence as an independent prognostic test.

After qPCR validation of the 32 gene set and their examination in LCM-procured carcinoma and stromal cells, as well as intact tissue, a total of 126 breast carcinoma specimens were evaluated for each gene by qPCR. To ensure that the sample population was representative of breast carcinoma in general, patient survival was examined as a function of known prognostic factors. The survival outcomes determined gave expected results, with the exception of nodal involvement, which was less significant than expected. This appears to be due to the selection of patients necessary for completion of the project described in Appendix I, which included equal numbers of patients with and without disease recurrence in lymph node negative and positive cancers.

Distribution of individual gene expression levels in the 126 breast cancers was examined. Those of thirteen genes (NAT1, ESR1, GABRP, IL6ST, CENPA, ATAD2, XBP1, MCM6, PTP4A2, LRBA, GATA3, GMPS, and SLC43A3) were indicative of non-Gaussian populations, which were investigated for bimodal distributions of expression. Seven of these genes appeared to have bimodal distribution, but the bimodality was insignificant in survival analyses.

Expression levels of several genes appeared to be highly correlated with other genes in the 32 gene seta Seven genes (NAT1, ESR1, SCUBE2, FUT8, PTP4A2, LRBA, and MAPRE2) had expression levels related to more than 20 of the other genes within the 32 gene set. In addition, expression levels of estrogen and progestin receptor mRNA were highly correlated with ER and PR protein levels of these known tumor markers using Pearson correlations and linear regressions.

Genes were analyzed association with known clinical characteristics, including race, menopausal status, family history, nodal status, ER, and PR status, prior to correlation of expression levels with clinical outcome (i.e., disease-free and overall survival). Genes were stratified by median expression level and subjected to Kaplan-Meier survival analyses. SCUBE2 exhibited a median expression level that significantly stratified patients into good and poor prognosis groups for DFS, while six additional genes (GABRP, TBC1D9, SLC39A6, MELK, MCM6, and PTP4A2) appeared to associate with DFS or OS (P value less than 0.10). Genes determined to be differentially expressed for a particular patient or cancer characteristic were evaluated in specific populations. Several genes (GABRP for nodal status; NAT1, CENPA, and BUB1 for tumor grade; ESR1, SCUBE2, RABEP1, SLC39A6, TCEAL1, and XBP1 for ER status; SLC39A6 and PTP4A2 for PR status) appear to distinguish between good and poor prognosis groups in specific patient populations better than the entire population.

Expression of 5 genes (TBC1D9, RABEP1, SLC39A6, FUT8, and PTP4A2) correlated independently with disease-free survival using univariate Cox Regression analyses (P less than 0.05). Expression of 4 genes (RABEP1, SLC39A6, FUT8, and PTP4A2) appeared to be related to overall survival using univariate analysis (P less than 0.05). Surprisingly, expression profiles of individual genes had predictive value although the level of confidence does not warrant their use in a single gene test.

Multivariate Cox proportional hazards models of DFS and OS were initially performed in a Training Set patient population and tested in a separate Test Set population using backwards stepwise selection. The DFS multivariate model predicted survival in the Test Set population (P values=0.16 for DFS and 0.36 for OS), and the OS model predicted survival in the Test Set population (P value=0.10 for DFS and 0.62 for OS).

Multivariate Cox proportional hazards models were performed with backwards stepwise selection in the entire population to predict disease-free survival using expression levels of 9 genes (ESR1, GABRP, RABEP1, SLC39A6, TCEAL1, ATAD2, PTP4A2, LRBA, and SLC43A3). ROC curves were composed to illustrate the sensitivity and specificity of the model for disease-free and overall survival with areas under the curves equal to 0.78 and 0.76, respectively. Although internal validation using Training and Test Sets is essential for model development, it is not a replacement for actual external validation using an independent patient population [242].

Small, biologically significant and clinically relevant gene sets that can be developed as a commercial test for assessing risk of breast cancer recurrence are described herein. These gene sets can be evaluated on a flow-thru chip (TIPCHIP™) for use in the ZIPLEX® Automated Workstation (Xceed Molecular Corp.), which allows for analyses in a clinical laboratory avoiding the necessity for a “send-out test.” Prediction of risk of recurrence of breast cancer at the time of surgical removal of the primary lesion, will facilitate improved treatment planning and disease surveillance resulting in improved care for these patients.

### Example 4

### Gene Expression in Breast Tissue Samples

### Gene List Comparisons

Genes were selected for subsequent analyses based on occurrence in multiple signatures. Utilizing studies examining pure carcinoma cell populations procured by LCM (e.g., [41; 57; 70; 71]), 14 candidate carcinoma-associated genes were selected. Studies from intact tissue sections (e.g., [47; 48; 54; 55; 62-65; 67]) provided an additional subset of 18 candidate genes with differential expression inferred in stromal cells with clinical relevance.

### Tissue Preparation

Using an IRB-approved study, frozen sections from de-identified specimens (Tables 42 and 43) from patients diagnosed with invasive ductal or lobular carcinoma were utilized [37; 38]. H & E staining was performed as described [37; 38; 41], and procedures were conducted under RNase-free conditions.

RNA Extraction, Purification and qPCR Analysis

Total RNA was extracted from frozen tissue sections [37; 38] with the RNEASY® Mini Kit (Qiagen Inc., Valencia, Calif.). Integrity of RNA was analyzed with the Bioanalyzer 2100 (Agilent Technologies, Palo Alto, Calif.). Total RNA was reverse transcribed in 50 mM Tris-HCl buffer containing 37.5 mM KCl, 1.5 mM MgCl₂, 10 mM DTT, 0.5 mM dNTPs (Invitrogen, Carlsbad, Calif.), 20 u RNASIN® (Promega, Madison, Wis.), 200 u SUPERSCRIPT RT III® (Invitrogen) and 5 ng of T7 primers or 166 ng of random hexamers.

TABLE 42
Patient population (qPCR).

| PATIENT PARAMETERS | n |
|---|---|
| Median Age (range) | |
| 56 years (26-89.5) | 102 |
| Median Observation time (range) | |
| 61 months (3-147) | 102 |
| Race | |
| white | 95 |
| black | 7 |
| Histology | |
| Invasive ductal carcinoma | 102 |
| Median Tumor Size (Range) | |
| 28 mm (4-85) | 95 |
| Stage | |
| 1 | 17 |
| 2A | 37 |
| 2B | 31 |
| 3A | 9 |
| 3B | 5 |
| 4 | 5 |
| Grade | |
| 1 | 5 |
| 2 | 29 |
| 3 | 44 |
| 4 | 2 |
| unknown | 22 |
| Lymph Node Status | |
| negative | 48 |
| positive | 52 |
| unknown | 2 |
| Recurrence Status | |
| yes | 37 |
| no | 65 |

TABLE 43
Patient population (ZIPLEX ®).

| PATIENT PARAMETERS | n |
|---|---|
| Median Age (range) | |
| 55 years (26-89.5) | 109 |
| Median Observation time (range) | |
| 59 months (3-147) | 109 |
| Race | |
| white | 94 |
| black | 14 |
| Histology | |
| Invasive ductal carcinoma | 99 |
| Lobular carcinoma | 9 |
| Mixed IDC/lobular | 1 |
| Median Tumor Size (Range) | |
| 30 mm (9-85) | 100 |
| Stage | |
| 1 | 20 |
| 2A | 40 |
| 2B | 28 |
| 3A | 9 |
| 3B | 4 |
| 4 | 3 |
| Grade | |
| 1 | 4 |
| 2 | 28 |
| 3 | 53 |
| 4 | 1 |
| unknown | 23 |
| Lymph Node Status | |
| negative | 59 |
| positive | 49 |
| unknown | 2 |
| Recurrence Status | |
| yes | 53 |
| no | 56 |

RNA quantification and analyses were performed using triplicate cDNA preparations with qPCR in duplicate wells using the ABI PRISM® 7900HT (Applied Biosystems, Foster City, Calif.) with POWER SYBR® Green (Applied Biosystems) for detection. Universal Human Reference RNA (Stratagene, La Jolla, Calif.) was reverse transcribed and amplified along with test samples as both a positive control and as standards for quantification of RNA using β-actin as a reference gene, and relative gene expression was calculated using the ΔΔCt method.

### Sample Hybridization on the ZIPLEX® Automated Workstation

Total RNA samples were analyzed for quality with the Agilent BIOANALYZER™, amplified and biotin-labeled by oligo-dT primed in vitro transcription. TipChip microarrays, samples, and reagents were loaded into specific microplate wells, and then hybridization, washing, chemiluminescent imaging and data reduction were performed automatically on the ZIPLEX® Automated Workstation.

The ZIPLEX® manifold picks up the TipChips and lowers them into specific wells where solutions are repeatedly aspirated and dispensed through the chips. Up to eight TipChips were hybridized and analyzed simultaneously in less than three hours. Tables of mean intensities and coefficients of variation of triplicate spots for each probe were output by the instrument and analyzed on an external computer.

### Statistical Analysis

Multivariate analyses were performed using PARTEK GENOMICS SUITE™, including K-nearest neighbor, shrinking centroid, and discriminant analysis to determine the best fit model for predicting breast cancer recurrence in a training set of each sample population. The best fit models were then applied to the remaining samples (test set). Kaplan-Meier regression analyses were performed using PARTEK GENOMICS SUITE™ and GRAPHPAD PRISM™.

### Results and Discussion

Clinical Correlations of Gene Expression Results Obtained by qPCR

Kaplan-Meier survival curves (FIG. 44) of gene expression measured by qPCR were generated for each gene of the 32 gene set, and two genes (X=RABEP1; Y=SLC39A6) were determined to statistically significant. These survival plots illustrate correlations of disease-free and overall survival of breast cancer patients (Table 42) as a function two clinically relevant genes.

Cox regression survival analyses (Table 44) on expression of individual genes measured by qPCR. P values represent the level of significance of expression for each gene, as a continuous variable. Expression of 4 genes (B=FUT8, D=MCM6, L=GATA3, and C=TPBG) appear to be related to disease-free survival using univariate analysis. Over-expression of each of these genes was correlated with a decreased likelihood of recurrence (HR=0.79, 0.81, 0.89, and 0.80, respectively).

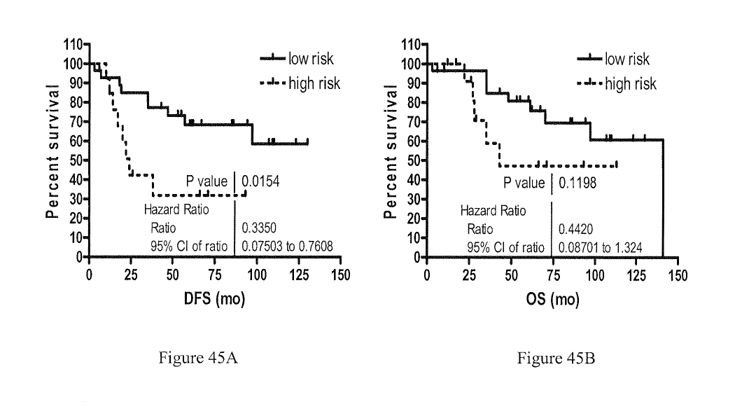

In order to predict breast cancer recurrence and survival, a multivariate model was developed using gene combinations from expression levels of the 32 gene set measured by qPCR. The multivariate model for disease-free survival (FIG. 45) was created using a K-Nearest Neighbor classification with a 61 sample training set, and applied to the 41 sample test set shown in FIG. 45. The model was able to separate patients into good or poor prognosis groups with a significance level of P=0.02 for disease-free survival. The poor prognosis group had a 3-fold greater likelihood of breast cancer recurrence than the good prognosis group.

TABLE 44
Cox regression analyses on individual genes measured by qPCR.

| GENE | | P VALUE | HAZARD RATIO |
|---|---|---|---|
| | B | 0.006 | 0.79 |
| | D | 0.008 | 0.81 |
| | L | 0.017 | 0.89 |
| | C | 0.017 | 0.80 |

P values represent the level of significance of expression for each gene, as a continuous variable. Expression of 4 genes (B=FUT8, D=MCM6, L=GATA3, and C=TPBG) appear to be related to disease-free survival using univariate analysis. Over-expression of each of these genes was correlated with a decreased likelihood of recurrence (HR=0.79, 0.81, 0.89, and 0.80, respectively).

Comparisons of Expression Results Obtained from qPCR and ZIPLEX®

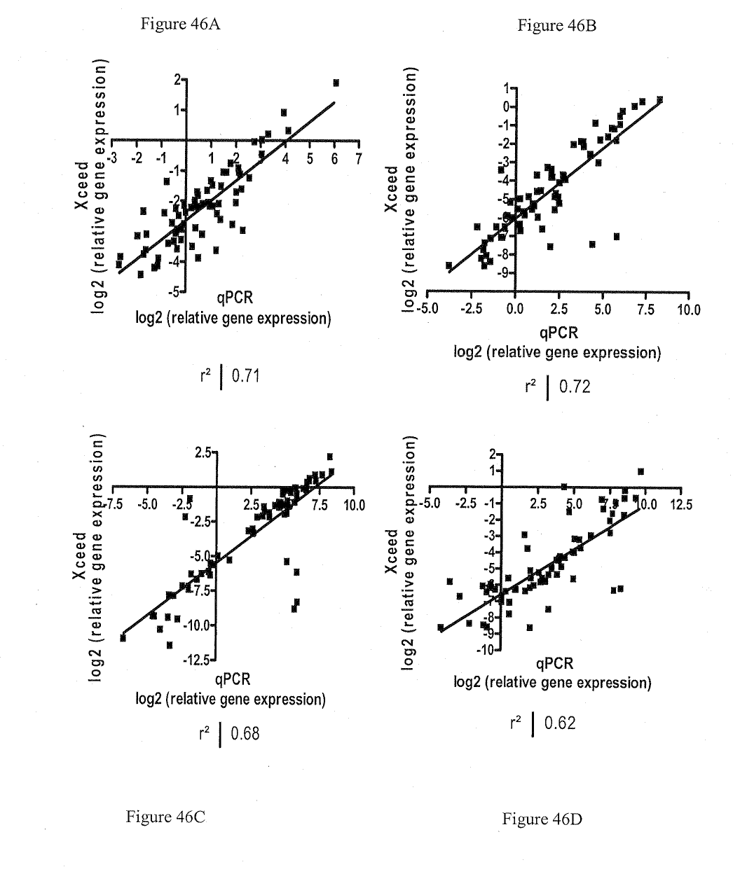

Gene expression results obtained from qPCR or the ZIPLEX® Automated Workstation were correlated. FIG. 46 illustrates four representative genes (EVL, NAT1, ESR1, and GABRP) illustrating similar gene expression results from both analysis platforms. After these results were obtained, similar clinical correlations were performed on the data obtained from the ZIPLEX® platform.

### Clinical Correlations of Gene Expression Results Obtained by ZIPLEX®

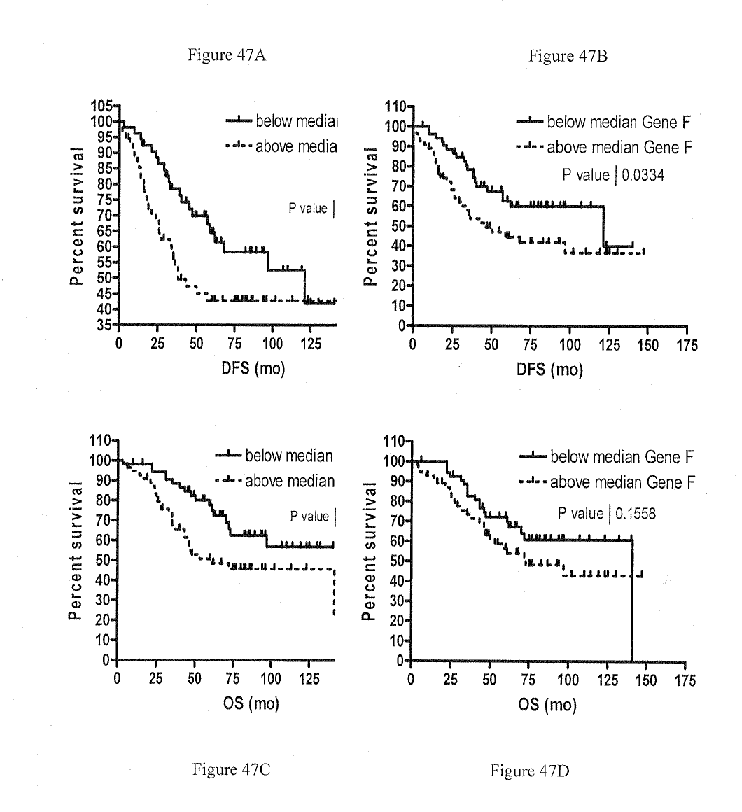

Kaplan-Meier survival curves (FIG. 47) were developed for each gene in the 32 gene set, and expression levels of two genes (S=DSC2; F=BUB1) measured by the ZIPLEX® Automated Workstation were determined to be clinically relevant. These plots illustrate correlations of disease-free and overall survival of breast cancer patients as a function of the two genes.

Cox regression analyses (Table 45) were then performed on expression levels of individual genes measured by the ZIPLEX® Automated Workstation. Expression levels detected by probes of four different genes (S=DSC2, N=PFKP probes 1 and 2, K=MELK, and AE=SLC43A3) appear to be related to disease-free survival using univariate analysis. Over-expression of each of these probes was correlated with an increased likelihood of recurrence (HR=1.27, 1.23, 1.24, 1.27, and 1.49, respectively).

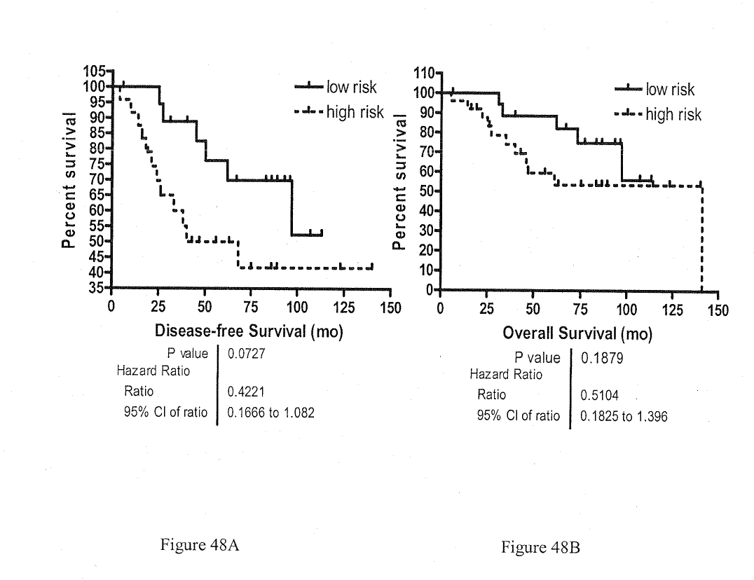

Probability of breast cancer recurrence and survival based on a model developed using gene combinations from the 32 gene set measured by the ZIPLEX® Automated Workstation (FIG. 48). The multivariate model for disease-free survival was created using K-Nearest Neighbor classification with a 65 sample training set, and applied to the 44 sample test set shown above. This model was able to separate patients into good or poor prognosis groups with a significance level of P=0.07 for disease-free survival.

The poor prognosis group had a 2.4-fold greater likelihood of breast cancer recurrence than the good prognosis group using this multivariate model based on gene expression levels determined by the ZIPLEX® platform.

TABLE 45
Cox regression analyses on individual genes measured by the ZIPLEX ®
Automated Workstation.

| GENE ID - PROBE # | P VALUE | HAZARD RATIO |
|---|---|---|
| S-2 | 0.032 | 1.27 |
| N-1 | 0.042 | 1.23 |
| K-3 | 0.043 | 1.27 |
| AE-2 | 0.043 | 1.49 |
| N-2 | 0.047 | 1.24 |

P values represent the level of significance of expression for each gene, as a continuous variable. Expression of probes from 4 different genes (S=DSC2, N=PFKP probes 1 and 2, K=MELK, and AE=SLC43A3) appear to be related to disease-free survival using univariate analysis. Over-expression of each of these probes was correlated with an increased likelihood of recurrence (HR=1.27, 1.23, 1.24, 1.27, and 1.49, respectively).

TABLE 46
Sequences of primers.

| | FORWARD PRIMERS | REVERSE PRIMERS |
|---|---|---|
| ACTB | AACTGGTCTCAAGTCAGTGTACAGG | TCCCCCAACTTGAGATGTATGAAG |
| | (SEQ ID NO: 1) | (SEQ ID NO: 2) |
| EVL | TTTCTAGAGACGCCCCTAAGTCA | CCAGCTGAGGCGCTAACAG |
| | (SEQ ID NO: 3) | (SEQ ID NO: 4) |
| NAT1 | CATTGATGGCAGGAACTACATTG | CTCCAGAGGCTGCCACATCT |
| | (SEQ ID NO: 5) | (SEQ ID NO: 6) |
| ESR1 | GCCAAATTGTGTTTGATGGATTAA | GACAAAACCGAGTCACATCAGTAATAG |
| | (SEQ ID NO: 7) | (SEQ ID NO: 8) |
| GABRP | TGGCCCTGAGTACTGAACTTTCT | ACCCGCAACCTGAACATAGG |
| | (SEQ ID NO: 9) | (SEQ ID NO: 10) |
| ST8SIA1 | AACCAGGGTATTTTTGTTAGGTTTTCT | CAAACTCATGAAACAACTTGACCAT |
| | (SEQ ID NO: 11) | (SEQ ID NO: 12) |
| TBC1D9 | TCCGGGCAGATTTGATTGA | CACGTTGCGTTTCGTAGTATCC |
| | (SEQ ID NO: 13) | (SEQ ID NO: 14) |
| TRIM29 | TCCGGCCTCTCCGACTTC | CTGAGGTCACAAGGCAGGAAAG |
| | (SEQ ID NO: 15) | (SEQ ID NO: 16) |
| SCUBE2 | GCTATAGGGTTGGTGGGACAGA | ACTGATACGGGAGGCAGCAA |
| | (SEQ ID NO: 17) | (SEQ ID NO: 18) |
| IL6ST | GTTCCGTCAGTCCAAGTCTTCTC | TCTGGCCGCTCCTCTGAA |
| | (SEQ ID NO: 19) | (SEQ ID NO: 20) |
| RABEP1 | CAGAAGATGGTGCTGGGTAATAAA | TTCCAACAGTTGGCATTTGC |
| | (SEQ ID NO: 21) | (SEQ ID NO: 22) |
| SLC39A6 | GCAGGCTGTCCTTTATAATGCA | TGAAAATTCCTGTTGCCATTCC |
| | (SEQ ID NO: 23) | (SEQ ID NO: 24) |
| TPBG | ATGGGCTTCTTGCTGTCTGTCT | TTGAATGCTATCTGTGTGGGTACA |
| | (SEQ ID NO: 25) | (SEQ ID NO: 26) |
| TCEAL1 | AAAGTTGAGGTTTCCCCCTAAAAT | TGCAAATGTGTAGGGCTCATG |
| | (SEQ ID NO: 27) | (SEQ ID NO: 28) |
| DSC2 | ATCTGCAAACCCACCATGTCA | AAAGGGTGGGCCATGGATAG |
| | (SEQ ID NO: 29) | (SEQ ID NO: 30) |
| FUT8 | CCAGAATGCCCACAATCAAA | ATCTCCAGGTTCCATGGGAAT |
| | (SEQ ID NO: 31) | (SEQ ID NO: 32) |
| CENPA | CCATTAAGTGGCAGCATCATGTAA | CCCCAATTAAGTTTCTGAAAAGCT |
| | (SEQ ID NO: 33) | (SEQ ID NO: 34) |
| MELK | AAGTGTGCCAGCTTCAAAAACC | CCCAGGCATCGCCCTTA |
| | (SEQ ID NO: 35) | (SEQ ID NO: 36) |
| PFKP | TTCATTTACCAGCTGTATTCAGAAGAG | CCACCCTGCTGCATGTGA |
| | (SEQ ID NO: 37) | (SEQ ID NO: 38) |
| PLK1 | GGATCACACCAAGCTCATCTTG | CCCGCTTCTCGTCGATGT |
| | (SEQ ID NO: 39) | (SEQ ID NO: 40) |
| ATAD2 | AAAGCCAGAGTGCAAGTCATGAT | GAATTGTGGTGCAGCCAGAA |
| | (SEQ ID NO: 41) | (SEQ ID NO: 42) |
| XBP1 | CCCCCTTTTTGGCATCCT | GCAGGTGTTCCCGTTGCTTA |
| | (SEQ ID NO: 43) | (SEQ ID NO: 44) |
| MCM6 | CGGATGCACTGCTGTGATG | TGTTTCCACACGGATGATTGA |
| | (SEQ ID NO: 45) | (SEQ ID NO: 46) |
| BUB1 | TGAGCAAGTGCATGACTGTGAA | TCATCATCCTGTTCCAAAAATCC |
| | (SEQ ID NO: 47) | (SEQ ID NO: 48) |
| PTP4A2 | CCCCCGATCCAAGTTGTAGA | GGGCTTAAGGCTGCCAGACT |
| | (SEQ ID NO: 49) | (SEQ ID NO: 50) |
| YBX1 | CCAGAAAACCCTAAACCACAAGA | GGGAGCGGACGAATTCTCA |
| | (SEQ ID NO: 51) | (SEQ ID NO: 52) |
| LRBA | GGAGGGACTCAGGCATTGG | AGATAGCACCTCGCTGATTGC |
| | (SEQ ID NO: 53) | (SEQ ID NO: 54) |
| GATA3 | AAGGATGCCAAGAAGTTTAAGGAA | ACTGGCAGTTTGTCCATTTGAA |
| | (SEQ ID NO: 55) | (SEQ ID NO: 56) |
| CX3CL1 | TTCTACCCAGGTGCTAGGAACAC | CACAGCGTCTTGCTCTCTATGG |
| | (SEQ ID NO: 57) | (SEQ ID NO: 58) |
| MAPRE2 | CATCAACGCACTGTTGCATATG | AGGGCCGTCCGCTAATACAC |
| | (SEQ ID NO: 59) | (SEQ ID NO: 60 |
| GMPS | GCCTTCTTGCTGCCAATTAAAA | CTTTACTGGAGATTCCACACACGTA |
| | (SEQ ID NO: 61) | (SEQ ID NO: 62) |
| CKS2 | CGCG CTCTCGTTTCATTTTC | TGTCCGAGTAGTAGATCTGCTTGTG |
| | (SEQ ID NO: 63) | (SEQ ID NO: 64) |
| SLC43A3 | TCAGCCCCGAGGATGGT | TGCTGGGATAGGCAAAGTCTTT |
| | (SEQ ID NO: 65) | (SEQ ID NO: 66) |

TABLE 47
Abbreviations

| | |
|---|---|
| MAQC | MicroArray Quality Contol |
| MGI | molecular grade index |
| MINDACT | Microarray In Node-negative and 1-3 positive lymph node |
| | Disease may Avoid ChemoTherapy |
| mRNA | messenger ribonucleic acid |
| NSABP | National Surgical Adjuvant Breast and Bowel Project |
| OCT | Optimum Cutting Temperature |
| OS | overall survival |
| PAGE | polyacrylamide gel electrophoresis |
| PCR | polymerase chain reaction |
| PMSF | phenylmethanesulfonylfloride |
| PR | progestin receptor |
| qPCR | quantitative polymerase chain reaction |
| RIN | RNA Integrity Number |
| RNA | ribonucleic acid |
| ROC | receiver operating characteristic |
| RQI | RNA quality indicator |
| rRNA | ribosomal ribonucleic acid |
| RS | Recurrence ScoreTM |
| RT | reverse transcriptase |
| RT-PCR | reverse transcription polymerase chain reaction |
| SAGE | serial analysis of gene expression |
| SNP | single nucleotide polymorphism |
| TAILORx | Trial Assigning IndividuaLized Options for Treatment |
| TRANSBIG | translational research of the Breast International Group |
| PAGE | polyacrylamide gel electrophoresis |
| PCR | polymerase chain reaction |
| PMSF | phenylmethanesulfonylfloride |
| PR | progestin receptor |
| qPCR | quantitative polymerase chain reaction |
| RIN | RNA Integrity Number |
| RNA | ribonucleic acid |
| ROC | receiver operating characteristic |
| RQI | RNA quality indicator |
| rRNA | ribosomal ribonucleic acid |
| RS | Recurrence Score |
| RT | reverse transcriptase |
| RT-PCR | reverse transcription polymerase chain reaction |
| SAGE | serial analysis of gene expression |
| SNP | single nucleotide polymorphism |
| SWOG | Southwest Oncology Group |
| TAILORx | Trial Assigning IndividuaLized Options for Treatment |
| TRANSBIG | translational research of the Breast International Group |

A custom designed a “flow-thru” chip (TIPCHIPip™) was created containing each of the 32 genes supra, as well as other genes identified in an independent study described in Patent Cooperation Treaty Application No: PCT/US2009/060506 (WO 2010/045234). Two independent molecular signatures were shown to be related to the clinical behavior of human breast cancer. One of these based upon the gene subset described in this dissertation predicts risk of breast cancer recurrence regardless of estrogen receptor status and nodal involvement.

### REFERENCES

1. McKusick V A. Genomics: structural and functional studies of genomes. Genomics 1997; 45(2):244-249.
2. DiMaio D, Miller G. Thirty years into the genomics era: tumor viruses led the way. Yale J Biol Med 2006; 79(3-4):179-185.
3. Mullis K B. The unusual origin of the polymerase chain reaction. Sci Am 1990; 262(4):56-5.
4. Alwine J C, Kemp D J, Stark G R. Method for detection of specific RNAs in agarose gels by transfer to diazobenzyloxymethyl-paper and hybridization with DNA probes. Proc Natl Acad Sci USA 1977; 74(12):5350-5354.
5. Ding, C., Cantor C R. Quantitative analysis of nucleic acids—the last few years of progress. J Biochem Mol Biol 2004; 37(1):1-10.
8. Kumar V, Abbas A K, Fausto N. Robbins and Cotran Pathological Basis of Disease, 7th ed. 2005. Philadelphia, Pa., Elsevier Saunders.
9. Greene F L. AJCC Staging Manual, 6th Ed. 2002. New York, N.Y., Springer Verlag.
10. Wittliff J L, Raffelsberger W. Mechanisms of signal transduction: sex hormones, their receptors and clinical utility. J Clin Ligand Assay 1995; 18:211-235.
11. Wittliff J L, Pasic R B K I. Steroid and peptide hormone receptors: methods, quality control, and clinical use. In: Bland K I, Copeland E M, editors. The Breast: Comprehensive Management of Benign and Malignant Diseases. Philadelphia: W.B. Saunders Co.; 1998. p. 458-498.
12. Shekhar M P, Werdell J, Santner S J, Pauley R J, Tait L. Breast stroma plays a dominant regulatory role in breast epithelial growth and differentiation: implications for tumor development and progression. Cancer Res 2001; 61(4):1320-1326.
13. Barlow J, Yandell D, Weaver D, Casey T, Plaut K. Higher stromal expression of transforming growth factor-beta type II receptors is associated with poorer prognosis breast tumors. Breast Cancer Res Treat 2003; 79(2):149-159.
14. Boersma B J, Reimers M, Yi M, Ludwig J A, Luke B T, Stephens R M et al. A stromal gene signature associated with inflammatory breast cancer. Int J Cancer 2008; 122(6):1324-1332.
15. Casey T, Bond J, Tighe S, Hunter T, Lintault L, Patel O et al. Molecular signatures suggest a major role for stromal cells in development of invasive breast cancer. Breast Cancer Res Treat 2009; 114(1):47-62.
16. Fiegl H, Millinger S, Goebel G, Muller-Holzner E, Marth C, Laird P W et al. Breast cancer DNA methylation profiles in cancer cells and tumor stroma: association with HER-2/neu status in primary breast cancer. Cancer Res 2006; 66(1):29-33.
17. Finak G, Bertos N, Pepin F, Sadekova S, Souleimanova M, Zhao H et al. Stromal gene expression predicts clinical outcome in breast cancer. Nat Med 2008; 14(5):518-527.
18. Fukino K, Shen L, Matsumoto S, Morrison C D, Mutter G L, Eng C. Combined total genome loss of heterozygosity scan of breast cancer stroma and epithelium reveals multiplicity of stromal targets. Cancer Res 2004; 64(20):7231-7236.
19. Hanson J A, Gillespie J W, Grover A, Tangrea M A, Chuaqui R F, Emmert-Buck M R et al. Gene promoter methylation in prostate tumor-associated stromal cells. J Natl Cancer Inst 2006; 98(4):255-261.
20. Hawsawi N M, Ghebeh H, Hendrayani S F, Tulbah A, Al-Eid M, Al-Tweigeri T et al. Breast carcinoma-associated fibroblasts and their counterparts display neoplastic-specific changes. Cancer Res 2008; 68(8):2717-2725.
21. Koukourakis M I, Giatromanolaki A, Bougioukas G, Spyridis E. Lung cancer: a comparative study of metabolism related protein expression in cancer cells and tumor associated stroma. Cancer Biol Ther 2007; 6(9):1476-1479.
22. Lewen S, Zhou H, Hu H D, Cheng T, Markowitz D, Reisfeld R A et al. A Legumain-based minigene vaccine targets the tumor stroma and suppresses breast cancer growth and angiogenesis. Cancer Immunol Immunother 2008; 57(4):507-515.
23. Mellick A S, Day C J, Weinstein S R, Griffiths L R, Morrison N A. Differential gene expression in breast cancer cell lines and stroma-tumor differences in microdissected breast cancer biopsies revealed by display array analysis. Int J Cancer 2002; 100(2):172-180.
24. Orimo A, Gupta P B, Sgroi D C, Arenzana-Seisdedos F, Delaunay T, Naeem R et al. Stromal fibroblasts present in invasive human breast carcinomas promote tumor growth and angiogenesis through elevated SDF-1/CXCL12 secretion. Cell 2005; 121(3):335-348.
25. Singer C F, Gschwantler-Kaulich D, Fink-Retter A, Haas C, Hudelist G, Czerwenka K et al. Differential gene expression profile in breast cancer-derived stromal fibroblasts. Breast Cancer Res Treat 2008; 110(2):273-281.
26. Smith R A, Lea R A, Weinstein S R, Griffiths L R. Progesterone, glucocorticoid, but not estrogen receptor mRNA is altered in breast cancer stroma. Cancer Lett 2007; 255(1):77-84.
27, Tang Y, Kesavan P, Nakada M T, Yan L. Tumor-stroma interaction: positive feedback regulation of extracellular matrix metalloproteinase inducer (EMMPRIN) expression and matrix metalloproteinase-dependent generation of soluble EMMPRIN. Mol Cancer Res 2004; 2(2):73-80.
28. Tuhkanen H, Anttila M, Kosma V M, Yla-Herttuala S, Heinonen S, Kuronen A et al. Genetic alterations in the peritumoral stromal cells of malignant and borderline epithelial ovarian tumors as indicated by allelic imbalance on chromosome 3p. Int J Cancer 2004; 109(2):247-252.
29. Santner S J, Pauley R J, Tait L, Kaseta J, Santen R J. Aromatase activity and expression in breast cancer and benign breast tissue stromal cells. J Clin Endocrinol Metab 1997; 82(1):200-208.
30. Mellick A S, Blackmore D, Weinstein S R, Griffiths L R. An assessment of MMP and TIMP gene expression in cell lines and stroma—tumour differences in microdissected breast cancer biopsies. Tumour Biol 2003; 24(5):258-270.
31. Ma X J, Dahiya S, Richardson E A, Erlander M, Sgroi D C. Gene expression profiling of tumor microenvironment during breast cancer progression. Breast Cancer Res 2009; 11(1):R7.
32. Matrisian L M, Cunha G R, Mohla S. Epithelial-stromal interactions and tumor progression: meeting summary and future directions. Cancer Res 2001; 61(9):3844-3846.
33. Burgemeister R. New aspects of laser microdissection in research and routine. J Histochem Cytochem 2005; 53(3):409-412.
34. Cole K A, Krizman D B, Emmert-Buck M R. The genetics of cancer—a 3D model. Nat Genet. 1999; 21(1 Suppl):38-41.
35. Emmert-Buck M R, Bonner R F, Smith P D, Chuaqui R F, Zhuang Z, Goldstein S R et al. Laser capture microdissection. Science 1996; 274(5289):998-1001.
36. Sluka P, O'Donnell L, McLachlan R I, Stanton P G. Application of laser-capture microdissection to analysis of gene expression in the testis. Prog Histochem Cytochem 2008; 42(4):173-201.
37. Wittliff J L, Kunitake S T, Chu S S, Travis J C. Applications of laser capture microdissection in genomics and proteomics. J. Clin. Ligand Assay 23, 66. 2000.
38. Wittliff J L, Erlander M G. Laser capture microdissection and its applications in genomics and proteomics. Methods Enzymol 2002; 356:12-25.
39. Bonner R F, Emmert-Buck M. Cole K, Pohida T, Chuaqui R, Goldstein S et al. Laser capture microdissection: molecular analysis of tissue. Science 1997; 278(5342):1481, 1483.
40. Simone N L, Bonner R F, Gillespie J W, Emmert-Buck M R, Liotta L A. Laser-capture microdissection: opening the microscopic frontier to molecular analysis. Trends Genet. 1998; 14(7):272-276.
41. Wittliff J L, Ma X J, Stecker K K, Salunga R C, Tuggle J T, Tran Y K et al. Gene expression profiles and tumor marker signatures of human breast carcinoma cells procured by laser capture microdissection. Endocrine Soc. Abs. 2002.
42. Hong S H, Nah H Y, Lee J Y, Gye M C, Kim C H, Kim M K. Analysis of estrogen-regulated genes in mouse uterus using cDNA microarray and laser capture microdissection. J Endocrinol 2004; 181(1):157-167.
43. Ellsworth D L, Shriver C D, Ellsworth R E, Deyarmin B, Somiari R I. Laser capture microdissection of paraffin-embedded tissues. Biotechniques 2003; 34(1):42-4, 46.
44. Gjerdrum L M, Lielpetere I, Rasmussen L M, Bendix K, Hamilton-Dutoit S. Laser-assisted microdissection of membrane-mounted paraffin sections for polymerase chain reaction analysis: identification of cell populations using immunohistochemistry and in situ hybridization. J Mol Diagn 2001; 3(3):105-110.
45. Gjerdrum L M, Sorensen B S, Kjeldsen E, Sorensen F B, Nexo E, Hamilton-Dutoit S. Real-time quantitative PCR of microdissected paraffin-embedded breast carcinoma: an alternative method for HER-2/neu analysis. J Mol Diagn 2004; 6(1):42-51.
46. Gu L H, Zhang C, Chen L K, Zhen H F, Cheng L, Zhou H G. [DNA genotyping of oral epithelial cells by laser capure microdissection]. Fa Yi Xue Za Zhi 2006; 22(3):196-7, 203.
47. Ma X J, Salunga R, Tuggle J T, Gaudet J, Enright E, McQuary P et al. Gene expression profiles of human breast cancer progression. Proc Natl Acad Sci USA 2003; 100(10):5974-5979.
48. Ma X J, Wang Z, Ryan P D, Isakoff S J, Barmettler A, Fuller A et al. A two-gene expression ratio predicts clinical outcome in breast cancer patients treated with tamoxifen. Cancer Cell 2004; 5(6):607-616.
49. Wang Z C, Lin M, Wei L J, Li C, Miron A, Lodeiro G et al. Loss of heterozygosity and its correlation with expression profiles in subclasses of invasive breast cancers. Cancer Res 2004; 64(1):64-71.
50. Acharya C R, Hsu D S, Anders C K, Anguiano A, Salter K H, Walters K S et al. Gene expression signatures, clinicopathological features, and individualized therapy in breast cancer. JAMA 2008; 299(13):1574-1587.
51. Bertucci F, Finetti P, Rougemont J, Charafe-Jauffret E, Nasser V, Loriod B et al. Gene expression profiling for molecular characterization of inflammatory breast cancer and prediction of response to chemotherapy. Cancer Res 2004; 64(23):8558-8565.
52. Gianni L, Zambetti M, Clark K, Baker J, Cronin M, Wu J et al. Gene expression profiles in paraffin-embedded core biopsy tissue predict response to chemotherapy in women with locally advanced breast cancer. J Clin Oncol 2005; 23(29):7265-7277.
53. Huang E, Cheng S H, Dressman H, Pittman J, Tsou M H, Horng C F et al. Gene expression predictors of breast cancer outcomes. Lancet 2003; 361(9369):1590-1596.
54. Jansen M P, Foekens J A, van Staveren I L, Dirkzwager-Kiel M M, Ritstier K, Look M P et al. Molecular classification of tamoxifen-resistant breast carcinomas by gene expression profiling. J Clin Oncol 2005; 23(4):732-740.
55. Kang Y, Siegel P M, Shu W, Drobnjak M, Kakonen S M, Cordon-Cardo C et al. A multigenic program mediating breast cancer metastasis to bone. Cancer Cell 2003; 3(6):537-549.
56. Korkola J E, Blayeri E, DeVries S, Moore D H, Hwang E S, Chen Y Y et al. Identification of a robust gene signature that predicts breast cancer outcome in independent data sets. BMC Cancer 2007; 7:61.
57. Ma X J, Wang W, Salunga R, Tuggle J T, Stecker K, Baer T M et al. Gene expression associated with clinical outcome in breast cancer via laser capture microdissection. Breast Cancer Res. Treat. 82. 2003.
58. Miller L D, Smeds J, George J, Vega V B, Vergara L, Ploner A et al. An expression signature for p53 status in human breast cancer predicts mutation status, transcriptional effects, and patient survival. Proc Natl Acad Sci USA 2005; 102(38):13550-13555.
59. Paik S, Tang G, Shak S, Kim C, Baker J, Kim W et al. Gene expression and benefit of chemotherapy in women with node-negative, estrogen receptor-positive breast cancer. J Clin Oncol 2006; 24(23):3726-3734.
60. Parker B S, Argani P, Cook B P, Liangfeng H, Chartrand S D, Zhang M et al. Alterations in vascular gene expression in invasive breast carcinoma. Cancer Res 2004; 64(21):7857-7866.
61. Perou C M, Sorlie T, Eisen M B, van de Rijn M, Jeffrey S S, Rees C A et al. Molecular portraits of human breast tumours. Nature 2000; 406(6797):747-752.
62. Ramaswamy S, Ross K N, Lander E S, Golub T R. A molecular signature of metastasis in primary solid tumors. Nat Genet. 2003; 33(1):49-54.
63. Sorlie T, Perou C M, Tibshirani R, Aas T, Geisler S, Johnsen H et al. Gene expression patterns of breast carcinomas distinguish tumor subclasses with clinical implications. Proc Natl Acad Sci USA 2001; 98(19):10869-10874.
64. Sotiriou C, Neo S Y, McShane L M, Korn E L, Long P M, Jazaeri A et al. Breast cancer classification and prognosis based on gene expression profiles from a population-based study. Proc Natl Acad Sci USA 2003; 100(18):10393-10398.
65. Van't Veer L J, Dai H, van de Vijver M J, He Y D, Hart A A, Mao M et al. Gene expression profiling predicts clinical outcome of breast cancer. Nature 2002; 415(6871):530-536.
66. van de Vijver M J, He Y D, van't Veer L J, Dai H, Hart A A, Voskuil D W et al. A gene-expression signature as a predictor of survival in breast cancer. N Engl Med 2002; 347(25):1999-2009.
67. Wang Y, Klijn J G, Zhang Y, Sieuwerts A M, Look M P, Yang F et al. Gene-expression profiles to predict distant metastasis of lymph-node-negative primary breast cancer. Lancet 2005; 365(9460):671-679.
68. Weigelt B, Hu Z, He X, Livasy C, Carey L A, Ewend M G et al. Molecular portraits and 70-gene prognosis signature are preserved throughout the metastatic process of breast cancer. Cancer Res 2005; 65(20):9155-9158.
69. West M, Blanchette C, Dressman H, Huang E, Ishida S, Spang R et al. Predicting the clinical status of human breast cancer by using gene expression profiles. Proc Natl Acad Sci USA 2001; 98(20):11462-11467.
70. Wittliff J L, Ma X J, Wang W, Salunga R, Tuggle J T, Stecker K et al, Expression of estrogen receptor-associated genes in breast cancer cells procured by laser capture microdissection. Jensen Symp. Abs. 81. 2003.
71. Wittliff J L, Kruer T L, Andres S A, Smolenkova I. Molecular signatures of estrogen receptor-associated genes in breast cancer predict clinical outcome. Adv Exp Med Biol 2008; 617:349-357.
72. Woelfle U, Cloos J, Sauter G, Riethdorf L, Janicke F, van D P et al. Molecular signature associated with bone marrow micrometastasis in human breast cancer. Cancer Res 2003; 63(18):5679-5684.
73. Zhao H, Langerod A, Ji Y, Nowels K W, Nesland J M, Tibshirani R et al. Different gene expression patterns in invasive lobular and ductal carcinomas of the breast. Mol Biol Cell 2004; 15(6):2523-2536.
74. Desmedt C. State of the art in the use of gene expression technologies for breast cancer management. Eur Oncol 2008; 4(1):66-70.
75. van't Veer L I, Bernards R. Enabling personalized cancer medicine through analysis of gene-expression patterns. Nature 2008; 452(7187):564-570.
76. Paik S, Shak S, Tang G, Kim C, Baker J, Cronin M et al. A multigene assay to predict recurrence of tamoxifen-treated, node-negative breast cancer. N Engl J Med 2004; 351(27):2817-2826.
77. Marshall E. Getting the noise out of gene arrays. Science 2004; 306(5696):630-631.
78. Michiels S, Koscielny S, Hill C. Prediction of cancer outcome with microarrays: a multiple random validation strategy. Lancet 2005; 365(9458):488-492.
79. Sherlock G. Of fish and chips. Nat Methods 2005; 2(5):329-330.
80. Fan C, Oh D S, Wessels L, Weigelt B, Nuyten D S, Nobel A B et al. Concordance among gene-expression-based predictors for breast cancer. N Engl J Med 2006; 355(6):560-569.
81. Wirapati P, Sotiriou C, Kunkel S, Farmer P, Pradervand S, Haibe-Kains B et al. Meta-analysis of gene expression profiles in breast cancer: toward a unified understanding of breast cancer subtyping and prognosis signatures. Breast Cancer Res 2008; 10(4):R65.
82. Bustin S A. Molecular medicine, gene expression profiling and molecular diagnostics: Putting the cart before the horse. Biomarkers Med. 2[3], 201-207. 2008.
83. Ma X J, Hilsenbeck S G, Wang W, Ding L, Sgroi D C, Bender R A et al. The HOXB13:IL17BR expression index is a prognostic factor in early-stage breast cancer. J Clin Oncol 2006; 24(28):4611-4619.
84. Hembruff S L, Villeneuve D J, Parissenti A M. The optimization of quantitative reverse transcription PCR for verification of cDNA microarray data. Anal Biochem 2005; 345(2):237-249.
85. Tan P K, Downey T J, Spitznagel E L, Jr., Xu P, Fu D, Dimitrov D S et al. Evaluation of gene expression measurements from commercial microarray platforms. Nucleic Acids Res 2003; 31(19):5676-5684.
86. Shi L, Reid L H, Jones W D, Shippy R, Warrington J A, Baker S C et al. The MicroArray Quality Control (MAQC) project shows inter- and intraplatform reproducibility of gene expression measurements. Nat Biotechnol 2006; 24(9):1151-1161.
87. Haybittle J L, Blamey R W, Elston C W, Johnson J, Doyle P J, Campbell F C et al. A prognostic index in primary breast cancer. Br J Cancer 1982; 45(3):361-366.
88. Ravdin P M, Siminoff L A, Davis G J, Mercer M B, Hewlett J, Gerson N et al. Computer program to assist in making decisions about adjuvant therapy for women with early breast cancer. J Clin Oncol 2001; 19(4):980-991.
91. Harris L, Fritsche H, Mennel R, Norton L, Ravdin P, Taube S et al. American Society of Clinical Oncology 2007 update of recommendations for the use of tumor markers in breast cancer. J Clin Oncol 2007; 25(33):5287-5312.
92. Cronin M, Sangli C, Liu M L, Pho M, Dutta D, Nguyen A et al. Analytical validation of the Oncotype DX genomic diagnostic test for recurrence prognosis and therapeutic response prediction in node-negative, estrogen receptor-positive breast cancer. Clin Chem 2007; 53(6):1084-1091.
93. Sparano J A, Paik S. Development of the 21-gene assay and its application in clinical practice and clinical trials. J Clin Oncol 2008; 26(5):721-728.
94. Ross J S, Hatzis C, Symmans W F, Pusztai L, Hortobagyi G N. Commercialized multigene predictors of clinical outcome for breast cancer. Oncologist 2008; 13(5):477-493.
96. Buyse M, Loi S, van't Veer L, Viale G, Delorenzi M, Glas A M et al. Validation and clinical utility of a 70-gene prognostic signature for women with node-negative breast cancer. J Natl Cancer Inst 2006; 98(17):1183-1192.
97. Wittner B S, Sgroi D C, Ryan P D, Bruinsma T J, Glas A M, Male A et al. Analysis of the MammaPrint breast cancer assay in a predominantly postmenopausal cohort. Clin Cancer Res 2008; 14(10):2988-2993.
98. Cardoso F, van't Veer L, Rutgers E, Loi S, Mook S, Piccart-Gebhart M J. Clinical application of the 70-gene profile: the MINDACT trial, J Clin Oncol 2008; 26(5):729-735.
102, Goetz M P, Suman V J, Ingle J N, Nibbe A M, Visscher D W, Reynolds C A et al. A two-gene expression ratio of homeobox 13 and interleukin-17B receptor for prediction of recurrence and survival in women receiving adjuvant tamoxifen. Clin Cancer Res 2006; 12(7 Pt 1):2080-2087.
103. Reid J F, Lusa L. De C L, Coradini D, Veneroni S, Daidone M G et al. Limits of predictive models using microarray data for breast cancer clinical treatment outcome. J Natl Cancer Inst 2005; 97(12):927-930.
104. Jansen M P, Foekens J A, Klijn J G, Berns E M. Re: Limits of predictive models using microarray data for breast cancer clinical treatment outcome. J Natl Cancer Inst 2005; 97(24):1851-1852.
105. Foekens J A, Atkins D, Zhang Y, Sweep F C, Harbeck N, Paradiso A et al. Multicenter validation of a gene expression-based prognostic signature in lymph node-negative primary breast cancer. J Clin Oncol 2006; 24(11):1665-1671.
107. Mullins M, Perreard L, Quackenbush J F, Gauthier N, Bayer S, Ellis M et al. Agreement in breast cancer classification between microarray and quantitative reverse transcription PCR from fresh-frozen and formalin-fixed, paraffin-embedded tissues. Clin Chem 2007; 53(7):1273-1279.
108. Perreard L, Fan C, Quackenbush J F, Mullins M, Gauthier N P, Nelson E et al. Classification and risk stratification of invasive breast carcinomas using a real-time quantitative RT-PCR assay. Breast Cancer Res 2006; 8(2):R23.
109. Sotiriou C, Wirapati P, Loi S, Harris A, Fox S, Smeds J et al. Gene expression profiling in breast cancer: understanding the molecular basis of histologic grade to improve prognosis. J Natl Cancer Inst 2006; 98(4):262-272.
110. Ma X J, Salunga R, Dahiya S, Wang W, Carney E, Durbecq V et al. A five-gene molecular grade index and HOXB13:IL17BR are complementary prognostic factors in early stage breast cancer. Clin Cancer Res 2008; 14(9):2601-2608.
111. Asgharzadeh S, Pique-Regi R, Sposto R, Wang H, Yang Y, Shimada H et al. Prognostic significance of gene expression profiles of metastatic neuroblastomas lacking MYCN gene amplification. J Natl Cancer Inst 2006; 98(17):1193-1203.
112. Asselah T, Bieche I, Laurendeau I, Paradis V, Vidaud D, Degott C et al. Liver gene expression signature of mild fibrosis in patients with chronic hepatitis C. Gastroenterology 2005; 129(6):2064-2075.
113. Charafe-Jauffret E, Ginestier C, Manville F, Finetti P, Adelaide J, Cervera N et al. Gene expression profiling of breast cell lines identifies potential new basal markers. Oncogene 2006; 25(15):2273-2284.
114. Chen Z M, Crone K G, Watson M A, Pfeifer J D, Wang H L. Identification of a unique gene expression signature that differentiates hepatocellular adenoma from well-differentiated hepatocellular carcinoma. Am J Surg Pathol 2005; 29(12):1600-1608.
115. Chung C H, Parker J S, Ely K, Carter J, Yi Y, Murphy B A et al. Gene expression profiles identify epithelial-to-mesenchymal transition and activation of nuclear factor-kappaB signaling as characteristics of a high-risk head and neck squamous cell carcinoma. Cancer Res 2006; 66(16):8210-8218.
116. Dyrskjot L, Kruhoffer M, Thykjaer T, Marcussen N, Jensen J L, Moller K et al. Gene expression in the urinary bladder: a common carcinoma in situ gene expression signature exists disregarding histopathological classification. Cancer Res 2004; 64(11):4040-4048.
117. Ginos M A, Page G P, Michalowicz B S, Patel K J, Volker S E, Pambuccian S E et al. Identification of a gene expression signature associated with recurrent disease in squamous cell carcinoma of the head and neck. Cancer Res 2004; 64(1):55-63.
118. Ippolito J E, Xu J, Jain S, Moulder K, Mennerick S, Crowley J R et al. An integrated functional genomics and metabolomics approach for defining poor prognosis in human neuroendocrine cancers. Proc Natl Acad Sci USA 2005; 102(28): 9901-9906.
119. Lee Y F, John M, Falconer A, Edwards S, Clark J, Flohr P et al. A gene expression signature associated with metastatic outcome in human leiomyosarcomas. Cancer Res 2004; 64(20):7201-7204.
120. Martinez N, Camacho F I, Algara P, Rodriguez A, Dopazo A, Ruiz-Ballesteros E et al. The molecular signature of mantle cell lymphoma reveals multiple signals favoring cell survival. Cancer Res 2003; 63(23):8226-8232.
121. Meireles S I, Cristo E B, Carvalho A F, Hirata R, Jr., Pelosof A, Gomes L I et al. Molecular classifiers for gastric cancer and nonmalignant diseases of the gastric mucosa. Cancer Res 2004; 64(4):1255-1265.
122. Nacht M, Dracheva T, Gao Y, Fujii T, Chen Y, Player A et al. Molecular characteristics of non-small cell lung cancer. Proc Natl Acad Sci USA 2001; 98(26):15203-15208.
123. Onken M D, Worley L A, Ehlers J P, Harbour J W. Gene expression profiling in uveal melanoma reveals two molecular classes and predicts metastatic death. Cancer Res 2004; 64(20):7205-7209.
124. Raponi M, Zhang Y, Yu J, Chen G, Lee G, Taylor J M et al. Gene expression signatures for predicting prognosis of squamous cell and adenocarcinomas of the lung. Cancer Res 2006; 66(15):7466-7472.
125. Rickman D S, Bobek M P, Misek D E, Kuick R, Blaivas M, Kurnit D M et al. Distinctive molecular profiles of high-grade and low-grade gliomas based on oligonucleotide microarray analysis. Cancer Res 2001; 61(18):6885-6891.
126. Risinger J I, Maxwell G L, Chandramouli G V, Aprelikova O, Litzi T, Umar A et al. Gene expression profiling of microsatellite unstable and microsatellite stable endometrial cancers indicates distinct pathways of aberrant signaling. Cancer Res 2005; 65(12):5031-5037.
127. Schwartz D R, Kardia S L, Shedden K A, Kuick R, Michailidis G, Taylor J M et al. Gene expression in ovarian cancer reflects both morphology and biological behavior, distinguishing clear cell from other poor-prognosis ovarian carcinomas. Cancer Res 2002; 62(16):4722-4729.
128. Tagliafico E, Tenedini E, Manfredini R, Grande A, Ferrari F, Roncaglia E et al. Identification of a molecular signature predictive of sensitivity to differentiation induction in acute myeloid leukemia. Leukemia 2006; 20(10):1751-1758.
129. Velazquez-Fernandez D, Laurell C, Geli J, Hoog A, Odeberg J, Kjellman M et al. Expression profiling of adrenocortical neoplasms suggests a molecular signature of malignancy. Surgery 2005; 138(6):1087-1094.
130. Wells S I, Aronow B J, Wise T M, Williams S S, Couget J A, Howley P M. Transcriptome signature of irreversible senescence in human papillomavirus-positive cervical cancer cells. Proc Natl Acad Sci USA 2003; 100(12):7093-7098.
131. Chalabi N, Delort L, Le C L, Satih S, Bignon Y J, Bernard-Gallon D. Gene signature of breast cancer cell lines treated with lycopene. Pharmacogenomics 2006; 7(5):663-672.
132. Giacomini C P, Leung S Y, Chen X, Yuen S T, Kim Y H, Bair E et al. A gene expression signature of genetic instability in colon cancer. Cancer Res 2005; 65(20):9200-9205.
133. Nanni S, Priolo C, Grasselli A, D'Eletto M, Merola R, Moretti F et al. Epithelial-restricted gene profile of primary cultures from human prostate tumors: a molecular approach to predict clinical behavior of prostate cancer. Mol Cancer Res 2006; 4(2):79-92.
134. Ross D T, Scherf U, Eisen M B, Perou C M, Rees C, Spellman P et al. Systematic variation in gene expression patterns in human cancer cell lines. Nat Genet. 2000; 24(3):227-235.
135. Fleisher M, Dnistrian A M, Sturgeon C M, Wittliff J L. Practice guidelines and recommendations for use of tumor markers in the clinic. In: Diamandis D P, Fritsche H A, Lilja H, Chan D W, Schwartz M K, editors. Tumor markers: physiology, pathobiology, technology, and clinical applications. Washington D.C.: AACC Press; 2002. p. 33-63.
136. Simone N L, Remaley A T, Charboneau L, Petricoin E F, III, Glickman J W, Emmert-Buck M R et al. Sensitive immunoassay of tissue cell proteins procured by laser capture microdissection. Am J Pathol 2000; 156(2):445-452.
137. Simone N L, Paweletz C P, Charboneau L, Petricoin E F, III, Liotta L A. Laser capture microdissection: beyond functional genomics to proteomics. Mol Diagn 2000; 5(4):301-307.
138. Kerr II D A, Eliason J F, Wittliff J L. Steroid receptor and growth factor receptor expression in human nonsmall cell lung cancers using cells procured by laser-capture microdissection. Adv Exp Med Biol 2008; 617:377-384.
139. Mikulowska-Mennis A, Taylor T B, Vishnu P, Michie S A, Raja R, Horner N et al. High-quality RNA from cells isolated by laser capture microdissection. Biotechniques 2002; 33(1):176-179.
140. Schroeder A, Mueller O, Stocker S, Salowsky R, Leiber M, Gassmann M et al. The RIN: an RNA integrity number for assigning integrity values to RNA measurements. BMC Mol Biol 2006; 7:3.
141. Pfaffl M W. A new mathematical model for relative quantification in real-time RT-PCR. Nucleic Acids Res 2001; 29(9):e45.
142. Suzuki T, Higgins P J, Crawford D R. Control selection for RNA quantitation. Biotechniques 2000; 29(2):332-337.
143. Vandesompele J, De P K, Pattyn F, Poppe B, Van R N, De P A et al. Accurate normalization of real-time quantitative RT-PCR data by geometric averaging of multiple internal control genes. Genome Biol 2002; 3(7): RESEARCH0034.
144. Bookout A L, Cummins C L, Mangelsdorf D J, Pesola J M, Kramer M F. High-throughput real-time quantitative reverse transcription PCR. Curr Protoc Mol Biol 2006; Chapter 15: Unit.
146. Ronnov-Jessen L, Petersen O W, Bissell M J. Cellular changes involved in conversion of normal to malignant breast: importance of the stromal reaction. Physiol Rev 1996; 76(1):69-125.
147. Taylor-Papadimitriou J, Stampfer M, Bartek J, Lewis A, Boshell M, Lane E B et al. Keratin expression in human mammary epithelial cells cultured from normal and malignant tissue: relation to in vivo phenotypes and influence of medium. J Cell Sci 1989; 94 (Pt 3):403-413.
148. Bergmann S, Royer-Pokora B, Fietze E, Jurchott K, Hildebrandt B, Trost D et al. YB-1 provokes breast cancer through the induction of chromosomal instability that emerges from mitotic failure and centrosome amplification. Cancer Res 2005; 65(10):4078-4087.
149. Garcia-Tunon I, Ricote M, Ruiz A, Fraile B, Paniagua R, Royuela M. IL-6, its receptors and its relationship with bcl-2 and bax proteins in infiltrating and in situ human breast carcinoma. Histopathology 2005; 47(1):82-89.
150. Hein D W. Molecular genetics and function of NAT1 and NAT2: role in aromatic amine metabolism and carcinogenesis. Mutat Res 2002; 506-507:65-77.
151. Lin M L, Park J H, Nishidate T, Nakamura Y, Katagiri T. Involvement of maternal embryonic leucine zipper kinase (MELK) in mammary carcinogenesis through interaction with Bcl-G, a pro-apoptotic member of the Bcl-2 family. Breast Cancer Res 2007; 9(1):R17.
152. Taylor K M, Morgan H E, Smart K, Zahari N M, Pumford S, Ellis 10 et al. The emerging role of the LIV-1 subfamily of zinc transporters in breast cancer. Mol Med 2007; 13(7-8):396-406.
153. Matsumoto K, Yokote H, Arao T, Maegawa M, Tanaka K, Fujita Y et al. N-Glycan fucosylation of epidermal growth factor receptor modulates receptor activity and sensitivity to epidermal growth factor receptor tyrosine kinase inhibitor. Cancer Sci 2008; 99(8):1611-1617.
154. Wang X, Gu J, Miyoshi E, Honke K, Taniguchi N. Phenotype changes of Fut8 knockout mouse: core fucosylation is crucial for the function of growth factor receptor(s). Methods Enzymol 2006; 417:11-22.
155. Ito Y, Miyauchi A, Yoshida H, Uruno T, Nakano K, Takamura Y et al. Expression of alpha-1,6-fucosyltransferase (FUT8) in papillary carcinoma of the thyroid: its linkage to biological aggressiveness and anaplastic transformation. Cancer Lett 2003; 200(2):167-172.
156. Janssens K, De K L, Balsamo M, Vandoninck S, Vandenheede J R, Gertler F et al. Characterization of EVL-I as a protein kinase D substrate. Cell Signal 2009; 21(2):282-292.
157. Lambrechts A, Kwiatkowski A V, Lanier L M, Bear J E, Vandekerckhove J, Ampe C et al. cAMP-dependent protein kinase phosphorylation of EVL, a Mena/VASP relative, regulates its interaction with actin and SH3 domains. J Biol Chem 2000; 275(46):36143-36151.
158. Hu L D, Zou H F, Zhan S X, Cao K M. EVL (Ena/VASP-like) expression is up-regulated in human breast cancer and its relative expression level is correlated with clinical stages. Oncol Rep 2008; 19(4):1015-1020.
159. Grady W M, Parkin R K, Mitchell P S, Lee J H, Kim Y H, Tsuchiya K D et al. Epigenetic silencing of the intronic microRNA hsa-miR-342 and its host gene EVL in colorectal cancer. Oncogene 2008; 27(27):3880-3888.
160. Ragunathan N, Dairou J, Pluvinage B, Martins M, Petit E, Janel N et al. Identification of the xenobiotic-metabolizing enzyme arylamine N-acetyltransferase 1 as a new target of cisplatin in breast cancer cells: molecular and cellular mechanisms of inhibition. Mol Pharmacol 2008; 73(6):1761-1768.
161. Kim S J, Kang H S, Chang H L, Jung Y C, Sim H B, Lee K S et al. Promoter hypomethylation of the N-acetyltransferase 1 gene in breast cancer. Oncol Rep 2008; 19(3):663-668.
162. Saxena A, Saffery R, Wong L H, Kalitsis P, Choo K H. Centromere proteins Cenpa, Cenpb, and Bub3 interact with poly(ADP-ribose) polymerase-1 protein and are poly(ADP-ribosyl)ated. J Biol Chem 2002; 277(30):26921-26926.
163. Lacroix M, Haibe-Kains B, Hennuy B, Laes J F, Lallemand F, Gonze I et al. Gene regulation by phorbol 12-myristate 13-acetate in MCF-7 and MDA-MB-231, two breast cancer cell lines exhibiting highly different phenotypes. Oncol Rep 2004; 12(4):701-707.
164. Biermann K, Heukamp L C, Steger K, Zhou H, Franke F E, Guetgemann I et al. Gene expression profiling identifies new biological markers of neoplastic germ cells. Anticancer Res 2007; 27(5A):3091-3100.
165. Nakano I, Masterman-Smith M, Saigusa K, Paucar A A, Horvath S, Shoemaker L et al. Maternal embryonic leucine zipper kinase is a key regulator of the proliferation of malignant brain tumors, including brain tumor stem cells. J Neurosci Res 2008; 86(1):48-60.
166. Payne S J, Bowen R L, Jones J L, Wells C A. Predictive markers in breast cancer—the present. Histopathology 2008; 52(1):82-90.
167. Hannemann A, Jandrig B, Gaunitz F, Eschrich K, Bigl M. Characterization of the human P-type 6-phosphofructo-1-kinase gene promoter in neural cell lines. Gene 2005; 345(2):237-247.
168. Spitz G A, Furtado C M, Sola-Penna M, Zancan P. Acetylsalicylic acid and salicylic acid decrease tumor cell viability and glucose metabolism modulating 6-phosphofructo-1-kinase structure and activity. Biochem Pharmacol 2009; 77(1):46-53.
169. Symmans W F, Fiterman D J, Anderson S K, Ayers M, Rouzier R, Dunmire V et al. A single-gene biomarker identifies breast cancers associated with immature cell type and short duration of prior breastfeeding. Endocr Relat Cancer 2005; 12(4):1059-1069.
170. Zafrakas M, Chorovicer M, Klaman I, Kristiansen G, Wild P J, Heindrichs U et al. Systematic characterisation of GABRP expression in sporadic breast cancer and normal breast tissue. Int J Cancer 2006; 118(6):1453-1459.
171. Takai N, Hamanaka R, Yoshimatsu J, Miyakawa I. Polo-like kinases (Plks) and cancer. Oncogene 2005; 24(2):287-291.
172. Rizki A, Mott J D, Bissell M J. Polo-like kinase 1 is involved in invasion through extracellular matrix. Cancer Res 2007; 67(23):11106-11110.
173. Spankuch B, Kurunci-Csacsko E, Kaufmann M, Strebhardt K. Rational combinations of siRNAs targeting Plk1 with breast cancer drugs. Oncogene 2007; 26(39):5793-5807.
174. Cheeseman I M, Desai A. Cell division: AAAtacking the mitotic spindle. Curr Biol 2004; 14(2):R70*R72.
175. Fellenberg J, Bernd L, Delling G, Witte D, Zahlten-Hinguranage A. Prognostic significance of drug-regulated genes in high-grade osteosarcoma. Mod Pathol 2007; 20(10):1085-1094.
176. Husain S, Yildirim-Toruner C, Rubio J P, Field J, Schwalb M, Cook S et al. Variants of ST8SIA1 are associated with risk of developing multiple sclerosis. PLoS ONE 2008; 3(7):e2653.
177. Ruckhaberle E, Rody A, Engels K, Gaetje R, von M G, Schiffmann S et al. Microarray analysis of altered sphingolipid metabolism reveals prognostic significance of sphingosine kinase 1 in breast cancer. Breast Cancer Res Treat 2008; 112(1):41-52.
178. Ruckhaberle E, Karn T, Rody A, Hanker L, Gatje R, Metzler D et al. Gene expression of ceramide kinase, galactosyl ceramide synthase and ganglioside GD3 synthase is associated with prognosis in breast cancer. J Cancer Res Clin Oncol 2009.
179. Gomez B P, Riggins R B, Shajahan A N, Klimach U, Wang A, Crawford A C et al. Human X-box binding protein-1 confers both estrogen independence and antiestrogen resistance in breast cancer cell lines. FASEB J 2007; 21(14):4013-4027.
180. Lacroix M, Leclercq G. About GATA3, HNF3A, and XBP1, three genes co-expressed with the estrogen receptor-alpha gene (ESR1) in breast cancer. Mol Cell Endocrinol 2004; 219(1-2):1-7.
181. Costa A, Onesti S. The MCM complex: (just) a replicative helicase? Biochem Soc Trans 2008; 36(Pt 1):136-140.
182. Dehan E, Ben-Dor A, Liao W, Lipson D, Frimer H, Rienstein S et al. Chromosomal aberrations and gene expression profiles in non-small cell lung cancer. Lung Cancer 2007; 56(2):175-184.
183. Ru H Y, Chen R L, Lu W C, Chen J H. hBUB1 defects in leukemia and lymphoma cells. Oncogene 2002; 21(30):4673-4679.
184. Vanoosthuyse V, Hardwick K G. Bubl and the multilayered inhibition of Cdc20-APC/C in mitosis. Trends Cell Biol 2005; 15(5):231-233.
185. Myrie K A, Percy M J, Azim J N, Neeley C K, Petty E M. Mutation and expression analysis of human BUB1 and BUB1B in aneuploid breast cancer cell lines. Cancer Lett 2000; 152(2):193-199.
186. Bessette D C, Qiu D, Pallen C J. PRL PTPs: mediators and markers of cancer progression. Cancer Metastasis Rev 2008; 27(2):231-252.
187. Stephens B J, Han H, Gokhale V, Von Hoff D D. PRL phosphatases as potential molecular targets in cancer. Mol Cancer Ther 2005; 4(11):1653-1661.
188. Radke I, Gotte M, Kersting C, Mattsson B, Kiesel L, Wulfing P. Expression and prognostic impact of the protein tyrosine phosphatases PRL-1, PRL-2, and PRL-3 in breast cancer. Br J Cancer 2006; 95(3):347-354.
189. Fiordalisi J J, Keller P J, Cox A D. PRL tyrosine phosphatases regulate rho family GTPases to promote invasion and motility. Cancer Res 2006; 66(6):3153-3161.
190, Habibi G, Leung S, Law J H, Gelmon K, Masoudi H, Turbin D et al. Redefining prognostic factors for breast cancer: YB-1 is a stronger predictor of relapse and disease-specific survival than estrogen receptor or HER-2 across all tumor subtypes. Breast Cancer Res 2008; 10(5):R86.
191. Hodzic D, Kong C, Wainszelbaum M J, Charron A J, Su X, Stahl P D. TBC1D3, a hominoid oncoprotein, is encoded by a cluster of paralogues located on chromosome 17q12. Genomics 2006; 88(6):731-736.
192. Cheng K W, Lahad J P, Gray J W, Mills G B. Emerging role of RAB GTPases in cancer and human disease. Cancer Res 2005; 65(7):2516-2519.
193. Wang J W, Gamsby J J, Highfill S L, Mora L B, Bloom G C, Yeatman T J et al. Deregulated expression of LRBA facilitates cancer cell growth. Oncogene 2004; 23(23):4089-4097.
194, Reymond A, Meroni G, Fantozzi A, Merla G, Cairo S, Luzi L et al. The tripartite motif family identifies cell compartments. EMBO J. 2001; 20(9):2140-2151.
195. Kosaka Y, Inoue H, Ohmachi T, Yokoe T, Matsumoto T, Mimori K et al. Tripartite motif-containing 29 (TRIM29) is a novel marker for lymph node metastasis in gastric cancer. Ann Surg Oncol 2007; 14(9):2543-2549.
196. Hollway G E, Maule J, Gautier P, Evans T M, Keenan D G, Lohs C et al. Scube2 mediates Hedgehog signalling in the zebrafish embryo. Dev Biol 2006; 294(1):104-118.
197. Kawakami A, Nojima Y, Toyoda A, Takahoko M, Satoh M, Tanaka H et al. The zebrafish-secreted matrix protein you/scube2 is implicated in long-range regulation of hedgehog signaling. Curr Biol 2005; 15(5):480-488.
198. Woods I G, Talbot W S. The you gene encodes an EGF-CUB protein essential for Hedgehog signaling in zebrafish. PLoS Biol 2005; 3(3):e66.
199. Evangelista M, Tian H, de Sauvage F J. The hedgehog signaling pathway in cancer. Clin Cancer Res 2006; 12(20 Pt 1):5924-5928.
200. Kubo M, Nakamura M, Tasaki A, Yamanaka N, Nakashima H, Nomura M et al. Hedgehog signaling pathway is a new therapeutic target for patients with breast cancer. Cancer Res 2004; 64(17):6071-6074.
201. Asselin-Labat M L, Sutherland K D, Barker H, Thomas R, Shackleton M, Forrest N C et al. Gata-3 is an essential regulator of mammary-gland morphogenesis and luminal-cell differentiation. Nat Cell Biol 2007; 9(2):201-209.
202. Kouros-Mehr H, Kim J W, Bechis S K, Werb Z. GATA-3 and the regulation of the mammary luminal cell fate. Curr Opin Cell Biol 2008; 20(2):164-170.
203. Wilson B J, Giguere V. Meta-analysis of human cancer microarrays reveals GATA3 is integral to the estrogen receptor alpha pathway. Mol Cancer 2008; 7:49,
204. Zeng Y, Jiang J, Huebener N, Wenkel J, Gaedicke G, Xiang R et al. Fractalkine gene therapy for neuroblastoma is more effective in combination with targeted IL-2. Cancer Lett 2005; 228(1-2):187-193.
205. Raffaghello L, Cocco C, Corrias M V, Airoldi I, Pistoia V. Chemokines in neuroectodermal tumour progression and metastasis. Semin Cancer Biol 2009; 19(2):97-102.
206. Andreasson U, Ek S, Merz H, Rosenquist R, Andersen N, Jerkeman M et al. B cell lymphomas express CX3CR1 a non-B cell lineage adhesion molecule. Cancer Lett 2008; 259(2):138-145.
207. Blum D L, Koyama T, M'koma A E, Iturregui J M, Martinez-Ferrer M, Uwamariya C et al. Chemokine markers predict biochemical recurrence of prostate cancer following prostatectomy. Clin Cancer Res 2008; 14(23):7790-7797.
208. Selander K S, Li L, Watson L, Merrell M, Dahmen H, Heinrich P C et al. Inhibition of gp130 signaling in breast cancer blocks constitutive activation of Stat3 and inhibits in vivo malignancy. Cancer Res 2004; 64(19):6924-6933.
209. Schafer Z T, Brugge J S. IL-6 involvement in epithelial cancers. J Clin Invest 2007; 117(12):3660-3663.
210. Su L K, Qi Y. Characterization of human MAPRE genes and their proteins. Genomics 2001; 71(2):142-149.
211. Bhat J Y, Shastri B G, Balaram H. Kinetic and biochemical characterization of Plasmodium falciparum GMP synthetase, Biochem J 2008; 409(1):263-273.
212. Nakamura J, Lou L. Biochemical characterization of human GMP synthetase. J Biol Chem 1995; 270(13):7347-7353.
213. Weber G. Enzymes of purine metabolism in cancer. Clin Biochem 1983; 16(1):57-63.
214. Grosshans B L, Novick P. Identification and verification of Sro7p as an effector of the Sec4p Rab GTPase. Methods Enzymol 2008; 438:95-108.
215. Taylor K M, Morgan H E, Johnson A, Hadley L J, Nicholson R1. Structure-function analysis of LIV-1, the breast cancer-associated protein that belongs to a new subfamily of zinc transporters. Biochem J 2003; 375(Pt 1):51-59.
216. Kasper G, Weiser A A, Rump A, Sparbier K, Dahl E, Hartmann A et al. Expression levels of the putative zinc transporter LIV-1 are associated with a better outcome of breast cancer patients. Int J Cancer 2005; 117(6):961-973.
217. Griffiths R W, Gilham D E, Dangoor A, Ramani V, Clarke N R, Stern P L et al. Expression of the 5T4 oncofoetal antigen in renal cell carcinoma: a potential target for T-cell-based immunotherapy. Br J Cancer 2005; 93(6):670-677.
218. Mulder W M, Stern P L, Stukart M J, de W E, Butzelaar R M, Meijer S et al. Low intercellular adhesion molecule 1 and high 5T4 expression on tumor cells correlate with reduced disease-free survival in colorectal carcinoma patients. Clin Cancer Res 1997; 3(11):1923-1930.
219. Pellagatti A, Jadersten M, Forsblom A M, Cattan H, Christensson B, Emanuelsson E K et al. Lenalidomide inhibits the malignant clone and up-regulates the SPARC gene mapping to the commonly deleted region in 5q-syndrome patients. Proc Natl Acad Sci USA 2007; 104(27):11406-11411.
220. Starzynska T, Wiechowska-Kozlowska A, Marlicz K, Bromley M, Roberts S A, Lawniczak M et al. 5T4 oncofetal antigen in gastric carcinoma and its clinical significance. Eur J Gastroenterol Hepatol 1998; 10(6):479-484.
221. Lan Y, Zhang Y, Wang J, Lin C, Ittmann M M, Wang F. Aberrant expression of Cks1 and Cks2 contributes to prostate tumorigenesis by promoting proliferation and inhibiting programmed cell death. Int J Cancer 2008; 123(3):543-551,
222. Pillutla R C, Shimamoto A, Furuichi Y, Shatkin A J. Genomic structure and chromosomal localization of TCEAL1, a human gene encoding the nuclear phosphoprotein p21/SIIR. Genomics 1999; 56(2):217-220.
223. Makino H, Tajifi T, Miyashita M, Sasajima K, Anbazhagan R, Johnston J et al. Differential expression of TCEAL1 in esophageal cancers by custom cDNA microarray analysis. Dis Esophagus 2005; 18(1):37-40.
224. Funakoshi S, Ezaki T, Kong J, Guo R J, Lynch J P. Repression of the desmocollin 2 gene expression in human colon cancer cells is relieved by the homeodomain transcription factors Cdx1 and Cdx2. Mol Cancer Res 2008; 6(9):1478-1490.
225. Khan K, Hardy R, Hag A, Ogunbiyi O, Morton D, Chidgey M. Desmocollin switching in colorectal cancer. Br J Cancer 2006; 95(10):1367-1370.
226. Hediger M A, Romero M F, Peng J B, Rolfs A, Takanaga H, Bruford E A. The ABCs of solute carriers: physiological, pathological and therapeutic implications of human membrane transport proteins Introduction. Pflugers Arch 2004; 447(5):465-468.
227. Stuart R O, Pavlova A, Beier D, Li Z, Krijanovski Y, Nigam S K. EEG1, a putative transporter expressed during epithelial organogenesis: comparison with embryonic transporter expression during nephrogenesis. Am J Physiol Renal Physiol 2001; 281(6):F1148-F1156.
228. Yang R B, Ng C K, Wasserman S M, Colman S D, Shenoy S, Mehraban F et al. Identification of a novel family of cell-surface proteins expressed in human vascular endothelium. J Biol Chem 2002; 277(48):46364-46373.
229. Greten F R, Karin M. The IKK/NF-kappaB activation pathway—a target for prevention and treatment of cancer. Cancer Lett 2004; 206(2):193-199.
230. Karin M, Cao Y, Greten F R, Li Z W. NF-kappaB in cancer: from innocent bystander to major culprit. Nat Rev Cancer 2002; 2(4):301-310.
231. Wittliff J L, Andres S A, inventors. Methods for identifying an increased likelihood of recurrence of breast cancer. KY/USA. 2009.
232. Motulsky H J. Prism 4 Statistics Guide—Statistical analyses for laboratory and clinical researchers. 4 ed. San Diego, Calif.: GraphPad Software Inc.; 2003.
233. Casabianca A, Orlandi C, Fraternale A, Magnani M. Development of a real-time PCR assay using SYBR Green I for provirus load quantification in a murine model of AIDS. J Clin Microbiol 2004; 42(9):4361-4364.
234. Pfaffl M W, Horgan G W, Dempfle L. Relative expression software tool (REST) for group-wise comparison and statistical analysis of relative expression results in real-time PCR. Nucleic Acids Res 2002; 30(9):e36.
235. Zini E, Franchini M, Osto M, Vogtlin A, Guscetti F, Linscheid P et al. Quantitative real-time PCR detection of insulin signalling-related genes in pancreatic islets isolated from healthy cats. Vet J 2008.
236. Katz M H, Hauck W W. Proportional hazards (Cox) regression. J Gen Intern Med 1993; 8(12):702-711.
237. Lee E T, Go O T. Survival analysis in public health research. Annu Rev Public Health 1997; 18:105-134.
238. Ohno-Machado L. Modeling medical prognosis: survival analysis techniques. J Biomed Inform 2001; 34(6):428-439.
239. Cox D R. Regression models and life tables. J R Stat Soc 1972; 34:187-220.
240. Kaplan E L M P. Nonparametric estimation from incomplete observations. J American Statisical Assoc 1958; 53(282):457-481.
241. Ahmed F E, Vos P W, Holbert D. Modeling survival in colon cancer: a methodological review. Mol Cancer 2007; 6:15.
242. Brenton J D, Carey L A, Ahmed A A, Caldas C. Molecular classification and molecular forecasting of breast cancer: ready for clinical application? J Clin Oncol 2005; 23(29):7350-7360.
243. DuPont MPD. [3H] Progestin Receptor Assay Kit Instruction Manual. 1988.
244. DuPont MPD. [125I] Estrogen Receptor Assay Kit Instruction Manual. 1989.
245. Abbott Laboratories DD. Abbott PgR-EIA Monoclonal Assay. 1998.
246. Abott Laboratories DD. Abbott ER-EIA Monoclonal Assay. 1988.
247. Soerjomataram I, Louwman M W, Ribot J G, Roukema J A, Coebergh J W. An overview of prognostic factors for long-term survivors of breast cancer. Breast Cancer Res Treat 2008; 107(3):309-330.
248. Jeruss J S, Mittendorf E A, Tucker S L, Gonzalez-Angulo A M, Buchholz T A, Sahin A A et al. Staging of breast cancer in the neoadjuvant setting. Cancer Res 2008; 68(16):6477-6481.
249. Dupont W D. Statisical modeling for biomedical researchers: a simple introduction to the analysis of complex data. Cambridge, UK: Cambridge University Press; 2002.
250. Kim C, Tang G, Baehner F L, Watson D, Constantino J P, Paik S et al. A comparison of estrogen receptor (ER) measurement by three methods in node negative, estrogen receptor (ER) positive breast cancer: ligand binding (LB), immunohistochemistry (IHC), and quantitative PCR. Breast Cancer Res. Treat. 100[Suppl 1], S162-S163. 2006.
251. Jeong J H, Costantino J P. Application of smoothing methods to evaluate treatment-prognostic factor interactions in breast cancer data. Cancer Invest 2006; 24(3):288-293.
252. Lange C A, Sartorius C A, Abdel-Hafiz H, Spillman M A, Horwitz K B, Jacobsen B M. Progesterone receptor action: translating studies in breast cancer models to clinical insights. Adv Exp Med Biol 2008; 630:94-111.
253. Lange C A. Integration of progesterone receptor action with rapid signaling events in breast cancer models. J Steroid Biochem Mol Biol 2008; 108(3-5):203-212.
254. Herynk M H, Fuqua S A. Estrogen receptor mutations in human disease. Endocr Rev 2004; 25(6):869-898.
255. Zweig M H, Campbell G. Receiver-operating characteristic (ROC) plots: a fundamental evaluation tool in clinical medicine. Clin Chem 1993; 39(4):561-577.
256. Biggerstaff B J. Comparing diagnostic tests: a simple graphic using likelihood ratios. Stat Med 2000; 19(5):649-663.
257. Wittliff J L, Kerr II D A, inventors. Methods of predicting disease-free and overall survival of estrogen receptor positive breast cancer. KY/USA. 2009.
258. Andres S A, Kerr II D A, Englert D F, Wilson D J, Wittliff J L. Expression of small sets of genes in carcinoma and stromal cells predict clinical behavior of human breast cancer. Cancer Res. 69(Suppl), 403s-404s. 2009.

While this invention has been particularly shown and described with references to example embodiments thereof, it will be understood by those skilled in the art that various changes in form and details may be made therein without departing from the scope of the invention encompassed by the appended claims.

What is claimed is:

1. A method of optimizing treatment of a human having breast cancer, comprising the step of measuring a level of expression of genes selected from the group consisting of ESR1, GABRP, RABEP1, SLC39A6, TCEAL1, ATAD2, PTP4A2, LRBA and SLC43A3 in a breast cancer tissue sample from the human, wherein underexpression of GABRP, RABEP1, SLC39A6 and PTP4A2 in the sample in combination with overexpression of ESR1, TCEAL1, ATAD2, LRBA and SLC43A3 in the sample identifies a human that has an increased likelihood of recurrence of the breast cancer that would potentially benefit from a therapy to decrease the likelihood of recurrence of the breast cancer.

2. The method of claim 1, wherein the breast cancer tissue sample is a laser capture microdissection sample.

3. The method of claim 1, wherein the breast cancer tissue sample is an intact tissue section sample.

4. The method of claim 1, wherein expression of the genes are identified by a nucleic acid amplification method.

5. The method of claim 1, wherein the breast cancer tissue sample is obtained from a pre-menopausal human.

6. The method of claim 1, wherein the breast cancer tissue sample is obtained from a post-menopausal human.

7. The method of claim 1, wherein expression is identified by measuring messenger RNA levels of the gene.

8. The method of claim 1, wherein treatment of the human increases the likelihood of survival of the human.

9. The method of claim 1, wherein the human has a progesterone-receptor positive breast cancer.

10. The method of claim 1, wherein the human is lymph node negative for the breast cancer.

11. The method of claim 1, wherein the human is lymph node positive for the breast cancer.

12. A method of optimizing treatment of a human having breast cancer, comprising the step of measuring a level of expression of genes selected from the group consisting of GABRP, TRIM29, RABEP1, SLC39A6, TCEAL1, PLK1 and CX3CL1 in a breast cancer tissue sample from the human, wherein underexpression of GABRP, TRIM29, RABEP1 and SLC39A6 in the sample in combination with overexpression of TCEAL1, PLK1 and CX3CL1 in the sample identifies a human that has an increased likelihood of recurrence of the breast cancer that would potentially benefit from a therapy to decrease the likelihood of recurrence of the breast cancer.

13. The method of claim 12, wherein the human has an estrogen-receptor positive breast cancer.

14. The method of claim 12, wherein the human has a progesterone-receptor positive breast cancer.

15. The method of claim 12, wherein the human is lymph node negative for the breast cancer.

16. The method of claim 12, wherein the human is lymph node positive for the breast cancer.

17. A method of optimizing treatment of a human having breast cancer, comprising the step of measuring a level of expression of genes selected from the group consisting of TBC1D9, RABEP1, SLC39A6, FUT8 and PTP4A2 in a breast cancer tissue sample from the human, wherein underexpression of TBC1D9, RABEP1, SLC39A6, FUT8 and PTP4A2 in the sample identifies a human that has an increased likelihood of recurrence of the breast cancer that would potentially benefit from a therapy to decrease the likelihood of recurrence of the breast cancer.

18. The method of claim 17, wherein underexpression of RABEP1, SLC39A6, FUT8 and PTP4A2 in the sample identifies a human that has an increased likelihood of recurrence of the breast cancer that would potentially benefit from a therapy to decrease the likelihood of recurrence of the breast cancer.

19. The method of claim 18, wherein treatment of the human increases the likelihood of survival of the human.

20. The method of claim 17, wherein the human has an estrogen-receptor positive breast cancer.

21. The method of claim 17, wherein the human has a progesterone-receptor positive breast cancer.

22. A method of optimizing treatment of a human having breast cancer, comprising the step of measuring a level of expression of genes selected from the group consisting of ESR1, GABRP, RABEP1, SLC39A6, TCEAL1, ATAD2, PTP4A2, LRBA and SLC43A3 in a breast cancer tissue sample from the human, wherein overexpression of GABRP, RABEP1, SLC39A6 and PTP4A2 in the sample in combination with underexpression of ESR1, TCEAL1, ATAD2, LRBA and SLC43A3 in the sample identifies a human that has a decreased likelihood of recurrence of the breast cancer that would potentially benefit from a therapy to treat the breast cancer.

23. A method of optimizing treatment of a human having breast cancer, comprising the step of measuring a level of expression of genes selected from the group consisting of GABRP, TRIM29, RABEP1, SLC39A6, TCEAL1, PLK1 and CX3CL1 in a breast cancer tissue sample from the human, wherein overexpression of GABRP, TRIM29, RABEP1 and SLC39A6 in the sample in combination with underexpression of TCEAL1, PLK1 and CX3CL1 in the sample identifies a human that has a decreased likelihood of recurrence of the breast cancer that would potentially benefit from a therapy to treat the breast cancer.

24. A method of optimizing treatment of a human having breast cancer, comprising the step of measuring a level of expression of genes selected from the group consisting of TBC1D9, RABEP1, SLC39A6, FUT8 and PTP4A2 in a breast cancer tissue sample from the human, wherein overexpression of TBC1D9, RABEP1, SLC39A6, FUT8 and PTP4A2 in the sample identifies a human that has a decreased likelihood of recurrence of the breast cancer that would potentially benefit from a therapy to treat the breast cancer.
